(19) United States
(12) Patent Application Publication
(10) Pub. No.: US 2022/0169660 A1
(43) Pub. Date: Jun. 2, 2022

(54) CYCLIC AMINO-PYRAZINECARBOXAMIDE COMPOUNDS AND USES THEREOF

(71) Applicant: SILVERBACK THERAPEUTICS, INC., Seattle, WA (US)

(72) Inventors: Sean Wesley SMITH, Seattle, WA (US); Craig Alan COBURN, Seattle, WA (US)

(21) Appl. No.: 17/435,841

(22) PCT Filed: Mar. 5, 2020
(86) PCT No.: PCT/US2020/021100

§ 371 Date: Sep. 2, 2021

Related U.S. Application Data
(60) Provisional application No. 62/939,383, filed on Nov. 22, 2019.
(60) Provisional application No. 62/814,728, filed on Mar. 6, 2019.

(51) Int. Cl. C07D 498/14; C07D 498/22; C07D 401/14

(52) U.S. Cl. CPC C07D 498/14; C07D 401/14; C07D 498/22

(57) ABSTRACT

Cyclic amino-pyrazinecarboxamide compounds, salts, and pharmaceutical compositions for use in the treatment of disease, such as cancer, are disclosed herein. The disclosed compounds are useful, among other things, in the treating of disease, for example cancer and/or fibrotic diseases, and modulating TGFβR2.

### CROSS-REFERENCE TO RELATED APPLICATIONS

This application claims the benefit of U.S. Provisional Application No. 62/814,728 filed on Mar. 6, 2019, and U.S. Provisional Application No. 62/939,383 filed on Nov. 22, 2019, each of which is incorporated herein by reference in its entirety.

### SEQUENCE LISTING

The instant application contains a Sequence Listing which has been submitted electronically in ASCII format and is hereby incorporated by reference in its entirety. Said ASCII copy, created on Feb. 25, 2020, is named 50358-738_601_SL.txt and is 161,269 bytes in size.

### BACKGROUND OF THE INVENTION

One of the leading causes of death in the United States is cancer. The conventional methods of cancer treatment, like chemotherapy, surgery, or radiation therapy, tend to be either highly toxic or nonspecific to a cancer, or both, resulting in limited efficacy and harmful side effects. Thus, there remains a considerable need for alternative or improved treatments for cancer.

Fibrosis is the formation of excess fibrous connective tissue or scar tissue in an organ or tissue in a reparative or reactive process. Fibrosis can occur in many tissues within the body, typically as a result of inflammation or damage, which include the lungs, liver, heart, and brain. Scar tissue blocks arteries, immobilizes joints and damages internal organs, wreaking havoc on the body's ability to maintain vital functions. Every year, millions of people are hospitalized due to the damaging effects of fibrosis. However, current therapeutics for treating fibrotic diseases are lacking or have drawbacks. Thus, there remains a considerable need for alternative or improved treatments for fibrotic diseases.

### SUMMARY OF THE INVENTION

The present disclosure generally relates to substituted cyclic amino-pyrazinecarboxamide compounds and pharmaceutical compositions. The substituted cyclic amino-pyrazinecarboxamide compounds may be used to treat or prevent disease, including, for example, cancer and/or fibrotic diseases. The disclosed cyclic amino-pyrazinecarboxamide compounds may inhibit TGFβR1 and/or TGFβR2, signaling by TGFβ1, or combinations thereof. The disclosed cyclic amino-pyrazinecarboxamide compounds may be incorporated into conjugates, such as antibody conjugates.

In one aspect, the disclosure provides a compound represented by Formula (I):

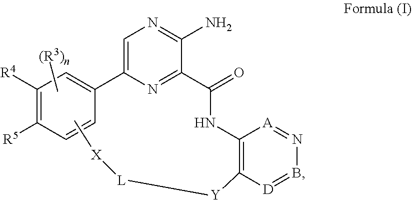

or a pharmaceutically acceptable salt thereof, wherein:
A, B, and D are each independently selected from N and C(R′);
   each R¹ is independently selected from hydrogen, halogen, cyano, —OH, —OR⁵⁰, —NR⁵¹R⁵¹, unsubstituted or substituted —C₁-C₆alkyl, unsubstituted or substituted cycloalkyl, and unsubstituted or substituted heterocycloalkyl;
each R³ is independently selected from R²⁰, R^{L}, and —O—R^{L};
n is 0, 1, or 2;
R⁴ is selected from hydrogen, R²⁰, R^{L}, and —O—R^{L};
R⁵ is selected from hydrogen, R²⁰, R^{L}, and —O—R^{L};
X is selected from —O—, —S—, —NR⁷—, —C(R⁸)₂—, —C(R⁸)₂—O—, —C(R⁸)₂—S—, —C(R⁸)₂—NR⁷—, —S(═O)₂—, —C(═O)—, —NR⁷—S(═O)₂—, and —NR⁷—C(═O)—;
   R⁷ is selected from hydrogen, unsubstituted or substituted —C₁-C₆alkyl, and R^{L};
   each R⁸ is independently selected from hydrogen, halogen, unsubstituted or substituted —C₁-C₆alkyl, and R^{L};
Y is selected from —O—, —S—, —NR⁹—, —C(R¹⁰)₂—, —S(═O)₂—, —C(═O)—, —S(═O)₂—NR⁹—, —C(═O)—NR⁹—, substituted or unsubstituted cycloalkylene, and substituted or unsubstituted heterocycloalkylene;
   R⁹ is selected from hydrogen and unsubstituted or substituted —C₁-C₆alkyl;
   each R¹⁰ is independently selected from hydrogen, halogen, and unsubstituted or substituted —C₁-C₆alkyl;
L is selected from a bond, substituted or unsubstituted C₁-C₁₀ alkylene, —[C(R¹¹)₂]_{q}—(W)—, substituted or unsubstituted C₂-C₁₀ alkenylene, substituted or unsubstituted C₂-C₁₀ alkynylene, and [(substituted or unsubstituted C₁-C₄ alkylene)-Z]ₚ-(substituted or unsubstituted C₁-C₄ alkylene);
   W is unsubstituted or substituted cycloalkylene or unsubstituted or substituted heterocycloalkylene;
   each Z is independently selected from —O—, —S—, and —NR¹¹—;
   each R¹¹ is independently selected from hydrogen and unsubstituted or substituted —C₁-C₆alkyl;
   p is 1-5;
   q is 0-10;
   wherein if L is a bond, then Y is selected from substituted or unsubstituted cycloalkylene and substituted or unsubstituted heterocycloalkylene;
R^{L} is selected from -(unsubstituted or substituted C₁-C₆ alkylene)-OR¹², or -(unsubstituted or substituted C₁-C₆ alkylene)-N(R¹³)₂,
   R¹² is selected from hydrogen, unsubstituted or substituted —C₁-C₆alkyl, unsubstituted or substituted —C₂-C₆ alkenyl, unsubstituted or substituted —C₂-C₆ alkynyl, unsubstituted or substituted cycloalkyl, and unsubstituted or substituted heterocycloalkyl;
   each R¹³ is independently selected from hydrogen, —C(═O)R⁵⁰, —C(═O)OR⁵¹, —C(═O)N⁵¹R⁵¹, unsubstituted or substituted —C₁-C₆alkyl, unsubstituted or substituted —C₂-C₆ alkenyl, unsubstituted or substituted —C₂-C₆ alkynyl, unsubstituted or substituted cycloalkyl, and unsubstituted or substituted heterocycloalkyl;
   or two R¹³ on the same N atom are taken together with the N atom to which they are attached to form an unsubstituted or substituted N-containing heterocycle;
   each R²⁰ is independently selected from halogen, —CN, —OH, —OR⁵⁰, —NR⁵¹R⁵¹, —C(═O)R⁵⁰, —OC(═O)R⁵⁰, —C(═O)OR⁵¹, —OC(═O)OR⁵¹, —C(═O)NR⁵¹R⁵¹, —OC(═O)NR⁵¹R⁵¹, —NR⁵¹C(═O)NR⁵¹R⁵¹, —NR⁵¹C(═O)R⁵⁰, —NR⁵¹C(═O)OR⁵¹, —SR⁵¹, —S(═O)R⁵⁰, —SO₂R⁵⁰, —SO₂NR⁵¹R⁵¹, —NHSO₂R⁵⁰, unsubstituted or substituted —C₁-C₆ alkyl, unsubstituted or substituted —C₂-C₆ alkenyl, unsubstituted or substituted —C₂-C₆ alkynyl, unsubstituted or substituted cycloalkyl, and unsubstituted or substituted heterocycloalkyl;
   each R⁵⁰ is independently selected from unsubstituted or substituted —C₁-C₆ alkyl, unsubstituted or substituted cycloalkyl, unsubstituted or substituted heterocycloalkyl, unsubstituted or substituted aryl, unsubstituted or substituted heteroaryl, -(unsubstituted or substituted C₁-C₆alkylene)-cycloalkyl, -(unsubstituted or substituted C₁-C₆alkylene)-heterocycloalkyl, -(unsubstituted or substituted C₁-C₆alkylene)-aryl, and -(unsubstituted or substituted C₁-C₆alkylene)-heteroaryl; and
   each R⁵¹ is independently selected from hydrogen, unsubstituted or substituted —C₁-C₆ alkyl, unsubstituted or substituted cycloalkyl, unsubstituted or substituted heterocycloalkyl, unsubstituted or substituted aryl, unsubstituted or substituted heteroaryl, -(unsubstituted or substituted C₁-C₆alkylene)-cycloalkyl, -(unsubstituted or substituted C₁-C₆alkylene)-heterocycloalkyl, -(unsubstituted or substituted C₁-C₆alkylene)-aryl, and -(unsubstituted or substituted C₁-C₆alkylene)-heteroaryl;
   or two R⁵¹ on the same N atom are taken together with the N atom to which they are attached to form an unsubstituted or substituted N-containing heterocycle;
wherein when any of L, W, Y, R^{L}, R¹, R⁷, R⁸, R⁹, R¹⁰, R¹¹, R¹², R¹³, R²⁰, R⁵⁰, and R⁵¹ are substituted, substituents on the L, W, Y, R^{L}, R¹, R⁷, R⁸, R⁹, R¹⁰, R¹¹, R¹², R¹³, R²⁰, R⁵⁰, and R⁵¹ are independently selected at each occurrence from halogen, —CN, —NO₂, —OR⁵², —CO₂R⁵², —C(═O)R⁵³, —C(═O)NR⁵²R⁵², —NR⁵²R⁵², —NR⁵²C(═O)R⁵³, —NR⁵²C(═O)OR⁵², —SR⁵², —S(═O)R⁵³, —SO₂R⁵³, —SO₂NR⁵²R⁵², unsubstituted C₁-C₆ alkyl, unsubstituted C₁-C₆ haloalkyl, unsubstituted phenyl, unsubstituted 5- or 6-membered heteroaryl, unsubstituted monocyclic cycloalkyl, and unsubstituted monocyclic heterocycloalkyl; or two substituents on the same carbon atom are taken together to form a ═O or ═S;
   each R⁵² is independently selected from hydrogen, unsubstituted C₁-C₆ alkyl, unsubstituted C₃-C₆ cycloalkyl, unsubstituted 3- to 6-membered heterocycloalkyl, unsubstituted phenyl, unsubstituted benzyl, unsubstituted 5-membered heteroaryl, and unsubstituted 6-membered heteroaryl;
   or two R⁵² groups are taken together with the N atom to which they are attached to form an unsubstituted N-containing heterocycle; and
   each R⁵³ is independently selected from unsubstituted C₁-C₆alkyl, unsubstituted C₃-C₆cycloalkyl, unsubstituted phenyl, unsubstituted benzyl, unsubstituted 5-membered heteroaryl, and unsubstituted 6-membered heteroaryl.

In some embodiments of a compound or salt described herein, the compound of Formula (I) is represented by Formula (II):

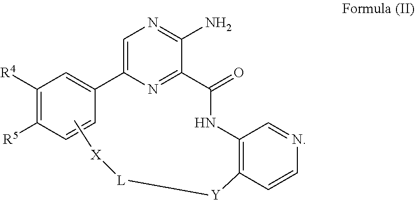

In some embodiments of a compound or salt described herein, the compound of Formula (II) is represented by Formula (III):

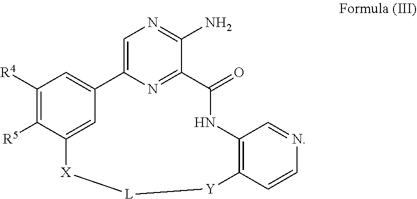

In some embodiments of a compound or salt described herein, the compound of Formula (II) is represented by Formula (IV):

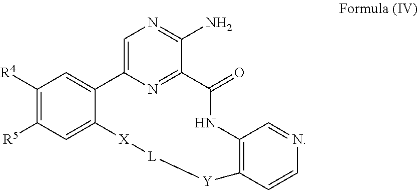

Also disclosed herein are pharmaceutical compositions of the compounds disclosed herein.

In some embodiments, a compound disclosed herein is attached to a linker to form compound-linker moiety.

In some embodiments, a compound disclosed herein is covalently bound to an antibody construct or a targeting moiety, optionally via a linker.

Also disclosed herein are pharmaceutical compositions of the compounds or conjugates described herein.

In some aspects, the present disclosure provides a method for treating cancer, comprising administering a compound, a conjugate, or a pharmaceutical composition as described herein to a subject in need thereof.

In some aspects, the present disclosure provides a method for treating fibrosis, comprising administering a compound, a conjugate, or a pharmaceutical composition as described herein to a subject in need thereof. In some aspects, the fibrosis is cancer-associated. In some aspects, the fibrosis is not cancer-associated. In some aspects, the fibrosis is idiopathic pulmonary fibrosis (IPF) or scleroderma. In other aspects, the fibrosis is systemic fibrosis. In one aspect, the fibrotic disease is steatohepatitis, e.g., non-alcoholic steatohepatitis (NASH).

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION OF THE INVENTION

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

Transforming growth factors (TGFs) and their receptors (TGFRs) are evolutionarily conserved molecules that play important, pleiotropic roles in the regulation of numerous development and physiological pathways, such as cell proliferation, cell differentiation, embryonic development, extracellular matrix formation, wound healing, bone development, immune responses, and inflammatory responses. Given the breadth of their biological functions, TGFs and TGFRs are also involved in many pathological processes, such as those underlying the development and progression of cancer, immune and inflammatory diseases, fibrosis, scarring, atherosclerosis, viral infections, and others.

Transforming growth factor beta-1 (TGFβ1) is the prototypical member of the TGF superfamily of ligands and shares receptor binding and largely overlapping biological activities with two other family members, TGFβ2 and TGFβ. The TGFβ ligands are growth factors and cytokines involved in signaling within a broad array of tissue types. Overexpression of TGFβ1, TGFβ2 and TGFβ3 have been shown to induce fibrotic disease pathology in a number of organ systems, including the kidney, liver, heart, lung, bone marrow, and skin.

TGFβ1 plays numerous roles in tumor progression. TGFβ1 can induce epithelial to mesenchymal transition, enhance the ability of tumor cells to grow, influence tumor cell fate, and modulate the composition of the tumor microenvironment so that it is more permissive to tumor growth.

TGFβ1 plays a role in the maintenance of peripheral tolerance in T-cells and in the prevention of maturation of dendritic cells. Further, TGFβ1 has been shown to regulate the antigen-presentation functions of dendritic cells by down-regulating expression of Major Histocompatibility Complex class II (MHC-II) and the secretion of Interleukin-12 (IL-12).

TGFβ1 signaling by its receptors in myeloid cells has been shown to play roles in tumor promotion and tumor immune suppression including in dendritic cells, myeloid-derived suppressor cells, tumor associated macrophages or combinations of these cells.

Transforming growth factor beta receptor 2 (TGFβR2) is one of two transmembrane serine/threonine kinase receptors that are required for TGFβ1, TGFβ2 and TGFβ3 signal transduction, with the other receptor being TGFβR1. For example, TGFβ1 first binds to TGFβR2 at the plasma membrane, inducing the formation of the TGFβR1-TGFβR2 complex, which leads to phosphorylation of Mothers Against Decapentaplegic homolog 2 (Smad2) and Mothers Against Decapentaplegic homolog 3 (Smad3), and subsequent modulation of a number of downstream signaling targets.

Given the wide range of pathological cellular and multicellular interactions in which the TGFβ ligands play a prominent role, pharmacological inhibition of the TGFβs or its receptors, TGFβR1 or TGFβR2, may prove to be useful in the treatment of several diseases.

Challenges to developing targeted therapies include achieving high selectivity for the primary pharmacological target and maintaining prolonged target inhibition. In overcoming these two challenges, it is possible to develop pharmaceutical products with increased therapeutic efficacy and reduced systemic toxicity. One approach to addressing these two challenges is developing covalent drugs, whereby a covalent interaction takes place between the pharmacological entity and a specific cysteine in the active site of the protein target.

There is a current need for therapeutics that can inhibit signaling by TGFβ1, or inhibit TGFβR1 and/or TGFβR2 function, or combinations thereof, to treat or prevent diseases, including, for example, cancer and fibrosis. The present disclosure provides compounds, conjugates, compositions and methods that address this need and related needs.

The present disclosure provides compounds, conjugates, and pharmaceutical compositions for use in the treatment or prevention of disease associated with TGFβ signaling pathway. In certain embodiments, the cyclic amino-pyrazinecarboxamide compounds of this disclosure, including substituted cyclic amino-pyrazinecarboxamide compounds, along with conjugates and pharmaceutical compositions thereof, are used in the treatment or prevention of disease, such as cancer and fibrotic diseases. The cyclic amino-pyrazinecarboxamide compounds and conjugates thereof may be useful, among other things, in treating and preventing cancer, treating and preventing fibrotic diseases, and modulating signaling by TGFβ1, TGFβ2, and/or TGFβ, or inhibit TGFβR1 and/or TGFβR2 function, or combinations thereof. The cyclic amino-pyrazinecarboxamide compounds may be useful in inhibiting signaling by TGFβ1, TGFβ2, and/or TGFβ, or inhibit TGFβR1 and/or TGFβR2 function or combinations thereof. The cyclic amino-pyrazinecarboxamide compounds of the instant disclosure may be incorporated into a conjugate, such as an antibody conjugate or other targeting moiety. In certain embodiments, a conjugate may provide a improved safety and exposure profile in vivo as compared to compounds alone.

In certain embodiments, the compounds have utility in the treatment of cancer either as single agents, as conjugates, or in combination therapy. In certain embodiments, the compounds have utility as single agent immunomodulators or in combination with conventional cancer therapies. In certain embodiments, the compounds are attached to an antibody construct to form a conjugate that can be utilized, for example, to enhance an immune response when treating cancer, or for treating fibrosis. In certain embodiments, the disclosure provides antibody construct-cyclic-amino-pyrazinecarboxamide compound conjugates (conjugates), and their use for treating cancer or fibrosis.

### Definitions

Unless defined otherwise, all technical and scientific terms used herein have the same meaning as is commonly understood by one of skill in the art to which this invention belongs.

As used in the specification and claims, the singular form “a”, “an” and “the” includes plural references unless the context clearly dictates otherwise. The use of the alternative (e.g., “or”) should be understood to mean either one, both, or any combination thereof of the alternatives. As used herein, the terms “include” and “comprise” are used synonymously.

As used herein, the term “about” used in the context of a number or value refers to a range centered on that number and spanning 15% less than that number and 15% more than that number. The term “about” used in the context of a range refers to an extended range spanning 15% less than that the lowest number listed in the range and 15% more than the greatest number listed in the range.

As used herein, the term “antibody” refers to an immunoglobulin molecule that specifically binds to, or is immunologically reactive toward, a specific antigen. Antibody can include, for example, polyclonal, monoclonal, genetically engineered, and antigen binding fragments thereof. An antibody can be, for example, murine, chimeric, humanized, heteroconjugate, bispecific, diabody, triabody, or tetrabody. The antigen binding fragment can include, for example, a Fab′, F(ab′)₂, Fab, Fv, rIgG, and scFv.

As used herein, an “antigen binding domain” refers to a region of a molecule that specifically binds to an antigen. An antigen binding domain can be an antigen-binding portion of an antibody or an antibody fragment. An antigen binding domain can be one or more fragments of an antibody that can retain the ability to specifically bind to an antigen. An antigen binding domain can be an antigen binding fragment. In some embodiments, an antigen binding domain can recognize a single antigen. An antigen binding domain can recognize, for example, two or three antigens.

As used herein, an “antibody construct” refers to a molecule, e.g., a protein, peptide, antibody or portion thereof, that contains an antigen binding domain and an Fc domain.

As used herein, the abbreviations for the natural L-enantiomeric amino acids are conventional and can be as follows: alanine (A, Ala); arginine (R, Arg); asparagine (N, Asn); aspartic acid (D, Asp); cysteine (C, Cys); glutamic acid (E, Glu); glutamine (Q, Gln); glycine (G, Gly); histidine (H, His); isoleucine (I, Ile); leucine (L, Leu); lysine (K, Lys); methionine (M, Met); phenylalanine (F, Phe); proline (P, Pro); serine (S, Ser); threonine (T, Thr); tryptophan (W, Trp); tyrosine (Y, Tyr); valine (V, Val).

As used herein, “conjugate” refers to an antibody construct or other targeting moiety (e.g., ligand or receptor) that is attached (e.g., conjugated) either directly or through a linker group to a compound described herein, e.g., a compound or salt of any one of Formulas (I), (II), (II-a), (II-b), (II-c), (II-d), (III), (III-a), (III-b), (III-c), (III-d), (IV), (IV-a), (IV-b), (IV-c), and (IV-d) and Table 1.

As used herein, an “Fc domain” can be an Fc domain from an antibody or from a non-antibody that can bind to an Fc receptor.

As used herein, “recognize” with regard to antibody interactions refers to the association or binding between an antigen binding domain of an antibody or portion thereof and an antigen.

As used herein, “sequence identity” refers to the identity between a DNA, RNA, nucleotide, amino acid, or protein sequence to another DNA, RNA, nucleotide, amino acid, or protein sequence, respectively, according to context. Sequence identity can be expressed in terms of a percentage of sequence identity of a first sequence to a second sequence. Percent (%) sequence identity with respect to a reference DNA sequence is the percentage of DNA nucleotides in a candidate sequence that are identical with the DNA nucleotides in the reference DNA sequence after aligning the sequences and introducing gaps, as necessary. Percent (%) sequence identity with respect to a reference amino acid sequence is the percentage of amino acid residues in a candidate sequence that are identical with the amino acid residues in the reference amino acid sequence after aligning the sequences and introducing gaps, if necessary, to achieve the maximum percent sequence identity, and not considering any conservative substitutions as part of the sequence identity. For example, the percent sequence identity values can be generated using the NCBI BLAST 2.0 software as defined by Altschul et al. (1997) “Gapped BLAST and PSI-BLAST: a new generation of protein database search programs”, Nucleic Acids Res. 25:3389-3402, with the parameters set to default values.

As used herein, “specifically binds” and the like refers to the specific association or specific binding between the antigen binding domain and the antigen, as compared with the interaction of the antigen binding domain with a different antigen (i.e., non-specific binding). In some embodiments, an antigen binding domain that recognizes or specifically binds to an antigen has a dissociation constant (K_{D}) of <<100 nM, <10 nM, <1 nM, <0.1 nM, <0.01 nM, or <0.001 nM (e.g., 10⁻⁸ M or less, e.g. from 10⁻⁸ M to 10⁻¹³ M, e.g., from 10⁻⁹ M to 10⁻¹³ M).

As used herein, a “target binding domain” refers to a construct that contains an antigen binding domain from an antibody or from a non-antibody that can bind to the antigen.

The term “targeting moiety” refers to a structure that has a selective affinity for a target molecule relative to other non-target molecules. The targeting moiety binds to a target molecule. A targeting moiety may include, for example, an antibody, a peptide or polypeptide, a carbohydrate, a polynucleotide, a ligand, a receptor, or a binding portion thereof. The target molecule may be an antigen, such as a biological receptor or other structure of a cell (e.g., tumor antigen). In certain embodiments, a targeting moiety comprises a GalNAc moiety. In further embodiments, a targeting moiety comprises a Display Element for display of one or more GalNAc moieties. In some embodiments, a targeting moiety comprises 1, 2, or 3 GalNAc moieties. In some embodiments, a targeting moiety comprises a Display Element for display of two or three GalNAc moieties. In still other embodiments, a targeting moiety comprises a structure of Formula (V):

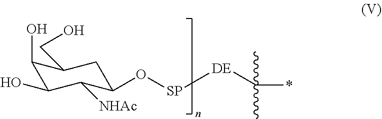

wherein n is 1, 2, or 3; SP is a spacer, wherein each SP is independently a heteroalkylene, heteroalkenylene, or heteroalkynylene comprising 5 to 30 components in the longest linear chain, wherein the components are selected from —CH₂—, —CH(C₁₋₄alkyl), —C(C₁₋₄alkyl)₂, —CH═CH—, —C≡C—, —C(O)—, —O—, —NH—, —N(C₁₋₄alkyl), —S—, —S(O)—, —S(O)₂—, and —P(O)(O⁻)—; and DE is a branched Display Element, wherein the asterisk (*) is the position of connection to the rest of the conjugate.

As used herein, a “tumor antigen” can be an antigenic substance associated with a tumor or cancer cell and can trigger an immune response in a host.

As used herein, a “TGFβR2 inhibitor” refers to a compound that reduces, minimizes, or inactivates serine/threonine kinase activity of TGFβR2 (e.g., directly inhibiting serine/threonine kinase activity or indirectly inhibiting downstream TGFβ-dependent signaling activity, such as SBE-mediated responsiveness to TGFβ and SMAD proteins) by about 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99%, or 100% as compared to untreated TGFβR2. TGFβR2 inhibitors of this disclosure, such as the cyclic amino-pyrazinecarboxamide compounds disclosed herein, may also inhibit activin receptor-linke kinase 5 (ALK5) activity of TGFβR1.

The term “C_{x-y}” or “Cₓ-C_{y}” when used in conjunction with a chemical moiety, such as alkyl, alkenyl, or alkynyl is meant to include groups that contain from x to y carbons in the chain. For example, the term “C₁₋₆ alkyl” refers to substituted or unsubstituted saturated hydrocarbon groups, including straight-chain alkyl and branched-chain alkyl groups that contain from 1 to 6 carbons. The term —C_{x-y} alkylene- refers to a substituted or unsubstituted alkylene chain with from x to y carbons in the alkylene chain. For example —C₁₋₆ alkylene- may be selected from methylene, ethylene, propylene, butylene, pentylene, and hexylene, any one of which is optionally substituted.

The terms “C_{x-y} alkenyl” and “C_{x-y} alkynyl” refer to substituted or unsubstituted unsaturated aliphatic groups analogous in length and possible substitution to the alkyls described above, but that contain at least one double or triple bond, respectively. The term —C_{x-y} alkenylene- refers to a substituted or unsubstituted alkenylene chain with from x to y carbons in the alkenylene chain. For example, —C₂₋₆alkenylene- may be selected from ethenylene, propenylene, butenylene, pentenylene, and hexenylene, any one of which is optionally substituted. An alkenylene chain may have one double bond or more than one double bond in the alkenylene chain. The term —C_{x-y} alkynylene- refers to a substituted or unsubstituted alkynylene chain with from x to y carbons in the alkenylene chain. For example, —C₂₋₆ alkenylene- may be selected from ethynylene, propynylene, butynylene, pentynylene, and hexynylene, any one of which is optionally substituted. An alkynylene chain may have one triple bond or more than one triple bond in the alkynylene chain.

The term “carbocycle” as used herein refers to a saturated, unsaturated or aromatic ring in which each atom of the ring is carbon. Carbocycle includes 3- to 10-membered monocyclic rings, 6- to 12-membered bicyclic rings, and 6- to 12-membered bridged rings. Each ring of a bicyclic carbocycle may be selected from saturated, unsaturated, and aromatic rings. In an exemplary embodiment, an aromatic ring, e.g., phenyl, may be fused to a saturated or unsaturated ring, e.g., cyclohexane, cyclopentane, or cyclohexene. A bicyclic carbocycle includes any combination of saturated, unsaturated and aromatic bicyclic rings, as valence permits. A bicyclic carbocycle includes any combination of ring sizes such as 4-5 fused ring systems, 5-5 fused ring systems, 5-6 fused ring systems, 6-6 fused ring systems, 5-7 fused ring systems, 6-7 fused ring systems, 5-8 fused ring systems, and 6-8 fused ring systems. Exemplary carbocycles include cyclopentyl, cyclohexyl, cyclohexenyl, adamantyl, phenyl, indanyl, and naphthyl. The term “unsaturated carbocycle” refers to carbocycles with at least one degree of unsaturation and excluding aromatic carbocycles. Examples of unsaturated carbocycles include cyclohexadiene, cyclohexene, and cyclopentene.

The term “aryl” refers to an aromatic monocyclic or aromatic multicyclic hydrocarbon ring system. The aromatic monocyclic or aromatic multicyclic hydrocarbon ring system contains only hydrogen and carbon and from five to eighteen carbon atoms, where the rings in the ring system are aromatic, i.e., it contains a cyclic, delocalized (4n+2) π-electron system in accordance with the Hüĉkel theory. The ring system from which aryl groups are derived include, but are not limited to, groups such as benzene, fluorene, indane, indene, tetralin and naphthalene. In some embodiments, the aryl is a monocyclic, bicyclic, tricyclic or tetracyclic ring system, which may include fused ring system (when fused with a cycloalkyl or heterocycloalkyl ring, the aryl is bonded through an aromatic ring atom).

The term “cycloalkyl” refers to a saturated ring in which each atom of the ring is carbon. Cycloalkyl includes monocyclic and polycyclic rings such as 3- to 10-membered monocyclic rings, 6- to 12-membered bicyclic rings, and 6- to 12-membered bridged rings. In certain embodiments, a cycloalkyl comprises three to ten carbon atoms. In other embodiments, a cycloalkyl comprises five to seven carbon atoms. The cycloalkyl may be attached to the rest of the molecule by a single bond. Examples of monocyclic cycloalkyls include, e.g., cyclopropyl, cyclobutyl, cyclopentyl, cyclohexyl, cycloheptyl, and cyclooctyl. Polycyclic cycloalkyl radicals include, for example, adamantyl, norbornyl (i.e., bicyclo[2.2.1]heptanyl), decalinyl, 7,7 dimethyl bicyclo[2.2.1]heptanyl, and the like. The term “cycloalkylene” refers to a bivalent cycloalkyl ring. Examples of monocyclic cycloalkylenes include, e.g., cyclopropylene, cyclobutylene, cyclopentylene, cyclohexylene, cycloheptylene, and cyclooctylene.

The term “halo” or, alternatively, “halogen” or “halide,” means fluoro, chloro, bromo or iodo. In some embodiments, halo is fluoro, chloro, or bromo.

The term “haloalkyl” refers to an alkyl radical, as defined above, that is substituted by one or more halo radicals, for example, trifluoromethyl, dichloromethyl, bromomethyl, 2,2,2-trifluoroethyl, 1-chloromethyl-2-fluoroethyl, and the like. In some embodiments, the alkyl part of the haloalkyl radical is optionally further substituted as described herein.

The term “heterocycle” as used herein refers to a stable saturated, unsaturated or aromatic ring comprising one or more ring heteroatoms. Exemplary heteroatoms include any atom other than carbon, valence permitting. A heteroatom is typically selected from N, O, Si, P, B, and S atoms. Heterocycles include 3- to 10-membered monocyclic rings, 6- to 12-membered bicyclic rings, and 6- to 12-membered bridged rings. A bicyclic heterocycle includes any combination of saturated, unsaturated and aromatic bicyclic rings, as valence permits. In an exemplary embodiment, an aromatic ring, e.g., pyridyl, may be fused to a saturated or unsaturated ring, e.g., cyclohexane, cyclopentane, morpholine, piperidine or cyclohexene. A bicyclic heterocycle includes any combination of ring sizes such as 4-5 fused ring systems, 5-5 fused ring systems, 5-6 fused ring systems, 6-6 fused ring systems, 5-7 fused ring systems, 6-7 fused ring systems, 5-8 fused ring systems, and 6-8 fused ring systems. The term “unsaturated heterocycle” refers to heterocycles with at least one degree of unsaturation and excluding aromatic heterocycles. Examples of unsaturated heterocycles include dihydropyrrole, dihydrofuran, oxazoline, pyrazoline, and dihydropyridine. An “N-containing heterocycle” is a heterocycle with at least one nitrogen ring atom.

The term “heteroaryl” includes aromatic single ring structures, preferably 5- to 7-membered rings, more preferably 5- to 6-membered rings, whose ring structures include at least one heteroatom, preferably one to four heteroatoms, more preferably one or two heteroatoms. The term “heteroaryl” also includes polycyclic ring systems having two or more rings in which two or more carbons are common to two adjoining rings wherein at least one of the rings is heteroaromatic, e.g., the other rings can be aromatic or non-aromatic carbocyclic, or heterocyclic (when fused with a cycloalkyl or heterocycloalkyl ring, the heteroaryl is bonded through an aromatic ring atom). Heteroaryl groups include, for example, pyrrole, furan, thiophene, imidazole, oxazole, thiazole, pyrazole, pyridine, pyrazine, pyridazine, and pyrimidine, and the like. A heteroaryl include monocylic and polycyclic rings having from 5 to 14 rings atoms.

The term “heterocycloalkyl” refers to a stable saturated ring with carbon atoms and at least one heteroatom. Exemplary heteroatoms include any atom other than carbon, valence permitting. In certain embodiments, a heteroatom is selected from N, O, Si, P, B, and S atoms. Heterocycloalkyl include monocyclic and polycyclic rings such as 3- to 10-membered monocyclic rings, 6- to 12-membered bicyclic rings, and 6- to 12-membered bridged rings. The heteroatoms in the heterocycloalkyl radical are optionally oxidized. One or more nitrogen atoms, if present, are optionally quaternized. The heterocycloalkyl is attached to the rest of the molecule through any atom of the heterocycloalkyl, valence permitting, such as any carbon or nitrogen atoms of the heterocycloalkyl. Examples of heterocycloalkyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. The term “heterocycloalkylene” refers to a bivalent heterocycloalkyl ring. Examples of heterocycloalkylenes include, but are not limited to, dioxolanylene, imidazolidinylene, morpholinylene, piperidinylene, piperazinylene, pyrrolidinylene, pyrazolidinylene, tetrahydrofurylene, tetrahydropyranylene, and thiomorpholinylene.

The term “substituted” refers to moieties having substituents replacing a hydrogen on one or more carbons or substitutable heteroatoms, e.g., an NH or NH₂ of a compound. It will be understood that “substitution” or “substituted with” includes the implicit proviso that such substitution is in accordance with permitted valence of the substituted atom and the substituent, and that the substitution results in a stable compound, i.e., a compound which does not spontaneously undergo transformation such as by rearrangement, cyclization, elimination, etc. In certain embodiments, substituted refers to moieties having substituents replacing two hydrogen atoms on the same carbon atom, such as substituting the two hydrogen atoms on a single carbon with an oxo, imino or thioxo group. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds.

In some embodiments, substituents may include any substituents described herein, for example: halogen, hydroxy, oxo (═O), thioxo (═S), cyano (—CN), nitro (—NO₂), imino (═N—H), oximo (═N—OH), hydrazino (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2), and —R^{b}—S(O)ₜN(R^{a})₂ (where t is 1 or 2); and alkyl, alkenyl, alkynyl, aryl, aralkyl, aralkenyl, aralkynyl, cycloalkyl, cycloalkylalkyl, heterocycloalkyl, heterocycloalkylalkyl, heteroaryl, and heteroarylalkyl, any of which may be optionally substituted by alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); wherein each R^{a} is independently selected from hydrogen, alkyl, cycloalkyl, cycloalkylalkyl, aryl, aralkyl, heterocycloalkyl, heterocycloalkylalkyl, heteroaryl, or heteroarylalkyl, wherein each R^{a}, valence permitting, may be optionally substituted with alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); and wherein each R^{b} is independently selected from a direct bond or a straight or branched alkylene, alkenylene, or alkynylene chain, and each R^{c} is a straight or branched alkylene, alkenylene or alkynylene chain.

It will be understood by those skilled in the art that substituents can themselves be substituted, if appropriate. Unless specifically stated as “unsubstituted,” references to chemical moieties herein are understood to include substituted variants. For example, reference to a “heteroaryl” group or moiety implicitly includes both substituted and unsubstituted variants, unless specified otherwise.

“Protecting group” refers to a moiety, except alkyl groups, that when attached to a reactive group in a molecule masks, reduces or prevents that reactivity. Examples of protecting groups can be found in T. W. Greene and P. G. M. Wuts, Protective Groups in Organic Synthesis, 3.sup.rd edition, John Wiley & Sons, New York, 1999, and Harrison and Harrison et al., Compendium of Synthetic Organic Methods, Vols. 1-8 (John Wiley and Sons, 1971-1996), which are incorporated herein by reference in their entirety. Representative amino or amine protecting groups include, formyl, acyl groups (such as acetyl, trifluoroacetyl, and benzoyl), benzyl, alkoxycarbonyl (such as benzyloxycarbonyl (CBZ), and tert-butoxycarbonyl (Boc)), trimethyl silyl (TMS), 2-trimethylsilyl-ethanesulfonyl (SES), trityl and substituted trityl groups, allyloxycarbonyl, 9-fluorenylmethyloxycarbonyl (FMOC), nitro-veratryloxycarbonyl (NVOC), sulfonyl, and the like. Compounds described herein can include protecting groups (e.g., a hydrogen on a reactive nitrogen atom of a compound described herein can be replaced by an amino protecting group).

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable excipient” or “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The term “salt” or “pharmaceutically acceptable salt” refers to salts derived from a variety of organic and inorganic counter ions well known in the art. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts.

### Conjugates

Disclosed herein are antibody constructs and targeting moieties that may be used together with compounds of the disclosure to form conjugates. In certain embodiments, compounds of the disclosure are conjugated either directly or through a linker group to an antibody construct or a targeting moiety to form conjugates.

In certain embodiments, a compound or salt of this disclosure, e.g., a compound or salt of Formulas (I), (II), (II-a), (II-b), (II-c), (II-d), (III), (III-a), (III-b), (III-c), (III-d), (IV), (IV-a), (IV-b), (IV-c), and (IV-d) and Table 1, also may be referred to herein as a TGFβR2 inhibitor, a drug, D, a cyclic amino-pyrazinecarboxamide compound, or a payload, particularly when referenced as part of a conjugate. “LP”, “linker-payload”, “L³-D”, or “compound-linker” may be used herein to refer to a compound or salt of the disclosure bound to a linker.

In certain embodiments, conjugates of the disclosure are represented by the following formula:

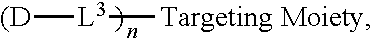

wherein L³ is a linker, D is a compound (TGFβR2 inhibitor) or salt disclosed herein, and n is from 1 to 20. In certain embodiments, n is about 3, about 4, about 5, about 6, about 7 or about 8, or ranges from 1 to about 10, from 1 to about 9, from 1 to about 8, from 2 to about 8, from 1 to about 6, from about 3 to about 5, or from 1 to about 3. In certain embodiments, n is about 4. In certain embodiments, each D is independently selected from Formulas (I), (II), (II-a), (II-b), (II-c), (II-d), (III), (III-a), (III-b), (III-c), (III-d), (IV), (IV-a), (IV-b), (IV-c), and (IV-d) and Table 1, respectively.

In certain embodiments, conjugates of the disclosure are represented by the following formula:

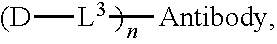

wherein Antibody is an antibody construct, L³ is a linker, D is a compound (TGFβR2 inhibitor) or salt disclosed herein, and n is from 1 to 20. In certain embodiments, n is about 3, about 4, about 5, about 6, about 7 or about 8, or ranges from 1 to about 10, from 1 to about 9, from 1 to about 8, from 2 to about 8, from 1 to about 6, from about 3 to about 5, or from 1 to about 3. In certain embodiments, n is about 4. In certain embodiments, each D is independently selected from Formulas (I), (II), (II-a), (II-b), (II-c), (II-d), (III), (III-a), (III-b), (III-c), (III-d), (IV), (IV-a), (IV-b), (IV-c), and (IV-d) and Table 1, respectively.

An antibody construct may contain, for example, two, three, four, five, six, seven, eight, nine, ten, or more antigen binding domains. An antibody construct may contain two antigen binding domains in which each antigen binding domain can recognize the same antigen. An antibody construct may contain two antigen binding domains in which each antigen binding domain can recognize different antigens. An antigen binding domain may be in a scaffold, in which a scaffold is a supporting framework for the antigen binding domain. An antigen binding domain may be in a non-antibody scaffold. An antigen binding domain may be in an antibody scaffold. An antibody construct may comprise an antigen binding domain in a scaffold. The antibody construct may comprise an Fc fusion protein. In some embodiments, the antibody construct is an Fc fusion protein. An antigen binding domain may specifically bind to a tumor antigen. An antigen binding domain may specifically bind to an antigen having at least 80%, at least 90%, at least 95%, at least 99%, or 100% sequence identity to a tumor antigen. An antigen binding domain may specifically bind to an antigen on an antigen presenting cell (APC). An antigen binding domain may specifically bind to an antigen having at least 80%, at least 90%, at least 95%, at least 99%, or 100% sequence identity to an antigen on an antigen presenting cell (APC).

An antibody construct may consist of two identical light protein chains and two identical heavy protein chains, all held together covalently by disulfide linkages. The N-terminal regions of the light and heavy chains together may form the antigen recognition site of an antibody. Structurally, various functions of an antibody may be confined to discrete protein domains. The sites that can recognize and can bind antigen may consist of three complementarities determining regions (CDRs) that may lie within the variable heavy chain region and variable light chain region at the N-terminal end of the heavy chain and the light chain. The constant domains may provide the general framework of the antibody and may not be involved directly in binding the antibody to an antigen, but may be involved in various effector functions, such as participation of the antibody in antibody-dependent cellular cytotoxicity, and may bind Fc receptors. The constant domains may include an Fc region. The constant domains may include an Fc domain. The variable regions of natural light and heavy chains may have the same general structures, and each domain may comprise four framework regions, whose sequences can be somewhat conserved, connected by three hyper-variable regions or CDRs. The four framework regions (FR) may largely adopt a β-sheet conformation and the CDRs can form loops connecting, and in some aspects forming part of, the β-sheet structure. The CDRs in each chain may be held in close proximity by the framework regions and with the CDRs from the other chain, may contribute to the formation of the antigen binding site.

An antibody construct may comprise a light chain of an amino acid sequence having at least one, two, three, four, five, six, seven, eight, nine or ten modifications and in certain embodiments, not more than 40, 35, 30, 25, 20, 15 or 10 modifications of the amino acid sequence relative to the natural or original amino acid sequence. An antibody construct may comprise a heavy chain of an amino acid sequence having at least one, two, three, four, five, six, seven, eight, nine or ten modifications and in certain embodiments, not more than 40, 35, 30, 25, 20, 15 or 10 modifications of the amino acid sequence relative to the natural or original amino acid sequence.

An antibody construct may be an antibody. Antibodies may be selected from different classes of immunoglobins, e.g., IgA, IgD, IgE, IgG, and IgM. The several different classes may be further divided into isotypes, e.g., IgG1, IgG2, IgG3, IgG4, IgA1, and IgA2. An antibody may further comprise a light chain and a heavy chain, often more than one chain. The heavy-chain constant regions (Fc) that corresponds to the different classes of immunoglobulins may be α, δ, ε, γ, and μ, respectively. The light chains may be one of either kappa (κ) or lambda (λ), based on the amino acid sequences of the constant domains. The Fc domain may further comprise an Fc region. An Fc receptor may bind an Fc domain. Antibody constructs may also include any fragment or recombinant forms thereof, including but not limited to, single chain variable fragments (scFvs).

An antibody construct may comprise an antigen-binding antibody fragment. An antibody fragment may include (i) a Fab fragment, a monovalent fragment consisting of the V_{L}, V_{H}, C_{L} and C_{H1} domains; (ii) a F(ab′)₂ fragment, a bivalent fragment comprising two Fab fragments linked by a disulfide bridge at the hinge region; and (iii) a Fv fragment consisting of the V_{L} and V_{H} domains of a single arm of an antibody. Although the two domains of the Fv fragment, V_{L} and V_{H}, may be coded for by separate genes, they may be linked by a synthetic linker to be made as a single protein chain in which the V_{L} and V_{H} regions pair to form monovalent molecules.

F(ab′)₂ and Fab′ moieties may be produced by genetic engineering or by treating immunoglobulin (e.g., monoclonal antibody) with a protease such as pepsin and papain, and may include an antibody fragment generated by digesting immunoglobulin near the disulfide bonds existing between the hinge regions in each of the two H chains. The Fab fragment may also contain the constant domain of the light chain and the first constant domain (C_{H1}) of the heavy chain. Fab′ fragments may differ from Fab fragments by the addition of a few residues at the carboxyl terminus of the heavy chain C_{H1} domain including one or more cysteine(s) from the antibody hinge region.

An Fv may be the minimum antibody fragment which contains a complete antigen-recognition and antigen-binding site. This region may consist of a dimer of one heavy chain and one light chain variable domain in tight, non-covalent association. In this configuration, the three CDRs of each variable domain may interact to define an antigen-binding site on the surface of the V_{H}-V_{L} dimer. A single variable domain (or half of an Fv comprising only three CDRs specific for an antigen) may recognize and bind to antigen, although the binding can be at a lower affinity than the affinity of the entire binding site.

An antibody construct may include an Fc domain comprising an Fc region or several Fc domains. The Fc domain of an antibody may interact with FcRs found on immune cells. The Fc domain may also mediate the interaction between effector molecules and cells, which may lead to activation of the immune system. In the IgG, IgA, and IgD antibody isotypes, the Fc region may comprise two identical protein fragments, which can be derived from the second and third constant domains of the antibody's heavy chains. In the IgM and IgE antibody isotypes, the Fc regions may comprise three heavy chain constant domains. In the IgG antibody isotype, the Fc regions may comprise a highly-conserved N-glycosylation site, which may be important for FcR-mediated downstream effects.

An antibody construct used herein may be “chimeric” or “humanized.” Chimeric and humanized forms of non-human (e.g., murine) antibodies can be chimeric immunoglobulins, immunoglobulin chains or fragments thereof (such as Fv, Fab, Fab′, F(ab′)₂ or other target-binding subdomains of antibodies), which may contain minimal sequences derived from non-human immunoglobulin. In general, the humanized antibody may comprise substantially all of at least one, and typically two, variable domains, in which all or substantially all of the CDRs correspond to those of a non-human immunoglobulin and all or substantially all of the framework regions are those of a human immunoglobulin sequence. The humanized antibody may also comprise at least a portion of an immunoglobulin constant region (Fc), such as that of a human immunoglobulin consensus sequence.

An antibody construct may be a human antibody. As used herein, “human antibodies” can include antibodies having, for example, the amino acid sequence of a human immunoglobulin and may include antibodies isolated from human immunoglobulin libraries or from animals transgenic for one or more human immunoglobulins that do not express endogenous immunoglobulins. Human antibodies may be produced using transgenic mice which are incapable of expressing functional endogenous immunoglobulins, but which may express human immunoglobulin genes. Completely human antibodies that recognize a selected epitope may be generated using guided selection. In this approach, a selected non-human monoclonal antibody, e.g., a mouse antibody, may be used to guide the selection of a completely human antibody recognizing the same epitope.

An antibody may be a bispecific antibody or a dual variable domain antibody (DVD). Bispecific and DVD antibodies may be monoclonal, often human or humanized, antibodies that can have binding specificities for at least two different antigens.

An antigen binding domain of an antibody may comprise one or more light chain (L) CDRs and one or more heavy chain (H) CDRs. For example, an antigen binding domain of an antibody may comprise one or more of the following: a light chain complementary determining region 1 (LCDR1), a light chain complementary determining region 2 (LCDR2), or a light chain complementary determining region 3 (LCDR3). For another example, an antigen binding domain may comprise one or more of the following: a heavy chain complementary determining region 1 (HCDR1), a heavy chain complementary determining region 2 (HCDR2), or a heavy chain complementary determining region 3 (HCDR3). As an additional example, an antigen binding domain of an antibody may comprise one or more of the following: LCDR1, LCDR2, LCDR3, HCDR1, HCDR2, and HCDR3. In some embodiments, an antigen binding domain of an antibody includes all six CDRs, (i.e., LCDR1, LCDR2, LCDR3, HCDR1, HCDR2, and HCDR3).

The antigen binding domain of an antibody construct may be selected from any domain that specifically binds the antigen including, but not limited to, from a monoclonal antibody, a polyclonal antibody, a recombinant antibody, or binding functional fragment thereof, for example, a heavy chain variable domain (V_{H}) and a light chain variable domain (V_{L}), or a DARPin, an affimer, an avimer, a knottin, a monobody, an affinity clamp, an ectodomain, a receptor ectodomain, a receptor, a cytokine, a ligand, an immunocytokine, a T cell receptor, or a recombinant T cell receptor.

The antigen binding domain of an antibody construct may be at least 80% identical to an antigen binding domain selected from, but not limited to, a monoclonal antibody, a polyclonal antibody, a recombinant antibody, or a functional fragment thereof, for example, a heavy chain variable domain (V_{H}) and a light chain variable domain (V_{L}), or a DARPin, an affimer, an avimer, a knottin, a monobody, an affinity clamp, an ectodomain, a receptor ectodomain, a receptor, a cytokine, a ligand, an immunocytokine, a T cell receptor, or a recombinant T cell receptor.

An antibody may be a derivatized antibody. For example, derivatized antibodies may be modified by glycosylation, acetylation, pegylation, phosphorylation, amidation, derivatization by known protecting/blocking groups, proteolytic cleavage, linkage to a cellular ligand or other protein.

An antibody may have a sequence that has been modified to alter at least one constant region-mediated biological effector function relative to the corresponding wild type sequence. For example, in some embodiments, the antibody can be modified to reduce at least one constant region-mediated biological effector function relative to an unmodified antibody, e.g., reduced binding to the Fc receptor (FcR). FcR binding may be reduced by, for example, mutating the immunoglobulin constant region segment of the antibody at particular regions necessary for FcR interactions.

An antibody or Fc domain may be modified to acquire or improve at least one constant region-mediated biological effector function relative to an unmodified antibody or Fc domain, e.g., to enhance FcγR interactions. For example, an antibody with a constant region that binds to FcγRIIA, FcγRIIB and/or FcγRIIIA with greater affinity than the corresponding wild type constant region may be produced according to the methods described herein. An Fc domain that binds to FcγRIIA, FcγRIIB and/or FcγRIIIA with greater affinity than the corresponding wild type Fc domain may be produced according to the methods described herein or known to the skilled artisan.

In certain embodiments, an antibody construct or conjugate of the disclosure comprises an Fc domain that may comprise an Fc region, in which the Fc domain may be the part of the Fc region that interacts with Fc receptors. An antibody construct can comprise an Fc domain in a scaffold. An antibody construct can comprise an Fc domain in an antibody scaffold. An antibody construct can comprise an Fc domain in a non-antibody scaffold. An antibody construct can comprise an Fc domain covalently attached to an antigen binding domain. The Fc domain of an antibody construct may interact with Fc-receptors (FcRs) found on immune cells. The Fc domain may also mediate the interaction between effector molecules and cells, which can lead to activation of the immune system. The Fc region may be derived from IgG, IgA, or IgD antibody isotypes, and may comprise two identical protein fragments, which are derived from the second and third constant domains of the antibody's heavy chains. In an Fc domain or region derived from an IgG antibody isotype, the Fc domain or region may comprise a highly-conserved N-glycosylation site, which may be essential for FcR-mediated downstream effects. The Fc domain or region may be derived from IgM or IgE antibody isotypes, in which the Fc domain or region may comprise three heavy chain constant domains. A conjugate can comprise an antibody construct comprising an Fc domain that can bind to an FcR when linked to a TGFβR2 inhibitor conjugate.

An antibody construct can comprise an Fc domain of an IgG1 isotype. An antibody construct can comprise an Fc domain of an IgG2 isotype. An antibody construct can comprise an Fc domain of an IgG3 isotype. An antibody construct can comprise an Fc domain of an IgG4 isotype. An antibody construct can have a hybrid isotype comprising constant regions from two or more isotypes. An Fc domain typically comprises C_{H}2 and C_{H}3 domains of a heavy chain constant region, but may comprise more or less of the heavy chain constant region as well.

The specificity of the Fc domain to an Fc receptor of a conjugate disclosed herein can be influenced by the presence of a TGFβR2 inhibitor. The Fc domain of the conjugate can bind to an Fc receptor with at least about 10%, about 20%, about 30%, about 40%, about 50%, about 60%, about 70%, about 80%, about 85%, about 90%, about 95%, or about 100% of a specificity of the Fc domain to the Fc receptor in the absence of the TGFβR2 inhibitor.

An Fc domain may interact with different types of FcRs. The different types of FcRs may include, for example, FcγRI (CD64), FcγRIIA (CD32a), FcγRIIB (CD32b), FcγRIIIA (CD16a), FcγRIIIB (CD16b), FcαRI (CD89), FcαμR, FcεRI, FcεRII, and FcRn. In certain embodiments, an FcγRIIIA (CD16a) can be an FcγRIIIA (CD16a) F158 variant or a V158 variant. The FcαR class binds to IgA and the FcγR class binds to IgG. Each FcγR isoform can differ in binding affinity to the Fc domain of the IgG antibody. For example, FcγRI can bind to IgG with greater affinity than FcγRII or FcγRIII. The affinity of a particular FcγR isoform to IgG can be controlled, in part, by a glycan (e.g., oligosaccharide) at position CH2 84.4 of the IgG antibody. For example, fucose containing CH2 84.4 glycans can reduce IgG affinity for FcγRIIIA. In addition, G0 glucans can have increased affinity for FcγRIIIA due to the lack of galactose and terminal GlcNAc moiety.

FcRs may be located on the membrane of certain immune cells including, for example, B lymphocytes, natural killer cells, macrophages, neutrophils, dendritic cells (DCs) (e.g., follicular DCs), eosinophils, basophils, platelets, and mast cells. Once the FcR is engaged by the Fe domain, the FcR may initiate functions including, for example, clearance of an antigen-antibody complex via receptor-mediated endocytosis, antibody-dependent cell-mediated cytotoxicity (ADCC), antibody dependent cell-mediated phagocytosis (ADCP), and ligand-triggered transmission of signals across the plasma membrane that can result in alterations in secretion, exocytosis, and cellular metabolism. FcRs may deliver signals when FcRs are aggregated by antibodies and multivalent antigens at the cell surface. The aggregation of FcRs with immunoreceptor tyrosine-based activation motifs (ITAMs) may sequentially activate SRC family tyrosine kinases and SYK family tyrosine kinases. ITAM comprises a twice-repeated YxxL sequence flanking seven variable residues. The SRC and SYK kinases may connect the transduced signals with common activation pathways.

The profile of FcRs on a DC can impact the ability of the DC to respond upon stimulation. For example, most DC can express both CD32A and CD32B, which can have opposing effects on IgG-mediated maturation and function of DCs: binding of IgG to CD32A can mature and activate DCs in contrast with CD32B, which can mediate inhibition due to phosphorylation of immunoreceptor tyrosine-based inhibition motif (ITIM), after CD32B binding of IgG. Therefore, the activity of these two receptors can establish a threshold of DC activation. Furthermore, difference in functional avidity of these receptors for IgG can shift their functional balance. Hence, altering the Fc domain binding to FcRs can also shift their functional balance, allowing for manipulation (either enhanced activity or enhanced inhibition) of the DC immune response.

A modification in the amino acid sequence of an Fc domain can alter the recognition of an FcR for the Fc domain. However, such modifications can still allow for FcR-mediated signaling. A modification can be a substitution of an amino acid at a residue of an Fc domain (e.g., wildtype) for a different amino acid at that residue. A modification can permit binding of an FcR to a site on the Fc domain that the FcR may not otherwise bind to. A modification can increase binding affinity of an FcR to the Fc domain. A modification can decrease binding affinity of an FcR to a site on the Fc domain that the FcR may have increased binding affinity for. A modification can increase the subsequent FcR-mediated signaling after Fc domain binding to an FcR.

An Fc domain of an antibody construct or a conjugate can be a naturally occurring or a variant of a naturally occurring Fc domain and can comprise at least one amino acid change as compared to the sequence of a wild-type Fc domain. An amino acid change in an Fc domain can allow the antibody or conjugate to bind to at least one Fc receptor with lessor affinity compared to a wild-type Fc domain.

In some embodiments, an Fc domain of an antibody construct or a conjugate exhibits increased binding affinity to one or more Fc receptors. In some embodiments, an Fc domain of an antibody construct or a conjugate exhibits increased binding affinity to one or more Fcγ receptors. In some embodiments, an Fc domain of an antibody construct or a conjugate exhibits increased binding affinity to FcRn receptors. In some embodiments, an Fc domain of an antibody construct or a conjugate exhibits increased binding affinity to Fcγ and FcRn receptors. In other embodiments, an Fc domain of an antibody construct or a conjugate exhibits the same or substantially similar binding affinity to Fcγ and/or FcRn receptors as compared to a wild-type Fc domain from an IgG antibody (e.g., IgG1 antibody).

In some embodiments, an Fc domain or region can exhibit reduced binding affinity to one or more Fc receptors. In some embodiments, an Fc domain or region of an antibody construct or a conjugate can exhibit reduced binding affinity to one or more Fcγ receptors. In some embodiments, an Fc domain or region of an antibody construct or a conjugate can exhibit reduced binding affinity to FcRn receptors. In some embodiments, an Fc domain or region of an antibody construct or a conjugate can exhibit reduced binding affinity to Fcγ and FcRn receptors. In some embodiments, an Fc domain of an antibody construct or a conjugate is an Fc null domain or region. As used herein, an “Fc null” refers to a domain that exhibits weak to no binding to any of the Fcγ receptors. In some embodiments, an Fc null domain or region of an antibody construct or a conjugate exhibits a reduction in binding affinity (e.g., increase in Kd) to Fcγ receptors of at least 1000-fold. In some embodiments, an Fc domain of an antibody construct or a conjugate exhibits decreased binding affinity to FcRn receptors, but exhibits the same or increased binding affinity to one or more Fcgamma receptors as compared to a wildtype Fc domain. In some embodiments, an Fc domain of an antibody construct or a conjugate exhibits increased binding affinity to FcRn receptors, but exhibits the same or decreased binding affinity to one or more Fcgamma receptors.

Binding of Fc receptors to an Fc domain can be affected by amino acid substitutions. The modification can be located in a portion of an antibody sequence which includes an Fc domain of the antibody and, in particular, can be located in portions of the Fc domain that can bind Fc receptors. The Fc domain may have one or more, two or more, three or more, or four or more amino acid substitutions that decrease binding of the Fc domain to an Fc receptor. In certain embodiments, an Fc domain exhibits decreased binding to FcγRI (CD64), FcγRIIA (CD32), FcγRIIIA (CD16a), FcγRIIIB (CD16b), or any combination thereof. In order to decrease binding affinity of an Fc domain or region to an Fc receptor, the Fc domain or region may comprise one or more amino acid substitutions that has the effect of reducing the affinity of the Fc domain or region to an Fc receptor. A modification can be substitution of E233, L234 and L235, such as E233P/L234V/L235A or E233P/L234V/L235A/AG236, according to the EU index of Kabat. A modification can be a substitution of P238, such as P238A, according to the EU index of Kabat. A modification can be a substitution of D265, such as D265A, according to the EU index of Kabat. A modification can be a substitution of N297, such as N297A, according to the EU index of Kabat. A modification can be a substitution of A327, such as A327Q, according to the EU index of Kabat. A modification can be a substitution of P329, such as P239A, according to the EU index of Kabat. In certain embodiments, the one or more substitutions comprise any one or more of IgG1 heavy chain mutations corresponding to E233P, L234V, L234A, L235A, L235E, AG236, G237A, E318A, K320A, K322A, A327G, A330S, or P331S according to the EU index of Kabat numbering.

In some embodiments, the Fc domain or region of an antibody construct or a conjugate can comprise a sequence of the IgG1 isoform that has been modified from the wild-type IgG1 sequence. A modification can comprise a substitution at more than one amino acid residue, such as at 5 different amino acid residues including L235V/F243L/R292P/Y300L/P396L (IgG1VLPLL) according to the EU index of Kabat numbering. A modification can comprise a substitution at more than one amino acid residue such as at 2 different amino acid residues including S239D/I332E (IgG1DE) according to the EU index of Kabat numbering. A modification can comprise a substitution at more than one amino acid residue such as at 3 different amino acid residues including S298A/E333A/K334A (IgG1AAA) according to the EU index of Kabat numbering.

In certain embodiments, binding of some Fc receptors to an Fc domain variant comprising the IgG1VLPLL modifications can be enhanced compared to wild-type by as result of the L235V/F243L/R292P/Y300L/P396L amino acid modifications. In other embodiments, binding of other Fc receptors to the Fc domain variant comprising the IgG1VLPLL modifications can be reduced compared to wild-type by the L235V/F243L/R292P/Y300L/P396L amino acid modifications. For example, the binding affinities of the Fc domain variant comprising the IgG1VLPLL modifications to FcγRIIIA and to FcγRIIA can be enhanced compared to wild-type whereas the binding affinity of the Fc domain variant comprising the IgG1VLPLL modifications to FcγRIIB can be reduced compared to wild-type.

In another example, binding of Fc receptors to an Fc domain variant comprising the IgG1DE modifications can be enhanced compared to wild-type as a result of the S239D/I332E amino acid modification. In certain embodiments, binding of some Fc receptors to the Fc domain variant comprising the IgG1DE modifications can be reduced compared to wild-type by S239D/I332E amino acid modification. For example, the binding affinities of the Fc domain variant comprising the IgG1DE modifications to FcγRIIIA and to FcγRIIB can be enhanced compared to wild-type.

In still another example, binding of Fc receptors to an Fc domain variant comprising the IgG1AAA modifications can be enhanced compared to wild-type as a result of the S298A/E333A/K334A amino acid modification. In certain embodiments, binding of some Fc receptors to Fc domain variant comprising the IgG1AAA modifications can be reduced compared to wild-type by S298A/E333A/K334A amino acid modification. Binding affinities of the Fc domain variant comprising the IgG1AAA modifications to FcγRIIIA can be enhanced compared to wild-type whereas the binding affinity of the Fc domain variant comprising the IgG1AAA modifications to FcγRIIB can be reduced compared to wildtype.

In some embodiments, the heavy chain of a human IgG2 antibody can be mutated at cysteines as positions 127, 232, or 233. In some embodiments, the light chain of a human IgG2 antibody can be mutated at a cysteine at position 214. The mutations in the heavy and light chains of the human IgG2 antibody can be from a cysteine residue to a serine residue.

While an antibody construct can comprise a first binding domain and a second binding domain with wild-type or modified amino acid sequences encoding the Fc domain, the modifications of the Fc domain from the wild-type sequence may not significantly alter binding and/or affinity of the Fc domain or the antigen binding domain(s). For example, binding and/or affinity of an antibody construct or a conjugate comprising a first binding domain and a second binding domain (or, in some cases, a third binding domain) and having the Fc domain modifications of IgG1VLPLL, IgG1DE, or IgG1AAA may not be significantly altered by modification of an Fc domain amino acid sequence compared to a wild-type sequence. Modifications of an Fc domain from a wild-type sequence may not alter binding and/or affinity of a first binding domain or target binding domain that binds, for example, to a tumor-associated antigen or a fibrosis-associated antigen. Additionally, the binding and/or affinity of the binding domains described herein, for example a first binding domain, a second binding domain (or, in some cases, a third binding domain), and an Fc domain variant selected from IgG1VLPLL, IgG1DE, and IgG1AAA, may be comparable to the binding and/or affinity of wild-type antibodies.

In some embodiments, an IgG Fc domain of an antibody construct or a conjugate comprises at least one amino acid substitution that reduces its binding affinity to FcγR1, as compared to a wild-type or reference IgG Fc domain. A modification can comprise a substitution at F241, such as F241A, according to the EU index of Kabat. A modification can comprise a substitution at F243, such as F243A, according to the EU index of Kabat. A modification can comprise a substitution at V264, such as V264A, according to the EU index of Kabat. A modification can comprise a substitution at D265, such as D265A according to the EU index of Kabat.

In some embodiments, an IgG Fc domain of an antibody construct or a conjugate comprises at least one amino acid substitution that increases its binding affinity to FcγR1, as compared to a wild-type or reference IgG Fc domain. A modification can comprise a substitution at A327 and P329, such as A327Q/P329A, according to the EU index of Kabat.

In some embodiments, the modification comprises substitution of one or more amino acids that reduce binding affinity of an IgG Fc domain to FcγRII and FcγRIIIA receptors. A modification can be a substitution of D270, such as D270A, according to the EU index of Kabat. A modification can be a substitution of Q295, such as Q295A, according to the EU index of Kabat. A modification can be a substitution of A327, such as A237S, according to the EU index of Kabat.

In some embodiments, the modification comprises substitution of one or more amino acids that increases binding affinity of an IgG Fc domain of an antibody construct or a conjugate to FcγRII and FcγRIIIA receptors. A modification can be a substitution of T256, such as T256A, according to the EU index of Kabat. A modification can be a substitution of K290, such as K290A, according to the EU index of Kabat.

In some embodiments, the modification comprises substitution of one or more amino acids that increases binding affinity of an IgG Fc domain of an antibody construct or a conjugate to FcγRII receptor. A modification can be a substitution of R255, such as R255A, according to the EU index of Kabat. A modification can be a substitution of E258, such as E258A, according to the EU index of Kabat. A modification can be a substitution of S267, such as S267A, according to the EU index of Kabat. A modification can be a substitution of E272, such as E272A, according to the EU index of Kabat. A modification can be a substitution of N276, such as N276A, according to the EU index of Kabat. A modification can be a substitution of D280, such as D280A, according to the EU index of Kabat. A modification can be a substitution of H285, such as H285A, according to the EU index of Kabat. A modification can be a substitution of N286, such as N286A, according to the EU index of Kabat. A modification can be a substitution of T307, such as T307A, according to the EU index of Kabat. A modification can be a substitution of L309, such as L309A, according to the EU index of Kabat. A modification can be a substitution of N315, such as N315A, according to the EU index of Kabat. A modification can be a substitution of K326, such as K326A, according to the EU index of Kabat. A modification can be a substitution of P331, such as P331A, according to the EU index of Kabat. A modification can be a substitution of S337, such as S337A, according to the EU index of Kabat. A modification can be a substitution of A378, such as A378A, according to the EU index of Kabat. A modification can be a substitution of E430, such as E430, according to the EU index of Kabat.

An amino acid change in an Fc domain can allow the antibody construct or conjugate to bind to at least one Fc receptor with greater affinity compared to a wild-type Fc domain. An Fc domain variant can comprise an amino acid sequence having at least one, two, three, four, five, six, seven, eight, nine or ten modifications but not more than 40, 35, 30, 25, 20, 15 or 10 modifications of the amino acid sequence relative to the natural or original amino acid sequence. An Fc domain variant of an antibody construct or a conjugate can comprise a sequence of the IgG1 isoform that has been modified from a wildtype IgG1 sequence to increase Fc receptor binding.

In some embodiments, the modification comprises substitution of one or more amino acids that increases binding affinity of an IgG Fc domai of an antibody construct or a conjugate n to FcγRII receptor and reduces the binding affinity to FcγRIIIA receptor. A modification can be a substitution of H268, such as H268A, according to the EU index of Kabat. A modification can be a substitution of R301, such as R301A, according to the EU index of Kabat. A modification can be a substitution of K322, such as K322A, according to the EU index of Kabat.

In some embodiments, the modification comprises substitution of one or more amino acids that decreases binding affinity of an IgG Fc domain of an antibody construct or a conjugate to FcγRII receptor but does not affect the binding affinity to FcγRIIIA receptor. A modification can be a substitution of R292, such as R292A, according to the EU index of Kabat. A modification can be a substitution of K414, such as K414A, according to the EU index of Kabat.

In some embodiments, the modification comprises substitution of one or more amino acids that decreases binding affinity of an IgG Fc domai of an antibody construct or a conjugate n to FcγRII receptor and increases the binding affinity to FcγRIIIA receptor. A modification can be a substitution of S298, such as S298A, according to the EU index of Kabat. A modification can be substitution of S239, 1332 and A330, such as S239D/I332E/A330L. A modification can be substitution of S239 and 1332, such as S239D/I332E.

In some embodiments, the modification comprises substitution of one or more amino acids that decreases binding affinity of an IgG Fc domain of an antibody construct or a conjugate to FcγRIIIA receptor. A modification can be substitution of F241 and F243, such as F241S/F243 S or F241I/F243I, according to the EU index of Kabat.

In some embodiments, the modification comprises substitution of one or more amino acids that decreases binding affinity of an IgG Fc domain of an antibody construct or a conjugate to FcγRIIIA receptor and does not affect the binding affinity to FcγRII receptor. A modification can be a substitution of S239, such as S239A, according to the EU index of Kabat. A modification can be a substitution of E269, such as E269A, according to the EU index of Kabat. A modification can be a substitution of E293, such as E293A, according to the EU index of Kabat. A modification can be a substitution of Y296, such as Y296F, according to the EU index of Kabat. A modification can be a substitution of V303, such as V303A, according to the EU index of Kabat. A modification can be a substitution of A327, such as A327G, according to the EU index of Kabat. A modification can be a substitution of K338, such as K338A, according to the EU index of Kabat. A modification can be a substitution of D376, such as D376A, according to the EU index of Kabat.

In some embodiments, the modification comprises substitution of one or more amino acids that increases binding affinity of an IgG Fc domain of an antibody construct or a conjugate to FcγRIIIA receptor and does not affect the binding affinity to FcγRII receptor. A modification can be a substitution of E333, such as E333A, according to the EU index of Kabat. A modification can be a substitution of K334, such as K334A, according to the EU index of Kabat. A modification can be a substitution of A339, such as A339T, according to the EU index of Kabat. A modification can be substitution of S239 and 1332, such as S239D/I332E.

In some embodiments, the modification comprises substitution of one or more amino acids that increases binding affinity of an IgG Fc domain to FcγRIIIA receptor. A modification can be substitution of L235, F243, R292, Y300 and P396, such as L235V/F243L/R292P/Y300L/P396L (IgG1VLPLL) according to the EU index of Kabat. A modification can be substitution of S298, E333 and K334, such as S298A/E333A/K334A, according to the EU index of Kabat. A modification can be substitution of K246, such as K246F, according to the EU index of Kabat.

Other substitutions in an IgG Fc domain of an antibody construct or a conjugate that affect its interaction with one or more Fcγ receptors are disclosed in U.S. Pat. Nos. 7,317,091 and 8,969,526 (the disclosures of which are incorporated by reference herein).

In some embodiments, an IgG Fc domain of an antibody construct or a conjugate comprises at least one amino acid substitution that reduces the binding affinity to FcRn, as compared to a wild-type or reference IgG Fc domain. A modification can comprise a substitution at H435, such as H435A according to the EU index of Kabat. A modification can comprise a substitution at 1253, such as I253A according to the EU index of Kabat. A modification can comprise a substitution at H310, such as H310A according to the EU index of Kabat. A modification can comprise substitutions at 1253, H310 and H435, such as I253A/H310A/H435A according to the EU index of Kabat.

A modification can comprise a substitution of one amino acid residue that increases the binding affinity of an IgG Fc domain of an antibody construct or a conjugate for FcRn, relative to a wildtype or reference IgG Fc domain. A modification can comprise a substitution at V308, such as V308P according to the EU index of Kabat. A modification can comprise a substitution at M428, such as M428L according to the EU index of Kabat. A modification can comprise a substitution at N434, such as N434A according to the EU index of Kabat or N434H according to the EU index of Kabat. A modification can comprise substitutions at T250 and M428, such as T250Q and M428L according to the EU index of Kabat. A modification can comprise substitutions at M428 and N434, such as M428L and N434S, N434A or N434H according to the EU index of Kabat. A modification can comprise substitutions at M252, S254 and T256, such as M252Y/S254T/T256E according to the EU index of Kabat. A modification can be a substitution of one or more amino acids selected from P257L, P257N, P257I, V279E, V279Q, V279Y, A281S, E283F, V284E, L306Y, T307V, V308F, Q311V, D376V, and N434H. Other substitutions in an IgG Fc domain of an antibody construct or a conjugate that affect its interaction with FcRn are disclosed in U.S. Pat. No. 9,803,023 (the disclosure of which is incorporated by reference herein).

In some embodiments, an antibody is a human IgG2 antibody, including an IgG2 Fc region. In some embodiments, the heavy chain of the human IgG2 antibody can be mutated at cysteines as positions 127, 232, or 233. In some embodiments, the light chain of a human IgG2 antibody can be mutated at a cysteine at position 214. In particular embodiments, the mutations in the heavy and light chains of the human IgG2 antibody can be from a cysteine residue to a serine residue.

In certain embodiments, the antibody construct comprises an antigen binding domain and an Fc domain.

In certain embodiments, the antigen binding domain specifically binds to an antigen that is at least 80% identical to an antigen on a T cell, a B cell, a stellate cell, an endothelial cell, a tumor cell, an APC, a fibroblast cell, a fibrocyte cell, or a cell associated with the pathogenesis of fibrosis. In certain embodiments, the antigen binding domain specifically binds to an antigen that is at least 80% identical to an antigen on a T cell, an APC, and/or a B cell. In certain embodiments, the antigen binding domain specifically binds to an antigen that is at least 80% identical to an antigen on a hepatocyte. In certain embodiments, the antigen binding domain may specifically bind to an antigen that is at least 80% identical to an antigen selected from the group consisting of CTLA4, PD-1, OX40, LAG-3, GITR, GARP, CD25, CD27, PD-L1, TNFR2, ICOS, 41BB, CD70, CD73, CD38, or VTCN1. In certain embodiments, the antigen binding domain may specifically bind to an antigen that is at least 80% identical to an antigen selected from the group consisting of ASGR1 and ASGR2 (asialoglycoprotein receptor 1 and 2). In certain embodiments, the antigen binding domain specifically binds to an antigen that is at least 80% identical to an antigen on a stellate cell, an endothelial cell, a fibroblast cell, a fibrocyte cell, or a cell associated with the pathogenesis of fibrosis or cancer. In certain embodiments, the antigen binding domain may specifically bind to an antigen that is at least 80% identical to an antigen selected from the group consisting of LRRC15, PDGFRβ, integrin αvβ1, integrin αvβ3, integrin αvβ6, integrin αvβ8, Endosialin, FAP, ADAM12, MMP14, PDPN, CDH11 and F2RL2, In certain embodiments, the antigen binding domain may specifically bind to an antigen that is at least 80% identical to an antigen selected from the group consisting of FAP, ADAM12, LRRC15, MMP14, PDPN, CDH11 and F2RL2. In certain embodiments, the antigen binding domain specifically binds to an antigen that is at least 80% identical to an antigen on a tumor cell, a tumor antigen. In certain embodiments, the antigen binding domain specifically binds to an antigen that is at least 80% identical to an antigen selected from the group consisting of MUC16, UPK1B, VTCN1, TMPRSS3, TMEM238, Clorf186, TMPRSS4, CLDN6, CLDN8, STRA6, MSLN or CD73.

In certain embodiments, the antigen binding domain specifically binds to an antigen on a T cell, a B cell, a stellate cell, an endothelial cell, a tumor cell, an APC, a fibroblast cell, a fibrocyte cell, or a cell associated with the pathogenesis of fibrosis. In certain embodiments, the antigen binding domain specifically binds to an antigen on a T cell, an APC, and/or a B cell. In certain embodiments, the antigen binding domain specifically binds to an antigen on a hepatocyte. In certain embodiments, the antigen binding domain may specifically bind to an antigen selected from the group consisting of CTLA4, PD-1, OX40, LAG-3, GITR, GARP, CD25, CD27, PD-L1, TNFR2, ICOS, 41BB, CD70, CD73, CD38 or VTCN1. In certain embodiments, the antigen binding domain may specifically bind to an antigen selected from the group consisting of ASGR1 and ASGR2. In certain embodiments, the antigen binding domain specifically binds to an antigen on a stellate cell, an endothelial cell, a fibroblast cell, a fibrocyte cell, or a cell associated with the pathogenesis of fibrosis or cancer. In certain embodiments, the antigen binding domain may specifically bind to an antigen selected from the group consisting of, PDGFRβ, integrin αvβ1, integrin αvβ3, integrin αvβ6, integrin αvβ8, Endosialin, FAP, ADAM12, LRRC15, MMP14, PDPN, CDH11 and F2RL2. In certain embodiments, the antigen binding domain may specifically bind to an antigen selected from the group consisting of FAP, ADAM12, LRRC15, MMP14, PDPN, CDH11 and F2RL2. In certain embodiments, the antigen is LRRC15. In certain embodiments, the antigen binding domain specifically binds to an antigen on a tumor cell, a tumor antigen. In certain embodiments, the antigen binding domain specifically binds to an antigen selected from the group consisting of MUC16, UPK1B, VTCN1, TMPRSS3, TMEM238, Clorf186, TMPRSS4, CLDN6, CLDN8, STRA6, MSLN or CD73.

An antibody construct may comprise an antibody with modifications of at least one amino acid residue. Modifications may be substitutions, additions, mutations, deletions, or the like. An antibody modification can be an insertion of an unnatural amino acid.

An antigen binding domain may comprise at least 80% sequence identity to any sequence in Table A. An antigen binding domain may comprise a set of CDRs set forth in Table A. An antibody construct may comprise an antigen binding domain that binds an antigen, wherein the antigen binding domain comprises at least at least 80%, at least 90%, at least 95%, at least 97%, at least 98%, at least 99%, or at least 100% sequence identity to: a) HCDR1 comprising an amino acid sequence of SEQ ID NO: 1, HCDR2 comprising an amino acid sequence of SEQ ID NO: 2, HCDR3 comprising an amino acid sequence of SEQ ID NO: 3, LCDR1 comprising an amino acid sequence of SEQ ID NO: 4, LCDR2 comprising an amino acid sequence of SEQ ID NO: 5, and LCDR3 comprising an amino acid sequence of SEQ ID NO: 6; b) HCDR1 comprising an amino acid sequence of SEQ ID NO: 7, HCDR2 comprising an amino acid sequence of SEQ ID NO: 8, HCDR3 comprising an amino acid sequence of SEQ ID NO: 9, LCDR1 comprising an amino acid sequence of SEQ ID NO: 10, LCDR2 comprising an amino acid sequence of SEQ ID NO: 11, and LCDR3 comprising an amino acid sequence of SEQ ID NO: 12; c) HCDR1 comprising an amino acid sequence of SEQ ID NO: 13, HCDR2 comprising an amino acid sequence of SEQ ID NO: 14, HCDR3 comprising an amino acid sequence of SEQ ID NO: 15, LCDR1 comprising an amino acid sequence of SEQ ID NO: 16, LCDR2 comprising an amino acid sequence of SEQ ID NO: 17, and LCDR3 comprising an amino acid sequence of SEQ ID NO: 18; d) HCDR1 comprising an amino acid sequence of SEQ ID NO: 19, HCDR2 comprising an amino acid sequence of SEQ ID NO: 20, HCDR3 comprising an amino acid sequence of SEQ ID NO: 21, LCDR1 comprising an amino acid sequence of SEQ ID NO: 22, LCDR2 comprising an amino acid sequence of SEQ ID NO: 23, and LCDR3 comprising an amino acid sequence of SEQ ID NO: 24; e) HCDR1 comprising an amino acid sequence of SEQ ID NO: 25, HCDR2 comprising an amino acid sequence of SEQ ID NO: 26, HCDR3 comprising an amino acid sequence of SEQ ID NO: 27, LCDR1 comprising an amino acid sequence of SEQ ID NO: 28, LCDR2 comprising an amino acid sequence of SEQ ID NO: 29, and LCDR3 comprising an amino acid sequence of SEQ ID NO: 30; f) HCDR1 comprising an amino acid sequence of SEQ ID NO: 31, HCDR2 comprising an amino acid sequence of SEQ ID NO: 32, HCDR3 comprising an amino acid sequence of SEQ ID NO: 33, LCDR1 comprising an amino acid sequence of SEQ ID NO: 34, LCDR2 comprising an amino acid sequence of SEQ ID NO: 35, and LCDR3 comprising an amino acid sequence of SEQ ID NO: 36; g) HCDR1 comprising an amino acid sequence of SEQ ID NO: 37, HCDR2 comprising an amino acid sequence of SEQ ID NO: 38, HCDR3 comprising an amino acid sequence of SEQ ID NO: 39, LCDR1 comprising an amino acid sequence of SEQ ID NO: 40, LCDR2 comprising an amino acid sequence of SEQ ID NO: 41, and LCDR3 comprising an amino acid sequence of SEQ ID NO: 42; h) HCDR1 comprising an amino acid sequence of SEQ ID NO: 43, HCDR2 comprising an amino acid sequence of SEQ ID NO: 44, HCDR3 comprising an amino acid sequence of SEQ ID NO: 45, LCDR1 comprising an amino acid sequence of SEQ ID NO: 46, LCDR2 comprising an amino acid sequence of SEQ ID NO: 47, and LCDR3 comprising an amino acid sequence of SEQ ID NO: 48; i) HCDR1 comprising an amino acid sequence of SEQ ID NO: 49, HCDR2 comprising an amino acid sequence of SEQ ID NO: 50, HCDR3 comprising an amino acid sequence of SEQ ID NO: 51, LCDR1 comprising an amino acid sequence of SEQ ID NO: 52, LCDR2 comprising an amino acid sequence of SEQ ID NO: 53, and LCDR3 comprising an amino acid sequence of SEQ ID NO: 54; j) HCDR1 comprising an amino acid sequence of SEQ ID NO: 55, HCDR2 comprising an amino acid sequence of SEQ ID NO: 56, HCDR3 comprising an amino acid sequence of SEQ ID NO: 57, LCDR1 comprising an amino acid sequence of SEQ ID NO: 58, LCDR2 comprising an amino acid sequence of SEQ ID NO: 59, and LCDR3 comprising an amino acid sequence of SEQ ID NO: 60; k) HCDR1 comprising an amino acid sequence of SEQ ID NO: 61, HCDR2 comprising an amino acid sequence of SEQ ID NO: 62, HCDR3 comprising an amino acid sequence of SEQ ID NO: 63, LCDR1 comprising an amino acid sequence of SEQ ID NO: 64, LCDR2 comprising an amino acid sequence of SEQ ID NO: 65, and LCDR3 comprising an amino acid sequence of SEQ ID NO: 66; 1) HCDR1 comprising an amino acid sequence of SEQ ID NO: 67, HCDR2 comprising an amino acid sequence of SEQ ID NO: 68, HCDR3 comprising an amino acid sequence of SEQ ID NO: 69, LCDR1 comprising an amino acid sequence of SEQ ID NO: 70, LCDR2 comprising an amino acid sequence of SEQ ID NO: 71, and LCDR3 comprising an amino acid sequence of SEQ ID NO: 72; m) HCDR1 comprising an amino acid sequence of SEQ ID NO: 73, HCDR2 comprising an amino acid sequence of SEQ ID NO: 74, HCDR3 comprising an amino acid sequence of SEQ ID NO: 75, LCDR1 comprising an amino acid sequence of SEQ ID NO: 76, LCDR2 comprising an amino acid sequence of SEQ ID NO: 77, and LCDR3 comprising an amino acid sequence of SEQ ID NO: 78; n) HCDR1 comprising an amino acid sequence of SEQ ID NO: 73, HCDR2 comprising an amino acid sequence of SEQ ID NO: 74, HCDR3 comprising an amino acid sequence of SEQ ID NO: 75, LCDR1 comprising an amino acid sequence of SEQ ID NO: 79, LCDR2 comprising an amino acid sequence of SEQ ID NO: 80, and LCDR3 comprising an amino acid sequence of SEQ ID NO: 81; o) HCDR1 comprising an amino acid sequence of SEQ ID NO: 199, HCDR2 comprising an amino acid sequence of SEQ ID NO: 200, HCDR3 comprising an amino acid sequence of SEQ ID NO: 201, LCDR1 comprising an amino acid sequence of SEQ ID NO: 202, LCDR2 comprising an amino acid sequence of SEQ ID NO: 203, and LCDR3 comprising an amino acid sequence of SEQ ID NO: 204; p) HCDR1 comprising an amino acid sequence of SEQ ID NO: 205, HCDR2 comprising an amino acid sequence of SEQ ID NO: 206, HCDR3 comprising an amino acid sequence of SEQ ID NO: 207, LCDR1 comprising an amino acid sequence of SEQ ID NO: 208, LCDR2 comprising an amino acid sequence of SEQ ID NO: 209, and LCDR3 comprising an amino acid sequence of SEQ ID NO: 210 q) HCDR1 comprising an amino acid sequence of SEQ ID NO: 211, HCDR2 comprising an amino acid sequence of SEQ ID NO: 212, HCDR3 comprising an amino acid sequence of SEQ ID NO: 213, LCDR1 comprising an amino acid sequence of SEQ ID NO: 214, LCDR2 comprising an amino acid sequence of SEQ ID NO: 215, and LCDR3 comprising an amino acid sequence of SEQ ID NO: 216 r) HCDR1 comprising an amino acid sequence of SEQ ID NO: 217, HCDR2 comprising an amino acid sequence of SEQ ID NO: 218, HCDR3 comprising an amino acid sequence of SEQ ID NO: 219, LCDR1 comprising an amino acid sequence of SEQ ID NO: 220, LCDR2 comprising an amino acid sequence of SEQ ID NO: 221, and LCDR3 comprising an amino acid sequence of SEQ ID NO: 222; s) HCDR1 comprising an amino acid sequence of SEQ ID NO: 223, HCDR2 comprising an amino acid sequence of SEQ ID NO: 224, HCDR3 comprising an amino acid sequence of SEQ ID NO: 225, LCDR1 comprising an amino acid sequence of SEQ ID NO: 226, LCDR2 comprising an amino acid sequence of SEQ ID NO: 227, and LCDR3 comprising an amino acid sequence of SEQ ID NO: 228; or t) HCDR1 comprising an amino acid sequence of SEQ ID NO: 229, HCDR2 comprising an amino acid sequence of SEQ ID NO: 230, HCDR3 comprising an amino acid sequence of SEQ ID NO: 231, LCDR1 comprising an amino acid sequence of SEQ ID NO: 232, LCDR2 comprising an amino acid sequence of SEQ ID NO: 233, and LCDR3 comprising an amino acid sequence of SEQ ID NO: 234.

An antibody construct may comprise an antigen binding domain comprising one or more variable domains. An antibody construct may comprise an antigen binding domain comprising a light chain variable domain (V_{L} domain). A binding domain may comprise at least 80%, at least 90%, at least 95%, at least 97%, at least 98%, at least 99%, or at least 100% sequence identity to any V_{L} sequence in Table B. An antibody construct may comprise an antigen binding domain comprising a heavy chain variable domain (V_{H} domain). An antigen binding domain may comprise at least 80%, at least 90%, at least 95%, at least 97%, at least 98%, at least 99%, or at least 100% sequence identity to any V_{H} sequence in Table B. An antigen binding domain can comprise a pair of V_{H} and V_{L} sequences in Table B. An antigen binding domain can comprise at least 80% sequence identity to any sequence in Table B.

An antibody construct may comprise an antigen binding domain that specifically binds an antigen, wherein the antigen binding domain comprises: a) a V_{H} sequence having at least 80%, at least 90%, at least 95%, at least 97%, at least 98%, at least 99%, or at least 100% sequence identity to an amino acid sequence of SEQ ID NO: 83, and a V_{L} sequence having at least 80%, at least 90%, at least 95%, at least 97%, at least 98%, at least 99%, or at least 100% sequence identity to an amino acid sequence of SEQ ID NO: 84; b) a V_{H} sequence having at least 80%, at least 90%, at least 95%, at least 97%, at least 98%, at least 99%, or at least 100% sequence identity to an amino acid sequence of SEQ ID NO: 85, and a V_{L} sequence having at least 80%, at least 90%, at least 95%, at least 97%, at least 98%, at least 99%, or at least 100% sequence identity to an amino acid sequence of SEQ ID NO: 86; c) a V_{H} sequence having at least 80%, at least 90%, at least 95%, at least 97%, at least 98%, at least 99%, or at least 100% sequence identity to an amino acid sequence of SEQ ID NO: 87, and a V_{L} sequence having at least 80%, at least 90%, at least 95%, at least 97%, at least 98%, at least 99%, or at least 100% sequence identity to an amino acid sequence of SEQ ID NO: 88; d) a V_{H} sequence having at least 80%, at least 90%, at least 95%, at least 97%, at least 98%, at least 99%, or at least 100% sequence identity to an amino acid sequence of SEQ ID NO: 89, and a V_{L} sequence having at least 80%, at least 90%, at least 95%, at least 97%, at least 98%, at least 99%, or at least 100% sequence identity to an amino acid sequence of SEQ ID NO: 90; e) a V_{H} sequence having at least 80%, at least 90%, at least 95%, at least 97%, at least 98%, at least 99%, or at least 100% sequence identity to an amino acid sequence of SEQ ID NO: 91, and a V_{L} sequence having at least 80%, at least 90%, at least 95%, at least 97%, at least 98%, at least 99%, or at least 100% sequence identity to an amino acid sequence of SEQ ID NO: 92; f) a V_{H} sequence having at least 80%, at least 90%, at least 95%, at least 97%, at least 98%, at least 99%, or at least 100% sequence identity to an amino acid sequence of SEQ ID NO: 93, and a V_{L} sequence having at least 80%, at least 90%, at least 95%, at least 97%, at least 98%, at least 99%, or at least 100% sequence identity to an amino acid sequence of SEQ ID NO: 94; g) a V_{H} sequence having at least 80%, at least 90%, at least 95%, at least 97%, at least 98%, at least 99%, or at least 100% sequence identity to an amino acid sequence of SEQ ID NO: 95, and a V_{L} sequence having at least 80%, at least 90%, at least 95%, at least 97%, at least 98%, at least 99%, or at least 100% sequence identity to an amino acid sequence of SEQ ID NO: 96; h) a V_{H} sequence having at least 80%, at least 90%, at least 95%, at least 97%, at least 98%, at least 99%, or at least 100% sequence identity to an amino acid sequence of SEQ ID NO: 97, and a V_{L} sequence having at least 80%, at least 90%, at least 95%, at least 97%, at least 98%, at least 99%, or at least 100% sequence identity to an amino acid sequence of SEQ ID NO: 98; i) a V_{H} sequence having at least 80%, at least 90%, at least 95%, at least 97%, at least 98%, at least 99%, or at least 100% sequence identity to an amino acid sequence of SEQ ID NO: 99, and a V_{L} sequence having at least 80%, at least 90%, at least 95%, at least 97%, at least 98%, at least 99%, or at least 100% sequence identity to an amino acid sequence of SEQ ID NO: 100; j) a V_{H} sequence having at least 80%, at least 90%, at least 95%, at least 97%, at least 98%, at least 99%, or at least 100% sequence identity to an amino acid sequence of SEQ ID NO: 101, and a V_{L} sequence having at least 80%, at least 90%, at least 95%, at least 97%, at least 98%, at least 99%, or at least 100% sequence identity to an amino acid sequence of SEQ ID NO: 102; k) a V_{H} sequence having at least 80%, at least 90%, at least 95%, at least 97%, at least 98%, at least 99%, or at least 100% sequence identity to an amino acid sequence of SEQ ID NO: 101, and a V_{L} sequence having at least 80%, at least 90%, at least 95%, at least 97%, at least 98%, at least 99%, or at least 100% sequence identity to an amino acid sequence of SEQ ID NO: 103; 1) a V_{H} sequence having at least 80%, at least 90%, at least 95%, at least 97%, at least 98%, at least 99%, or at least 100% sequence identity to an amino acid sequence of SEQ ID NO: 104, and a V_{L} sequence having at least 80%, at least 90%, at least 95%, at least 97%, at least 98%, at least 99%, or at least 100% sequence identity to an amino acid sequence of SEQ ID NO: 105; m) a V_{H} sequence having at least 80%, at least 90%, at least 95%, at least 97%, at least 98%, at least 99%, or at least 100% sequence identity to an amino acid sequence of SEQ ID NO: 106, and a V_{L} sequence having at least 80%, at least 90%, at least 95%, at least 97%, at least 98%, at least 99%, or at least 100% sequence identity to an amino acid sequence of SEQ ID NO: 107; n) a V_{H} sequence having at least 80%, at least 90%, at least 95%, at least 97%, at least 98%, at least 99%, or at least 100% sequence identity to an amino acid sequence of SEQ ID NO: 109, and a V_{L} sequence having at least 80%, at least 90%, at least 95%, at least 97%, at least 98%, at least 99%, or at least 100% sequence identity to an amino acid sequence of SEQ ID NO: 108; o) a V_{H} sequence having at least 80%, at least 90%, at least 95%, at least 97%, at least 98%, at least 99%, or at least 100% sequence identity to an amino acid sequence of SEQ ID NO: 110, and a V_{L} sequence having at least 80%, at least 90%, at least 95%, at least 97%, at least 98%, at least 99%, or at least 100% sequence identity to an amino acid sequence of SEQ ID NO: 108; p) a V_{H} sequence having at least 80%, at least 90%, at least 95%, at least 97%, at least 98%, at least 99%, or at least 100% sequence identity to an amino acid sequence of SEQ ID NO: 111, and a V_{L} sequence having at least 80%, at least 90%, at least 95%, at least 97%, at least 98%, at least 99%, or at least 100% sequence identity to an amino acid sequence of SEQ ID NO: 112; q) a V_{H} sequence having at least 80%, at least 90%, at least 95%, at least 97%, at least 98%, at least 99%, or at least 100% sequence identity to an amino acid sequence of SEQ ID NO: 113, and a V_{L} sequence having at least 80%, at least 90%, at least 95%, at least 97%, at least 98%, at least 99%, or at least 100% sequence identity to an amino acid sequence of SEQ ID NO: 114; r) a V_{H} sequence having at least 80%, at least 90%, at least 95%, at least 97%, at least 98%, at least 99%, or at least 100% sequence identity to an amino acid sequence of SEQ ID NO: 115, and a V_{L} sequence having at least 80%, at least 90%, at least 95%, at least 97%, at least 98%, at least 99%, or at least 100% sequence identity to an amino acid sequence of SEQ ID NO: 116; s) a V_{H} sequence having at least 80%, at least 90%, at least 95%, at least 97%, at least 98%, at least 99%, or at least 100% sequence identity to an amino acid sequence of SEQ ID NO: 117, and a V_{L} sequence having at least 80%, at least 90%, at least 95%, at least 97%, at least 98%, at least 99%, or at least 100% sequence identity to an amino acid sequence of SEQ ID NO: 118; t) a V_{H} sequence having at least 80%, at least 90%, at least 95%, at least 97%, at least 98%, at least 99%, or at least 100% sequence identity to an amino acid sequence of SEQ ID NO: 117, and a V_{L} sequence having at least 80%, at least 90%, at least 95%, at least 97%, at least 98%, at least 99%, or at least 100% sequence identity to an amino acid sequence of SEQ ID NO: 119; u) a V_{H} sequence having at least 80%, at least 90%, at least 95%, at least 97%, at least 98%, at least 99%, or at least 100% sequence identity to an amino acid sequence of SEQ ID NO: 117, and a V_{L} sequence having at least 80%, at least 90%, at least 95%, at least 97%, at least 98%, at least 99%, or at least 100% sequence identity to an amino acid sequence of SEQ ID NO: 120; v) a V_{H} sequence having at least 80%, at least 90%, at least 95%, at least 97%, at least 98%, at least 99%, or at least 100% sequence identity to an amino acid sequence of SEQ ID NO: 117, and a V_{L} sequence having at least 80%, at least 90%, at least 95%, at least 97%, at least 98%, at least 99%, or at least 100% sequence identity to an amino acid sequence of SEQ ID NO: 121; w) a V_{H} sequence having at least 80%, at least 90%, at least 95%, at least 97%, at least 98%, at least 99%, or at least 100% sequence identity to an amino acid sequence of SEQ ID NO: 117, and a V_{L} sequence having at least 80%, at least 90%, at least 95%, at least 97%, at least 98%, at least 99%, or at least 100% sequence identity to an amino acid sequence of SEQ ID NO: 122; x) a V_{H} sequence having at least 80%, at least 90%, at least 95%, at least 97%, at least 98%, at least 99%, or at least 100% sequence identity to an amino acid sequence of SEQ ID NO: 123, and a V_{L} sequence having at least 80%, at least 90%, at least 95%, at least 97%, at least 98%, at least 99%, or at least 100% sequence identity to an amino acid sequence of SEQ ID NO: 124; y) a V_{H} sequence having at least 80%, at least 90%, at least 95%, at least 97%, at least 98%, at least 99%, or at least 100% sequence identity to an amino acid sequence of SEQ ID NO: 125, and a V_{L} sequence having at least 80%, at least 90%, at least 95%, at least 97%, at least 98%, at least 99%, or at least 100% sequence identity to an amino acid sequence of SEQ ID NO: 126; z) a V_{H} sequence having at least 80%, at least 90%, at least 95%, at least 97%, at least 98%, at least 99%, or at least 100% sequence identity to an amino acid sequence of SEQ ID NO: 127, and a V_{L} sequence having at least 80%, at least 90%, at least 95%, at least 97%, at least 98%, at least 99%, or at least 100% sequence identity to an amino acid sequence of SEQ ID NO: 128; aa) a V_{H} sequence having at least 80%, at least 90%, at least 95%, at least 97%, at least 98%, at least 99%, or at least 100% sequence identity to an amino acid sequence of SEQ ID NO: 130, and a V_{L} sequence having at least 80%, at least 90%, at least 95%, at least 97%, at least 98%, at least 99%, or at least 100% sequence identity to an amino acid sequence of SEQ ID NO: 129; bb) a V_{H} sequence having at least 80%, at least 90%, at least 95%, at least 97%, at least 98%, at least 99%, or at least 100% sequence identity to an amino acid sequence of SEQ ID NO: 131, and a V_{L} sequence having at least 80%, at least 90%, at least 95%, at least 97%, at least 98%, at least 99%, or at least 100% sequence identity to an amino acid sequence of SEQ ID NO: 132; cc) a V_{H} sequence having at least 80%, at least 90%, at least 95%, at least 97%, at least 98%, at least 99%, or at least 100% sequence identity to an amino acid sequence of SEQ ID NO: 133, and a V_{L} sequence having at least 80%, at least 90%, at least 95%, at least 97%, at least 98%, at least 99%, or at least 100% sequence identity to an amino acid sequence of SEQ ID NO: 134; dd) a V_{H} sequence having at least 80%, at least 90%, at least 95%, at least 97%, at least 98%, at least 99%, or at least 100% sequence identity to an amino acid sequence of SEQ ID NO: 135, and a V_{L} sequence having at least 80%, at least 90%, at least 95%, at least 97%, at least 98%, at least 99%, or at least 100% sequence identity to an amino acid sequence of SEQ ID NO: 136; ee) a V_{H} sequence having at least 80%, at least 90%, at least 95%, at least 97%, at least 98%, at least 99%, or at least 100% sequence identity to an amino acid sequence of SEQ ID NO: 137, and a V_{L} sequence having at least 80%, at least 90%, at least 95%, at least 97%, at least 98%, at least 99%, or at least 100% sequence identity to an amino acid sequence of SEQ ID NO: 138; ff) a V_{H} sequence having at least 80%, at least 90%, at least 95%, at least 97%, at least 98%, at least 99%, or at least 100% sequence identity to an amino acid sequence of SEQ ID NO: 140, and a V_{L} sequence having at least 80%, at least 90%, at least 95%, at least 97%, at least 98%, at least 99%, or at least 100% sequence identity to an amino acid sequence of SEQ ID NO: 139; gg) a V_{H} sequence having at least 80%, at least 90%, at least 95%, at least 97%, at least 98%, at least 99%, or at least 100% sequence identity to an amino acid sequence of SEQ ID NO: 141, and a V_{L} sequence having at least 80%, at least 90%, at least 95%, at least 97%, at least 98%, at least 99%, or at least 100% sequence identity to an amino acid sequence of SEQ ID NO: 142; hh) a V_{H} sequence having at least 80%, at least 90%, at least 95%, at least 97%, at least 98%, at least 99%, or at least 100% sequence identity to an amino acid sequence of SEQ ID NO: 143, and a V_{L} sequence having at least 80%, at least 90%, at least 95%, at least 97%, at least 98%, at least 99%, or at least 100% sequence identity to an amino acid sequence of SEQ ID NO: 144; ii) a V_{H} sequence having at least 80%, at least 90%, at least 95%, at least 97%, at least 98%, at least 99%, or at least 100% sequence identity to an amino acid sequence of SEQ ID NO: 145, and a V_{L} sequence having at least 80%, at least 90%, at least 95%, at least 97%, at least 98%, at least 99%, or at least 100% sequence identity to an amino acid sequence of SEQ ID NO: 146; jj) a V_{H} sequence having at least 80%, at least 90%, at least 95%, at least 97%, at least 98%, at least 99%, or at least 100% sequence identity to an amino acid sequence of SEQ ID NO: 147, and a V_{L} sequence having at least 80%, at least 90%, at least 95%, at least 97%, at least 98%, at least 99%, or at least 100% sequence identity to an amino acid sequence of SEQ ID NO: 148; kk) a V_{H} sequence having at least 80%, at least 90%, at least 95%, at least 97%, at least 98%, at least 99%, or at least 100% sequence identity to an amino acid sequence of SEQ ID NO: 149, and a V_{L} sequence having at least 80%, at least 90%, at least 95%, at least 97%, at least 98%, at least 99%, or at least 100% sequence identity to an amino acid sequence of SEQ ID NO: 150; ll) a V_{H} sequence having at least 80%, at least 90%, at least 95%, at least 97%, at least 98%, at least 99%, or at least 100% sequence identity to an amino acid sequence of SEQ ID NO: 151, and a V_{L} sequence having at least 80%, at least 90%, at least 95%, at least 97%, at least 98%, at least 99%, or at least 100% sequence identity to an amino acid sequence of SEQ ID NO: 153; mm) a V_{H} sequence having at least 80%, at least 90%, at least 95%, at least 97%, at least 98%, at least 99%, or at least 100% sequence identity to an amino acid sequence of SEQ ID NO: 152, and a V_{L} sequence having at least 80%, at least 90%, at least 95%, at least 97%, at least 98%, at least 99%, or at least 100% sequence identity to an amino acid sequence of SEQ ID NO: 153; nn) a V_{H} sequence having at least 80%, at least 90%, at least 95%, at least 97%, at least 98%, at least 99%, or at least 100% sequence identity to an amino acid sequence of SEQ ID NO: 154, and a V_{L} sequence having at least 80%, at least 90%, at least 95%, at least 97%, at least 98%, at least 99%, or at least 100% sequence identity to an amino acid sequence of SEQ ID NO: 155; oo) a V_{H} sequence having at least 80%, at least 90%, at least 95%, at least 97%, at least 98%, at least 99%, or at least 100% sequence identity to an amino acid sequence of SEQ ID NO: 156, and a V_{L} sequence having at least 80%, at least 90%, at least 95%, at least 97%, at least 98%, at least 99%, or at least 100% sequence identity to an amino acid sequence of SEQ ID NO: 157; pp) a V_{H} sequence having at least 80%, at least 90%, at least 95%, at least 97%, at least 98%, at least 99%, or at least 100% sequence identity to an amino acid sequence of SEQ ID NO: 158, and a V_{L} sequence having at least 80%, at least 90%, at least 95%, at least 97%, at least 98%, at least 99%, or at least 100% sequence identity to an amino acid sequence of SEQ ID NO: 159; qq) a V_{H} sequence having at least 80%, at least 90%, at least 95%, at least 97%, at least 98%, at least 99%, or at least 100% sequence identity to an amino acid sequence of SEQ ID NO: 160, and a V_{L} sequence having at least 80%, at least 90%, at least 95%, at least 97%, at least 98%, at least 99%, or at least 100% sequence identity to an amino acid sequence of SEQ ID NO: 161; rr) a V_{H} sequence having at least 80%, at least 90%, at least 95%, at least 97%, at least 98%, at least 99%, or at least 100% sequence identity to an amino acid sequence of SEQ ID NO: 162, and a V_{L} sequence having at least 80%, at least 90%, at least 95%, at least 97%, at least 98%, at least 99%, or at least 100% sequence identity to an amino acid sequence of SEQ ID NO: 163; ss) a V_{H} sequence having at least 80%, at least 90%, at least 95%, at least 97%, at least 98%, at least 99%, or at least 100% sequence identity to an amino acid sequence of SEQ ID NO: 164, and a V_{L} sequence having at least 80%, at least 90%, at least 95%, at least 97%, at least 98%, at least 99%, or at least 100% sequence identity to an amino acid sequence of SEQ ID NO: 167; tt) a V_{H} sequence having at least 80%, at least 90%, at least 95%, at least 97%, at least 98%, at least 99%, or at least 100% sequence identity to an amino acid sequence of SEQ ID NO: 164, and a V_{L} sequence having at least 80%, at least 90%, at least 95%, at least 97%, at least 98%, at least 99%, or at least 100% sequence identity to an amino acid sequence of SEQ ID NO: 168; uu) a V_{H} sequence having at least 80%, at least 90%, at least 95%, at least 97%, at least 98%, at least 99%, or at least 100% sequence identity to an amino acid sequence of SEQ ID NO: 165, and a V_{L} sequence having at least 80%, at least 90%, at least 95%, at least 97%, at least 98%, at least 99%, or at least 100% sequence identity to an amino acid sequence of SEQ ID NO: 167; vv) a V_{H} sequence having at least 80%, at least 90%, at least 95%, at least 97%, at least 98%, at least 99%, or at least 100% sequence identity to an amino acid sequence of SEQ ID NO: 165, and a V_{L} sequence having at least 80%, at least 90%, at least 95%, at least 97%, at least 98%, at least 99%, or at least 100% sequence identity to an amino acid sequence of SEQ ID NO: 168; ww) a V_{H} sequence having at least 80%, at least 90%, at least 95%, at least 97%, at least 98%, at least 99%, or at least 100% sequence identity to an amino acid sequence of SEQ ID NO: 166, and a V_{L} sequence having at least 80%, at least 90%, at least 95%, at least 97%, at least 98%, at least 99%, or at least 100% sequence identity to an amino acid sequence of SEQ ID NO: 167; xx) a V_{H} sequence having at least 80%, at least 90%, at least 95%, at least 97%, at least 98%, at least 99%, or at least 100% sequence identity to an amino acid sequence of SEQ ID NO: 166, and a V_{L} sequence having at least 80%, at least 90%, at least 95%, at least 97%, at least 98%, at least 99%, or at least 100% sequence identity to an amino acid sequence of SEQ ID NO: 168; yy) a V_{H} sequence having at least 80%, at least 90%, at least 95%, at least 97%, at least 98%, at least 99%, or at least 100% sequence identity to an amino acid sequence of SEQ ID NO: 169, and a V_{L} sequence having at least 80%, at least 90%, at least 95%, at least 97%, at least 98%, at least 99%, or at least 100% sequence identity to an amino acid sequence of SEQ ID NO:170; zz) a V_{H} sequence having at least 80%, at least 90%, at least 95%, at least 97%, at least 98%, at least 99%, or at least 100% sequence identity to an amino acid sequence of SEQ ID NO: 171, and a V_{L} sequence having at least 80%, at least 90%, at least 95%, at least 97%, at least 98%, at least 99%, or at least 100% sequence identity to an amino acid sequence of SEQ ID NO: 172; aaa) a V_{H} sequence having at least 80%, at least 90%, at least 95%, at least 97%, at least 98%, at least 99%, or at least 100% sequence identity to an amino acid sequence of SEQ ID NO: 174, and a V_{L} sequence having at least 80%, at least 90%, at least 95%, at least 97%, at least 98%, at least 99%, or at least 100% sequence identity to an amino acid sequence of SEQ ID NO: 173; bbb) a V_{H} sequence having at least 80%, at least 90%, at least 95%, at least 97%, at least 98%, at least 99%, or at least 100% sequence identity to an amino acid sequence of SEQ ID NO: 175, and a V_{L} sequence having at least 80%, at least 90%, at least 95%, at least 97%, at least 98%, at least 99%, or at least 100% sequence identity to an amino acid sequence of SEQ ID NO: 176; ccc) a V_{H} sequence having at least 80%, at least 90%, at least 95%, at least 97%, at least 98%, at least 99%, or at least 100% sequence identity to an amino acid sequence of SEQ ID NO: 177, and a V_{L} sequence having at least 80%, at least 90%, at least 95%, at least 97%, at least 98%, at least 99%, or at least 100% sequence identity to an amino acid sequence of SEQ ID NO: 178; ddd) a V_{H} sequence having at least 80%, at least 90%, at least 95%, at least 97%, at least 98%, at least 99%, or at least 100% sequence identity to an amino acid sequence of SEQ ID NO: 179, and a V_{L} sequence having at least 80%, at least 90%, at least 95%, at least 97%, at least 98%, at least 99%, or at least 100% sequence identity to an amino acid sequence of SEQ ID NO: 180; eee) a V_{H} sequence having at least 80%, at least 90%, at least 95%, at least 97%, at least 98%, at least 99%, or at least 100% sequence identity to an amino acid sequence of SEQ ID NO: 181, and a V_{L} sequence having at least 80%, at least 90%, at least 95%, at least 97%, at least 98%, at least 99%, or at least 100% sequence identity to an amino acid sequence of SEQ ID NO: 182; fff) a V_{H} sequence having at least 80%, at least 90%, at least 95%, at least 97%, at least 98%, at least 99%, or at least 100% sequence identity to an amino acid sequence of SEQ ID NO: 183, and a V_{L} sequence having at least 80%, at least 90%, at least 95%, at least 97%, at least 98%, at least 99%, or at least 100% sequence identity to an amino acid sequence of SEQ ID NO: 184; ggg) a V_{H} sequence having at least 80%, at least 90%, at least 95%, at least 97%, at least 98%, at least 99%, or at least 100% sequence identity to an amino acid sequence of SEQ ID NO: 185, and a V_{L} sequence having at least 80%, at least 90%, at least 95%, at least 97%, at least 98%, at least 99%, or at least 100% sequence identity to an amino acid sequence of SEQ ID NO: 186; hhh) a V_{H} sequence having at least 80%, at least 90%, at least 95%, at least 97%, at least 98%, at least 99%, or at least 100% sequence identity to an amino acid sequence of SEQ ID NO: 187, and a V_{L} sequence having at least 80%, at least 90%, at least 95%, at least 97%, at least 98%, at least 99%, or at least 100% sequence identity to an amino acid sequence of SEQ ID NO: 188; iii) a V_{H} sequence having at least 80%, at least 90%, at least 95%, at least 97%, at least 98%, at least 99%, or at least 100% sequence identity to an amino acid sequence of SEQ ID NO: 189, and a V_{L} sequence having at least 80%, at least 90%, at least 95%, at least 97%, at least 98%, at least 99%, or at least 100% sequence identity to an amino acid sequence of SEQ ID NO: 190; jjj) a V_{H} sequence having at least 80%, at least 90%, at least 95%, at least 97%, at least 98%, at least 99%, or at least 100% sequence identity to an amino acid sequence of SEQ ID NO: 191, and a V_{L} sequence having at least 80%, at least 90%, at least 95%, at least 97%, at least 98%, at least 99%, or at least 100% sequence identity to an amino acid sequence of SEQ ID NO: 192; kkk) a V_{H} sequence having at least 80%, at least 90%, at least 95%, at least 97%, at least 98%, at least 99%, or at least 100% sequence identity to an amino acid sequence of SEQ ID NO: 193, and a V_{L} sequence having at least 80%, at least 90%, at least 95%, at least 97%, at least 98%, at least 99%, or at least 100% sequence identity to an amino acid sequence of SEQ ID NO: 194; lll) a V_{H} sequence having at least 80%, at least 90%, at least 95%, at least 97%, at least 98%, at least 99%, or at least 100% sequence identity to an amino acid sequence of SEQ ID NO: 195, and a V_{L} sequence having at least 80%, at least 90%, at least 95%, at least 97%, at least 98%, at least 99%, or at least 100% sequence identity to an amino acid sequence of SEQ ID NO: 196; or mmm) a V_{H} sequence having at least 80%, at least 90%, at least 95%, at least 97%, at least 98%, at least 99%, or at least 100% sequence identity to an amino acid sequence of SEQ ID NO: 197, and a V_{L} sequence having at least 80%, at least 90%, at least 95%, at least 97%, at least 98%, at least 99%, or at least 100% sequence identity to an amino acid sequence of SEQ ID NO: 198.

An antibody construct may comprise a sequence from Table A and/or Table B. An antibody construct may comprise a set of CDR sequences from Table A and/or a pair of V_{H} and V_{L} sequences from Table B.

TABLE A
Antibody CDRs

| ANTIBODY | REGION | SEQ ID NO: | SEQUENCE: |
|---|---|---|---|
| Ipilumumab | HCDR1 | 1 | GFTFSSYT |
| | HCDR2 | 2 | ISYDGNNK |
| | HCDR3 | 3 | ARTGWLGPFDY |
| | LCDR1 | 4 | QSVGSSY |
| | LCDR2 | 5 | SSY |
| | LCDR3 | 6 | QQYGSSPWT |
| Opdivo® | HCDR1 | 7 | GITFSNSG |
| (nivolumab) | HCDR2 | 8 | IWYDGSKR |
| | HCDR3 | 9 | ATNDDY |
| | LCDR1 | 10 | QSVSSYL |
| | LCDR2 | 11 | DAS |
| | LCDR3 | 12 | QQSSNWPRT |
| Keytruda® | HCDR1 | 13 | GYTFTNYY |
| (pembrolizumab) | HCDR2 | 14 | INPSNGGT |
| | HCDR3 | 15 | ARRDYRFDMGFDY |
| | LCDR1 | 16 | KGVSTSGYSY |
| | LCDR2 | 17 | LAS |
| | LCDR3 | 18 | QHSRDLPLT |
| Vonlerolizumab | HCDR1 | 19 | GYTFTDSY |
| | HCDR2 | 20 | MYPDNGDS |
| | HCDR3 | 21 | VLAPRWYFSV |
| | LCDR1 | 22 | QDISNY |
| | LCDR2 | 23 | YTS |
| | LCDR3 | 24 | QQGHTLPPT |
| Varlilumab | HCDR1 | 25 | GFTFSSYD |
| | HCDR2 | 26 | IWYDGSNK |
| | HCDR3 | 27 | ARGSGNWGFFDY |
| | LCDR1 | 28 | QGISRW |
| | LCDR2 | 29 | AAS |
| | LCDR3 | 30 | QQYNTYPRT |
| Zinbryta® | HCDR1 | 31 | GYTFTSYR |
| (Daclizumab) | HCDR2 | 32 | INPSTGYT |
| | HCDR3 | 33 | ARGGGVFDY |
| | LCDR1 | 34 | SSSISY |
| | LCDR2 | 35 | TTS |
| | LCDR3 | 36 | HQRSTYPLT |
| Antibody to GITR | HCDR1 | 37 | SYGMH |
| | HCDR2 | 38 | VIWYEGSNKYYADSVKG |
| | HCDR3 | 39 | GGSMVRGDYYYGMDV |
| | LCDR1 | 40 | RASQGISSALA |
| | LCDR2 | 41 | DASSLES |
| | LCDR3 | 42 | QQFNSYPYT |
| Antibody to LAG-3 | HCDR1 | 43 | DYYWN |
| | HCDR2 | 44 | EINHRGSTNSNPSLKS |
| | HCDR3 | 45 | GYSDYEYNWFDP |
| | LCDR1 | 46 | RASQSISSYLA |
| | LCDR2 | 47 | DASNRAT |
| | LCDR3 | 48 | QQRSNWPLT |
| Utomilumab | HCDR1 | 50 | GYSFSTYW |
| | HCDR2 | 51 | IYPGDSYT |
| | HCDR3 | 52 | ARGYGIFDY |
| | LCDR1 | 53 | NIGDQY |
| | LCDR2 | 54 | QDK |
| | LCDR3 | 55 | ATYTGFGSLAV |
| Antibody to | HCDR1 | 56 | GYTFTDYN |
| TNFR2 variant 1 | HCDR2 | 57 | INPNYEST |
| | HCDR3 | 58 | RDKGWYFDV |
| | LCDR1 | 59 | SSVKN |
| | LCDR2 | 60 | YTS |
| | LCDR3 | 61 | QQFTSSPYT |
| Antibody to | HCDR1 | 62 | GFSLSTSGMG |
| TNFR2 variant 2 | HCDR2 | 63 | IWWDDDK |
| | HCDR3 | 64 | ARLTGTRYFDY |
| | LCDR1 | 65 | QDINKF |
| | LCDR2 | 66 | YTS |
| | LCDR3 | 67 | LQYGNLWT |
| Antibody to | HCDR1 | 68 | GYTFTDYS |
| TNFR2 variant 3 | HCDR2 | 69 | INTETGEP |
| | HCDR3 | 70 | ATYYGSSYVPDY |
| | LCDR1 | 71 | QNVGTA |
| | LCDR2 | 72 | WTS |
| | LCDR3 | 73 | QYSDYPYT |
| Antibody to | HCDR1 | 74 | GYTFTDY |
| TNFR2 variant 4 | HCDR2 | 75 | WVDPEYGS |
| | HCDR3 | 76 | ARDDGSYSPFDY |
| | LCDR1 (major) | 77 | QNINKY |
| | LCDR2 (major) | 78 | YTS |
| | LCDR3 (major) | 79 | LQYVNLLT |
| | LCDR1 (minor) | 80 | ENVVTY |
| | LCDR2 (minor) | 81 | GAS |
| | LCDR3 (minor) | 82 | QGYSYPYT |
| Antibody | HCDR1 | 199 | DYYIH |
| huAD208.4.1 to | | | |
| LRRC15 | | | |
| | HCDR2 | 200 | LVYPYIGGTNYNQKFKG |
| | HCDR3 | 201 | GDNKYDAMDY |
| | LCDR1 | 202 | RASQSVSTSSYSYMH |
| | LCDR2 | 203 | YASSLES |
| | LCDR3 | 204 | EQSWEIRT |
| Antibody | HCDR1 | 205 | NYWMH |
| huAD208.12.1 to | | | |
| LRRC15 | | | |
| | HCDR2 | 206 | MIHPNSGSTKHNEKFRG |
| | HCDR3 | 207 | SDFGNYRWYFDV |
| | LCDR1 | 208 | RASQSSSNNLH |
| | LCDR2 | 209 | YVSQSIS |
| | LCDR3 | 210 | QQSNSWPFT |
| Antibody | HCDR1 | 211 | DYYIH |
| huAD208.14.1 to | | | |
| LRRC15 | | | |
| | HCDR2 | 212 | LVYPYIGGSSYNQQFKG |
| | HCDR3 | 213 | GDNNYDAMDY |
| | LCDR1 | 214 | RASQSVSTSTYNYMH |
| | LCDR2 | 215 | YASNLES |
| | LCDR3 | 216 | HHTWEIRT |
| Antibody hu139.10 | HCDR1 | 217 | SYGVH |
| to LRRC15 | | | |
| | HCDR2 | 218 | VIWAGGSTNYNSALMS |
| | HCDR3 | 219 | HMITEDYYGMDY |
| | LCDR1 | 220 | KSSQSLLNSRTRKNYLA |
| | LCDR2 | 221 | WASTRES |
| | LCDR3 | 222 | KQSYNLPT |
| Antibody | HCDR1 | 223 | NYWLG |
| muAD210.40.9 to | | | |
| LRRC15 | | | |
| | HCDR2 | 224 | DIYPGGGNTYYNEKLKG |
| | HCDR3 | 225 | WGDKKGNYFAY |
| | LCDR1 | 226 | TASSSVYSSYLH |
| | LCDR2 | 227 | STSNLAS |
| | LCDR3 | 228 | HQYHRSPT |
| Antibody | HCDR1 | 229 | NFGMN |
| muAD209.9.1 to | | | |
| LRRC15 | | | |
| | HCDR2 | 230 | WINLYTGEPTFADDFKG |
| | HCDR3 | 231 | KGETYYRYDGFAY |
| | LCDR1 | 232 | RSSKSLLHSNGNTHLY |
| | LCDR2 | 233 | RMSNLAS |
| | LCDR3 | 234 | MQLLEYPYT |

TABLE B
Antibody V_{H} sequence and V_{L} sequences

| | | SEQ ID | |
|---|---|---|---|
| ANTIBODY | REGION | NO: | SEQUENCE: |
| Ipilumumab | V_{H} | 83 | QVQLVESGGGVVQPGRSLRLSCAASGFTFSSYT |
| | | | MHWVRQAPGKGLEWVTFISYDGNNKYYADSV |
| | | | KGRFTISRDNSKNTLYLQMNSLRAEDTAIYYCA |
| | | | RTGWLGPFDYWGQGTLVTVSS |
| | V_{L} | 84 | EIVLTQSPGTLSLSPGERATLSCRASQSVGSSYL |
| | | | AWYQQKPGQAPRLLIYGAFSRATGIPDRFSGSG |
| | | | SGTDFTLTISRLEPEDFAVYYCQQYGSSPWTFG |
| | | | QGTKVEIK |
| Opdivo® | V_{H} | 85 | QVQLVESGGGVVQPGRSLRLDCKASGITFSNSG |
| (nivolumab) | | | MHWVRQAPGKGLEWVAVIWYDGSKRYYADS |
| | | | VKGRFTISRDNSKNTLFLQMNSLRAEDTAVYYC |
| | | | ATNDDYWGQGTLVTVSS |
| | V_{L} | 86 | EIVLTQSPATLSLSPGERATLSCRASQSVSSYLA |
| | | | WYQQKPGQAPRLLIYDASNRATGIPARFSGSGS |
| | | | GTDFTLTISSLEPEDFAVYYCQQSSNWPRTFGQ |
| | | | GTKVEIK |
| Keytruda® | V_{H} | 87 | QVQLVQSGVEVKKPGASVKVSCKASGYTFTNY |
| (pembrolizumab) | | | YMYWVRQAPGQGLEWMGGINPSNGGTNFNEK |
| | | | FKNRVTLTTDSSTTTAYMELKSLQFDD |
| | | | TAVYYCARRDYRFDMGFDYWGQGTTVTVSS |
| | V_{L} | 88 | EIVLTQSPATLSLSPGERATLSCRASKGVSTSGY |
| | | | SYLHWYQQKPGQAPRLLIYLASYLESGVPARFS |
| | | | GSGSGTDFTLTISSLEPEDFAVYYCQHSRDLPLT |
| | | | FGGGTKVEIK |
| Atezolizumab | V_{H} | 89 | EVQLVESGGGLVQPGGSLRLSCAASGFTFSDSW |
| | | | IHWVRQAPGKGLEWVAWISPYGGSTYYADSVK |
| | | | GRFTISADTSKNTAYLQMNSLRAEDTAVYYCA |
| | | | RRHWPGGFDYWGQGTLVTVSS |
| | V_{L} | 90 | DIQMTQSPSSLSASVGDRVTITCRASQDVSTAV |
| | | | AWYQQKPGKAPKLLIYSASFLYSGVPSRFSGSG |
| | | | SGTDFTLTISSLQPEDFATYYCQQYLYHPATFGQ |
| | | | GTKVEIK |
| Durvalumab | V_{H} | 91 | EVQLVESGGGLVQPGGSLRLSCAASGFTFSRYW |
| | | | MSWVRQAPGKGLEWVANIKQDGSEKYYVDSV |
| | | | KGRFTISRDNAKNSLYLQMNSLRAEDTAVYYC |
| | | | AREGGWFGELAFDYWGQGTLVTVSS |
| | V_{L} | 92 | EIVLTQSPGTLSLSPGERATLSCRASQRVSSSYL |
| | | | AWYQQKPGQAPRLLIYDASSRATGIPDRFSGSG |
| | | | SGTDFTLTISRLEPEDFAVYYCQQYGSLPWTFG |
| | | | QGTKVEIK |
| MDX-1106 | V_{H} | 93 | QVQLVQSGAEVKKPGSSVKVSCKTSGDTFSTY |
| | | | AISWVRQAPGQGLEWMGGIIPIFGKAHYAQKFQ |
| | | | GRVTITADESTSTAYMELSSLRSEDTAVYFCAR |
| | | | KFHFVSGSPFGMDVWGQGTTVTVSS |
| | V_{L} | 94 | EIVLTQSPATLSLSPGERATLSCRASQSVSSYLA |
| | | | WYQQKPGQAPRLLIYDASNRATGIPARFSGSGS |
| | | | GTDFTLTISSLEPEDFAVYYCQQRSNWPTFGQG |
| | | | TKVEIK |
| Antibody to | V_{H} | 95 | EVQLQQSGAELVKPGASVKISCKASGYTFTDYN |
| TNFR2 variant 1 | | | MDWVKQSHGKSLEWIGDINPNYESTSYNQKFK |
| | | | GKATLTVDKSSSTAYMEVRSLTSEDTAVFYCA |
| | | | RDKGWYFDVWGAGTTVTVSS |
| | V_{L} | 96 | ENVLTQSPAIMSASLGEKVTMSCRASSSVKNM |
| | | | YWYQQKSDASPKLWIYYTSNLAPGVPARFSGS |
| | | | GSGNSYSLTISSMEGEDAATYYCQQFTSSPYTF |
| | | | GGGTKLELK |
| Antibody to | V_{H} | 97 | QVTLKESGPGILQPSQTLSLTCSFSGFSLSTSGM |
| TNFR2 variant 2 | | | GVGWIRQPSGKGLEWLAHIWWDDDKFYNPSL |
| | | | KSQLTISKDTSRNQVFLKLTSVVTADTATYYCA |
| | | | RLTGTRYFDYWGQGTTLTVSS |
| | V_{L} | 98 | DVQMTQSPSSLSASLGGKVTITCKASQDINKFIA |
| | | | WYQHKPGKGPRLLIHYTSTLQPGIPSKFSGSGSG |
| | | | RDYSFSISNLEPEDIATYYCLQYGNLWTFGGGT |
| | | | KLEIT |
| Antibody to | V_{H} | 99 | QIQLVQSGPELKKPGETVKISCKASGYTFTDYS |
| TNFR2 variant 3 | | | MHWVKQAPGKGLKWMGWINTETGEPTYADD |
| | | | FKGRFAFSSETSTSTAYLQINNLKNDDTTTYFCA |
| | | | TYYGSSYVPDYWGQGTSLTVSS |
| | V_{L} | 100 | DIVMTQSHKFMSTSVGDRVSITCKASQNVGTA |
| | | | VAWYQHKPGQSPKLLIYWTSSRHTGVPDRFTG |
| | | | SGSGTEFTLTISNVQSEDLADYFCHQYSDYPYTF |
| | | | GGGTKLEIK |
| Antibody to | V_{H} | 101 | EVQLQQSGPEVGRPGSSVKISCKASGYTFTDYI |
| TNFR2 variant 4 | | | MHWVKQSPGQGLEWIGWVDPEYGSTDYAEKF |
| | | | KKKATLTADTSSNTAYIQLSSLTSEDTATYFCA |
| | | | RDDGSYSPFDYWGQGVMVTVSS |
| | V_{L} | 102 | DIQMTQSPPSLSASLGDKVTITCQASQNINKYIA |
| | (major) | | WYQQKPGKAPRLLIRYTSTLESGTPSRFSGSGSG |
| | | | RDYSFSISNVESEDIASYYCLQYVNLLTFGAGTK |
| | | | LEIK |
| | V_{L} | 103 | NIVMTQSPKSMSMSVGERVTLTCKASENVVTY |
| | (minor) | | VSWYQQKPEQSPKLLIYGASNRYTGVPDRFTGS |
| | | | GSATDFTLTISSVQAEDLADYHCGQGYSYPYTF |
| | | | GGGTKLEIK |
| Antibody to | V_{H} | 104 | EVQLVESGGGLVQPGGSLRLSCAASGFTFSDYA |
| TNFR2 variant 5 | | | MSWVRQAPGKGLEWVAVISENGSDTYYADSV |
| | | | KGRFTISRDDSKNTLYLQMNSLRAEDTAVYYC |
| | | | ARDRGGAVSYFDVWGQGTLVTVSS |
| | V_{L} | 105 | DIQMTQSPSSLSASVGDRVTITCRASQDVSSYLA |
| | | | WYQQKPGKAPKLLIYAASSLESGVPSRFSGSGS |
| | | | GTDFTLTISSLQPEDFATYYCQQYNSLPYTFGQG |
| | | | TKVEIKRT |
| Vonlerolizumab | V_{H} | 106 | EVQLVQSGAEVKKPGASVKVSCKASGYTFTDS |
| | | | YMSWVRQAPGQGLEWIGDMYPDNGDSSYNQK |
| | | | FRERVTITRDTSTSTAYLELSSLRSEDTAVYYCV |
| | | | LAPRWYFSVWGQGTLVTVSS |
| | V_{L} | 107 | DIQMTQSPSSLSASVGDRVTITCRASQDISNYLN |
| | | | WYQQKPGKAPKLLIYYTSRLRSGVPSRFSGSGS |
| | | | GTDFTLTISSLQPEDFATYYCQQGHTLPPTFGQG |
| | | | TKVEIK |
| TRX518 | V_{L} | 108 | EIVMTQSPATLSVSPGERATLSCKASQNVGTNV |
| | | | AWYQQKPGQAPRLLIYSASYRYSGIPARFSGSG |
| | | | SGTEFTLTISSLQSEDFAVYYCQQYNTDPLTFGG |
| | | | GTKVEIK |
| | V_{H} | 109 | QVTLRESGPALVKPTQTLTLTCTFSGFSLSTSGM |
| | | | GVGWIRQPPGKALEWLAHIWWDDDKYYNPSL |
| | | | KSRLTISKDTSKNQVVLTMTNMDPVDTATYYC |
| | | | ARTRRYFPFAYWGQGTLVTVSS |
| | V_{H} | 110 | QVTLRESGPALVKPTQTLTLTCTFSGFSLSTSGM |
| | | | GVGWIRQPPGKALEWLAHIWWDDDKYYQPSL |
| | | | KSRLTISKDTSKNQVVLTMTNMDPVDTATYYC |
| | | | ARTRRYFPFAYWGQGTLVTVSS |
| Antibody to | V_{H} | 111 | QVQLQQWGAGLLKPSETLSLTCAVYGGSFSDY |
| LAG-3 | | | YWNWIRQPPGKGLEWIGEINHRGSTNSNPSLKS |
| | | | RVTLSLDTSKNQFSLKLRSVTAADTAVYYCAFG |
| | | | YSDYEYNWFDPWGQGTLVTVSS |
| | V_{L} | 112 | EIVLTQSPATLSLSPGERATLSCRASQSISSYLAW |
| | | | YQQKPGQAPRLLIYDASNRATGIPARFSGSGSG |
| | | | TDFTLTISSLEPEDFAVYYCQQRSNWPLTFGQG |
| | | | TNLEIK |
| Antibody to | V_{H} | 113 | MAVLALLFCLVTFPSCILSQVQLKESGPGLVAPS |
| GARP variant 1 | | | QSLSITCTVSGFSLTGYGINWVRQPPGKGLEWL |
| | | | GMIWSDGSTDYNSVLTSRLRISKDNSNSQVFLK |
| | | | MNSLQVDDTARYYCARDRNYYDYDGAMDYW |
| | | | GQGTSVTVSS |
| | V_{L} | 114 | QVQLKESGPGLVAPSQSLSITCTVSGFSLTGYGI |
| | | | NWVRQPPGKGLEWLGMIWSDGSTDYNSVLTSR |
| | | | LRISKDNSNSQVFLKMNSLQVDDTARYYCARD |
| | | | RNYYDYDGAMDYWGQGTSVTVSS |
| Antibody to | V_{H} | 115 | MKFPSQLLLFLLFRITGIICDIQVTQSSSYLSVSL |
| GARP variant 2 | | | GDRVTITCKASDHIKNWLAWYQQKPGIAPRLL |
| | | | VSGATSLEAGVPSRFSGSGSGKNFTLSITSLQTE |
| | | | DVATYYCQQYWSTPWTFGGGTTLEIR |
| | V_{L} | 116 | DIQVTQSSSYLSVSLGDRVTITCKASDHIKNWL |
| | | | AWYQQKPGIAPRLLVSGATSLEAGVPSRFSGSG |
| | | | SGKNFTLSITSLQTEDVATYYCQQYWSTPWTFG |
| | | | GGTTLEIR |
| Antibody to | V_{H} | 117 | EVQLVQPGAELRNSGASVKVSCKASGYRFTSY |
| GARP variant 3 | | | YIDWVRQAPGQGLEWMGRIDPEDGGTKYAQK |
| | | | FQGRVTFTADTSTSTAYVELSSLRSEDTAVYYC |
| | | | ARNEWETVVVGDLMYEYEYWGQGTQVTVSS |
| | V_{L} | 118 | DIQMTQSPTSLSASLGDRVTITCQASQSISSYLA |
| | | | WYQQKPGQAPKLLIYGASRLQTGVPSRFSGSGS |
| | | | GTSFTLTISGLEAEDAGTYYCQQYDSLPVTFGQ |
| | | | GTKVELK |
| | V_{L} | 119 | DIQMTQSPSSLSASLGDRVTITCQASQSIVSYLA |
| | | | WYQQKPGQAPKLLIYGASRLQTGVPSRFSGSGS |
| | | | GTSFTLTISGLEAEDAGTYYCQQYASAPVTFGQ |
| | | | GTGVELK |
| | V_{L} | 120 | DIQMTQSPSSLSASLGDRVTITCQASQSISSYLA |
| | | | WYQQKPGQAPKLLIYGTSRLKTGVPSRFSGSGS |
| | | | GTSFTLTISGLEAEDAGTYYCQQYYSAPVTFGQ |
| | | | GTKVELK |
| | V_{L} | 121 | DIQMTQSPSSLSPSLGDRVTITCQASQTISSFLAW |
| | | | YHQKPGQPPKLLIYRASIPQTGVPSRFSGSGSGT |
| | | | SFTLTIGGLEAEDAGTYYCQQYVSAPPTFGQGT |
| | | | KVELK |
| | V_{L} | 122 | DIQMTQSPSSLSASLGDRVTITCQASQSISSYLA |
| | | | WYQQKPGQAPNILIYGASRLKTGVPSRFSGSGS |
| | | | GTSFTLTISGLEAEDAGTYYCQQYASVPVTFGQ |
| | | | GTKVELK |
| Antibody to 4- | V_{H} | 123 | QVQLQQWGAGLLKPSETLSLTCAVYGGSFSGY |
| 1BB variant 1 | | | YWSWIRQSPEKGLEWIGEINHGGYVTYNPSLES |
| | | | RVTISVDTSKNQFSLKLSSVTAADTAVYYCARD |
| | | | YGPGNYDWYFDLWGRGTLVTVSS |
| | V_{L} | 124 | EIVLTQSPATLSLSPGERATLSCRASQSVSSYLA |
| | | | WYQQKPGQAPRLLIYDASNRATGIPARFSGSGS |
| | | | GTDFTLTISSLEPEDFAVYYCQQRSNWPPALTFG |
| | | | GGTKVEIK |
| Antibody to 4- | V_{H} | 125 | EVQLVESGGGLVQPGGSLRLSCAASGFTFSDY |
| 1BB variant 2 | | | WMSWVRQAPGKGLEWVADIKNDGSYTNYAPS |
| | | | LTNRFTISRDNAKNSLYLQMNSLRAEDTAVYY |
| | | | CARELTGTWGQGTMVTVSS |
| | V_{L} | 126 | DIVMTQSPDSLAVSLGERATINCKSSQSLLSSGN |
| | | | QKNYLAWYQQKPGQPPKLLIYYASTRQSGVPD |
| | | | RFSGSGSGTDFTLTISSLQAEDVAVYYCLQYDR |
| | | | YPFTFGQGTKLEIK |
| Utomilumab | V_{H} | 127 | EVQLVQSGAEVKKPGESLRISCKGSGYSFSTYW |
| | | | ISWVRQMPGKGLEWMGKIYPGDSYTNYSPSFQ |
| | | | GQVTISADKSISTAYLQWSSLKASDTAMYYCAR |
| | | | GYGIFDYWGQGTLVTVSS |
| | V_{L} | 128 | SYELTQPPSVSVSPGQTASITCSGDNIGDQYAH |
| | | | WYQQKPGQSPVLVIYQDKNRPSGIPERFSGSNS |
| | | | GNTATLTISGTQAMDEADYYCATYTGFGSLAV |
| | | | FGGGTKLTVL |
| Antibody to | V_{L} | 129 | DIQMTQSPSSVSASVGDRVTITCRASQGISRLLA |
| ICOS variant 1 | | | WYQQKPGKAPKLLIYVASSLQSGVPSRFSGSGS |
| | | | GTDFTLTISSLQPEDFATYYCQQANSFPWTFGQ |
| | | | GTKVEIK |
| | V_{H} | 130 | QVQLVQSGAEVKKPGASVKVSCKASGYTFTGY |
| | | | YMHWVRQAPGQGLEWMGWINPHSGGTNYAQ |
| | | | KFQGRVTMTRDTSISTAYMELSRLRSDDTAVY |
| | | | YCARTYYYDSSGYYHDAFDIWGQGTMVTVSS |
| Antibody to | V_{H} | 131 | EVQLVESGGGLVQPGGSLRLSCAASGFTFSDY |
| ICOS variant 2 | | | WMDWVRQAPGKGLVWVSNIDEDGSITEYSPFV |
| | | | KGRFTISRDNAKNTLYLQMNSLRAEDTAVYYC |
| | | | TRWGRFGFDSWGQGTLVTVSS |
| | V_{L} | 132 | DIVMTQSPDSLAVSLGERATINCKSSQSLLSGSF |
| | | | NYLTWYQQKPGQPPKLLIFYASTRHTGVPDRFS |
| | | | GSGSGTDFTLTISSLQAEDVAVYYCHHHYNAPP |
| | | | TFGPGTKVDIK |
| Vorsetuzumab | V_{H} | 133 | QVQLVQSGAEVKKPGASVKVSCKASGYTFTNY |
| | | | GMNWVRQAPGQGLKWMGWINTYTGEPTYAD |
| | | | AFKGRVTMTRDTSISTAYMELSRLRSDDTAVY |
| | | | YCARDYGDYGMDYWGQGTTVTVSS |
| | V_{L} | 134 | DIVMTQSPDSLAVSLGERATINCRASKSVSTSGY |
| | | | SFMHWYQQKPGQPPKLLIYLASNLESGVPDRFS |
| | | | GSGSGTDFTLTISSLQAEDVAVYYCQHSREVPW |
| | | | TFGQGTKVEIK |
| Rinucumab | V_{H} | 135 | QLQLQESGPGLVKPSETLSLTCTVSGGSITSSSY |
| | | | YWGWIRQPPGKGLEWIGSIYYRGSTNYNPSLKS |
| | | | RVTISVDSSKNQFYLKVSSVTAVDTAVYYCAR |
| | | | QNGAARPSWFDPWGQGTLVTVSS |
| | V_{L} | 136 | EIVLTQSPDTISLSPGERATLSCRASQSISSIYLA |
| | | | WYQQKPGQAPRLLIYGASSRVTGIPDRFSVSGS |
| | | | GTDFTLTISRLEPEDFAVYYCQHYGISPFTFGPG |
| | | | TKVDIR |
| Oleclumab | V_{H} | 137 | EVQLLESGGGLVQPGGSLRLSCAASGFTFSSYA |
| | | | YSWVRQAPGKGLEWVSAISGSGGRTYYADSVK |
| | | | GRFTISRDNSKNTLYLQMNSLRAEDTAVYYCA |
| | | | RLGYGRVDEWGRGTLVTVSS |
| | V_{L} | 138 | QSVLTQPPSASGTPGQRVTISCSGSLSNIGRNPV |
| | | | NWYQQLPGTAPKLLIYLDNLRLSGVPDRFSGSK |
| | | | SGTSASLAISGLQSEDEADYYCATWDDSHPGW |
| | | | TFGGGTKLTVL |
| Antibody to | V_{L} | 139 | DIQMTQSPSSLSASVGDRVTITCRASQGISSWLA |
| CD73 | | | WYQQKPEKAPKSLIYAASSLQSGVPSRFSGSGS |
| | | | GTDFTLTISSLQPEDFATYYCQQYNSYPLTFGGG |
| | | | TKVEIK |
| | V_{H} | 140 | QVQLVESGGGVVQPGRSLRLSCAASGFTFSNYG |
| | | | MHWVRQAPGKGLEWVAVILYDGSNKYYPDSV |
| | | | KGRFTISRDNSKNTLYLQMNSLRAEDTAVYYC |
| | | | ARGGSSWYPDSFDIWGQGTMVTVSS |
| Daratumumab | V_{H} | 141 | EVQLLESGGGLVQPGGSLRLSCAVSGFTFNSFA |
| | | | MSWVRQAPGKGLEWVSAISGSGGGTYYADSV |
| | | | KGRFTISRDNSKNTLYLQMNSLRAEDTAVYFCA |
| | | | KDKILWFGEPVFDYWGQGTLVTVSS |
| | V_{L} | 142 | EIVLTQSPATLSLSPGERATLSCRASQSVSSYLA |
| | | | WYQQKPGQAPRLLIYDASNRATGIPARFSGSGS |
| | | | GTDFTLTISSLEPEDFAVYYCQQRSNWPPTFGQ |
| | | | GTKVEIK |
| Etaracizumab | V_{H} | 143 | QVQLVESGGGVVQPGRSLRLSCAASGFTFSSYD |
| | | | MSWVRQAPGKGLEWVAKVSSGGGSTYYLDTV |
| | | | QGRFTISRDNSKNTLYLQMNSLRAEDTAVYYC |
| | | | ARHLHGSFASWGQGTTVTVSS |
| | V_{L} | 144 | EIVLTQSPATLSLSPGERATLSCQASQSISNFLH |
| | | | WYQQRPGQAPRLLIRYRSQSISGIPARFSGSGSG |
| | | | TDFTLTISSLEPEDFAVYYCQQSGSWPLTFGGGT |
| | | | KVEIK |
| Intetumumab | V_{H} | 145 | QVQLVESGGGVVQPGRSRRLSCAASGFTFSRYT |
| | | | MHWVRQAPGKGLEWVAVISFDGSNKYYVDSV |
| | | | KGRFTISRDNSENTLYLQVNILRAEDTAVYYCA |
| | | | REARGSYAFDIWGQGTMVTVSS |
| | V_{L} | 146 | EIVLTQSPATLSLSPGERATLSCRASQSVSSYLA |
| | | | WYQQKPGQAPRLLIYDASNRATGIPARFSGSGS |
| | | | GTDFTLTISSLEPEDFAVYYCQQRSNWPPFTFGP |
| | | | GTKVDIK |
| Antibody to | V_{H} | 147 | EVQLVESGGGLVQPGGSLRLSCAVSGFVFSRY |
| Integrin αvβ8 | | | WMSWVRQAPGKGLEWIGEINPDSSTINYTSSLK |
| | | | DRFTISRDNAKNSLYLQMNSLRAEDTAVYYCA |
| | | | SLITTEDYWGQGTTVTVSS |
| | V_{L} | 148 | EIVLTQSPSSLSLSPGERVTITCKASQDINSYLSW |
| | | | YQQKPGKAPKLLIYYANRLVDGVPARFSGSGS |
| | | | GQDYTLTISSLEPEDFAVYYCLQYDEFPYTFGG |
| | | | GTKLEIKR |
| Ontuxizumab | V_{H} | 149 | QVQLQESGPGLVRPSQTLSLTCTASGYTFTDYVI |
| | | | HWVKQPPGRGLEWIGYINPYDDDTTYNQKFKG |
| | | | RVTMLVDTSSNTAYLRLSSVTAEDTAVYYCAR |
| | | | RGNSYDGYFDYSMDYWGSGTPVTVSS |
| | V_{L} | 150 | DIQMTQSPSSLSASVGDRVTITCRASQNVGTAV |
| | | | AWLQQTPGKAPKLLIYSASNRYTGVPSRFSGSG |
| | | | SGTDYTFTISSLQPEDIATYYCQQYTNYPMYTF |
| | | | GQGTKVQIK |
| Antibody to | V_{H} | 151 | QVQLQESGPGLVKPSQTLSLTCAISGDSVSSNSV |
| FAP variant 1 | | | TWNWIRQSPSRGLEWLGRTYYRSKWYNDYAV |
| | | | SVKGRITINPDTSKNQFYLQLKSVTPEDAAVYY |
| | | | CARDSSILYGDYWGQGTLVTVSS |
| | V_{H} | 152 | QVQLQQSGPGLVKPSQTLSLTCAISGDSVSSNSV |
| | | | TWNWIRQSPSRGLEWLGRTYYRSKWYNDYAV |
| | | | SVKGRITINPDTSKNQFYLQLKSVTPEDAAVYY |
| | | | CARDSSILYGDYWGQGTLVTVS |
| | V_{L} | 153 | QAVLTQPSSLSASPGASASLTCTLPSGINVGTYR |
| | | | IFWFQQKPGSPPQYLLSYKSDSDNHQGSGVPSR |
| | | | FSGSKDASANAGILLISGLQSEDEADYYCMIWH |
| | | | SSAWVFGGGTKLTVL |
| Antibody to | V_{H} | 154 | QVQLVQSGAEVKKPGASVKVSCKTSGYTFTDY |
| FAP variant 2 | | | YIHWVRQAPGQGLEWMGWINPNRGGTNYAQK |
| | | | FQGRVTMTRDTSIATAYMELSRLRSDDTAVYY |
| | | | CATASLKIAAVGTFDCWGQGTLVTVSS |
| | V_{L} | 155 | SYELTQPPSVSVSPGQTARITCSGDALSKQYAF |
| | | | WFQQKPGQAPILVIYQDTKRPSGIPGRFSGSSSG |
| | | | TTVTLTISGAQADDEADYYCQSADSSGTYVFGT |
| | | | GTKVTVL |
| Antibody to | V_{H} | 156 | EVQLVETGGGVVQPGRSLRLSCAASGFSFSTHG |
| FAP variant 3 | | | MYWVRQPPGKGLEWVAVISYDGSDKKYADSV |
| | | | KGRFTISRDNSKNTVYLEMSSVRAEDTALYYCF |
| | | | CRRDAFDLWGQGTMVTVSS |
| | V_{L} | 157 | SYVLTQPPSVSVSPGQTARITCSGDALPKKYAY |
| | | | WYQQKSGQAPVLVIYEDTKRPSGIPERFSGSSSG |
| | | | TMATLTISGAQVEDEADYYCYSTDSSGNYWVF |
| | | | GGGTEVTVL |
| Antibody to | V_{H} | 158 | EVQLVESGGGLVEPGGSLRLSCAASGFTFSDAW |
| FAP variant 4 | | | MNWVRQAPGKGLEWVGRIKTKSDGGTTDYAA |
| | | | PVRGRFSISRDDSKNTLFLEMNSLKTEDTAIYYC |
| | | | FITVIVVSSESPLDHWGQGTLVTVSS |
| | V_{L} | 159 | SYELTQPPSVSVSPGQTARITCSGDELPKQYAY |
| | | | WYQQKPGQAPVLVIYKDRQRPSGIPERFSGSSS |
| | | | GTTVTLTISGVQAEDEADYYCQSAYSINTYVIFG |
| | | | GGTKLTVL |
| Antibody to | V_{H} | 160 | EVQLVESGGGLVKPGGSLRLSCAASGFTFSDYY |
| FAP variant 5 | | | MSWIRQAPGKGLEWISYISSGSSYTNYADSVKG |
| | | | RFTISRDNAKKSVYLEVNGLTVEDTAVYYCAR |
| | | | VRYGDREMATIGGFDFWGQGTLVTVSS |
| | V_{L} | 161 | SYELTQPPSVSVSPGQTARITCSGDALPKQYAY |
| | | | WYQQSPGQAPVLVIYKDSERPSGIPERFSGSSSG |
| | | | TTVTLTISGVQAEDEADYYCQSADSGGTSRIFG |
| | | | GGTKLTVL |
| Antibody to | V_{H} | 162 | QVQLQESGPGLVRSTETLSLTCLVSGDSINSHY |
| FAP variant 6 | | | WSWLRQSPGRGLEWIGYIYYTGPTNYNPSLKSR |
| | | | VSISLGTSKDQFSLKLSSVTAADTARYYCARNK |
| | | | VFWRGSDFYYYMDVWGKGTTVTVSS |
| | V_{L} | 163 | EIVLTQSPGTLSLSLGERATLSCRASQSLANNYL |
| | | | AWYQQKPGQAPRLLMYDASTRATGIPDRFSGS |
| | | | GSGTDFTLTISRLEPEDFAVYYCQQFVTSHHMYI |
| | | | FGQGTKVEIK |
| Antibody to | V_{H} | 164 | HVQLQESGPGLVKPSETLSLTCTVSGGSISSNNY |
| FAP variant 7 | | | YWGWIRQTPGKGLEWIGSIYYSGSTNYNPSLKS |
| | | | RVTISVDTSKNQFSLKLSSVTAADTAVYYCARG |
| | | | ARWQARPATRIDGVAFDIWGQGTMVTVSS |
| | V_{H} | 165 | QVQLQESGPGLVKPSETLSLTCTVSGGSISSNNY |
| | | | YWGWIRQTPGKGLEWIGSIYYSGSTNYNPSLKS |
| | | | RVTISVDTSKNQFSLKLSSVTAADTAVYYCARG |
| | | | ARWQARPATRIDGVAFDIWGQGTMVTVSS |
| | V_{H} | 166 | EVQLVQSGAEVKKPGASVKVSCKASGYTFTSY |
| | | | GISWVRQAPGQGLEWMGWISAYNGNTNYAQK |
| | | | LQGRVTMTTDTSTSTAYMELRSLRSDDTAVYY |
| | | | CARDWSRSGYYLPDYWGQGTLVTVSS |
| | V_{L} | 167 | ETTLTQSPGTLSLSPGERATLSCRASQTVTRNYL |
| | | | AWYQQKPGQAPRLLMYGASNRAAGVPDRFSG |
| | | | SGSGTDFTLTISRLEPEDFAVYYCQQFGSPYTFG |
| | | | QGTKVEIK |
| | V_{L} | 168 | DVVMTQSPLSLPVTLGQPASISCRSSQSLLHSNG |
| | | | YNYLDWYLQRPGQSPHLLIFLGSNRASGVPDRF |
| | | | SGSGSGTDFTLKISRVEAEDVGIYYCMQALQTP |
| | | | PTFGQGTKVEIK |
| Antibody to | V_{H} | 171 | QVQLVESGGGVVQPGRSRRLSCAASGFTFSRYT |
| Integrin αv | | | MHWVRQAPGKGLEWVAVISFDGSNKYYVDSV |
| variant 1 | | | KGRFTISRDNSENTLYLQVNILRAEDTAVYYCA |
| | | | REARGSYAFDIWGQGTMVTVSS |
| | V_{L} | 172 | EIVLTQSPATLSLSPGERATLSCRASQSVSSYLA |
| | | | WYQQKPGQAPRLLIYDASNRATGIPARFSGSGS |
| | | | GTDFTLTISSLEPEDFAVYYCQQRSNWPPFTFGP |
| | | | GTKVDIK |
| Antibody to | V_{L} | 173 | DIQMTQSPSSLSASVGDRVTITCRASQDISNYLA |
| Integrin αv | | | WYQQKPGKAPKLLIYYTSKIHSGVPSRFSGSGS |
| variant 2 | | | GTDYTFTISSLQPEDIATYYCQQGNTFPYTFGQG |
| | | | TKVEIK |
| | V_{H} | 174 | QVQLQQSGGELAKPGASVKVSCKASGYTFSSF |
| | | | WMHWVRQAPGQGLEWIGYINPRSGYTEYNEIF |
| | | | RDKATMTTDTSTSTAYMELSSLRSEDTAVYYC |
| | | | ASFLGRGAMDYWGQGTTVTVSS |
| Antibody to | V_{H} | 175 | QVQLQESGPGLVKPSQTLSLTCTVSGGSISSGVY |
| Integrin αvβ6 | | | YWTWIRQHPGNGLEWIGYIYYSGSTSYNPSLKS |
| variant 1 | | | RVTISVDTSKKQFSLNLTSVTAADTAVYYCARE |
| | | | GPLRGDYYYGLDVWGQGTTVTVSS |
| | V_{L} | 176 | EIVLTQSPGTLSLSPGERATLSCRAGQTISSRYLA |
| | | | WYQQKPGQAPRPLIYGASSRATGIPDRFSGSGS |
| | | | GTDFTLTISRLEPEDFAVYYCQQYGSSPRTFGQG |
| | | | TKVEIK |
| Antibody to | V_{H} | 177 | QVQLQESGPGLVKPSQTLSLTCTVSGGSISSGGY |
| Integrin αvβ6 | | | YWSWIRQHPGKGLEWIGYIYYSGSTYYNPSLKS |
| variant 2 | | | RVTISVDTSKNQFSLKLSSVTAADTAMYYCARY |
| | | | RGPAAGRGDFYYFGMDVWGQGTTVTVSS |
| | V_{L} | 178 | DIVMTQTPLSLSVTPGQPASIFCKSSQSLLNSDG |
| | | | KTYLCWYLQKPGQPPQLLIYEVSNRFSGVPDRF |
| | | | SGSGSGTDFTLKISRVEAEDVGVYYCMQGIQLP |
| | | | WAFFGQGTKVEIK |
| Antibody to | V_{H} | 179 | QVQLVESGGGVVQPGRSLRLSCAASGFTFSSYG |
| Integrin αvβ6 | | | MHWVRQAPGKGLEWVAVIWYGGSNKYYADS |
| variant 3 | | | VKGRFTISRDNSKNTLYLQMNSLRAEDTAVYY |
| | | | CARDLAARRGDYYYYGMDVWGQGTTVTVSS |
| | V_{L} | 180 | SSELTQDPVVSVALGQTVRITCQGDSLRSYYLS |
| | | | WYQQKPGQAPVLVIYGKNNRPSGIPDRFSGSNS |
| | | | GNTASLTITGAQAEDEADYYCNSRDSSGNHLFG |
| | | | GGTKLTVL |
| Antibody to | V_{H} | 181 | QVQLQESGPGLVKPSQTLSLTCTVSGGSISSGGY |
| Integrin αvβ6 | | | YWSWIRQHPGKGLEWIGYIYYSGRTYNNPSLKS |
| variant 4 | | | RVTISVDTSKNQFSLKLSSVTAADTAVYYCARV |
| | | | ATGRADYHFYAMDVWGQGTTVTVSS |
| | V_{L} | 182 | SYELTQPSSVSVSPGQTARITCSGDVLAKKSAR |
| | | | WFHQKPGQAPVLVIYKDSERPSGIPERFSGSSSG |
| | | | TTVTLTISGAQVEDEAAYYCYSAADNNLVFGG |
| | | | GTKLTVL |
| Varlilumab | V_{H} | 183 | QVQLVESGGGVVQPGRSLRLSCAASGFTFSSYD |
| | | | MHWVRQAPGKGLEWVAVIWYDGSNKYYADS |
| | | | VKGRFTISRDNSKNTLYLQMNSLRAEDTAVYY |
| | | | CARGSGNWGFFDYWGQGTLVTVSS |
| | V_{L} | 184 | DIQMTQSPSSLSASVGDRVTITCRASQGISRWLA |
| | | | WYQQKPEKAPKSLIYAASSLQSGVPSRFSGSGS |
| | | | GTDFTLTISSLQPEDFATYYCQQYNTYPRTFGQ |
| | | | GTKVEIK |
| Zinbryta® | V_{H} | 185 | QVQLVQSGAEVKKPGSSVKVSCKASGYTFTSY |
| (Daclizumab) | | | RMHWVRQAPGQGLEWIGYINPSTGYTEYNQKF |
| | | | KDKATITADESTNTAYMELSSLRSEDTAVYYCA |
| | | | RGGGVFDYWGQGTLVTVSS |
| | V_{L} | 186 | DIQMTQSPSTLSASVGDRVTITCSASSSISYMHW |
| | | | YQQKPGKAPKLLIYTTSNLASGVPARFSGSGSG |
| | | | TEFTLTISSLQPDDFATYYCHQRSTYPLTFGQGT |
| | | | KVEVK |
| Antibody to | V_{H} | 187 | QVQLVESGGGVVQPGRSLRLSCAASGFTFSSYG |
| GITR | | | MHWVRQAPGKGLEWVAVIWYEGSNKYYADS |
| | | | VKGRFTISRDNSKNTLYLQMNSLRAEDTAVYY |
| | | | CARGGSMVRGDYYYGMDVWGQGTTVTVSS |
| | V_{L} | 188 | AIQLTQSPSSLSASVGDRVTITCRASQGISSALA |
| | | | WYQQKPGKAPKLLIYDASSLESGVPSRFSGSGS |
| | | | GTDFTLTISSLQPEDFATYYCQQFNSYPYTFGQG |
| | | | TKLEIK |
| Antibody | V_{H} | 189 | EVQLVQSGAEVKKPGASVKVSCKASGYKFSSY |
| huM25 to | | | WIEWVKQAPGQGLEWIGEILPGSDTTNYNEKFK |
| LRRC15 | | | DRATFTSDTSINTAYMELSRLRSDDTAVYYCAR |
| | | | DRGNYRAWFGYWGQGTLVTVSS |
| | V_{L} | 190 | DIQMTQSPSSLSASVGDRVTITCRASQDISNYLN |
| | | | WYQQKPGGAVKFLIYYTSRLHSGVPSRFSGSGS |
| | | | GTDYTLTISSLQPEDF |
| | | | ATYFCQQGEALPWTFGGGTKVEIK |
| Antibody | V_{H} | 191 | EVQLVQSGAEVKKPGSSVKVSCKASGFTFTDY |
| huAD208.4.1 to | | | YIHWVKQAPGQGLEWIGLVYPYIGGTNYNQKF |
| LRRC15 | | | KGKATLTVDTSTTTAYMEMSSLRSEDTAVYYC |
| | | | ARGDNKYDAMDYWGQGTTVTVSS |
| | V_{L} | 192 | DIVLTQSPDSLAVSLGERATINCRASQSVSTSSY |
| | | | SYMHWYQQKPGQPPKLLIKYASSLESGVPDRFS |
| | | | GSGSGTDFTLTISSLQ |
| | | | AEDVAVYYCEQSWEIRTFGGGTKVEIK |
| Antibody | V_{H} | 193 | EVQLVQSGAEVKKPGSSVKVSCKASGYTFTNY |
| huAD208.12.1 | | | WMHWVKQAPGQGLEWIGMIHPNSGSTKHNEK |
| to LRRC15 | | | FRGKATLTVDESTTTAYMELSSLRSEDTAVYYC |
| | | | ARSDFGNYRWYFDVWGQGTTVTVSS |
| | V_{L} | 194 | EIVLTQSPATLSLSPGERATLSCRASQSSSNNLH |
| | | | WYQQKPGQAPRVLIKYVSQSISGIPARFSGSGSG |
| | | | TDFTLTISSLEPEDFA |
| | | | VYFCQQSNSWPFTFGQGTKLEIK |
| Antibody | V_{H} | 195 | EVQLVQSGAEVKKPGSSVKVSCKASGFTFTDY |
| huAD208.14.1 | | | YIHWVKQAPGQGLEWIGLVYPYIGGSSYNQQF |
| to LRRC15 | | | KGKATLTVDTSTSTAYMELSSLRSEDTAVYYCA |
| | | | RGDNNYDAMDYWGQGTTVTVSS |
| | V_{L} | 196 | DIVLTQSPDSLAVSLGERATISCRASQSVSTSTY |
| | | | NYMHWYQQKPGQPPKLLVKYASNLESGVPDRF |
| | | | SGSGSGTDFTLTISSL |
| | | | QAEDVAVYYCHHTWEIRTFGGGTKVEIK |
| Antibody | V_{H} | 197 | EVQLVESGGGLVQPGGSLRLSCAVSGFSLTSYG |
| hu139.10 to | | | VHWVRQATGKGLEWLGVIWAGGSTNYNSALM |
| LRRC15 | | | SRLTISKENAKSSVYLQMNSLRAGDTAMYYCA |
| | | | THMITEDYYGMDYWGQGTTVTVSS |
| | V_{L} | 198 | DIVMTQSPDSLAVSLGERATINCKSSQSLLNSRT |
| | | | RKNYLAWYQQKPGQSPKLLIYWASTRESGVPD |
| | | | RFSGSGSGTDFTLTISS |
| | | | LQAEDVAVYYCKQSYNLPTFGGGTKVEIK |

### Target Binding Domain

An antibody construct may further comprise a target binding domain. A target binding domain may comprise a domain that binds to a target. A target may be an antigen. A target binding domain may comprise an antigen binding domain. A target binding domain may be a domain that can specifically bind to an antigen. A target binding domain may be an antigen-binding portion of an antibody or an antibody fragment. A target binding domain may be one or more fragments of an antibody that can retain the ability to specifically bind to an antigen. A target binding domain may be any antigen binding fragment. A target binding domain may be in a scaffold, in which a scaffold is a supporting framework for the antigen binding domain. A target binding domain may comprise an antigen binding domain in a scaffold.

A target binding domain may comprise an antigen binding domain which refers to a portion of an antibody comprising the antigen recognition portion, i.e., an antigenic determining variable region of an antibody sufficient to confer recognition and binding of the antigen recognition portion to a target, such as an antigen, i.e., the epitope. A target binding domain may comprise an antigen binding domain of an antibody.

An Fv can be the minimum antibody fragment which contains a complete antigen-recognition and antigen-binding site. This region may consist of a dimer of one heavy chain and one light chain variable domain in tight, non-covalent association. In this configuration, the three CDRs of each variable domain may interact to define an antigen-binding site on the surface of the V_{H}-V_{L} dimer. A single variable domain (or half of an Fv comprising only three CDRs specific for an antigen) can recognize and bind antigen, although at a lower affinity than the entire binding site.

A target binding domain may be at least 80% identical to an antigen binding domain selected from, but not limited to, a monoclonal antibody, a polyclonal antibody, a recombinant antibody, or a functional fragment thereof, for example, a heavy chain variable domain (V_{H}) and a light chain variable domain (V_{L}), a single chain variable fragment (scFv), or a DARPin, an affimer, an avimer, a knottin, a monobody, an affinity clamp, an ectodomain, a receptor ectodomain, a receptor, a cytokine, a ligand, an immunocytokine, a T cell receptor, or a recombinant T cell receptor.

A target binding domain may be attached to an antibody construct. For example, an antibody construct may be fused with a target binding domain to create an antibody construct target binding domain fusion. The antibody construct-target binding domain fusion may be the result of the nucleic acid sequence of the target binding domain being expressed in frame with the nucleic acid sequence of the antibody construct. The antibody construct-target binding domain fusion may be the result of an in-frame genetic nucleotide sequence or a contiguous peptide sequence encoding the antibody construct with the target binding domain. As another example, a target binding domain may be linked to an antibody construct. A target binding domain may be linked to an antibody construct by a chemical conjugation. A target binding domain may be attached to a terminus of an Fc region. A target binding domain may be attached to a terminus of an Fc domain. A target binding domain may be attached to a terminus of an antibody construct. A target binding domain may be attached to a terminus of an antibody. A target binding domain may be attached to a light chain of an antibody. A target binding domain may be attached to a terminus of a light chain of an antibody. A target binding domain may be attached to a heavy chain of an antibody. A target binding domain may be attached to a terminus of a heavy chain of an antibody. The terminus may be a C-terminus. An antibody construct may be attached to 1, 2, 3, and/or 4 target binding domains. The target binding domain may direct the antibody construct to, for example, a particular cell or cell type. A target binding domain of an antibody construct may be selected in order to recognize an antigen, e.g., an antigen expressed on an immune cell. An antigen can be a peptide or fragment thereof. An antigen may be expressed on an antigen-presenting cell. An antigen may be expressed on a dendritic cell, a macrophage, or a B cell. As another example, an antigen may be a tumor antigen. The tumor antigen may be any tumor antigen described herein. When multiple target binding domains are attached to an antibody construct, the target binding domains may bind to the same antigen. When multiple target binding domains are attached to an antibody construct, the target binding domains may bind different antigens.

In certain embodiments, an antibody construct specifically binds a second antigen. In certain embodiments, the target binding domain is linked to said antibody construct at a C-terminal end of said Fc domain.

In certain embodiments, the target binding domain specifically binds to an antigen that is at least 80% identical to an antigen on a T cell, a B cell, a stellate cell, an endothelial cell, a tumor cell, an APC, a fibroblast cell, a fibrocyte cell, or a cell associated with the pathogenesis of fibrosis. In certain embodiments, the target binding domain specifically binds to an antigen that is at least 80% identical to an antigen on a T cell, an APC, and/or a B cell. In certain embodiments, the target binding domain may specifically bind to an antigen that is at least 80% identical to an antigen selected from the group consisting of CTLA4, PD-1, OX40, LAG-3, GITR, GARP, CD25, CD27, PD-L1, TNFR2, ICOS, 41BB, CD70, CD73, CD38, or VTCN1. In certain embodiments, the target binding domain specifically binds to an antigen that is an antigen on a T cell, a B cell, a stellate cell, an endothelial cell, a tumor cell, an APC, a fibroblast cell, a fibrocyte cell, or a cell associated with the pathogenesis of fibrosis. In certain embodiments, the target binding domain specifically binds to an antigen that is an antigen on a T cell, an APC, and/or a B cell. In certain embodiments, the target binding domain may specifically bind to an antigen that is at least 80% identical to an antigen selected from the group consisting of CTLA4, PD-1, OX40, LAG-3, GITR, GARP, CD25, CD27, PD-L1, TNFR2, ICOS, 41BB, CD70, CD73, CD38, or VTCN1.

### Attachment of Linkers to Antibody Construct or Other Targeting Moiety

The conjugates described herein may comprise a linker, e.g., a peptide linker. Linkers of the conjugates and methods may not affect the binding of active portions of a conjugate (e.g., active portions include antigen binding domains, Fc domains, target binding domains, antibodies, cyclic amino-pyrazinecarboxamide compounds, inhibitors or the like) to a target, which can be a cognate binding partner, such as an antigen, ligand, or receptor. A linker can form a linkage between different parts of a conjugate, e.g., between an antibody construct or targeting moiety and a compound of the disclosure. In certain embodiments, a conjugate comprises multiple linkers. In certain embodiments, wherein a conjugate comprises multiple linkers, the linkers may be the same linkers or different linkers.

A linker may be bound to an antibody construct or targeting moiety by a bond between the antibody construct or targeting moiety and the linker. A linker may be bound to an anti-tumor antigen antibody construct or targeting moiety by a bond between the anti-tumor antigen antibody construct or targeting moiety and the linker. A linker may be bound to a terminus of an amino acid sequence of an antibody construct or targeting moiety, or could be bound to a side chain modification to the antibody construct or targeting moiety, such as the side chain of a lysine, serine, threonine, cysteine, tyrosine, aspartic acid, glutamine, a non-natural amino acid residue, or glutamic acid residue. A linker may be bound to a terminus of an amino acid sequence of an Fc region of an antibody construct, or may be bound to a side chain modification of an Fc region of an antibody construct, such as the side chain of a lysine, serine, threonine, cysteine, tyrosine, aspartic acid, glutamine, a non-natural amino acid residue, or glutamic acid residue. A linker may be bound to a terminus of an amino acid sequence of an Fc domain of an antibody construct, or may be bound to a side chain modification of an Fc domain of an antibody construct, such as the side chain of a lysine, serine, threonine, cysteine, tyrosine, aspartic acid, glutamine, a non-natural amino acid residue, or glutamic acid residue.

A linker may be bound to an antibody construct at a hinge cysteine. A linker may be bound to an antibody construct at a light chain constant domain lysine. A linker may be bound to an antibody construct at an engineered cysteine in the light chain. A linker may be bound to an antibody construct at an Fc region lysine. A linker may be bound to an antibody construct at an Fc domain lysine. A linker may be bound to an antibody construct at an Fc region cysteine. A linker may be bound to an antibody construct at an Fc domain cysteine. A linker may be bound to an antibody construct at a light chain glutamine, such as an engineered glutamine. A linker may be bound to an antibody construct at an unnatural amino acid engineered into the light chain. A linker may be bound to an antibody construct at an unnatural amino acid engineered into the heavy chain. Amino acids can be engineered into an amino acid sequence of an antibody construct, for example, a linker of a conjugate. Engineered amino acids may be added to a sequence of existing amino acids. Engineered amino acids may be substituted for one or more existing amino acids of a sequence of amino acids.

A linker may be conjugated to an antibody construct or targeting moiety via a sulfhydryl group on the antibody construct or targeting moiety. A linker may be conjugated to an antibody construct or targeting moiety via a primary amine on the antibody construct or targeting moiety. A linker may be conjugated to an antibody construct or targeting moiety via residue of an unnatural amino acid on an antibody construct or targeting moiety, e.g., a ketone moiety.

In certain embodiments, when one or more linkers are bound, e.g., covalently, to an antibody construct at sites on the construct, an Fc domain of the antibody construct can bind to Fc receptors. In certain embodiments, an antibody construct or targeting moiety bound to a linker or an antibody construct or targeting moiety bound to a linker bound to a TGFβR2 inhibitor (such as a cyclic amino-pyrazinecarboxamide compound), retains the ability of the Fc domain of the antibody to bind to Fc receptors. In certain embodiments, when a linker is connected to an antibody construct or targeting moiety, the antigen binding domain of an antibody construct or targeting moiety bound to a linker, or an antibody construct or targeting moiety bound to a linker bound to a TGFβR2 inhibitor (such as a cyclic amino-pyrazinecarboxamide compound of this disclosure), can bind its antigen. In certain embodiments, when a linker is connected to an antibody construct or targeting moiety at the sites described herein, a target binding domain of an antibody construct or targeting moiety bound to a linker, or an antibody construct or targeting moiety bound to a linker bound to a TGFβR2 inhibitor (such as a cyclic amino-pyrazinecarboxamide compound of this disclosure), can bind its antigen.

In certain embodiments, a linker or linker bound to a TGFβR2 inhibitor (such as a cyclic amino-pyrazinecarboxamide compound of this disclosure) may be attached to an amino acid residue of an IgG Fc domain selected from: 221, 222, 224, 227, 228, 230, 231, 223, 233, 234, 235, 236, 237, 238, 239, 240, 241, 243, 244, 245, 246, 247, 249, 250, 258, 262, 263, 264, 265, 266, 267, 268, 269, 270, 271, 272, 273, 274, 275, 276, 278, 280, 281, 283, 285, 286, 288, 290, 291, 292, 293, 294, 295, 296, 297, 298, 299, 300, 302, 305, 313, 317, 318, 320, 322, 323, 324, 325, 326, 327, 328, 329, 330, 331, 332, 333, 334, 335, 336, 396, 428, or any subset thereof, wherein numbering of amino acid residues in the Fc domain is according to the EU index as in Kabat.

In certain embodiments, a linker or linker bound to a TGFβR2 inhibitor (such as a cyclic amino-pyrazinecarboxamide compound of this disclosure) is not attached to an amino acid residue of an IgG Fc domain selected from: 221, 222, 224, 227, 228, 230, 231, 223, 233, 234, 235, 236, 237, 238, 239, 240, 241, 243, 244, 245, 246, 247, 249, 250, 258, 262, 263, 264, 265, 266, 267, 268, 269, 270, 271, 272, 273, 274, 275, 276, 278, 280, 281, 283, 285, 286, 288, 290, 291, 292, 293, 294, 295, 296, 297, 298, 299, 300, 302, 305, 313, 317, 318, 320, 322, 323, 324, 325, 326, 327, 328, 329, 330, 331, 332, 333, 334, 335, 336, 396, 428, or any subset thereof, wherein numbering of amino acid residues in the Fc domain is according to the EU index as in Kabat.

### Lysine-Based Bioconjugation

An antibody construct or targeting moiety can be conjugated to a linker via lysine-based bioconjugation. An antibody construct or targeting moiety can be exchanged into an appropriate buffer, for example, phosphate, borate, PBS, histidine, Tris-Acetate at a concentration of about 2 mg/mL to about 10 mg/mL. An appropriate number of equivalents of a construct of a cyclic amino-pyrazinecarboxamide compound, and a linker, linker-payload, as described herein, can be added as a solution with stirring. Dependent on the physical properties of the linker-payload, a co-solvent can be introduced prior to the addition of the linker-payload to facilitate solubility. The reaction can be stirred at room temperature for 2 hours to about 12 hours depending on the observed reactivity. The progression of the reaction can be monitored by LC-MS. Once the reaction is deemed complete, the remaining linker-payloads can be removed by applicable methods and the antibody conjugate can be exchanged into the desired formulation buffer. Lysine-linked conjugates can be synthesized starting with ab antibody (mAb) and linker-payload, e.g., 10 equivalents, following Scheme A below (Conjugate=antibody conjugate). Monomer content and drug-antibody construct ratios (molar ratios) can be determined by methods described herein.

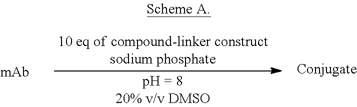

### Cysteine-Based Bioconjugation

An antibody construct or targeting moiety can be conjugated to a linker via cysteine-based bioconjugation. An antibody construct or targeting moiety can be exchanged into an appropriate buffer, for example, phosphate, borate, PBS, histidine, Tris-Acetate at a concentration of about 2 mg/mL to about 10 mg/mL with an appropriate number of equivalents of a reducing agent, for example, dithiothreitol or tris(2-carboxyethyl)phosphine. The resultant solution can be stirred for an appropriate amount of time and temperature to effect the desired reduction. A construct of a cyclic amino-pyrazinecarboxamide compound and a linker can be added as a solution with stirring. Dependent on the physical properties of the linker-payload, a co-solvent can be introduced prior to the addition of the linker-payload to facilitate solubility. The reaction can be stirred at room temperature for about 1 hour to about 12 hours depending on the observed reactivity. The progression of the reaction can be monitored by liquid chromatography-mass spectrometry (LC-MS). Once the reaction is deemed complete, the remaining free linker-payload can be removed by applicable methods and the antibody conjugate can be exchanged into the desired formulation buffer. Such cysteine-based conjugates can be synthesized starting with an antibody (mAb) and linker-payload, e.g., 7 equivalents, using the conditions described in Scheme B below (Conjugate=antibody conjugate). Monomer content and drug-antibody ratios can be determined by methods described herein.

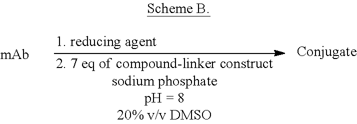

### Cyclic Amino-Pyrazinecarboxamide Compounds

The following is a discussion of compounds and salts thereof that may be used in the compositions and methods of the disclosure.

In one aspect, disclosed herein is a compound represented by Formula (I):

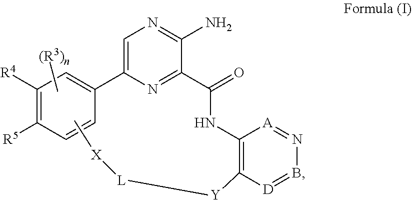

or a pharmaceutically acceptable salt thereof, wherein:
A, B, and D are each independently selected from N and C(R′);
   each R¹ is independently selected from hydrogen, halogen, cyano, —OH, —OR⁵⁰, —NR⁵¹R⁵¹, unsubstituted or substituted —C₁-C₆alkyl, unsubstituted or substituted cycloalkyl, and unsubstituted or substituted heterocycloalkyl;
each R³ is independently selected from R²⁰, R^{L}, and —O—R^{L};
n is 0, 1, or 2;
R⁴ is selected from hydrogen, R²⁰, R^{L}, and —O—R^{L};
R⁵ is selected from hydrogen, R²⁰, R^{L}, and —O—R^{L};
X is selected from —O—, —S—, —NR⁷—, —C(R⁸)₂—, —C(R⁸)₂—O—, —C(R⁸)₂—S—, —C(R⁸)₂—NR⁷—, —S(═O)₂—, —C(═O)—, —NR⁷—S(═O)₂—, and —NR⁷—C(═O)—;
   R⁷ is selected from hydrogen, unsubstituted or substituted —C₁-C₆alkyl, and R^{L};
   each R⁸ is independently selected from hydrogen, halogen, unsubstituted or substituted —C₁-C₆alkyl, and R^{L};
Y is selected from —O—, —S—, —NR⁹—, —C(R¹⁰)₂—, —S(═O)₂—, —C(═O)—, —S(═O)₂—NR⁹—, —C(═O)—NR⁹—, substituted or unsubstituted cycloalkylene, and substituted or unsubstituted heterocycloalkylene;
   R⁹ is selected from hydrogen and unsubstituted or substituted —C₁-C₆alkyl;
   each R¹⁰ is independently selected from hydrogen, halogen, and unsubstituted or substituted —C₁-C₆alkyl;
L is selected from a bond, substituted or unsubstituted C₁-C₁₀ alkylene, —[C(R¹¹)₂]_{q}—(W)—, substituted or unsubstituted C₂-C₁₀ alkenylene, substituted or unsubstituted C₂-C₁₀ alkynylene, and [(substituted or unsubstituted C₁-C₄ alkylene)-Z-]ₚ-(substituted or unsubstituted C₁-C₄ alkylene);
   W is unsubstituted or substituted cycloalkylene or unsubstituted or substituted heterocycloalkylene;
   each Z is independently selected from —O—, —S—, and —NR¹¹—;
   each R¹¹ is independently selected from hydrogen and unsubstituted or substituted —C₁-C₆alkyl;
   p is 1-5;
   q is 0-10;
   wherein if L is a bond, then Y is selected from substituted or unsubstituted cycloalkylene and substituted or unsubstituted heterocycloalkylene;
R^{L} is selected from -(unsubstituted or substituted C₁-C₆ alkylene)-OR¹², or -(unsubstituted or substituted C₁-C₆ alkylene)-N(R¹³)₂,
   R¹² is selected from hydrogen, unsubstituted or substituted —C₁-C₆alkyl, unsubstituted or substituted —C₂-C₆ alkenyl, unsubstituted or substituted —C₂-C₆ alkynyl, unsubstituted or substituted cycloalkyl, and unsubstituted or substituted heterocycloalkyl;
   each R¹³ is independently selected from hydrogen, —C(═O)R⁵⁰, —C(═O)OR⁵¹, —C(═O)NR⁵¹R⁵¹, unsubstituted or substituted —C₁-C₆alkyl, unsubstituted or substituted —C₂-C₆ alkenyl, unsubstituted or substituted —C₂-C₆ alkynyl, unsubstituted or substituted cycloalkyl, and unsubstituted or substituted heterocycloalkyl;
   or two R¹³ on the same N atom are taken together with the N atom to which they are attached to form an unsubstituted or substituted N-containing heterocycle;
each R²⁰ is independently selected from halogen, —CN, —OH, —OR⁵⁰, —NR⁵¹R⁵¹, —C(═O)R⁵⁰, —OC(═O)R⁵⁰, —C(═O)OR⁵¹, —OC(═O)OR⁵¹, —C(═O)NR⁵¹R⁵¹, —OC(═O)NR⁵¹R⁵¹, —NR⁵¹C(═O)NR⁵¹R⁵¹, —NR⁵¹C(═O)R⁵⁰, —NR⁵¹C(═O)OR⁵¹, —SR⁵¹, —S(═O)R⁵⁰, —SO₂R⁵⁰, —SO₂NR⁵¹R⁵¹, —NHSO₂R⁵⁰, unsubstituted or substituted —C₁-C₆ alkyl, unsubstituted or substituted —C₂-C₆ alkenyl, unsubstituted or substituted —C₂-C₆ alkynyl, unsubstituted or substituted cycloalkyl, and unsubstituted or substituted heterocycloalkyl;
   each R⁵⁰ is independently selected from unsubstituted or substituted —C₁-C₆ alkyl, unsubstituted or substituted cycloalkyl, unsubstituted or substituted heterocycloalkyl, unsubstituted or substituted aryl, unsubstituted or substituted heteroaryl, -(unsubstituted or substituted C₁-C₆alkylene)-cycloalkyl, -(unsubstituted or substituted C₁-C₆alkylene)-heterocycloalkyl, -(unsubstituted or substituted C₁-C₆alkylene)-aryl, and -(unsubstituted or substituted C₁-C₆alkylene)-heteroaryl; and
   each R⁵¹ is independently selected from hydrogen, unsubstituted or substituted —C₁-C₆ alkyl, unsubstituted or substituted cycloalkyl, unsubstituted or substituted heterocycloalkyl, unsubstituted or substituted aryl, unsubstituted or substituted heteroaryl, -(unsubstituted or substituted C₁-C₆alkylene)-cycloalkyl, -(unsubstituted or substituted C₁-C₆alkylene)-heterocycloalkyl, -(unsubstituted or substituted C₁-C₆alkylene)-aryl, and -(unsubstituted or substituted C₁-C₆alkylene)-heteroaryl;
   or two R⁵¹ on the same N atom are taken together with the N atom to which they are attached to form an unsubstituted or substituted N-containing heterocycle;
wherein when any of L, W, Y, R^{L}, R¹, R⁷, R⁸, R⁹, R¹⁰, R¹¹, R¹², R¹³, R²⁰, R⁵⁰, and R⁵¹ are substituted, substituents on the L, W, Y, R^{L}, R¹, R⁷, R⁸, R⁹, R¹⁰, R¹¹, R¹², R¹³, R²⁰, R⁵⁰, and R⁵¹ are independently selected at each occurrence from halogen, —CN, —NO₂, —OR⁵², —CO₂R⁵², —C(═O)R⁵³, —C(═O)NR⁵²R⁵², —NR⁵²R⁵², —NR⁵²C(═O)R⁵³, —NR⁵²C(═O)OR⁵², —SR⁵², —S(═O)R⁵³, —SO₂R⁵³, —SO₂NR⁵²R⁵², unsubstituted C₁-C₆ alkyl, unsubstituted C₁-C₆ haloalkyl, unsubstituted phenyl, unsubstituted 5- or 6-membered heteroaryl, unsubstituted monocyclic cycloalkyl, and unsubstituted monocyclic heterocycloalkyl; or two substituents on the same carbon atom are taken together to form a ═O or ═S;
   each R⁵² is independently selected from hydrogen, unsubstituted C₁-C₆ alkyl, unsubstituted C₃-C₆ cycloalkyl, unsubstituted 3- to 6-membered heterocycloalkyl, unsubstituted phenyl, unsubstituted benzyl, unsubstituted 5-membered heteroaryl, and unsubstituted 6-membered heteroaryl;
   or two R⁵² groups are taken together with the N atom to which they are attached to form an unsubstituted N-containing heterocycle; and
   each R⁵³ is independently selected from unsubstituted C₁-C₆alkyl, unsubstituted C₃-C₆cycloalkyl, unsubstituted phenyl, unsubstituted benzyl, unsubstituted 5-membered heteroaryl, and unsubstituted 6-membered heteroaryl.

In some embodiments of a compound of Formula (I), or a pharmaceutically acceptable salt thereof, A, B, and D each independently selected from N and C(R¹); wherein one of A, B, and D is N. In some embodiments, A and D are C(R¹); and B is N. In some embodiments, A is N; and B and D are C(R¹). In some embodiments, D is N; and B and A are C(R¹). In some embodiments, A, B and D are C(R¹). In some embodiments, A, B and D are CH.

In some embodiments of a compound of Formula (I), each R¹ is independently selected from hydrogen, halogen, cyano, —OH, —OR⁵⁰, —NR⁵¹R⁵¹, unsubstituted or substituted —C₁-C₆alkyl, unsubstituted or substituted cycloalkyl, and unsubstituted or substituted heterocycloalkyl or any combination thereof. In some embodiments, each R¹ is independently selected from hydrogen, halogen, cyano, —OH, —OR⁵⁰, —NR⁵¹R⁵¹, and unsubstituted or substituted —C₁-C₆alkyl. In some embodiments, each R¹ is independently selected from hydrogen, halogen, cyano, and unsubstituted —C₁-C₆alkyl. In some embodiments, each R¹ is independently selected from hydrogen and halogen. In some embodiments, each R¹ is hydrogen.

In some embodiments of a compound of Formula (I), or a pharmaceutically acceptable salt thereof, R³ can be present or absent. In embodiments wherein R³ is absent, n is 0. In embodiments wherein R³ is present, n is 1 or 2.

In embodiments of a compound of Formula (I), or a pharmaceutically acceptable salt thereof, wherein n is 1 or 2, R³ is independently selected from R²⁰, R^{L}, and —O—R^{L}. In some aspects, each R³ is independently selected from halogen, —CN, —OH, —OR⁵⁰, —NR⁵¹R⁵¹, —C(═O)R⁵⁰, —C(═O)N⁵¹R⁵¹, —NR⁵¹C(═O)R⁵⁰, unsubstituted or substituted C₁-C₆ alkyl, unsubstituted or substituted cycloalkyl, and unsubstituted or substituted heterocycloalkyl. In some embodiments, each R³ is independently selected from halogen, —CN, —OH, —OR⁵⁰, —NR⁵¹R⁵¹, and unsubstituted or substituted C₁-C₆ alkyl.

In some embodiments of a compound of Formula (I), or a pharmaceutically acceptable salt thereof, at least one of R³, R⁴, and R⁵ is halogen.

In some embodiments of a compound of Formula (I), or a pharmaceutically acceptable salt thereof, the compound is represented by Formula (II):

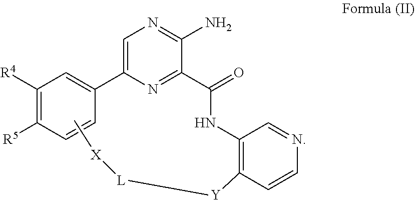

In some embodiments of a compound of Formula (I), or a pharmaceutically acceptable salt thereof, the compound is represented by Formula (II-a):

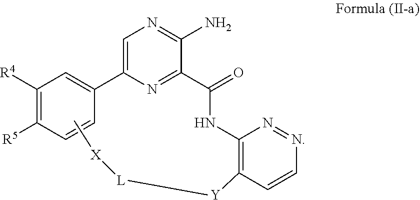

In some embodiments of a compound of Formula (I), or a pharmaceutically acceptable salt thereof, the compound is represented by Formula (II-b):

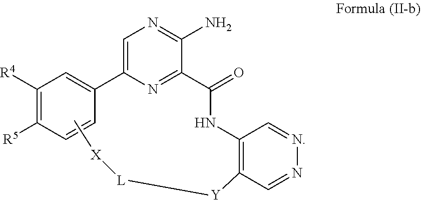

In some embodiments of a compound of Formula (I), or a pharmaceutically acceptable salt thereof, the compound is represented by Formula (II-c):

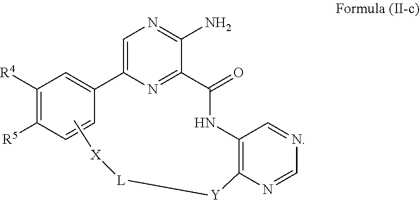

In some embodiments of a compound of Formula (I) or (II), or a pharmaceutically acceptable salt thereof, the compound is represented by Formula (III):

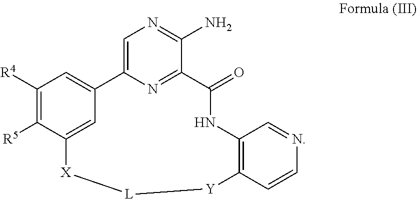

In some embodiments of a compound of Formula (I) or (II-a), or a pharmaceutically acceptable salt thereof, the compound is represented by Formula (III-a):

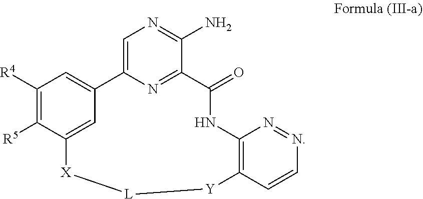

In some embodiments of a compound of Formula (I) or (II-b), or a pharmaceutically acceptable salt thereof, the compound is represented by Formula (III-b):

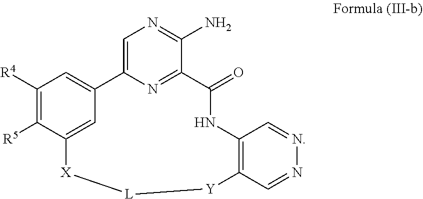

In some embodiments of a compound of Formula (I) or (II-b), or a pharmaceutically acceptable salt thereof, the compound is represented by Formula (III-c):

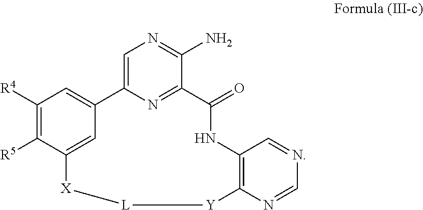

In some embodiments of a compound of Formula (I) or (II), or a pharmaceutically acceptable salt thereof, the compound is represented by Formula (IV):

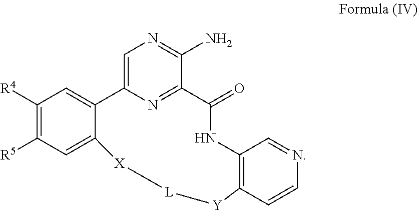

In some embodiments of a compound of Formula (I) or (II-a), or a pharmaceutically acceptable salt thereof, the compound is represented by Formula (IV-a):

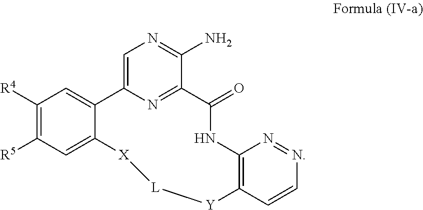

In some embodiments of a compound of Formula (I) or (II-b), or a pharmaceutically acceptable salt thereof, the compound is represented by Formula (IV-b):

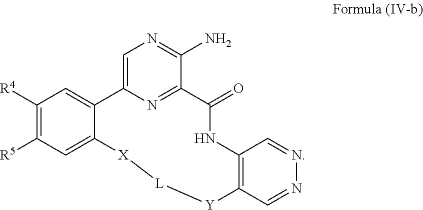

In some embodiments of a compound of Formula (I) or (II-b), or a pharmaceutically acceptable salt thereof, the compound is represented by Formula (IV-c):

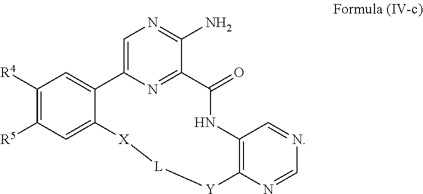

In some embodiments of a compound of Formula (I), (II), (II-a), (II-b), (II-c), (III), (III-a), (III-b), (III-c), (IV), (IV-a), (IV-b), or (IV-c), or a pharmaceutically acceptable salt thereof, Y is selected from —O—, —S—, —S(═O)₂—, —NR⁹—, —C(R¹⁰)₂—, and substituted or unsubstituted heterocycloalkylene. In some embodiments, Y is selected from —O—, —S—, —S(═O)₂—, —NR⁹—, and —C(R¹⁰)₂—. In some embodiments, Y is selected from —S—, —NR⁹—, and —CH₂—. In some embodiments, Y is —NR⁹—. In some embodiments, Y is —O—. In some embodiments, Y is —S—. In some embodiments, Y is —S(═O)₂—. In some embodiments, Y is —C(R¹⁰)₂—.

In some embodiments of a compound of Formula (I), (II), (II-a), (II-b), (II-c), (III), (III-a), (III-b), (III-c), (IV), (IV-a), (IV-b), or (IV-c), or a pharmaceutically acceptable salt thereof, Y is selected from *—NR⁷—C(═O)—# and #—NR⁷—C(═O)—*; wherein # is the attachment point to L and * is the attachment point to the rest of the molecule.

In some embodiments of a compound of Formula (I), (II), (II-a), (II-b), (II-c), (III), (III-a), (III-b), (III-c), (IV), (IV-a), (IV-b), or (IV-c), or a pharmaceutically acceptable salt thereof, when Y is —NR⁹—, —S(═O)₂—NR⁹—, —C(═O)—NR⁹—, —NR⁹—S(═O)₂—, or —NR⁹—C(═O)—, R⁹ is selected from hydrogen, unsubstituted —C₁-C₆alkyl. In some embodiments, R⁹ is selected from hydrogen and unsubstituted —C₁-C₄alkyl, and R^{L}. In some embodiments, R⁹ is R^{L}. In some embodiments, R⁹ is hydrogen. In some embodiments, R⁹ is unsubstituted —C₁-C₆alkyl. In some embodiments, R⁹ is unsubstituted —C₁-C₄alkyl. In some embodiments, R⁹ is methyl or ethyl. In some embodiments, R⁹ is methyl. In some embodiments, R⁹ is ethyl.

In some embodiments of a compound of Formula (I), (II), (II-a), (II-b), (II-c), (III), (III-a), (III-b), (III-c), (IV), (IV-a), (IV-b), or (IV-c), or a pharmaceutically acceptable salt thereof, when Y is -cycloalkylene or heterocycloalkylene, the cycloalkylene or heterocycloalkylene is a 5 membered ring. In some embodiments, when Y is cycloalkylene or heterocycloalkylene, L is —CH₂—.

In some embodiments of a compound of Formula (I), (II), (II-a), (II-b), (II-c), (III), (III-a), (III-b), (III-c), (IV), (IV-a), (IV-b), or (IV-c), or a pharmaceutically acceptable salt thereof, when Y is —C(R¹⁰)₂—, each R¹⁰ is independently selected from hydrogen, halogen, and unsubstituted —C₁-C₆alkyl. In some embodiments, each R¹⁰ is independently selected from hydrogen and unsubstituted —C₁-C₆alkyl. In some embodiments, each R¹⁰ is hydrogen.

In some embodiments of a compound of Formula (I), (II), (II-a), (II-b), (II-c), (III), (III-a), (III-b), (III-c), (IV), (IV-a), (IV-b), or (IV-c), or a pharmaceutically acceptable salt thereof, Y is selected from —O—, —S—, —S(═O)₂—, —NR⁹—, —C(R¹⁰)₂—, and substituted or unsubstituted heterocycloalkylene; R⁹ is selected from hydrogen and unsubstituted —C₁-C₆alkyl; and each R¹⁰ is independently selected from hydrogen and unsubstituted —C₁-C₆alkyl. In some embodiments, each R¹⁰ is hydrogen.

In some embodiments of a compound of Formula (I), (II), (II-a), (II-b), (II-c), (III), (III-a), (III-b), (III-c), (IV), (IV-a), (IV-b), or (IV-c), or a pharmaceutically acceptable salt thereof, Y is selected from —O—, —S—, —NR⁹—, and —CH₂—; and R⁹ is selected from hydrogen and unsubstituted —C₁-C₆alkyl.

In some embodiments of a compound of Formula (I), (II), (II-a), (II-b), (II-c), (III), (III-a), (III-b), (III-c), (IV), (IV-a), (IV-b), or (IV-c), or a pharmaceutically acceptable salt thereof, Y is —NR⁹—; and R⁹ is unsubstituted —C₁-C₆alkyl. In some embodiments, Y is —NR⁹—; and R⁹ is unsubstituted —C₁-C₄alkyl. In some embodiments, Y is selected from —N(Et)- and —N(Me)-. In some embodiments, Y is —N(Me)-. In some embodiments, Y is —NH— or —N(Me)-.

In some embodiments of a compound of Formula (I), (II), (II-a), (II-b), (II-c), (III), (III-a), (III-b), (III-c), (IV), (IV-a), (IV-b), or (IV-c), or a pharmaceutically acceptable salt thereof, Y is substituted or unsubstituted heterocycloalkylene. In some embodiments, Y is unsubstituted heterocycloalkylene. In some embodiments, Y is substituted or unsubstituted monocyclic heterocycloalkylene. In some embodiments, Y is substituted or unsubstituted monocyclic heterocycloalkylene, wherein the heterocycloalkylene contains a nitrogen atom. In some embodiments, Y is substituted or unsubstituted monocyclic heterocycloalkylene, wherein the heterocycloalkylene contains a nitrogen atom and optionally one other heteroatom selected from a nitrogen atom, oxygen atom, and sulfur atom.

In some embodiments of a compound of Formula (I), (II), (II-a), (II-b), (II-c), (III), (III-a), (III-b), (III-c), (IV), (IV-a), (IV-b), or (IV-c), or a pharmaceutically acceptable salt thereof, Y is represented by

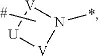

wherein # is the attachment point to L and * is the attachment point to the rest of the molecule; each V is independently —(C(R²¹)₂)ᵣ—; wherein each r is independently 1-3; each R²¹ is independently selected from hydrogen, halogen, —CN, —NO₂, —OR⁵², —CO₂R⁵², —C(═O)R⁵³, —C(═O)NR⁵²R⁵², —NR⁵²R⁵², —NR⁵²C(═O)R⁵³, —NR⁵²C(═O)OR^{52′}—SR⁵², —S(═O)R⁵³, —SO₂R⁵³, —SO₂NR⁵²R⁵², C₁-C₆ alkyl, C₁-C₆ haloalkyl, phenyl, 5- or 6-membered heteroaryl, monocyclic cycloalkyl, and monocyclic heterocycloalkyl; or two R²¹ on the same carbon atom are taken together to form a ═O or ═S; and U is selected from bond, —O—, —S—, and —NR²²—; wherein R²² is selected from hydrogen and unsubstituted —C₁-C₆alkyl. In some embodiments, each R²¹ is independently selected from hydrogen, halogen, —OR⁵², —NR⁵²R⁵², C₁-C₆ alkyl, and C₁-C₆ haloalkyl; or two R²¹ on the same carbon atom are taken together to form a ═O.

In some embodiments, each R²¹ is independently selected from hydrogen, halogen, —OR⁵², —NR⁵²R⁵², C₁-C₆ alkyl, and C₁-C₆ haloalkyl. In some embodiments, each R²¹ is independently selected from hydrogen and halogen. In some embodiments, each R²¹ is hydrogen.

In some embodiments, each r is independently 1 to 2 or 2 to 3. In some embodiments, each r is independently 1, 2, or 3.

In some embodiments, U is selected from bond, —O—, —S—, and —NR²²—. In some embodiments, U is a bond. In some embodiments, U is —O—. In some embodiments, U is —S—. In some embodiments, U is —NR²²—.

In some embodiments, when U is —NR²²—, R²² is selected from hydrogen and unsubstituted —C₁-C₄alkyl. In some embodiments, R²² is selected from hydrogen, methyl, ethyl, n-propyl, i-propyl, n-butyl, i-butyl, s-butyl and t-butyl. In some embodiments, R²² is selected from hydrogen, methyl and ethyl. In some embodiments, R²² is selected from hydrogen and methyl. In some embodiments, R²² is hydrogen. In some embodiments, R²² is methyl.

In some embodiments of a compound of Formula (I), (II), (II-a), (II-b), (II-c), (III), (III-a), (III-b), (III-c), (IV), (IV-a), (IV-b), or (IV-c), or a pharmaceutically acceptable salt thereof, Y

is represented by

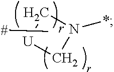

each r is independently 1-3; U is selected from bond, —O—, —S—, —NH— and —NMe-.

In some embodiments, each r is independently 1 to 2. In some embodiments, each r is 1. In some embodiments, each r is 2.

In some embodiments, U is a bond. In some embodiments, U is —O—. In some embodiments, U is —S—. In some embodiments, U is —NH—. In some embodiments, U is —NMe-

In some embodiments of a compound of Formula (I), (II), (II-a), (II-b), (II-c), (III), (III-a), (III-b), (III-c), (IV), (IV-a), (IV-b), or (IV-c), or a pharmaceutically acceptable salt thereof, Y is selected from —NH—, —NMe-, —NEt-, —N(n-Pr)-, —CH₂—, —S—, —O—, —S(═O)₂—,

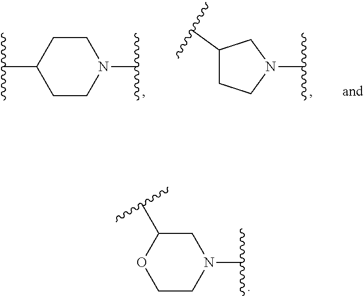 [SMILES: *C1CCN(*)CC1 ; *C1CCN(*)C1 ; *C1CN(*)CCO1]

In some embodiments of a compound of Formula (II), (III), and (IV), or a pharmaceutically acceptable salt thereof, the compound is represented by Formulas (II-d), (III-d), and (IV-d):

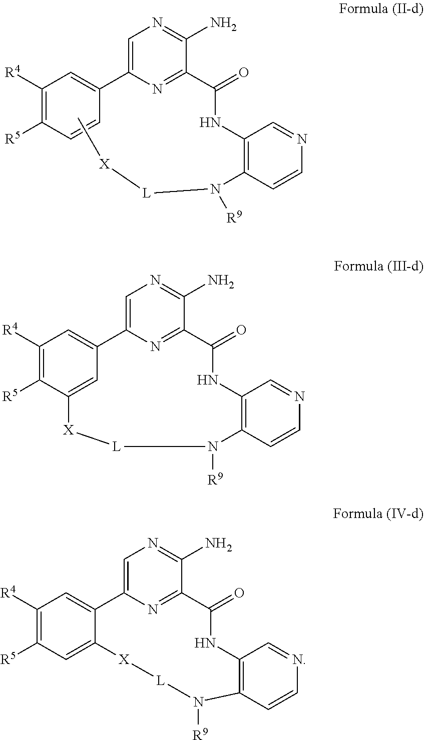

wherein R⁹ is methyl or ethyl; or a pharmaceutically acceptable salt thereof.

In some embodiments of a compound of Formula (I), (II), (II-a), (II-b), (II-c), (II-d), (III), (III-a), (III-b), (III-c), (III-d), (IV), (IV-a), (IV-b), (IV-c), or (IV-d), or a pharmaceutically acceptable salt thereof, X is selected from —O—, —NR⁷—, —C(R⁸)₂—, —C(R⁸)₂—O—, —S(═O)₂—, and —NR⁷—C(═O)—. In some embodiments, X is selected from —O—, —CH₂—, —CH₂—O—, —CH(R′)—O—, and —NR⁷—C(═O)—. In some embodiments, X is selected from *—NR⁷—C(═O)—# and #—NR⁷—C(═O)—*, wherein # is the attachment point to L and * is the attachment point to the rest of the molecule. In some embodiments, X is selected from *—NR⁷—C(═O)—#, wherein # is the attachment point to L and * is the attachment point to the rest of the molecule. In some embodiments, X is selected from *—CH(R⁸)—O—# and #—CH(R⁸)—O—*, wherein # is the attachment point to L and * is the attachment point to the rest of the molecule. In some embodiments, X is #—CH(R⁸)—O—*, wherein # is the attachment point to L and * is the attachment point to the rest of the molecule. In some embodiments, when X is #—CH(R⁸)—O—* and # is the attachment point to L and * is the attachment point to the rest of the molecule, R⁸ is R^{L}. In some embodiments, X is selected from —O— and —CH₂—O—. In some embodiments, X is —O— or —S(═O)₂—. In some embodiments, X is —O—. In some embodiments, X is —CH₂—O—. In some embodiments, X is —NR⁷—C(═O)—. In some embodiments, X is —NH—C(═O)—.

In some embodiments of a compound of Formula (I), (II), (II-a), (II-b), (II-c), (II-d), (III), (III-a), (III-b), (III-c), (III-d), (IV), (IV-a), (IV-b), (IV-c), or (IV-d), or a pharmaceutically acceptable salt thereof, when X is —NR⁷—, —C(R⁸)₂—NR⁷—, —NR⁷—S(═O)₂—, or NR⁷—C(═O)—, R⁷ is selected from hydrogen, unsubstituted —C₁-C₆alkyl, and R^{L}. In some embodiments, R⁷ is selected from hydrogen and unsubstituted —C₁-C₆alkyl. In some embodiments, R⁷ is selected from hydrogen and unsubstituted —C₁-C₄alkyl. In some embodiments, R⁷ is hydrogen. In some embodiments, R⁷ is unsubstituted —C₁-C₆alkyl. In some embodiments, R⁷ is unsubstituted —C₁-C₄alkyl. In some embodiments, R⁷ is selected from hydrogen and methyl. In some embodiments, R⁷ is —CH₃. In some embodiments, R⁷ is R^{L}.

In some embodiments of a compound of Formula (I), (II), (II-a), (II-b), (II-c), (II-d), (III), (III-a), (III-b), (III-c), (III-d), (IV), (IV-a), (IV-b), (IV-c), or (IV-d), or a pharmaceutically acceptable salt thereof, when X is —C(R⁸)₂—, —C(R⁸)₂—O—, —C(R⁸)₂—S—, or —C(R⁸)₂—NR⁷—, each R⁸ is independently selected from hydrogen, unsubstituted —C₁-C₆alkyl, and R^{L}. In some embodiments, each R⁸ is independently selected from hydrogen, unsubstituted —C₁-C₆alkyl and R^{L}. In some embodiments, each R⁸ is independently selected from hydrogen and unsubstituted —C₁-C₆alkyl. In some embodiments, each R⁸ is independently selected from hydrogen and unsubstituted —C₁-C₄alkyl. In some embodiments, each R⁸ is independently selected from hydrogen and methyl. In some embodiments, each R⁸ is hydrogen. In some embodiments, each R⁸ is independently selected from hydrogen or R^{L}. In some embodiments, one R⁸ is hydrogen, and the other R⁸ is R^{L}.

In some embodiments of a compound of Formula (I), (II), (II-a), (II-b), (II-c), (II-d), (III), (III-a), (III-b), (III-c), (III-d), (IV), (IV-a), (IV-b), (IV-c), or (IV-d), or a pharmaceutically acceptable salt thereof, X is selected from —O—, —NR⁷—, —C(R⁸)₂—, —C(R⁸)₂—O—, —S(═O)₂—, and —NR⁷—C(═O)—; R⁷ is selected from hydrogen, unsubstituted —C₁-C₆alkyl, and R^{L}; and each R⁸ is independently selected from hydrogen, unsubstituted —C₁-C₆alkyl, and R^{L}.

In some embodiments of a compound of Formula (I), (II), (II-a), (II-b), (II-c), (II-d), (III), (III-a), (III-b), (III-c), (III-d), (IV), (IV-a), (IV-b), (IV-c), or (IV-d), or a pharmaceutically acceptable salt thereof, X is selected from —O—, —CH₂—, —CH₂—O—, —CH(R′)—O—, and —NR⁷—C(═O)—; R⁷ is selected from hydrogen, unsubstituted —C₁-C₆alkyl, and R^{L}; and each R⁸ is selected from hydrogen, unsubstituted —C₁-C₆alkyl and R^{L}.

In some embodiments of a compound of Formula (I), (II), (II-a), (II-b), (II-c), (II-d), (III), (III-a), (III-b), (III-c), (III-d), (IV), (IV-a), (IV-b), (IV-c), or (IV-d), or a pharmaceutically acceptable salt thereof, X is selected from —O—, —CH₂—, —CH₂—O—, —CH(R′)—O—, and —NR⁷—C(═O)—; R⁷ is selected from hydrogen and unsubstituted —C₁-C₆alkyl; and R⁸ is unsubstituted —C₁-C₆alkyl.

In some embodiments of a compound of Formula (I), (II), (II-a), (II-b), (II-c), (II-d), (III), (III-a), (III-b), (III-c), (III-d), (IV), (IV-a), (IV-b), (IV-c), or (IV-d), or a pharmaceutically acceptable salt thereof, each R⁷ and R⁸ is independently selected from hydrogen and —C₁-C₆alkyl. In some embodiments, each R⁷ and R⁸ is independently selected from hydrogen and —C₁-C₄alkyl. In some embodiments, each R⁷ and R⁸ is independently selected from hydrogen and —CH₃. In some embodiments, each R⁷ and R⁸ is independently selected from hydrogen, CH₃ or R^{L}.

In some embodiments of a compound of Formula (I), (II), (II-a), (II-b), (II-c), (II-d), (III), (III-a), (III-b), (III-c), (III-d), (IV), (IV-a), (IV-b), (IV-c), or (IV-d), or a pharmaceutically acceptable salt thereof, X is selected from *—NR⁷—C(═O)—# and #—NR⁷—C(═O)—*; wherein # is the attachment point to L and * is the attachment point to the rest of the molecule. In some embodiments of a compound of Formula (I), (II), (II-a), (II-b), (II-c), (II-d), (III), (III-a), (III-b), (III-c), (III-d), (IV), (IV-a), (IV-b), (IV-c), or (IV-d), or a pharmaceutically acceptable salt thereof, X is selected from *—NR⁷—C(═O)—# wherein # is the attachment point to L and * is the attachment point to the rest of the molecule.

In some embodiments of a compound of Formula (I), (II), (II-a), (II-b), (II-c), (II-d), (III), (III-a), (III-b), (III-c), (III-d), (IV), (IV-a), (IV-b), (IV-c), or (IV-d), or a pharmaceutically acceptable salt thereof, X is selected from —O—, —C(R⁸)₂—O—, and *—NR⁷—C(═O)—#; wherein # is the attachment point to L and * is the attachment point to the rest of the molecule; and R⁷ and R⁸ are independently selected from hydrogen and unsubstituted —C₁-C₆alkyl. In some such aspects, R⁷ and R⁸ are independently selected from hydrogen and —CH₃.

In some embodiments of a compound of Formula (I), (II), (II-a), (II-b), (II-c), (II-d), (III), (III-a), (III-b), (III-c), (III-d), (IV), (IV-a), (IV-b), (IV-c), or (IV-d), or a pharmaceutically acceptable salt thereof, X is selected from —O— and —CH₂—O—.

In some embodiments of a compound of Formula (I), (II), (II-a), (II-b), (II-c), (II-d), (III), (III-a), (III-b), (III-c), (III-d), (IV), (IV-a), (IV-b), (IV-c), or (IV-d), or a pharmaceutically acceptable salt thereof, X is —O— or —S(═O)₂—.

In some embodiments of a compound of Formula (I), (II), (II-a), (II-b), (II-c), (II-d), (III), (III-a), (III-b), (III-c), (III-d), (IV), (IV-a), (IV-b), (IV-c), or (IV-d), or a pharmaceutically acceptable salt thereof, L is selected from substituted or unsubstituted C₁-C₁₀ alkylene, —[C(R¹¹)₂]_{q}(W)—, substituted or unsubstituted C₂-C₁₀ alkenylene, or substituted or unsubstituted C₂-C₁₀ alkynylene, and -[(substituted or unsubstituted C₁-C₄ alkylene)-Z-]ₚ-(substituted or unsubstituted C₁-C₄ alkylene). In some embodiments, L is selected from substituted or unsubstituted C₁-C₁₀ alkylene, —[C(R¹¹)₂]_{q}—(W)ₜ— and -[(substituted or unsubstituted C₁-C₄ alkylene)-Z]ₚ-(substituted or unsubstituted C₁-C₄ alkylene)-.

In some embodiments of a compound of Formula (I), (II), (II-a), (II-b), (II-c), (II-d), (III), (III-a), (III-b), (III-c), (III-d), (IV), (IV-a), (IV-b), (IV-c), or (IV-d), or a pharmaceutically acceptable salt thereof, L is substituted or unsubstituted C₁-C₁₀ alkylene, substituted or unsubstituted C₂-C₁₀ alkenylene, or substituted or unsubstituted C₂-C₁₀ alkynylene. In some embodiments, L is substituted or unsubstituted C₁-C₁₀ alkylene. In some embodiments, L is a substituted or unsubstituted C₁-C₆ alkylene; or L is a C₁-C₆ alkylene which is substituted by 1, 2, or 3 groups selected from halogen, —CN, —O—(C₁-C₆ alkyl), C₁-C₆ alkyl, and C₁-C₆ haloalkyl. In some embodiments, L is an unsubstituted C₁-C₁₀ alkylene. In some embodiments, L is an unsubstituted C₁-C₆ alkylene.

In some embodiments of a compound of Formula (I), (II), (II-a), (II-b), (II-c), (II-d), (III), (III-a), (III-b), (III-c), (III-d), (IV), (IV-a), (IV-b), (IV-c), or (IV-d), or a pharmaceutically acceptable salt thereof, L is selected from substituted or unsubstituted C₁-C₁₀ alkylene, —[C(R¹¹)₂]_{q}—(W)ₜ— and -[(substituted or unsubstituted C₁-C₄ alkylene)-Z]ₚ-(substituted or unsubstituted C₁-C₄ alkylene)-; each Z is —O—; p is 1-5; and q is 1 to 10.

In some embodiments of a compound of Formula (I), (II), (II-a), (II-b), (II-c), (II-d), (III), (III-a), (III-b), (III-c), (III-d), (IV), (IV-a), (IV-b), (IV-c), or (IV-d), or a pharmaceutically acceptable salt thereof, L is selected from *—[C(R¹¹)₂]_{q}—(W)ₜ—# and #—[C(R¹¹)₂]_{q}—(W)ₜ—*, wherein # is the attachment point to L and * is the attachment point to the rest of the molecule. In some embodiments, L is —[(CH₂CH₂)—O]ₚ—(CH₂CH₂)—; and p is 1-5. In some embodiments, L is —[(CH₂CH₂)—O]ₚ—(CH₂CH₂)—; and p is 1-3.

In some embodiments of a compound of Formula (I), (II), (II-a), (II-b), (II-c), (II-d), (III), (III-a), (III-b), (III-c), (III-d), (IV), (IV-a), (IV-b), (IV-c), or (IV-d), or a pharmaceutically acceptable salt thereof, when L is —[C(R¹¹)₂]_{q}—(W)ₜ—, W is unsubstituted or substituted cycloalkylene or unsubstituted or substituted heterocycloalkylene. In some embodiments, W is unsubstituted or substituted cycloalkylene. In some embodiments, W is unsubstituted or substituted heterocycloalkylene. In some embodiments, W is unsubstituted cycloalkylene or unsubstituted heterocycloalkylene. In some embodiments, W is unsubstituted cycloalkylene. In some embodiments, W is unsubstituted heterocycloalkylene.

In some embodiments of a compound of Formula (I), (II), (II-a), (II-b), (II-c), (II-d), (III), (III-a), (III-b), (III-c), (III-d), (IV), (IV-a), (IV-b), (IV-c), or (IV-d), or a pharmaceutically acceptable salt thereof, when L is -[(substituted or unsubstituted C₁-C₄ alkylene)-Z]ₚ-(substituted or unsubstituted C₁-C₄ alkylene)-, each Z is independently selected from —O—, —S—, and —NR¹¹—. In some embodiments, each Z is independently selected from —O— and —NR¹¹—. In some embodiments, each Z is —O—.

In some embodiments of a compound of Formula (I), (II), (II-a), (II-b), (II-c), (II-d), (III), (III-a), (III-b), (III-c), (III-d), (IV), (IV-a), (IV-b), (IV-c), or (IV-d), or a pharmaceutically acceptable salt thereof, when L is —[C(R¹¹)₂]_{q}—(W)ₜ, each R¹¹ is independently selected from hydrogen and unsubstituted or substituted —C₁-C₆alkyl. In some embodiments, each R¹¹ is independently selected from hydrogen and unsubstituted —C₁-C₆alkyl. In some embodiments, each R¹¹ is independently selected from hydrogen and unsubstituted —C₁-C₄alkyl. In some embodiments, each R¹¹ is hydrogen. In some embodiments, each R¹¹ is independently unsubstituted —C₁-C₆alkyl. In some embodiments, each R¹¹ is independently unsubstituted —C₁-C₄alkyl. In some embodiments, each R¹¹ is independently selected from hydrogen and methyl. In some embodiments, one R¹¹ is —CH₃.

In some embodiments, when X is in the ortho position, L is substituted or unsubstituted C₁-C₃ alkylene. In other embodiments, wherein X is in the meta positon, L is substituted or unsubstituted C₁-C₆ alkylene.

In some embodiments of a compound of Formula (I), (II), (II-a), (II-b), (II-c), (II-d), (III), (III-a), (III-b), (III-c), (III-d), (IV), (IV-a), (IV-b), (IV-c), or (IV-d), or a pharmaceutically acceptable salt thereof, when L is -[(substituted or unsubstituted C₁-C₄ alkylene)-Z]ₚ-(substituted or unsubstituted C₁-C₄ alkylene)-, p is 1 to 3. In some embodiments, p is 1 to 2, 1 to 3, or 2 to 3. In some embodiments, p is 1, 2, or 3.

In some embodiments of a compound of Formula (I), (II), (II-a), (II-b), (II-c), (II-d), (III), (III-a), (III-b), (III-c), (III-d), (IV), (IV-a), (IV-b), (IV-c), or (IV-d), or a pharmaceutically acceptable salt thereof, q is 1 to 2, 1 to 3, 1 to 3, or 1 to 4. In some embodiments, q is 1.

In some embodiments of a compound of Formula (I), (II), (II-a), (II-b), (II-c), (II-d), (III), (III-a), (III-b), (III-c), (III-d), (IV), (IV-a), (IV-b), (IV-c), or (IV-d), or a pharmaceutically acceptable salt thereof, L is a bond; and Y is selected from substituted or unsubstituted cycloalkylene and substituted or unsubstituted heterocycloalkylene. In some embodiments, L is a bond, and Y is substituted or unsubstituted heterocycloalkylene. In some embodiments, L is a bond, and Y is unsubstituted heterocycloalkylene. In some embodiments, L is a bond, and Y is monocyclic heterocycloalkylene. In some embodiments of a compound of Formula (I), (II), (II-a), (II-b), (II-c), (II-d), (III), (III-a), (III-b), (III-c), (III-d), (IV), (IV-a), (IV-b), (IV-c), or (IV-d), or a pharmaceutically acceptable salt thereof, L is not a bond.

In some embodiments of a compound of Formula (I), (II), (II-a), (II-b), (II-c), (II-d), (III), (III-a), (III-b), (III-c), (III-d), (IV), (IV-a), (IV-b), (IV-c), or (IV-d), or a pharmaceutically acceptable salt thereof, L is an unsubstituted C₁-C₆ alkylene; or L is a C₁-C₆ alkylene which is substituted by 1, 2, or 3 groups selected from halogen, —CN, —O—(C₁-C₆ alkyl), —C₁-C₆ alkyl, —C₁-C₆ haloalkyl, —OH, —NH₂, or —NHCH₃.

In some embodiments of a compound of Formula (I), (II), (II-a), (II-b), (II-c), (II-d), (III), (III-a), (III-b), (III-c), (III-d), (IV), (IV-a), (IV-b), (IV-c), or (IV-d), or a pharmaceutically acceptable salt thereof, L is selected from bond,

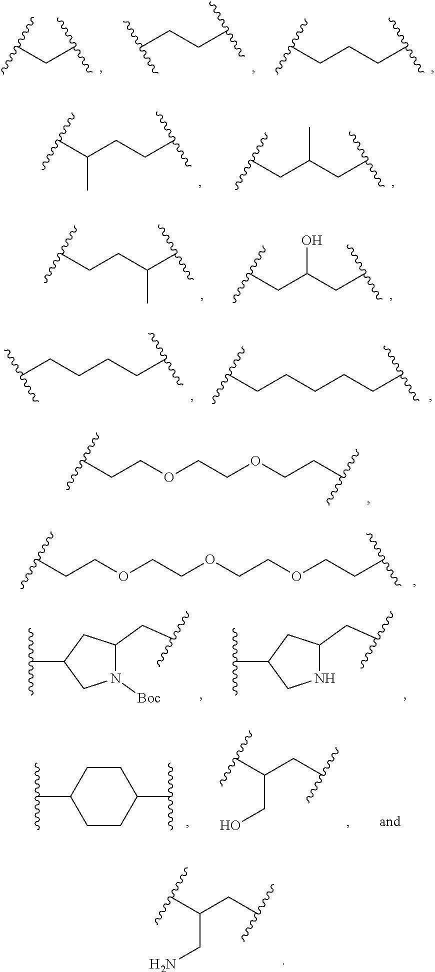

In some embodiments of a compound of Formula (I), (II), (II-a), (II-b), (II-c), (II-d), (III), (III-a), (III-b), (III-c), (III-d), (IV), (IV-a), (IV-b), (IV-c), or (IV-d), or a pharmaceutically acceptable salt thereof, L is selected from bond,

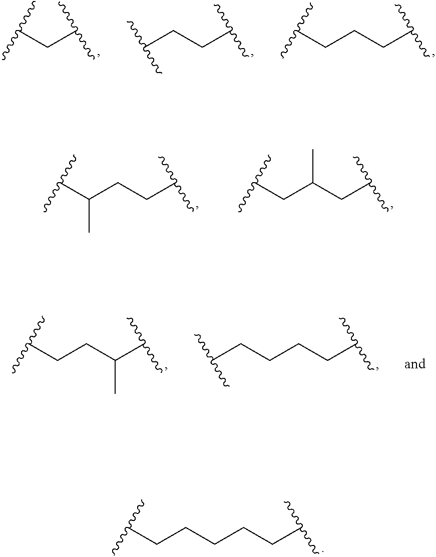

In some embodiments of a compound of Formula (I), (II), (II-a), (II-b), (II-c), (II-d), (III), (III-a), (III-b), (III-c), (III-d), (IV), (IV-a), (IV-b), (IV-c), or (IV-d), or a pharmaceutically acceptable salt thereof, —X-L-Y— is selected from

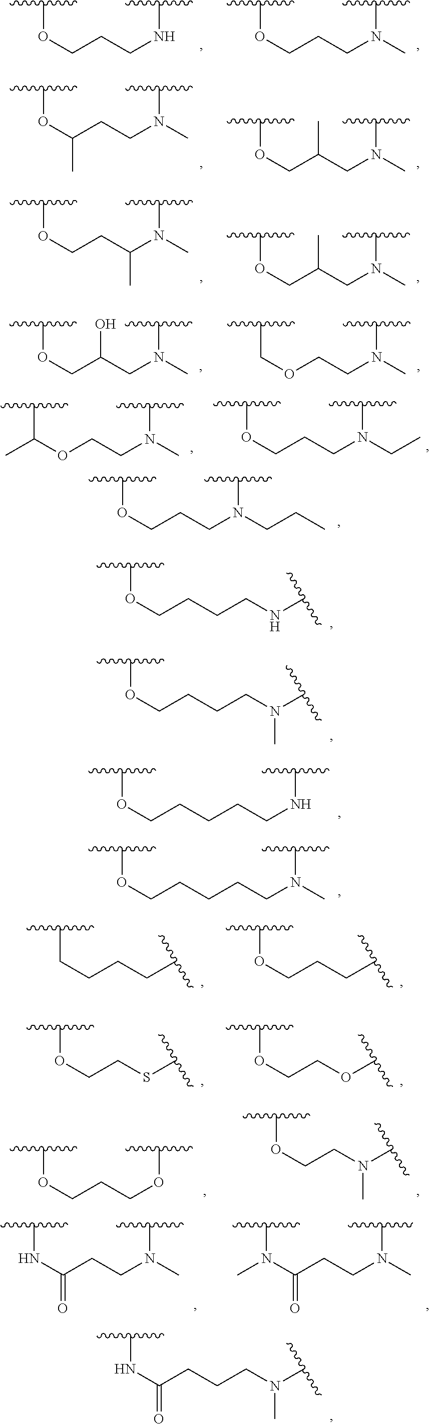

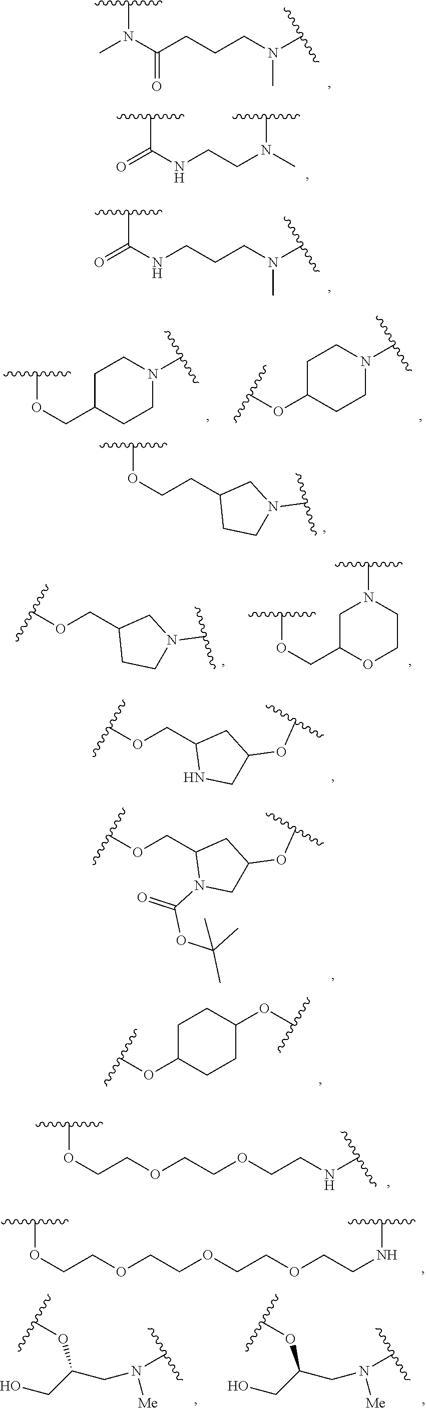

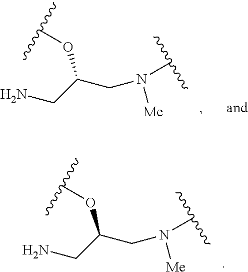 [SMILES: *O[C@@H](CN)CN(*)C ; *O[C@H](CN)CN(*)C]

In some embodiments of a compound of Formula (I), (II), (II-a), (II-b), (II-c), (II-d), (III), (III-a), (III-b), (III-c), (III-d), (IV), (IV-a), (IV-b), (IV-c), or (IV-d), or a pharmaceutically acceptable salt thereof, —X-L-Y— is selected from —X-L-Y— is selected from

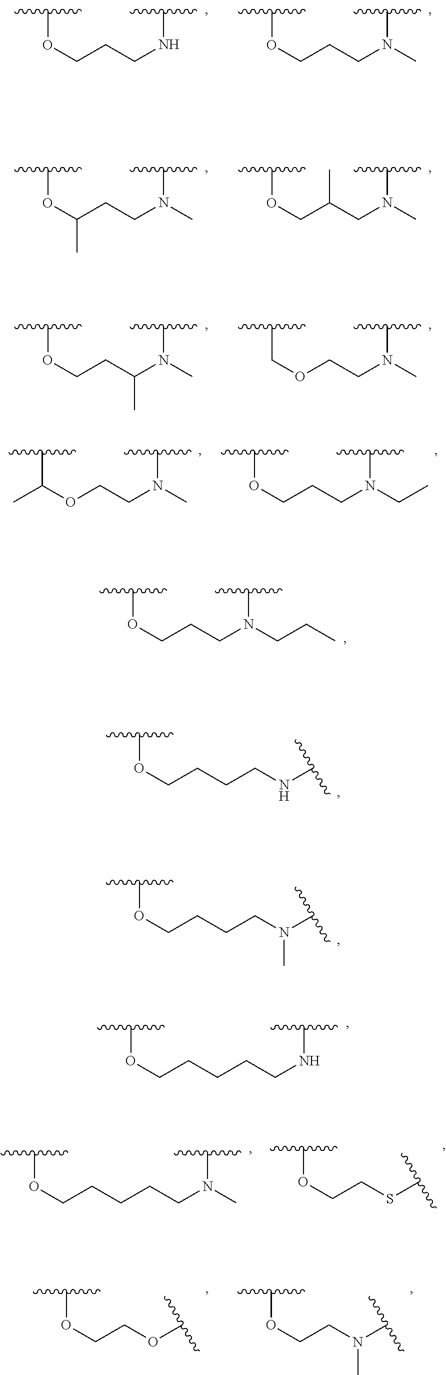

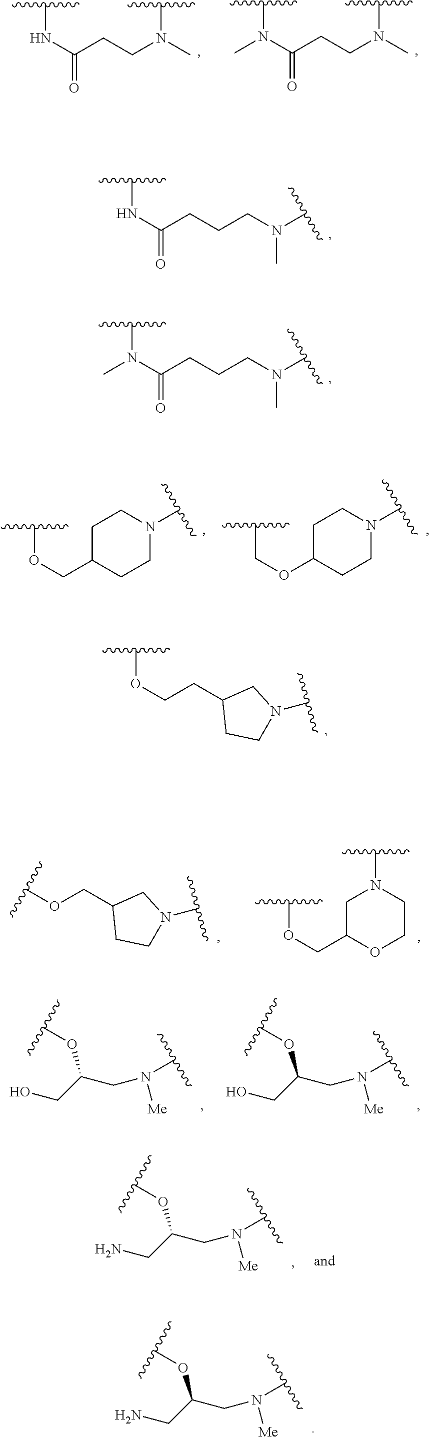

In some embodiments of a compound of Formula (I), (II), (II-a), (II-b), (II-c), (II-d), (III), (III-a), (III-b), (III-c), (III-d), (IV), (IV-a), (IV-b), (IV-c), or (IV-d), or a pharmaceutically acceptable salt thereof, —X-L-Y— is selected from —X-L-Y— is selected from,

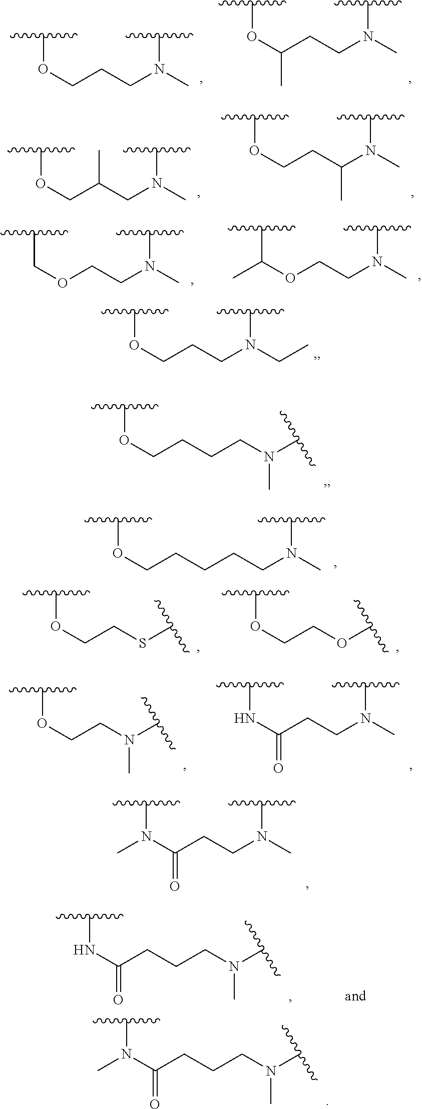

In some embodiments of a compound of Formula (I), (II), (II-a), (II-b), (II-c), (II-d), (III), (III-a), (III-b), (III-c), (III-d), (IV), (IV-a), (IV-b), (IV-c), or (IV-d), or a pharmaceutically acceptable salt thereof, —X-L-Y— is selected from —X-L-Y— is selected from

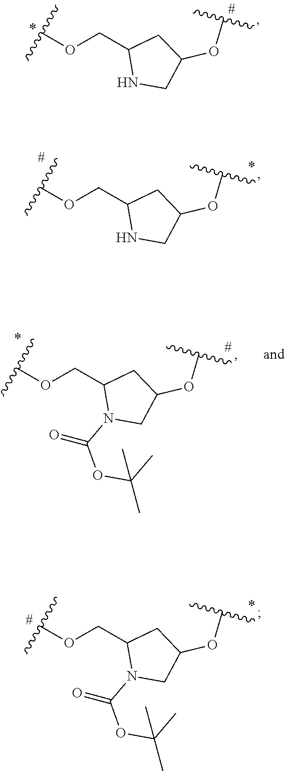 [SMILES: *OCC1CC(O*)CN1 ; *OCC1CC(O*)CN1 ; *OCC1CC(O*)CN1C(=O)OC(C)(C)C ; *OCC1CC(O*)CN1C(=O)OC(C)(C)C]

wherein # is the attachment point to L and * is the attachment point to the rest of the molecule.

In some embodiments of a compound of Formula (I), (II), (II-a), (II-b), (II-c), (II-d), (III), (III-a), (III-b), (III-c), (III-d), (IV), (IV-a), (IV-b), (IV-c), or (IV-d), or a pharmaceutically acceptable salt thereof:
X is selected from —O—, —C(R⁸)₂—, —C(R⁸)₂—O—, and —NR⁷—C(═O)—;
   R⁷ is selected from hydrogen and —C₁-C₆alkyl, e.g., methyl;
   each R⁸ is independently selected from R^{L}, hydrogen and —C₁-C₆alkyl, e.g., methyl;
Y is selected from —O—, —S—, —NR⁹—, —C(R¹⁰)₂—, substituted or unsubstituted heterocycloalkylene, e.g., substituted or unsubstituted morpholinylene, substituted or unsubstituted pyrrolidinylene, substituted or unsubstituted piperidinylene;
   R⁹ is selected from hydrogen and —C₁-C₆alkyl, e.g., methyl, ethyl and propyl;
   each R¹⁰ is hydrogen;
L is selected from a bond, substituted or unsubstituted C₁-C₆ alkylene, —[C(R¹¹)₂]_{q}—(W)—, and [(substituted or unsubstituted C₁-C₄ alkylene)-Z-]ₚ-(substituted or unsubstituted C₁-C₄ alkylene);
   W is unsubstituted or substituted cyclohexylene, or substituted or unsubstituted pyrrolidinylene;
   each Z is —O—;
   each R¹¹ is hydrogen;
   p is 1-5; and
   q is 1;
wherein if L is a bond, then Y is substituted or unsubstituted heterocycloalkylene.

In such embodiments R⁴ and R⁵ are independently selected from hydrogen, R²⁰, —O—R^{L}, and R^{L} and any combinations thereof. In certain exemplary embodiments, R⁴ and R⁵ are independently selected from hydrogen, unsubstituted —C₁-C₆ alkyl, —O—R^{L}, and R^{L}. In certain embodiments, R⁴ and R⁵ are independently selected from hydrogen, unsubstituted —C₁-C₆ alkyl, —O—R^{L}, and R^{L} wherein R^{L} is -(unsubstituted C₁-C₆ alkylene)-N(R¹³)₂; and each R¹³ is independently selected from hydrogen and unsubstituted or substituted —C₁-C₆alkyl; or two R¹³ on the same N atom are taken together with the N atom to which they are attached to form an unsubstituted or substituted N-containing heterocycle. In certain exemplary embodiments, R⁴ and R⁵ are independently selected from hydrogen, unsubstituted —C₁-C₆ alkyl, —O—R^{L}, and R^{L} wherein R^{L} is -(unsubstituted C₁-C₆ alkylene)-N(R¹³)₂; and each R¹³ is independently selected from hydrogen and methyl; or two R¹³ on the same N atom are taken together with the N atom to which they are attached to form an unsubstituted or substituted N-containing heterocycle. In certain exemplary embodiments, R⁴ and R⁵ are independently selected from hydrogen, unsubstituted —C₁-C₆ alkyl, —O—R^{L}, and R^{L} wherein R^{L} is -(unsubstituted C₁-C₆ alkylene)-N(R¹³)₂; and each R¹³ is independently selected from hydrogen and methyl.

In some embodiments of a compound of Formula (I), (II), (II-a), (II-b), (II-c), (II-d), (III), (III-a), (III-b), (III-c), (III-d), (IV), (IV-a), (IV-b), (IV-c), or (IV-d), or a pharmaceutically acceptable salt thereof:
X is selected from —O—, —C(R⁸)₂—O—, and *—NR⁷—C(═O)—# wherein # is the attachment point to L and * is the attachment point to the rest of the molecule;
   R⁷ is selected from R^{L}, hydrogen and —C₁-C₆alkyl, e.g., methyl;
   each R⁸ is independently selected from R^{L}, hydrogen or —C₁-C₆alkyl, e.g., methyl;
Y is selected from —O—, —S—, and —NR⁹—;
   R⁹ is selected from methyl, ethyl and propyl;
L is selected from substituted or unsubstituted C₁-C₁₀ alkylene (preferably C₁-C₆ alkylene). In some aspects, wherein X is in the ortho position, L is selected from substituted or unsubstituted C₁-C₃ alkylene.

In such embodiments R⁴ and R⁵ are independently selected from hydrogen, R²⁰, —O—R^{L}, and R^{L} and any combinations thereof. In certain exemplary embodiments, R⁴ and R⁵ are independently selected from hydrogen, unsubstituted —C₁-C₆ alkyl, —O—R^{L}, and R^{L}. In certain embodiments, R⁴ and R⁵ are independently selected from hydrogen, unsubstituted —C₁-C₆ alkyl, —O—R^{L}, and R^{L} wherein R^{L} is -(unsubstituted C₁-C₆ alkylene)-N(R¹³)₂; and each R¹³ is independently selected from hydrogen and unsubstituted or substituted —C₁-C₆alkyl; or two R¹³ on the same N atom are taken together with the N atom to which they are attached to form an unsubstituted or substituted N-containing heterocycle. In certain exemplary embodiments, R⁴ and R⁵ are independently selected from hydrogen, unsubstituted —C₁-C₆ alkyl, —O—R^{L}, and R^{L} wherein R^{L} is -(unsubstituted C₁-C₆ alkylene)-N(R¹³)₂; and each R¹³ is independently selected from hydrogen and methyl; or two R¹³ on the same N atom are taken together with the N atom to which they are attached to form an unsubstituted or substituted N-containing heterocycle. In certain exemplary embodiments, R⁴ and R⁵ are independently selected from hydrogen, unsubstituted —C₁-C₆ alkyl, —O—R^{L}, and R^{L} wherein R^{L} is -(unsubstituted C₁-C₆ alkylene)-N(R¹³)₂; and each R¹³ is independently selected from hydrogen and methyl.

In some embodiments of a compound of Formula (I), (II), (II-a), (II-b), (II-c), (II-d), (III), (III-a), (III-b), (III-c), (III-d), (IV), (IV-a), (IV-b), (IV-c), or (IV-d), or a pharmaceutically acceptable salt thereof:
X is selected from —O—, —C(R⁸)₂—O—, and *—NR⁷—C(═O)—# wherein # is the attachment point to L and * is the attachment point to the rest of the molecule;
   R⁷ is selected from hydrogen and —C₁-C₆alkyl, e.g., methyl;
   each R⁸ is independently selected from R^{L}, hydrogen and —C₁-C₆alkyl, e.g., methyl;
Y is selected from —O—, —S—, and —NR⁹—;
   R⁹ is selected from methyl, ethyl and propyl;
L is selected from substituted or unsubstituted C₁-C₁₀ alkylene (preferably C₁-C₆ unsubstituted alkylene). In some aspects, wherein X is in the ortho position, L is selected from substituted or unsubstituted C₁-C₃ alkylene.

In such embodiments R⁴ and R⁵ are independently selected from hydrogen, R²⁰, —O—R^{L}, and R^{L} and any combinations thereof. In certain exemplary embodiments, R⁴ and R⁵ are independently selected from hydrogen, unsubstituted —C₁-C₆ alkyl, —O—R^{L}, and R^{L}. In certain embodiments, R⁴ and R⁵ are independently selected from hydrogen, unsubstituted —C₁-C₆ alkyl, —O—R^{L}, and R^{L} wherein R^{L} is -(unsubstituted C₁-C₆ alkylene)-N(R¹³)₂; and each R¹³ is independently selected from hydrogen and unsubstituted or substituted —C₁-C₆alkyl; or two R¹³ on the same N atom are taken together with the N atom to which they are attached to form an unsubstituted or substituted N-containing heterocycle. In certain exemplary embodiments, R⁴ and R⁵ are independently selected from hydrogen, unsubstituted —C₁-C₆ alkyl, —O—R^{L}, and R^{L} wherein R^{L} is -(unsubstituted C₁-C₆ alkylene)-N(R¹³)₂; and each R¹³ is independently selected from hydrogen and methyl; or two R¹³ on the same N atom are taken together with the N atom to which they are attached to form an unsubstituted or substituted N-containing heterocycle. In certain exemplary embodiments, R⁴ and R⁵ are independently selected from hydrogen, unsubstituted —C₁-C₆ alkyl, —O—R^{L}, and R^{L} wherein R^{L} is -(unsubstituted C₁-C₆ alkylene)-N(R¹³)₂; and each R¹³ is independently selected from hydrogen and methyl.

In some embodiments of a compound of Formula (I), (II), (II-a), (II-b), (II-c), (II-d), (III), (III-a), (III-b), (III-c), (III-d), (IV), (IV-a), (IV-b), (IV-c), or (IV-d), or a pharmaceutically acceptable salt thereof:
X is selected from —O— and —C(R⁸)₂—O—;
   each R⁸ is hydrogen;
Y is —NR⁹;
   R⁹ is selected from methyl and ethyl
L is selected from unsubstituted C₁-C₆ alkylene, e.g., ethylene, propylene, butylene, and pentylene.

In such embodiments R⁴ and R⁵ are independently selected from hydrogen, R²⁰, —O—R^{L}, and R^{L} and any combinations thereof. In certain exemplary embodiments, R⁴ and R⁵ are independently selected from hydrogen, unsubstituted —C₁-C₆ alkyl, —O—R^{L}, and R^{L}. In certain embodiments, R⁴ and R⁵ are independently selected from hydrogen, unsubstituted —C₁-C₆ alkyl, —O—R^{L}, and R^{L} wherein R^{L} is -(unsubstituted C₁-C₆ alkylene)-N(R¹³)₂; and each R¹³ is independently selected from hydrogen and unsubstituted or substituted —C₁-C₆alkyl; or two R¹³ on the same N atom are taken together with the N atom to which they are attached to form an unsubstituted or substituted N-containing heterocycle. In certain exemplary embodiments, R⁴ and R⁵ are independently selected from hydrogen, unsubstituted —C₁-C₆ alkyl, —O—R^{L}, and R^{L} wherein R^{L} is -(unsubstituted C₁-C₆ alkylene)-N(R¹³)₂; and each R¹³ is independently selected from hydrogen and methyl; or two R¹³ on the same N atom are taken together with the N atom to which they are attached to form an unsubstituted or substituted N-containing heterocycle. In certain exemplary embodiments, R⁴ and R⁵ are independently selected from hydrogen, unsubstituted —C₁-C₆ alkyl, —O—R^{L}, and R^{L} wherein R^{L} is -(unsubstituted C₁-C₆ alkylene)-N(R¹³)₂; and each R¹³ is independently selected from hydrogen and methyl.

In some embodiments of a compound of Formula (I), (II), (II-a), (II-b), (II-c), (II-d), (III), (III-a), (III-b), (III-c), (III-d), (IV), (IV-a), (IV-b), (IV-c), or (IV-d), or a pharmaceutically acceptable salt thereof:
X is —C(R⁸)₂—O—;
   each R⁸ is hydrogen or R^{L};
Y is —NR⁹;
   R⁹ is selected from methyl and ethyl
L is selected from unsubstituted C₁-C₆ alkylene, e.g., ethylene, propylene, butylene, and pentylene.

In some embodiments of a compound of Formula (I), (II), (II-a), (II-b), (II-c), (II-d), (III), (III-a), (III-b), (III-c), (III-d), (IV), (IV-a), (IV-b), (IV-c), or (IV-d), or a pharmaceutically acceptable salt thereof:
X is —C(R⁸)₂—O—;
   each R⁸ is hydrogen or methyl;
Y is —NR⁹;
   R⁹ is selected from methyl, ethyl, or R^{L}
L is selected from unsubstituted C₁-C₆ alkylene, e.g., ethylene, propylene, butylene, and pentylene.

In such embodiments R⁴ and R⁵ are independently selected from hydrogen, R²⁰, —O—R^{L}, and R^{L} and any combinations thereof. In certain exemplary embodiments, R⁴ and R⁵ are independently selected from hydrogen, unsubstituted —C₁-C₆ alkyl, —O—R^{L}, and R^{L}. In certain embodiments, R⁴ and R⁵ are independently selected from hydrogen, unsubstituted —C₁-C₆ alkyl, —O—R^{L}, and R^{L} wherein R^{L} is -(unsubstituted C₁-C₆ alkylene)-N(R¹³)₂; and each R¹³ is independently selected from hydrogen and unsubstituted or substituted —C₁-C₆alkyl; or two R¹³ on the same N atom are taken together with the N atom to which they are attached to form an unsubstituted or substituted N-containing heterocycle. In certain exemplary embodiments, R⁴ and R⁵ are independently selected from hydrogen, unsubstituted —C₁-C₆ alkyl, —O—R^{L}, and R^{L} wherein R^{L} is -(unsubstituted C₁-C₆ alkylene)-N(R¹³)₂; and each R¹³ is independently selected from hydrogen and methyl; or two R¹³ on the same N atom are taken together with the N atom to which they are attached to form an unsubstituted or substituted N-containing heterocycle. In certain exemplary embodiments, R⁴ and R⁵ are independently selected from hydrogen, unsubstituted —C₁-C₆ alkyl, —O—R^{L}, and R^{L} wherein R^{L} is -(unsubstituted C₁-C₆ alkylene)-N(R¹³)₂; and each R¹³ is independently selected from hydrogen and methyl.

In some embodiments of a compound of Formula (I), (II), (II-a), (II-b), (II-c), (II-d), (III), (III-a), (III-b), (III-c), (III-d), (IV), (IV-a), (IV-b), (IV-c), or (IV-d), or a pharmaceutically acceptable salt thereof, —X-L-Y— is selected from

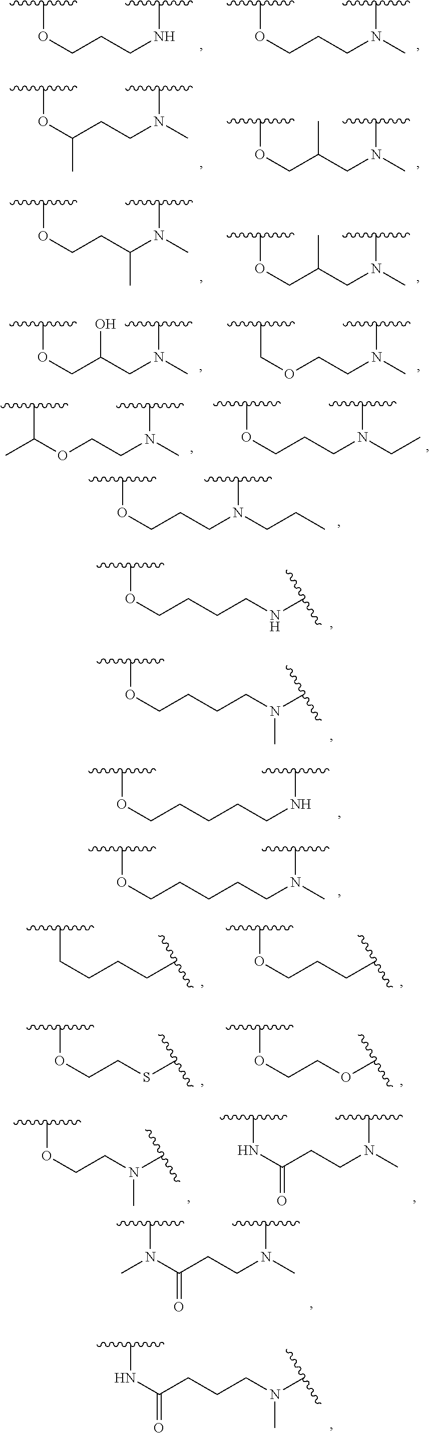

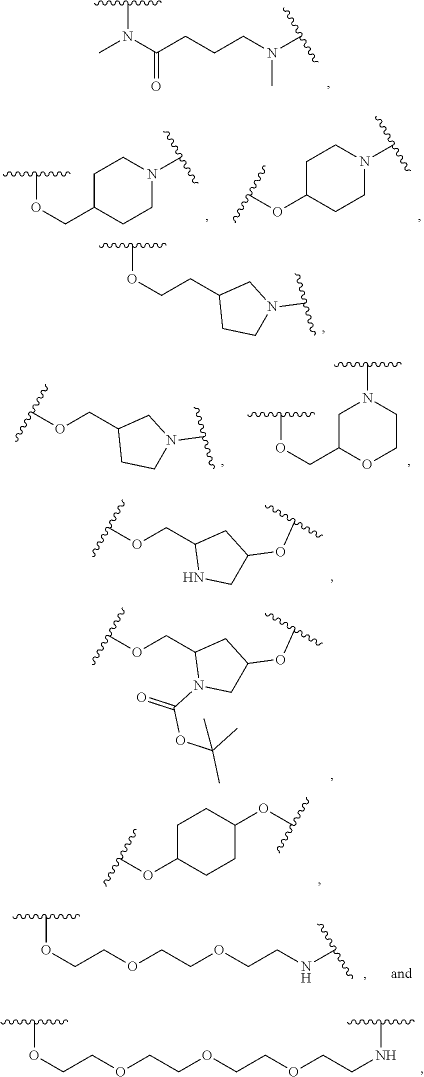

In some embodiments of a compound of Formula (I), (II), (II-a), (II-b), (II-c), (II-d), (III), (III-a), (III-b), (III-c), (III-d), (IV), (IV-a), (IV-b), (IV-c), or (IV-d), or a pharmaceutically acceptable salt thereof, —X-L-Y— is selected from,

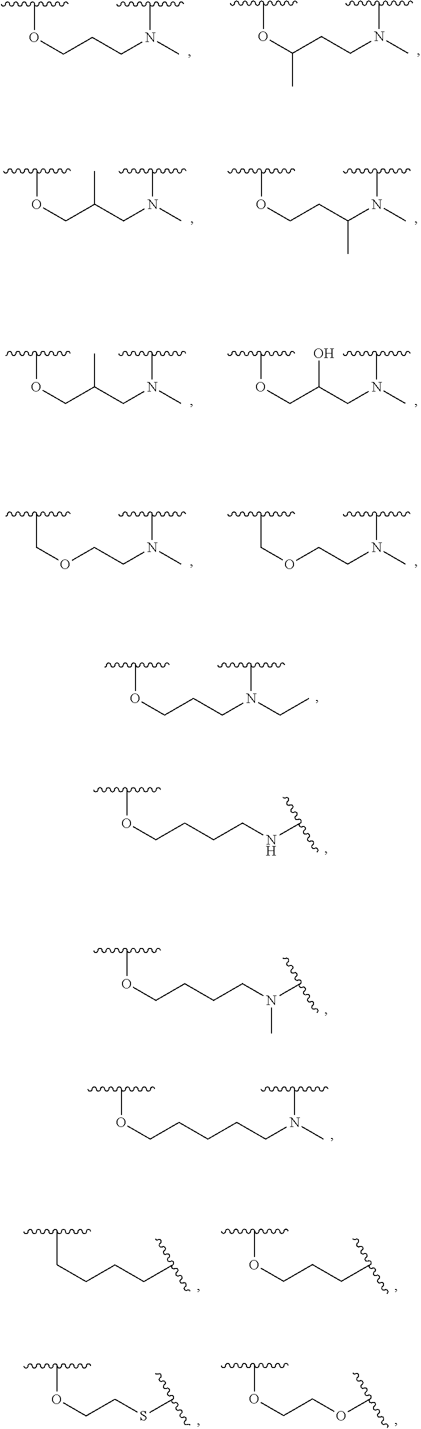

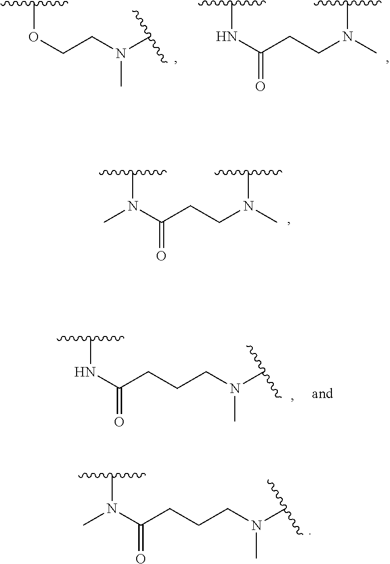

In some embodiments of a compound of Formula (I), (II), (II-a), (II-b), (II-c), (II-d), (III), (III-a), (III-b), (III-c), (III-d), (IV), (IV-a), (IV-b), (IV-c), or (IV-d), or a pharmaceutically acceptable salt thereof, —X-L-Y— is selected from

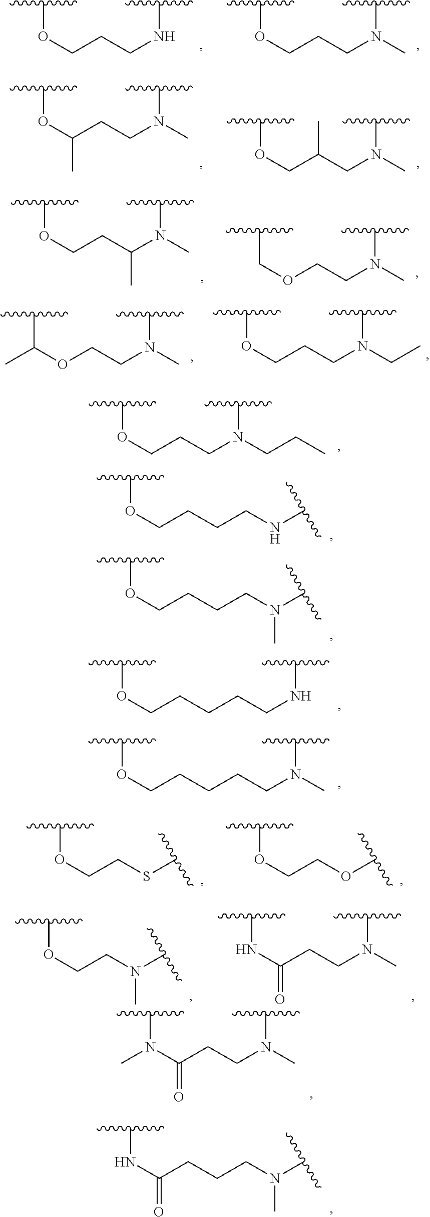

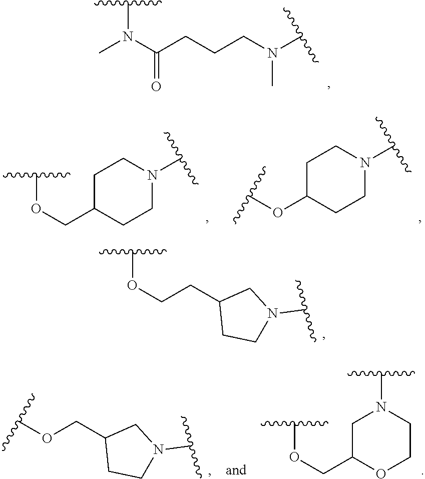

In some embodiments of a compound of Formula (I), (II), (II-a), (II-b), (II-c), (II-d), (III), (III-a), (III-b), (III-c), (III-d), (IV), (IV-a), (IV-b), (IV-c), or (IV-d), or a pharmaceutically acceptable salt thereof, —X-L-Y— is selected from

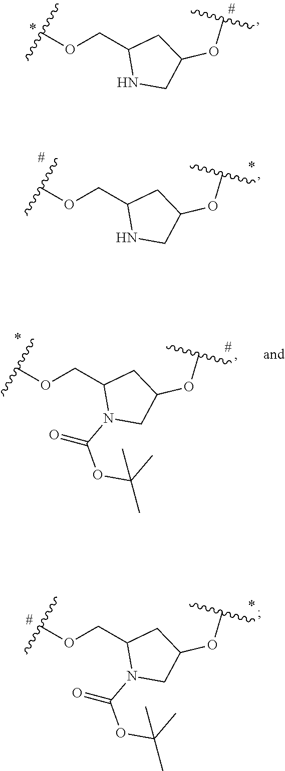 [SMILES: *OCC1CC(O*)CN1 ; *OCC1CC(O*)CN1 ; *OCC1CC(O*)CN1C(=O)OC(C)(C)C ; *OCC1CC(O*)CN1C(=O)OC(C)(C)C]

wherein # is the attachment point to L and * is the attachment point to the rest of the molecule.

In some embodiments of a compound of Formula (I), (II), (II-a), (II-b), (II-c), (II-d), (III), (III-a), (III-b), (III-c), (III-d), (IV), (IV-a), (IV-b), (IV-c), or (IV-d), or a pharmaceutically acceptable salt thereof, R⁴ is selected from hydrogen, halogen, —CN, —OH, —OR⁵⁰, —NR⁵¹R⁵¹, unsubstituted or substituted C₁-C₆ alkyl, unsubstituted or substituted cycloalkyl, unsubstituted or substituted heterocycloalkyl, —O—R^{L}, and R^{L}. In some embodiments, R⁴ is selected from hydrogen, unsubstituted C₁-C₆ alkyl, —O—R^{L}, and R^{L}. In some embodiments, R⁴ is hydrogen. In some embodiments, R⁴ is R²⁰. In some embodiments, R⁴ is R^{L}. In some embodiments, R⁴ is —O—R^{L}.

In some embodiments of a compound of Formula (I), (II), (II-a), (II-b), (II-c), (II-d), (III), (III-a), (III-b), (III-c), (III-d), (IV), (IV-a), (IV-b), (IV-c), or (IV-d), or a pharmaceutically acceptable salt thereof, R⁵ is selected from hydrogen, halogen, —CN, —OH, —OR⁵⁰, —NR⁵¹R⁵¹, unsubstituted or substituted C₁-C₆ alkyl, unsubstituted or substituted cycloalkyl, unsubstituted or substituted heterocycloalkyl, —O—R^{L}, and R^{L}. In some embodiments, R⁵ is selected from hydrogen, unsubstituted C₁-C₆ alkyl, —O—R^{L}, and R^{L}. In some embodiments, R⁵ is hydrogen. In some embodiments, R⁵ is R²⁰. In some embodiments, R⁵ is R^{L}. In some embodiments, R⁵ is —O—R^{L}.

In some embodiments of a compound of Formula (I), (II), (II-a), (II-b), (II-c), (II-d), (III), (III-a), (III-b), (III-c), (III-d), (IV), (IV-a), (IV-b), (IV-c), or (IV-d), or a pharmaceutically acceptable salt thereof, R^{L} is -(unsubstituted or substituted C₁-C₆ alkylene)-OR¹². In some embodiments, R^{L} is -(unsubstituted C₁-C₆ alkylene)-OR¹².

In some embodiments of a compound of Formula (I), (II), (II-a), (II-b), (II-c), (III), (III-a), (III-b), (III-c), (IV), (IV-a), (IV-b), or (IV-c), or a pharmaceutically acceptable salt thereof, R^{L} is -(unsubstituted or substituted C₁-C₆ alkylene)-N(R¹³)₂. In some embodiments, R^{L} is -(unsubstituted C₁-C₆ alkylene)-N(R¹³)₂.

In some embodiments of a compound of Formula (I), (II), (II-a), (II-b), (II-c), (III), (III-a), (III-b), (III-c), (IV), (IV-a), (IV-b), or (IV-c), or a pharmaceutically acceptable salt thereof, R¹² is selected from hydrogen, —C(═O)R⁵⁰, —C(═O)OR⁵¹, —C(═O)NR⁵¹R⁵¹, and unsubstituted or substituted —C₁-C₆alkyl. In some embodiments, R¹² is selected from hydrogen and unsubstituted —C₁-C₆alkyl. In some embodiments, R¹² is hydrogen.

In some embodiments of a compound of Formula (I), (II), (II-a), (II-b), (II-c), (II-d), (III), (III-a), (III-b), (III-c), (III-d), (IV), (IV-a), (IV-b), (IV-c), or (IV-d), or a pharmaceutically acceptable salt thereof, each R¹³ is independently selected from hydrogen, —C(═O)R⁵⁰, —C(═O)OR⁵¹, —C(═O)NR⁵¹R⁵¹, and unsubstituted or substituted —C₁-C₆alkyl; or two R¹³ on the same N atom are taken together with the N atom to which they are attached to form an unsubstituted or substituted N-containing heterocycle. In some embodiments, each R¹³ is independently selected from hydrogen, —C(═O)R⁵⁰, —C(═O)OR⁵¹, —C(═O)NR⁵¹R⁵¹, and unsubstituted or substituted —C₁-C₆alkyl. In some embodiments, each R¹³ is independently selected from hydrogen and unsubstituted —C₁-C₆alkyl. In some embodiments, each R¹³ is hydrogen. In some embodiments, two R¹³ on the same N atom are taken together with the N atom to which they are attached to form an unsubstituted or substituted N-containing heterocycle. In some embodiments, two R¹³ on the same N atom are taken together with the N atom to which they are attached to form a phthalimide.

In some embodiments of a compound of Formula (I), (II), (II-a), (II-b), (II-c), (II-d), (III), (III-a), (III-b), (III-c), (III-d), (IV), (IV-a), (IV-b), (IV-c), or (IV-d), or a pharmaceutically acceptable salt thereof, R^{L} is -(unsubstituted C₁-C₆ alkylene)-N(R¹³)₂; and each R¹³ is independently selected from hydrogen, —C(═O)R⁵⁰, —C(═O)OR⁵¹, —C(═O)NR⁵¹R⁵¹, and unsubstituted or substituted —C₁-C₆alkyl; or two R¹³ on the same N atom are taken together with the N atom to which they are attached to form an unsubstituted or substituted N-containing heterocycle.

In some embodiments of a compound of Formula (I), (II), (II-a), (II-b), (II-c), (II-d), (III), (III-a), (III-b), (III-c), (III-d), (IV), (IV-a), (IV-b), (IV-c), or (IV-d), or a pharmaceutically acceptable salt thereof, R^{L} is -(unsubstituted C₁-C₆ alkylene)-NH₂.

In some embodiments of a compound of Formula (I), (II), (II-a), (II-b), (II-c), (II-d), (III), (III-a), (III-b), (III-c), (III-d), (IV), (IV-a), (IV-b), (IV-c), or (IV-d), or a pharmaceutically acceptable salt thereof, R^{L} is -(unsubstituted C₁-C₆ alkylene)-N(R¹³)₂; and two R¹³ on the same N atom are taken together with the N atom to which they are attached to form a phthalimide.

In some embodiments of a compound of Formula (I), (II), (II-a), (II-b), (II-c), (II-d), (III), (III-a), (III-b), (III-c), (III-d), (IV), (IV-a), (IV-b), (IV-c), or (IV-d), or a pharmaceutically acceptable salt thereof, R^{L} is selected from

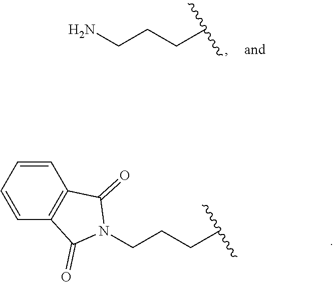 [SMILES: *CCCN ; *CCCN1C(=O)c2ccccc2C1=O]

In some embodiments, R^{L} is

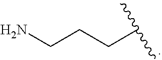 [SMILES: *CCCN]

In some embodiments of a compound of Formula (I), (II), (II-a), (II-b), (II-c), (II-d), (III), (III-a), (III-b), (III-c), (III-d), (IV), (IV-a), (IV-b), (IV-c), or (IV-d), or a pharmaceutically acceptable salt thereof, one of R⁴ or R⁵ is selected from

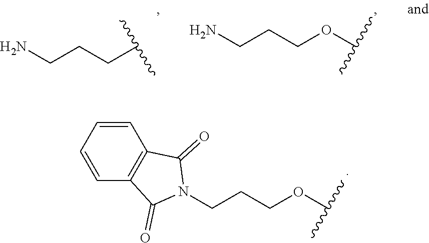 [SMILES: *CCCN ; *OCCCN ; *OCCCN1C(=O)c2ccccc2C1=O]

In some embodiments, one of R⁴ or R⁵ is selected from

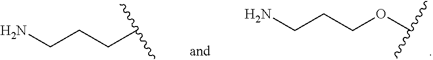 [SMILES: *CCCN ; *OCCCN]

In some embodiments, the other of R⁴ and R⁵ is hydrogen.

In some embodiments of a compound of Formula (I), (II), (II-a), (II-b), (II-c), (II-d), (III), (III-a), (III-b), (III-c), (III-d), (IV), (IV-a), (IV-b), (IV-c), or (IV-d), or a pharmaceutically acceptable salt thereof, at least one of R⁴ and R⁵ is —O—R^{L}. In alternative exemplary embodiments, R⁴ and R⁵ are independently selected from hydrogen or methyl. In some embodiments, R⁴ is selected from hydrogen, —C₁-C₆alkyl, and —O—R^{L}; R⁵ is selected from hydrogen, —C₁-C₆alkyl, and —O—R^{L}. In some such embodiments, R^{L} is selected from -(unsubstituted C₁-C₆ alkylene)-NH₂ and -(unsubstituted C₁-C₆ alkylene)-OH.

In some embodiments of a compound of Formula (I), (II), (II-a), (II-b), (II-c), (II-d), (III), (III-a), (III-b), (III-c), (III-d), (IV), (IV-a), (IV-b), (IV-c), or (IV-d), or a pharmaceutically acceptable salt thereof, each R²⁰ is independently selected from halogen, —CN, —OH, —OR⁵⁰, —NR⁵¹R⁵¹, —C(═O)OR⁵⁰, —C(═O)N⁵¹R⁵¹, —NR⁵¹C(═O)R⁵⁰, C₁-C₆alkyl, C₁-C₆haloalkyl, monocyclic carbocycle, and monocyclic heterocycle. In some embodiments, each R²⁰ is independently selected from halogen, —CN, —OH, —OR⁵⁰, —NR⁵¹R⁵¹, —C(═O)OR⁵⁰, —C(═O)NR⁵¹R⁵¹, —NR⁵¹C(═O)R⁵⁰, —SR⁵¹, —S(═O)R⁵⁰, —SO₂R⁵⁰, —SO₂NR⁵¹R⁵¹, —NHSO₂R⁵⁰, C₁-C₆alkyl, C₁-C₆haloalkyl, phenyl and monocyclic cycloalkyl. In some embodiments, each R²⁰ is independently selected from halogen, —CN, —OH, —OR⁵⁰, —NR⁵¹R⁵¹, —C(═O)OR⁵⁰, —C(═O)NR⁵¹R⁵¹, C₁-C₆alkyl, and C₁-C₆haloalkyl. In some embodiments, each R²⁰ is independently selected from halogen, —CN, —OH, —OR⁵⁰, —NR⁵¹R⁵¹. In some embodiments, each R²⁰ is independently selected from —F, —Cl, —Br, —CN, —OH, —OR⁵⁰, —NR⁵¹R⁵¹.

In some embodiments of a compound of Formula (I), (II), (II-a), (II-b), (II-c), (II-d), (III), (III-a), (III-b), (III-c), (III-d), (IV), (IV-a), (IV-b), (IV-c), or (IV-d), or a pharmaceutically acceptable salt thereof, each R⁵⁰ is independently selected from unsubstituted or substituted C₁-C₆alkyl, unsubstituted or substituted carbocycle, and unsubstituted or substituted heterocycle. In some embodiments, each R⁵⁰ is independently selected from unsubstituted or substituted C₁-C₆alkyl.

In some embodiments of a compound of Formula (I), (II), (II-a), (II-b), (II-c), (II-d), (III), (III-a), (III-b), (III-c), (III-d), (IV), (IV-a), (IV-b), (IV-c), or (IV-d), or a pharmaceutically acceptable salt thereof, each R⁵¹ is independently selected from hydrogen, unsubstituted or substituted C₁-C₆alkyl, unsubstituted or substituted carbocycle, and unsubstituted or substituted heterocycle. In some embodiments, each R⁵¹ is independently selected from hydrogen, unsubstituted or substituted C₁-C₆alkyl.

In some embodiments of a compound of Formula (I), (II), (II-a), (II-b), (II-c), (II-d), (III), (III-a), (III-b), (III-c), (III-d), (IV), (IV-a), (IV-b), (IV-c), or (IV-d), or a pharmaceutically acceptable salt thereof, two R⁵¹ on the same N atom are taken together with the N atom to which they are attached to form an unsubstituted or substituted N-containing heterocycle.

In some embodiments of a compound of Formula (I), (II), (II-a), (II-b), (II-c), (II-d), (III), (III-a), (III-b), (III-c), (III-d), (IV), (IV-a), (IV-b), (IV-c), or (IV-d), or a pharmaceutically acceptable salt thereof when any of L, Y, R^{L}, R¹, R⁷, R⁸, R⁹, R¹⁰, R¹¹, R¹, R¹³, R²⁰, R⁵⁰, and R⁵¹ are substituted, substituents on the L, Y, R^{L}, R¹, R⁷, R⁸, R⁹, R¹⁰, R¹¹, R¹², R¹³, R²⁰, R⁵⁰, and R⁵¹ are independently selected at each occurrence from halogen, —CN, —NO₂, —OR⁵², —CO₂R⁵², —C(═O)NR⁵²R⁵², —NR⁵²R⁵², —NR⁵²C(═O)R⁵³, —SR⁵², —S(═O)R⁵³, —SO₂R⁵³, —SO₂NR⁵²R⁵², unsubstituted C₁-C₆alkyl, unsubstituted C₁-C₆haloalkyl, unsubstituted monocyclic carbocycle, and unsubstituted monocyclic heterocycle. In some embodiments, substituents are independently selected at each occurrence from halogen, —CN, —NO₂—OR⁵², —CO₂R⁵², —C(═O)N⁵²R⁵², —NR⁵²R⁵², —NR⁵²C(═O)R⁵³, —S(═O)R⁵³, —SO₂R⁵³, —SO₂NR⁵²R⁵², unsubstituted C₁-C₆alkyl, unsubstituted C₁-C₆haloalkyl, unsubstituted phenyl and unsubstituted monocyclic cycloalkyl. In some embodiments, substituents are independently selected at each occurrence from halogen, —CN, —OR⁵², —CO₂R⁵², —C(═O)NR⁵²R⁵², —NR⁵²R⁵², unsubstituted C₁-C₆alkyl, and unsubstituted C₁-C₆haloalkyl.

In some embodiments of a compound of Formula (I), (II), (II-a), (II-b), (II-c), (II-d), (III), (III-a), (III-b), (III-c), (III-d), (IV), (IV-a), (IV-b), (IV-c), or (IV-d), or a pharmaceutically acceptable salt thereof, each R⁵² is independently selected from hydrogen, unsubstituted C₁-C₆alkyl, unsubstituted C₃-C₆cycloalkyl, unsubstituted phenyl, unsubstituted benzyl, unsubstituted 5-membered heteroaryl, and unsubstituted 6-membered heteroaryl. In some embodiments, each R⁵² is independently selected from hydrogen, unsubstituted C₁-C₆alkyl, unsubstituted C₃-C₆cycloalkyl, and unsubstituted phenyl. In some embodiments, each R⁵² is independently selected from hydrogen and unsubstituted C₁-C₆alkyl.

In some embodiments of a compound of Formula (I), (II), (II-a), (II-b), (II-c), (III), (III-a), (III-b) or (III-c), or a pharmaceutically acceptable salt thereof, two R⁵² groups are taken together with the N atom to which they are attached to form an unsubstituted N-containing heterocycle.

In some embodiments of a compound of Formula (I), (II), (II-a), (II-b), (II-c), (II-d), (III), (III-a), (III-b), (III-c), (III-d), (IV), (IV-a), (IV-b), (IV-c), or (IV-d), or a pharmaceutically acceptable salt thereof, each R⁵³ is independently selected from unsubstituted C₁-C₆alkyl, unsubstituted C₃-C₆cycloalkyl, unsubstituted phenyl, unsubstituted benzyl, unsubstituted 5-membered heteroaryl, and unsubstituted 6-membered heteroaryl. In some embodiments, each R⁵³ is independently selected from unsubstituted C₁-C₆alkyl, unsubstituted C₃-C₆cycloalkyl, and unsubstituted phenyl. In some embodiments, each R⁵³ is independently selected from unsubstituted C₁-C₆alkyl.

In certain embodiments, the compound is represented by Formula (III):

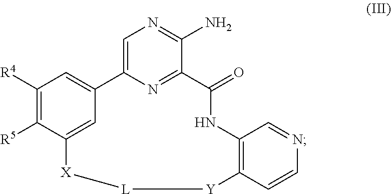

or a pharmaceutically acceptable salt thereof, wherein:
X is selected from —O—, —C(R⁸)₂—, —C(R⁸)₂—O—, and —NR⁷—C(═O)—;
   R⁷ is selected from hydrogen and —C₁-C₆alkyl, e.g., methyl;
   each R⁸ is independently selected from hydrogen or —C₁-C₆alkyl, e.g., methyl;
Y is selected from —O—, —S—, —NR⁹—, —C(R¹⁰)₂—, and substituted or unsubstituted heterocycloalkylene, e.g., substituted or unsubstituted morpholinylene, substituted or unsubstituted pyrrolidinylene, substituted or unsubstituted piperidinylene;
   R⁹ is selected from hydrogen and —C₁-C₆alkyl, e.g., methyl, ethyl and propyl;
   each R¹⁰ is hydrogen;
L is selected from a bond, substituted or unsubstituted C₁-C₆ alkylene, —[C(R¹¹)₂]_{q}—(W)—, and [(substituted or unsubstituted C₁-C₄ alkylene)-Z-]ₚ-(substituted or unsubstituted C₁-C₄ alkylene);
   W is unsubstituted or substituted cyclohexylene, or substituted or unsubstituted pyrrolidinylene;
   each Z is —O—;
   each R¹¹ is hydrogen;
   p is 1-5;
   q is 1;
   wherein if L is a bond, then Y is substituted or unsubstituted heterocycloalkylene;
R⁴ is selected from hydrogen, —C₁-C₆alkyl, e.g., methyl, and —O—R^{L};
R⁵ is selected from hydrogen, —C₁-C₆alkyl, e.g., methyl, and —O—R^{L};
R^{L} is selected from -(unsubstituted C₁-C₆ alkylene)-N(R¹³)₂; and
each R¹³ is independently selected from hydrogen, and —C₁-C₆alkyl, e.g., methyl;
or two R¹³ on the same N atom are taken together with the N atom to which they are attached to form an unsubstituted or substituted N-containing heterocycle;
wherein when any of L, Y, and W are substituted, substituents on the L, Y, and W are independently selected at each occurrence from halogen, —CN, —NO₂, —OR⁵², —CO₂R⁵², —C(═O)R⁵³, —C(═O)N⁵²R⁵², —NR⁵²R⁵², —NR⁵²C(═O)R⁵³, unsubstituted C₁-C₆ alkyl, unsubstituted C₁-C₆ haloalkyl, unsubstituted phenyl, unsubstituted 5- or 6-membered heteroaryl, unsubstituted monocyclic cycloalkyl, and unsubstituted monocyclic heterocycloalkyl; or two substituents on the same carbon atom are taken together to form a ═O or ═S;
   each R⁵² is independently selected from hydrogen, unsubstituted C₁-C₆ alkyl, unsubstituted C₃-C₆ cycloalkyl, unsubstituted 3- to 6-membered heterocycloalkyl, unsubstituted phenyl, unsubstituted benzyl, unsubstituted 5-membered heteroaryl, and unsubstituted 6-membered heteroaryl;
   or two R⁵² groups are taken together with the N atom to which they are attached to form an unsubstituted N-containing heterocycle; and
   each R⁵³ is independently selected from unsubstituted C₁-C₆alkyl, unsubstituted C₃-C₆cycloalkyl, unsubstituted phenyl, unsubstituted benzyl, unsubstituted 5-membered heteroaryl, and unsubstituted 6-membered heteroaryl.

In certain embodiments, the compound is represented by Formula (III):

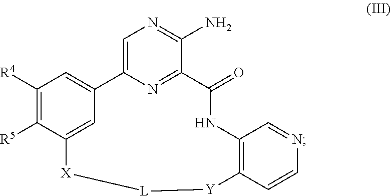

or a pharmaceutically acceptable salt thereof, wherein:
X is selected from —O—, —C(R⁸)₂—O—, and *—NR⁷—C(═O)—# wherein # is the attachment point to L and * is the attachment point to the rest of the molecule;
   each R⁸ is independently selected from hydrogen and methyl;
   R⁷ is hydrogen or methyl;
Y is —NR⁹;
   R⁹ is —C₁-C₆alkyl, e.g., methyl, ethyl, and propyl;
L is unsubstituted C₁-C₆ alkylene, e.g., ethylene, propylene, butylene, and pentylene;
R⁴ is selected from hydrogen, —C₁-C₆alkyl, e.g., methyl, and —O—R^{L};
R⁵ is selected from hydrogen, —C₁-C₆alkyl, e.g., methyl, and —O—R^{L}; and
R^{L} is selected from -(unsubstituted C₁-C₆ alkylene)-NH₂ and -(unsubstituted C₁-C₆ alkylene)-OH.

In some exemplary embodiments at least one of R⁴ and R⁵ is —O—R^{L}. In alternative exemplary embodiments, R⁴ and R⁵ are independently selected from hydrogen or methyl. In some embodiments, R⁴ is selected from hydrogen, —C₁-C₆alkyl, and —O—R^{L}; R⁵ is selected from hydrogen, —C₁-C₆alkyl, and —O—R^{L}. In some such embodiments, R^{L} is selected from -(unsubstituted C₁-C₆ alkylene)-NH₂ and -(unsubstituted C₁-C₆ alkylene)-OH.

In certain embodiments, the compound is represented by Formula (IV):

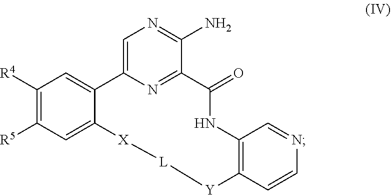

or a pharmaceutically acceptable salt thereof, wherein:
X is selected from #—C(R⁸)₂—O—*, wherein # is the attachment point to L and * is the attachment point to the rest of the molecule;
   each R⁸ is independently selected from hydrogen, methyl, and R^{L};
   R⁷ is hydrogen or methyl;
Y is —NR⁹;
   R⁹ is —C₁-C₆alkyl, e.g., methyl, ethyl, and propyl;
L is unsubstituted C₁-C₃ alkylene;
R⁴ is selected from hydrogen, —C₁-C₆alkyl, e.g., methyl, and —O—R^{L};
R⁵ is selected from hydrogen, —C₁-C₆alkyl, e.g., methyl, and —O—R^{L};
R^{L} is -(unsubstituted C₁-C₆ alkylene)-N(R¹³)₂; and
each R¹³ is independently selected from hydrogen, and —C₁-C₆alkyl, e.g., methyl;
or two R¹³ on the same N atom are taken together with the N atom to which they are attached to form an unsubstituted or substituted N-containing heterocycle.

In some exemplary embodiments at least one of R⁴ and R⁵ is —O—R^{L}. In alternative exemplary embodiments, R⁴ and R⁵ are independently selected from hydrogen or methyl. In some embodiments, R⁴ is selected from hydrogen, —C₁-C₆alkyl, and —O—R^{L}; R⁵ is selected from hydrogen, —C₁-C₆alkyl, and —O—R^{L}. In some such embodiments, R^{L} is selected from -(unsubstituted C₁-C₆ alkylene)-NH₂ and -(unsubstituted C₁-C₆ alkylene)-OH.

In one aspect, disclosed herein is a compound having the following structure:

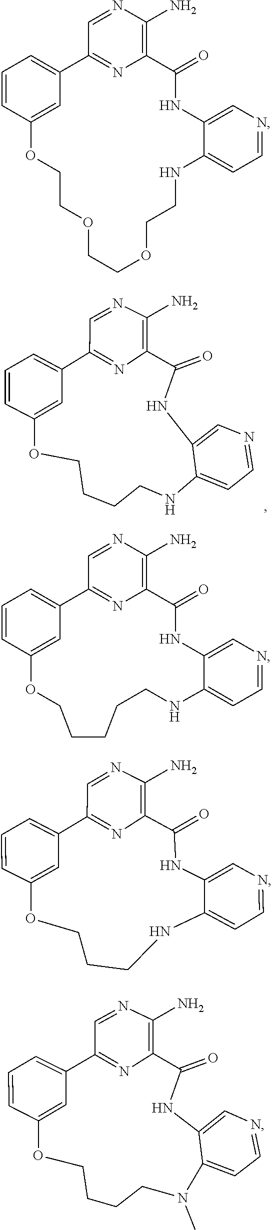

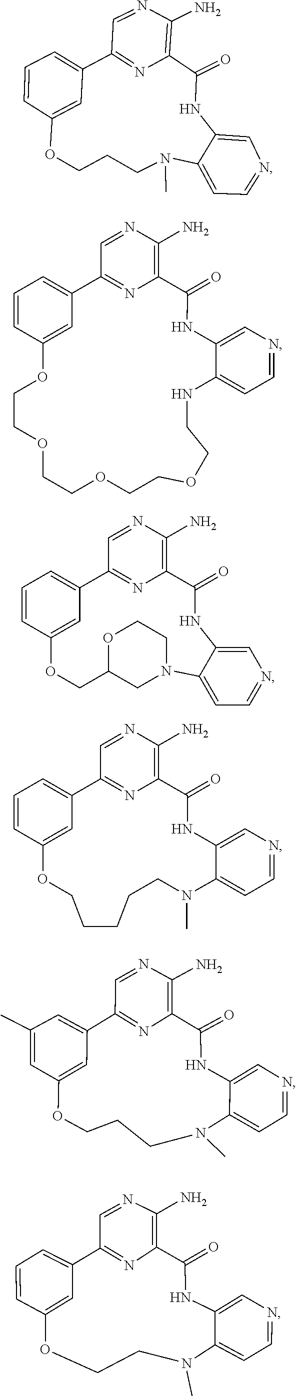

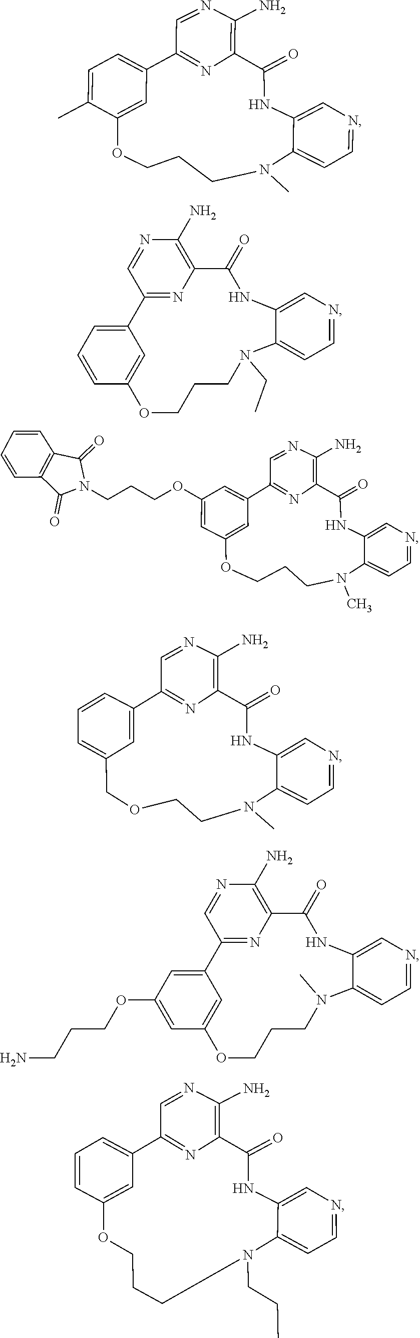

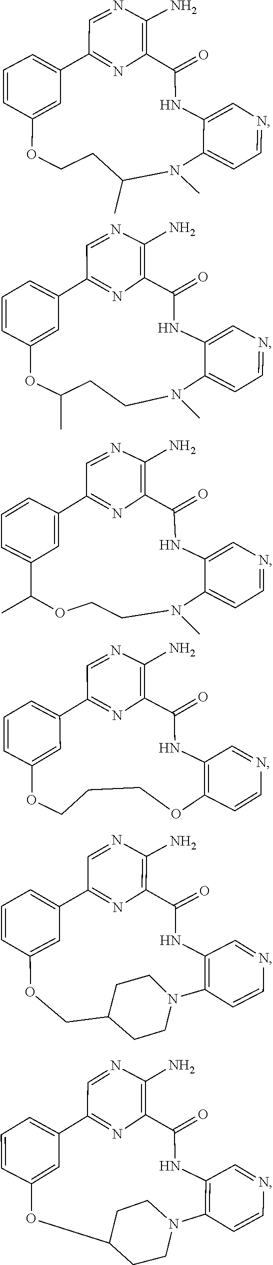

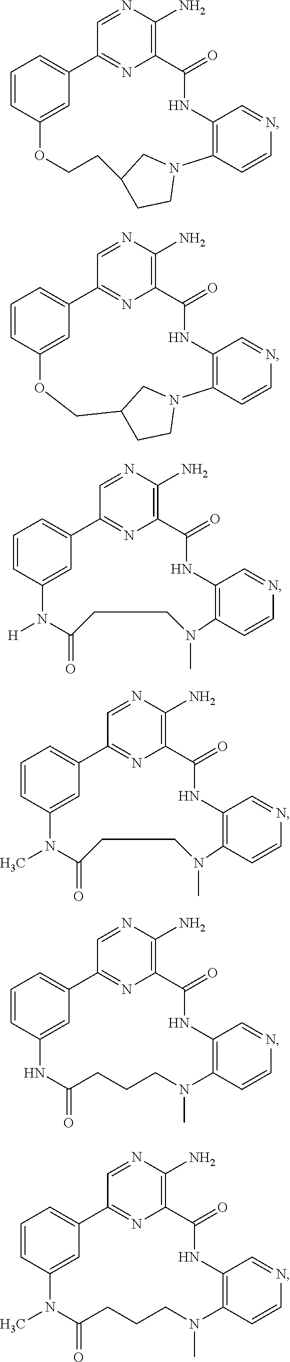

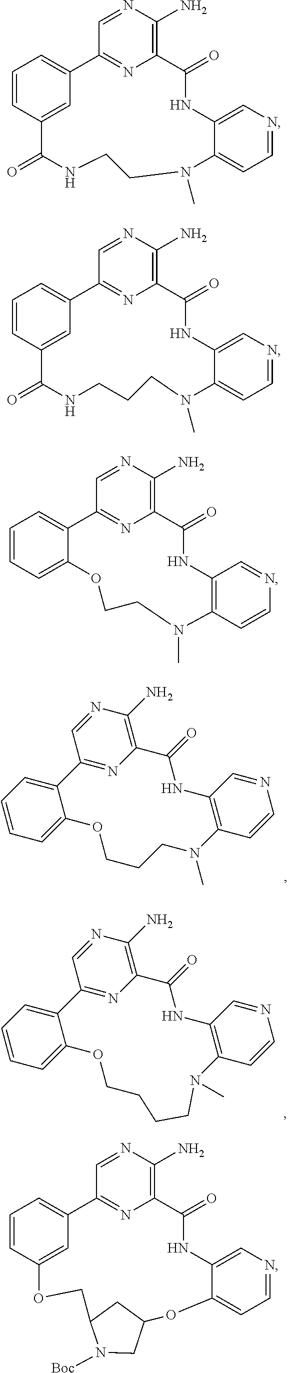

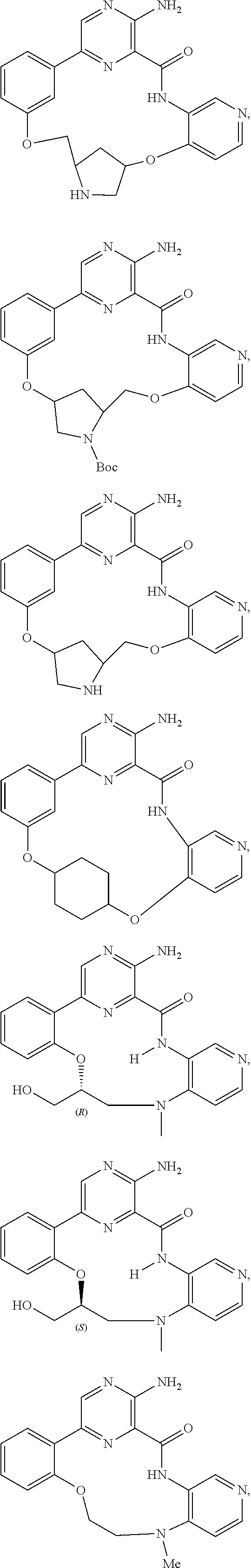

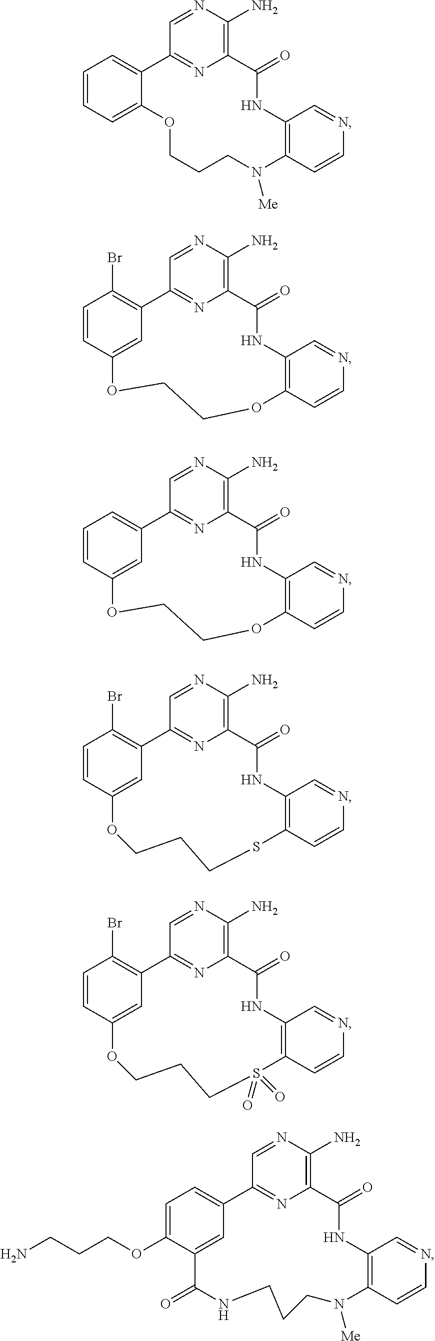

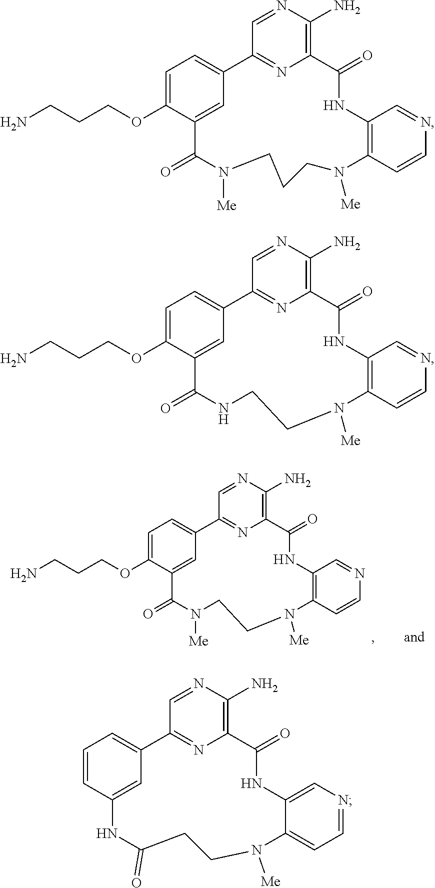

or a pharmaceutically acceptable salt of any one thereof.

In some embodiments, disclosed herein is a compound having the following structure:

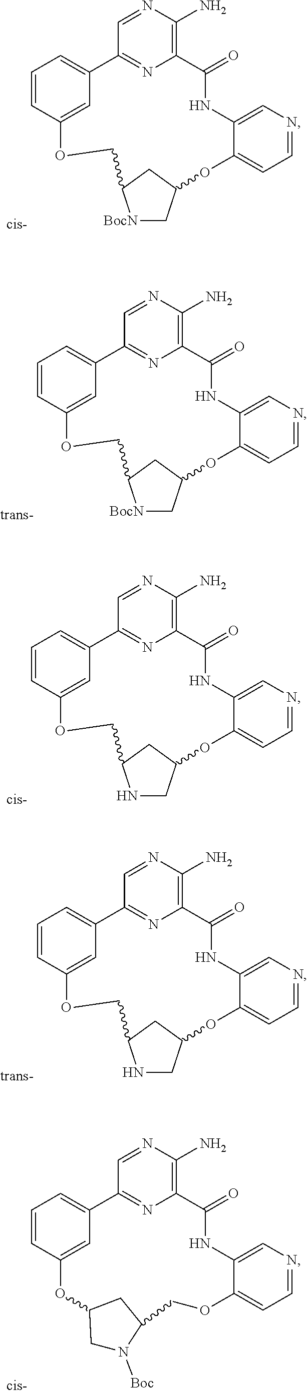

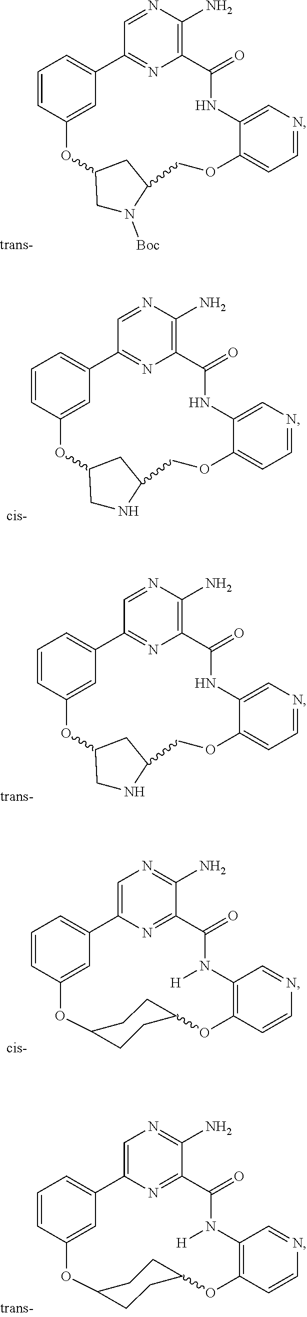 [SMILES: CC(C)(C)OC(=O)N1CC2CC1COc1ccncc1NC(=O)c1nc(cnc1N)-c1cccc(c1)O2 ; Nc1ncc2nc1C(=O)Nc1cnccc1OCC1CC(CN1)Oc1cccc-2c1 ; Nc1ncc2nc1C(=O)Nc1cnccc1OCC1CC(CN1)Oc1cccc-2c1 ; Nc1ncc2nc1C(=O)Nc1cnccc1OC1CCC(CC1)Oc1cccc-2c1 ; Nc1ncc2nc1C(=O)Nc1cnccc1OC1CCC(CC1)Oc1cccc-2c1]

or a pharmaceutically acceptable salt of any one thereof.

In one aspect, disclosed herein is a compound having a structure described in Table 1, or a pharmaceutically acceptable salt thereof.

TABLE 1

| Cmpd | Name | Structure |
|---|---|---|
| 1 | 2⁵-amino-9,12,15-trioxa-4,6- diaza-2(2,6)-pyrazina- 5(3,4)-pyridina-1(1,3)- benzenacyclopentadecaphan- 3-one | 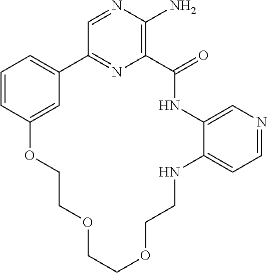 [SMILES: Nc1ncc2nc1C(=O)Nc1cnccc1NCCOCCOCCOc1cccc-2c1] |
| 2 | 2⁵-amino-11-oxa-4,6-diaza- 2(2,6)-pyrazina-5(3,4)- pyridina-1(1,3)- benzenacycloundecaphan-3- one | 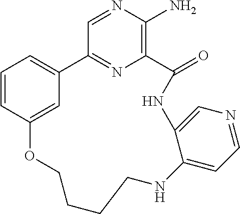 [SMILES: Nc1ncc2nc1C(=O)Nc1cnccc1NCCCCOc1cccc-2c1] |
| 3 | 2⁵-amino-12-oxa-4,6-diaza- 2(2,6)-pyrazina-5(3,4)- pyridina-1(1,3)- benzenacyclododecaphan-3- one | 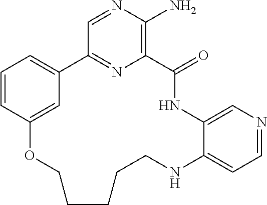 [SMILES: Nc1ncc2nc1C(=O)Nc1cnccc1NCCCCCOc1cccc-2c1] |
| 4 | 2⁵-amino-10-oxa-4,6-diaza- 2(2,6)-pyrazina-5(3,4)- pyridina-1(1,3)- benzenacyclodecaphan-3- one | 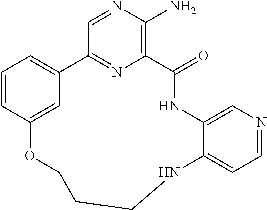 [SMILES: Nc1ncc2nc1C(=O)Nc1cnccc1NCCCOc1cccc-2c1] |
| 5 | 2⁵-amino-6-methyl-11-oxa- 4,6-diaza-2(2,6)-pyrazina- 5(3,4)-pyridina-1(1,3)- benzenacycloundecaphan-3- one | 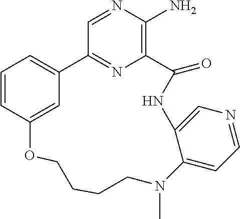 [SMILES: CN1CCCCOc2cccc(c2)-c2cnc(N)c(n2)C(=O)Nc2cnccc21] |
| 6 | 2⁵-amino-6-methyl-10-oxa- 4,6-diaza-2(2,6)-pyrazina- 5(3,4)-pyridina-1(1,3)- benzenacyclodecaphan-3- one | 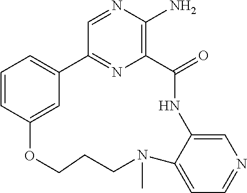 [SMILES: CN1CCCOc2cccc(c2)-c2cnc(N)c(n2)C(=O)Nc2cnccc21] |
| 7 | 2⁵-amino-9,12,15,18- tetraoxa-4,6-diaza-2(2,6)- pyrazina-5(3,4)-pyridina- 1(1,3)- benzenacyclooctadecaphan- 3-one | 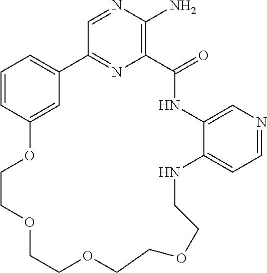 [SMILES: Nc1ncc2nc1C(=O)Nc1cnccc1NCCOCCOCCOCCOc1cccc-2c1] |
| 8 | 5³-amino-7-oxa-3-aza- 1(4,2)-morpholina-5(2,6)- pyrazina-2(4,3)-pyridina- 6(1,3)- benzenacyclooctaphan-4-one | 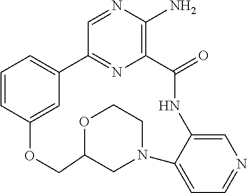 [SMILES: Nc1ncc2nc1C(=O)Nc1cnccc1N1CCOC(COc3cccc-2c3)C1] |
| 9 | 2⁵-amino-6-methyl-12-oxa- 4,6-diaza-2(2,6)-pyrazina- 5(3,4)-pyridina-1(1,3)- benzenacyclododecaphan-3- one | 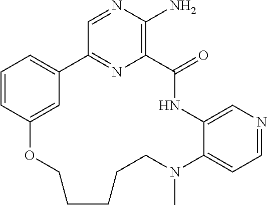 [SMILES: CN1CCCCCOc2cccc(c2)-c2cnc(N)c(n2)C(=O)Nc2cnccc21] |
| 10 | 2⁵-amino-1⁵,6-dimethyl-10- oxa-4,6-diaza-2(2,6)- pyrazina-5(3,4)-pyridina- 1(1,3)- benzenacyclodecaphan-3- one | 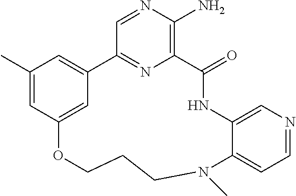 [SMILES: Cc1cc2cc(c1)-c1cnc(N)c(n1)C(=O)Nc1cnccc1N(C)CCCO2] |
| 11 | 2⁵-amino-6-methyl-9-oxa- 4,6-diaza-2(2,6)-pyrazina- 5(3,4)-pyridina-1(1,3)- benzenacyclononaphan-3- one | 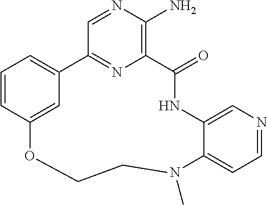 [SMILES: CN1CCOc2cccc(c2)-c2cnc(N)c(n2)C(=O)Nc2cnccc21] |
| 12 | 2⁵-amino-1⁴,6-dimethyl-10- oxa-4,6-diaza-2(2,6)- pyrazina-5(3,4)-pyridina- 1(1,3)- benzenacyclodecaphan-3- one | 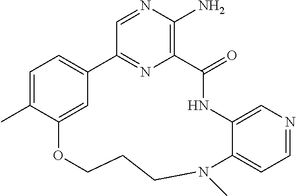 [SMILES: Cc1ccc2cc1OCCCN(C)c1ccncc1NC(=O)c1nc-2cnc1N] |
| 13 | 2⁵-amino-6-ethyl-10-oxa- 4,6-diaza-2(2,6)-pyrazina- 5(3,4)-pyridina-1(1,3)- benzenacyclodecaphan-3- one | 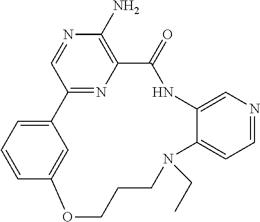 [SMILES: CCN1CCCOc2cccc(c2)-c2cnc(N)c(n2)C(=O)Nc2cnccc21] |
| 14 | 2-(3-((2⁵-amino-6-methyl-3- oxo-10-oxa-4,6-diaza- 2(2,6)-pyrazina-5(3,4)- pyridina-1(1,3)- benzenacyclodecaphane-1⁵- yl)oxy)propyl)isoindoline- 1,3-dione | 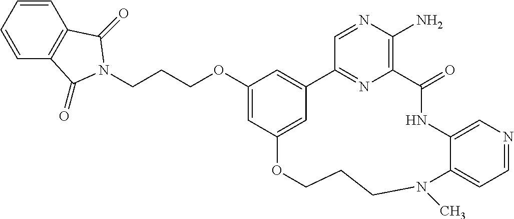 [SMILES: CN1CCCOc2cc(OCCCN3C(=O)c4ccccc4C3=O)cc(c2)-c2cnc(N)c(n2)C(=O)Nc2cnccc21] |
| 15 | 2⁵-amino-6-methyl-9-oxa- 4,6-diaza-2(2,6)-pyrazina- 5(3,4)-pyridina-1(1,3)- benzenacyclodecaphan-3- one | 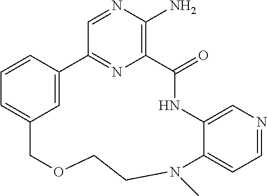 [SMILES: CN1CCOCc2cccc(c2)-c2cnc(N)c(n2)C(=O)Nc2cnccc21] |
| 16 | 2⁵-amino-1⁵-(3- aminopropoxy)-6-methyl- 10-oxa-4,6-diaza-2(2,6)- pyrazina-5(3,4)-pyridina- 1(1,3)- benzenacyclodecaphan-3- one | 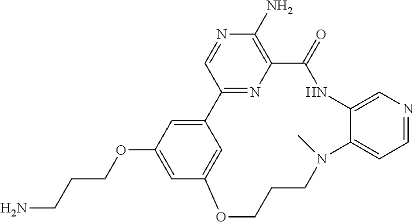 [SMILES: CN1CCCOc2cc(OCCCN)cc(c2)-c2cnc(N)c(n2)C(=O)Nc2cnccc21] |
| 17 | 2⁵-amino-6-propyl-10-oxa- 4,6-diaza-2(2,6)-pyrazina- 5(3,4)-pyridina-1(1,3)- benzenacyclodecaphan-3- one | 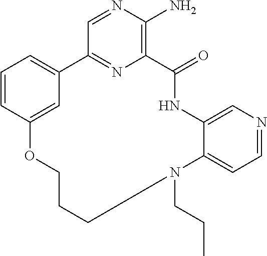 [SMILES: CCCN1CCCOc2cccc(c2)-c2cnc(N)c(n2)C(=O)Nc2cnccc21] |
| 18 | 2⁵-amino-6,7-dimethyl-10- oxa-4,6-diaza-2(2,6)- pyrazina-5(3,4)-pyridina- 1(1,3)- benzenacyclodecaphan-3- one | 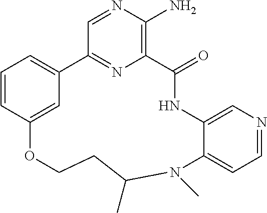 [SMILES: CC1CCOc2cccc(c2)-c2cnc(N)c(n2)C(=O)Nc2cnccc2N1C] |
| 19 | 2⁵-amino-6,9-dimethyl-10- oxa-4,6-diaza-2(2,6)- pyrazina-5(3,4)-pyridina- 1(1,3)- benzenacyclodecaphan-3- one | 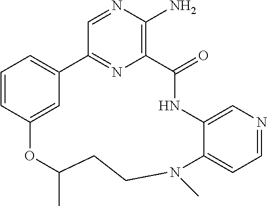 [SMILES: CC1CCN(C)c2ccncc2NC(=O)c2nc(cnc2N)-c2cccc(c2)O1] |
| 21 | 2⁵-amino-6,10-dioxa-4-aza- 2(2,6)-pyrazina-5(3,4)- pyridina-1(1,3)- benzenacyclodecaphan-3- one | 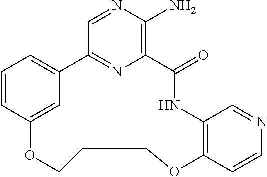 [SMILES: Nc1ncc2nc1C(=O)Nc1cnccc1OCCCOc1cccc-2c1] |
| 22 | 1⁵-amino-3-oxa-7-aza- 1(2,6)-pyrazina-6(4,3)- pyridina-5(4,1)-piperidina- 2(1,3)- benzenacyclooctaphan-8-one | 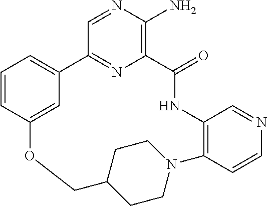 [SMILES: Nc1ncc2nc1C(=O)Nc1cnccc1N1CCC(CC1)COc1cccc-2c1] |
| 23 | 1⁵-amino-3-oxa-6-aza- 1(2,6)-pyrazina-5(4,3)- pyridina-4(4,1)-piperidina- 2(1,3)- benzenacycloheptaphan-7- one | 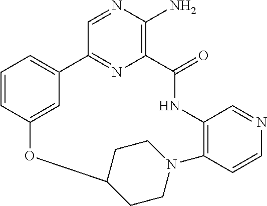 [SMILES: Nc1ncc2nc1C(=O)Nc1cnccc1N1CCC(CC1)Oc1cccc-2c1] |
| 24 | 2⁵-amino-9-oxa-4-aza- 2(2,6)-pyrazina-5(3,4)- pyridina-6(1,3)-pyrrolidina- 1(1,3)- benzenacyclononaphan-3- one | 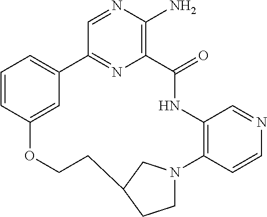 [SMILES: Nc1ncc2nc1C(=O)Nc1cnccc1N1CCC(CCOc3cccc-2c3)C1] |
| 25 | 1⁵-amino-3-oxa-7-aza- 1(2,6)-pyrazina-6(4,3)- pyridina-5(3,1)-pyrrolidina- 2(1,3)- benzenacyclooctaphan-8-one | 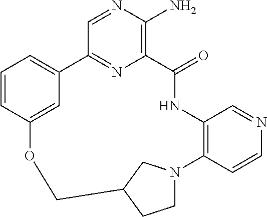 [SMILES: Nc1ncc2nc1C(=O)Nc1cnccc1N1CCC(COc3cccc-2c3)C1] |
| 26 | 2⁵-amino-6-methyl-4,6,10- triaza-2(2,6)-pyrazina- 5(3,4)-pyridina-1(1,3)- benzenacyclodecaphene-3,9- dione | 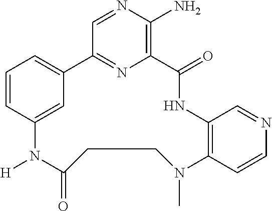 [SMILES: CN1CCC(=O)Nc2cccc(c2)-c2cnc(N)c(n2)C(=O)Nc2cnccc21] |
| 27 | 2⁵-amino-6,10-dimethyl- 4,6,10-triaza-2(2,6)- pyrazina-5(3,4)-pyridina- 1(1,3)- benzenacyclodecaphane-3,9- dione | 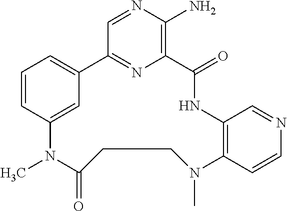 [SMILES: CN1CCC(=O)N(C)c2cccc(c2)-c2cnc(N)c(n2)C(=O)Nc2cnccc21] |
| 28 | 2⁵-amino-6-methyl-4,6,11- triaza-2(2,6)-pyrazina- 5(3,4)-pyridina-1(1,3)- benzenacycloundecaphane- 3,10-dione | 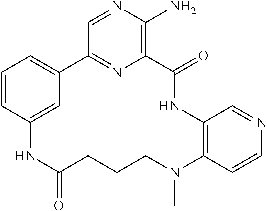 [SMILES: CN1CCCC(=O)Nc2cccc(c2)-c2cnc(N)c(n2)C(=O)Nc2cnccc21] |
| 29 | 2⁵-amino-6,11-dimethyl- 4,6,11-triaza-2(2,6)- pyrazina-5(3,4)-pyridina- 1(1,3)- benzenacycloundecaphane- 3,10-dione | 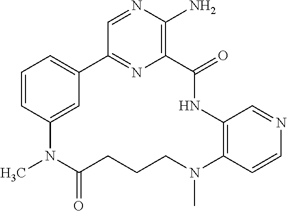 [SMILES: CN1CCCC(=O)N(C)c2cccc(c2)-c2cnc(N)c(n2)C(=O)Nc2cnccc21] |
| 30 | 2⁵-amino-6-methyl-4,6,9- triaza-2(2,6)-pyrazina- 5(3,4)-pyridina-1(1,3)- benzenacyclodecaphane- 3,10-dione | 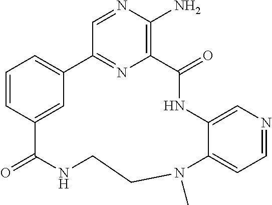 [SMILES: CN1CCNC(=O)c2cccc(c2)-c2cnc(N)c(n2)C(=O)Nc2cnccc21] |
| 31 | 2⁵-amino-6-methyl-4,6,10- triaza-2(2,6)-pyrazina- 5(3,4)-pyridina-1(1,3)- benzenacycloundecaphane- 3,11-dione | 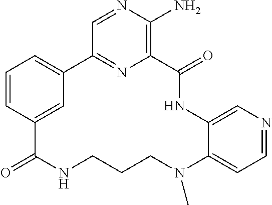 [SMILES: CN1CCCNC(=O)c2cccc(c2)-c2cnc(N)c(n2)C(=O)Nc2cnccc21] |
| 32 | 2⁵-amino-6-methyl-9-oxa- 4,6-diaza-2(2,6)-pyrazina- 5(3,4)-pyridina-1(1,2)- benzenacyclononaphan-3- one | 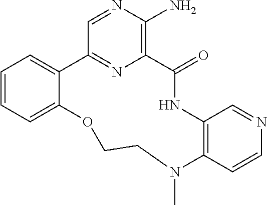 [SMILES: CN1CCOc2ccccc2-c2cnc(N)c(n2)C(=O)Nc2cnccc21] |
| 33 | 2⁵-amino-6-methyl-10-oxa- 4,6-diaza-2(2,6)-pyrazina- 5(3,4)-pyridina-1(1,2)- benzenacyclodecaphan-3- one | 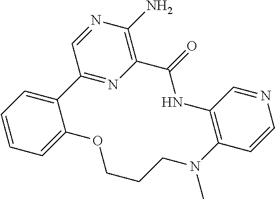 [SMILES: CN1CCCOc2ccccc2-c2cnc(N)c(n2)C(=O)Nc2cnccc21] |
| 34 | 2⁵-amino-6-methyl-11-oxa- 4,6-diaza-2(2,6)-pyrazina- 5(3,4)-pyridina-1(1,2)- benzenacycloundecaphan-3- one | 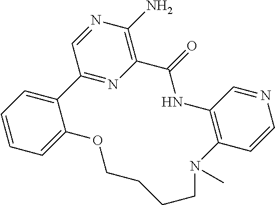 [SMILES: CN1CCCCOc2ccccc2-c2cnc(N)c(n2)C(=O)Nc2cnccc21] |
| 35 | (±)-tert-butyl(5²,5⁴-trans)- 1⁵-amino-9-oxo-3,6-dioxa-8- aza-1(2,6)-pyrazina-7(4,3)- pyridina-5(2,4)-pyrrolidina- 2(1,3)- benzenacyclononaphane-51- carboxylate | 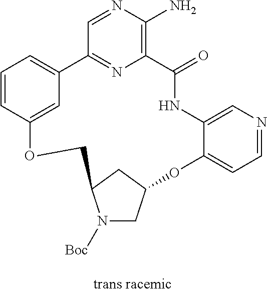 [SMILES: CC(C)(C)OC(=O)N1C[C@@H]2C[C@@H]1COc1cccc(c1)-c1cnc(N)c(n1)C(=O)Nc1cnccc1O2] |
| 36 | (±)-(5²,5⁴-trans)-1⁵-amino- 3,6-dioxa-8-aza-1(2,6)- pyrazina-7(4,3)-pyridina- 5(2,4)-pyrrolidina-2(1,3)- benzenacyclononaphan-9- one | 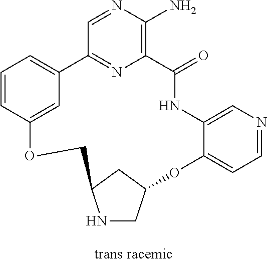 [SMILES: Nc1ncc2nc1C(=O)Nc1cnccc1O[C@@H]1CN[C@@H](COc3cccc-2c3)C1] |
| 37 | (±)-tert-butyl(5²,5⁴-cis)-1⁵- amino-9-oxo-3,6-dioxa-8- aza-1(2,6)-pyrazina-7(4,3)- pyridina-5(2,4)-pyrrolidina- 2(1,3)- benzenacyclononaphane-51- carboxylate | 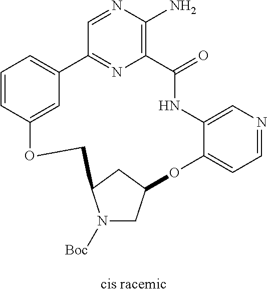 |
| 38 | (±)-(5²,5⁴-cis)-1⁵-amino-3,6- dioxa-8-aza-1(2,6)-pyrazina- 7(4,3)-pyridina-5(2,4)- pyrrolidina-2(1,3)- benzenacyclononaphan-9- one | 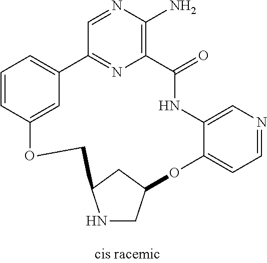 |
| 39 | (±)-tert-butyl(4³,4⁵-trans)- 1⁵-amino-9-oxo-3,6-dioxa-8- aza-1(2,6)-pyrazina-7(4,3)- pyridina-4(3,5)-pyrrolidina- 2(1,3)- benzenacyclononaphane-41- carboxylate | 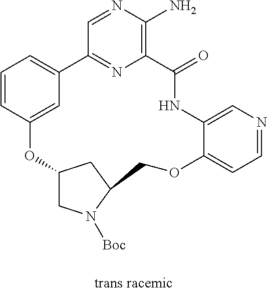 [SMILES: CC(C)(C)OC(=O)N1C[C@H]2C[C@H]1COc1ccncc1NC(=O)c1nc(cnc1N)-c1cccc(c1)O2] |
| 40 | (±)-(4³,4⁵-trans)-1⁵-amino- 3,6-dioxa-8-aza-1(2,6)- pyrazina-7(4,3)-pyridina- 4(3,5)-pyrrolidina-2(1,3)- benzenacyclononaphan-9- one | 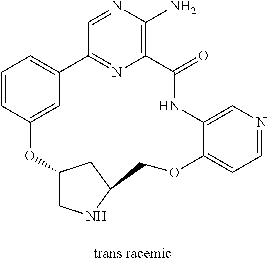 [SMILES: Nc1ncc2nc1C(=O)Nc1cnccc1OC[C@@H]1C[C@H](CN1)Oc1cccc-2c1] |
| 41 | (±)-tert-butyl(4³,4⁵-cis)-1⁵- amino-9-oxo-3,6-dioxa-8- aza-1(2,6)-pyrazina-7(4,3)- pyridina-4(3,5)-pyrrolidina- 2(1,3)- benzenacyclononaphane-41- carboxylate | 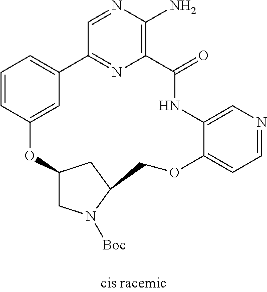 [SMILES: CC(C)(C)OC(=O)N1C[C@@H]2C[C@H]1COc1ccncc1NC(=O)c1nc(cnc1N)-c1cccc(c1)O2] |
| 42 | (±)-(4³,4⁵-cis)-1⁵-amino-3,6- dioxa-8-aza-1(2,6)-pyrazina- 7(4,3)-pyridina-4(3,5)- pyrrolidina-2(1,3)- benzenacyclononaphan-9- one | 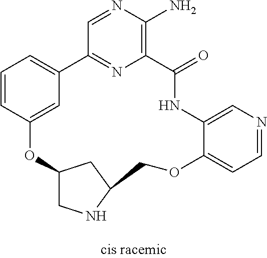 [SMILES: Nc1ncc2nc1C(=O)Nc1cnccc1OC[C@@H]1C[C@@H](CN1)Oc1cccc-2c1] |
| 43 | (4¹,4⁴-cis)-1⁵-amino-3,5- dioxa-7-aza-1(2,6)-pyrazina- 6(4,3)-pyridina-2(1,3)- benzena-4(1,4)- cyclohexanacyclooctaphan- 8-one | 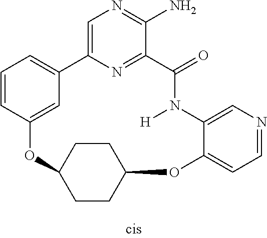 [SMILES: Nc1ncc2nc1C(=O)Nc1cnccc1O[C@H]1CC[C@H](CC1)Oc1cccc-2c1] |
| 44 | (4¹,4⁴-trans)-1⁵-amio-3,5- dioxa-7-aza-1(2,6)-pyrazina- 6(4,3)-pyridina-2(1,3)- benzena-4(1,4)- cyclohexanacyclooctaphan- 8-one | 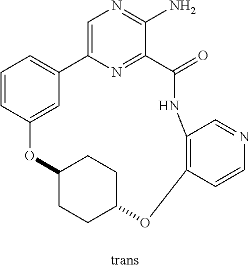 |
| 45 | (R)-2⁵-amino-8- (hydroxymethyl)-6-methyl- 9-oxa-4,6-diaza-2(2,6)- pyrazina-5(3,4)-pyridina- 1(1,2)- benzenacyclononaphan-3- one | 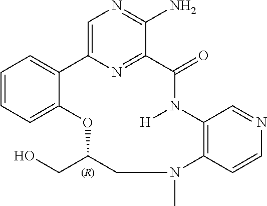 |
| 46 | (S)-2⁵-amino-8- (hydroxymethyl)-6-methyl- 9-oxa-4,6-diaza-2(2,6)- pyrazina-5(3,4)-pyridina- 1(1,2)- benzenacyclononaphan-3- one | 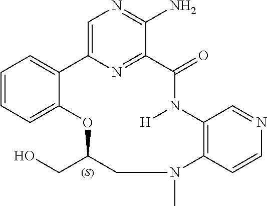 |
| 49 | 2⁵-amino-16-bromo-6,9- dioxa-4-aza-2(2,6)-pyrazina- 5(3,4)-pyridina-1(1,3)- benzenacyclononaphan-3- one | 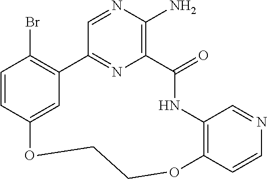 [SMILES: Nc1ncc2nc1C(=O)Nc1cnccc1OCCOc1ccc(Br)c-2c1] |
| 50 | 2⁵-amino-6,9-dioxa-4-aza- 2(2,6)-pyrazina-5(3,4)- pyridina-1(1,3)- benzenacyclononaphan-3- one | 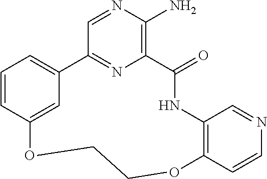 [SMILES: Nc1ncc2nc1C(=O)Nc1cnccc1OCCOc1cccc-2c1] |
| 51 | 2⁵-amino-1⁶-bromo-10-oxa- 6-thia-4-aza-2(2,6)-pyrazina- 5(3,4)-pyridina-1(1,3)- benzenacyclodecaphan-3- one | 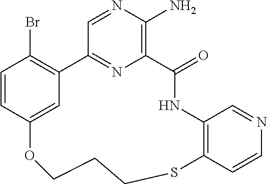 [SMILES: Nc1ncc2nc1C(=O)Nc1cnccc1SCCCOc1ccc(Br)c-2c1] |
| 52 | 2⁵-amino-1⁶-bromo-10-oxa- 6-thia-4-aza-2(2,6)-pyrazina- 5(3,4)-pyridina-1(1,3)- benzenacyclodecaphan-3- one 6,6-dioxide | 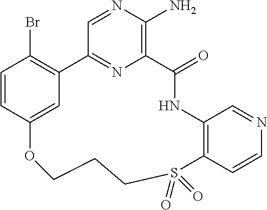 [SMILES: Nc1ncc2nc1C(=O)Nc1cnccc1S(=O)(=O)CCCOc1ccc(Br)c-2c1] |
| 53 | 2⁵-amino-1⁴-(3- aminopropoxy)-6-methyl- 4,6,10-triaza-2(2,6)- pyrazina-5(3,4)-pyridina- 1(1,3)- benzenacycloundecaphane- 3,11-dione | 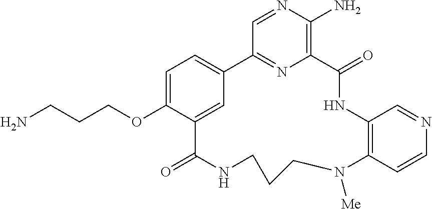 [SMILES: CN1CCCNC(=O)c2cc(ccc2OCCCN)-c2cnc(N)c(n2)C(=O)Nc2cnccc21] |
| 54 | 2⁵-amino-1⁴-(3- aminopropoxy)-6,10- dimethyl-4,6,10-triaza- 2(2,6)-pyrazina-5(3,4)- pyridina-1(1,3)- benzenacycloundecaphane- 3,11-dione | 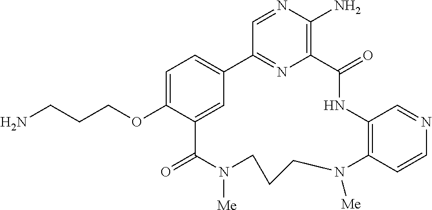 [SMILES: CN1CCCN(C)c2ccncc2NC(=O)c2nc(cnc2N)-c2ccc(OCCCN)c(c2)C1=O] |
| 55 | 2⁵-amino-1⁴-(3- aminopropoxy)-6-methyl- 4,6,9-triaza-2(2,6)-pyrazina- 5(3,4)-pyridina-1(1,3)- benzenacyclodecaphane- 3,10-dione | 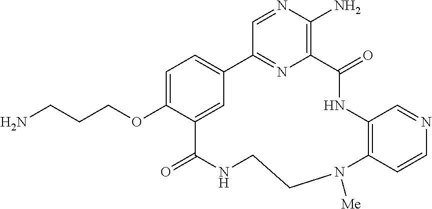 [SMILES: CN1CCNC(=O)c2cc(ccc2OCCCN)-c2cnc(N)c(n2)C(=O)Nc2cnccc21] |
| 56 | 2⁵-amino-1⁴-(3- aminopropoxy)-6,9- dimethyl-4,6,9-triaza-2(2,6)- pyrazina-5(3,4)-pyridina- 1(1,3)- benzenacyclodecaphene- 3,10-dione | 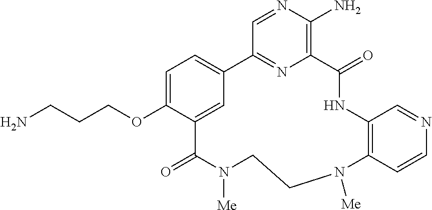 [SMILES: CN1CCN(C)c2ccncc2NC(=O)c2nc(cnc2N)-c2ccc(OCCCN)c(c2)C1=O] |
| 57 | (R)-2⁵-amino-8- ((benzylamino)methyl)-6- methyl-9-oxa-4,6-diaza- 2(2,6)-pyrazin-5(3,4)- pyridina-1(1,2)- benzenacyclononaphan-3- one | 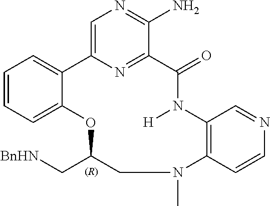 |
| 58 | (S)-2⁵-amino-8- (aminomethyl)-6-methyl-9- oxa-4,6-diaza-2(2,6)- pyrazina-5(3,4)-pyridina- 1(1,2)- benzenacyclononaphan-3- one | 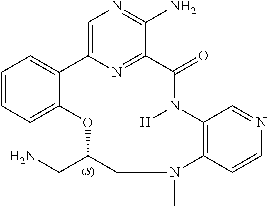 |
| 59 | (S)-2⁵-amino-8- ((benzylamino)methyl)-6- methyl-9-oxa-4,6-diaza- 2(2,6)-pyrazina-5(3,4)- pyridina-1(1,2)- benzenacyclononaphan-3- one | 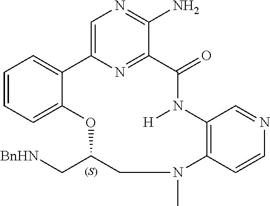 |
| 60 | (R)-2⁵-amino-8- (aminomethyl)-6-methyl-9- oxa-4,6-diaza-2(2,6)- pyrazina-5(3,4)-pyridina- 1(1,2)- benzenacyclononaphan-3- one | 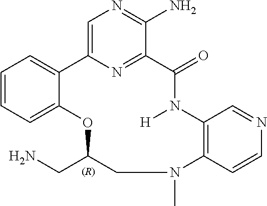 |
| 61 | 2⁵-amino-1⁴-(3- aminopropoxy)-6-methyl- 10-oxa-4,6-diaza-2(2,6)- pyrazina-5(3,4)-pyridina- 1(1,3)- benzenacyclodecaphan-3- one | 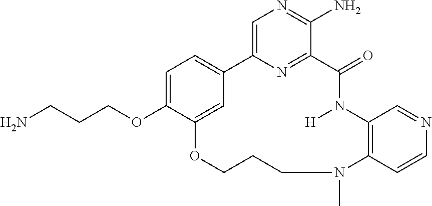 [SMILES: CN1CCCOc2cc(ccc2OCCCN)-c2cnc(N)c(n2)C(=O)Nc2cnccc21] |
| 62 | 2⁵-amino-1⁴-(4-aminobutyl)- 6-methyl-10-oxa-4,6-diaza- 2(2,6)-pyrazin-5(3,4)- pyridina-1(1,3)- benzenacyclodecaphan-3- one | 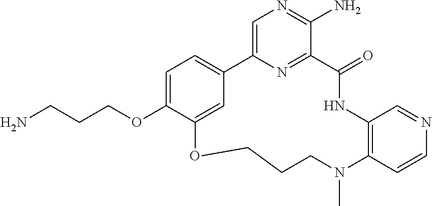 [SMILES: CN1CCCOc2cc(ccc2OCCCN)-c2cnc(N)c(n2)C(=O)Nc2cnccc21] |
| 63 | 2⁵-amino-1⁴-(3- aminopropxy)-6-methyl- 4,6-diaza-2(2,6)-pyrazina- 5(3,4)-pyridina-1(1,3)- benzenacyclodecaphan-3- one | 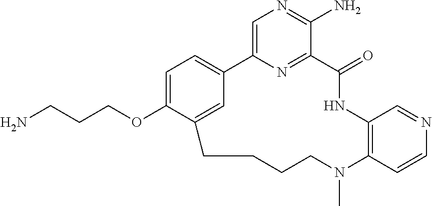 [SMILES: CN1CCCCc2cc(ccc2OCCCN)-c2cnc(N)c(n2)C(=O)Nc2cnccc21] |
| 64 | 2⁵-amino-1⁵-(3- aminopropoxy)-6-methyl-9- oxa-4,6-diaza-2(2,6)- pyrazina-5(3,4)-pyridina- 1(1,2)- benzenacyclononaphan-3- one | 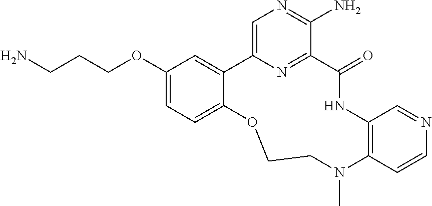 [SMILES: CN1CCOc2ccc(OCCCN)cc2-c2cnc(N)c(n2)C(=O)Nc2cnccc21] |
| 65 | 2⁵-amino-1⁴-(3- aminopropoxy)-6-methyl-9- oxa-4,6-diaza-2(2,6)- pyrazina-5(3,4)-pyridina- 1(1,2)- benzenacyclononaphan-3- one | 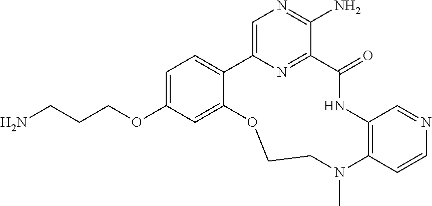 [SMILES: CN1CCOc2cc(OCCCN)ccc2-c2cnc(N)c(n2)C(=O)Nc2cnccc21] |

Chemical entities having carbon-carbon double bonds or carbon-nitrogen double bonds may exist in Z- or E-form (or cis- or trans-form). Furthermore, some chemical entities may exist in various tautomeric forms. Unless otherwise specified, compounds described herein are intended to include all Z-, E- and tautomeric forms as well.

A “tautomer” refers to a molecule wherein a proton shift from one atom of a molecule to another atom of the same molecule is possible. The compounds presented herein, in certain embodiments, exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibrium include:

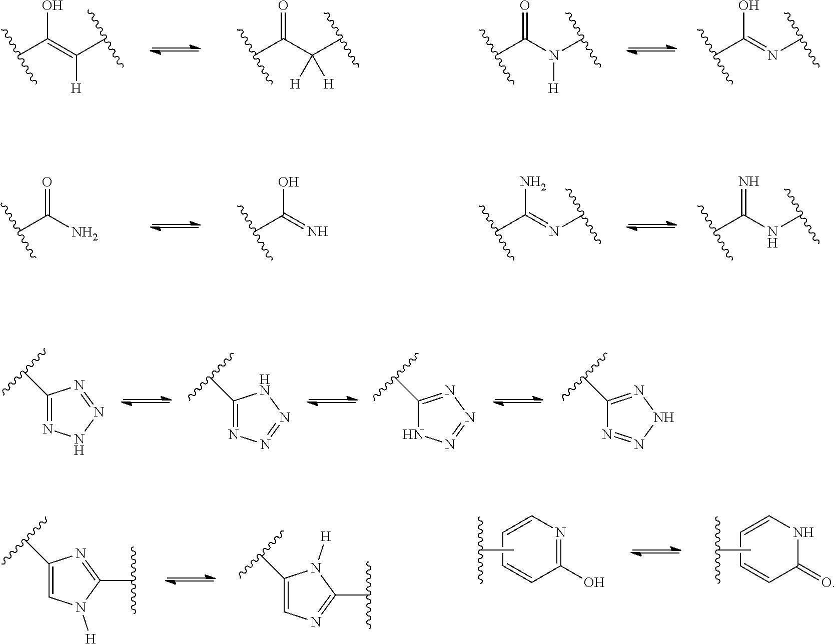

The compounds disclosed herein, in some embodiments, are used in different enriched isotopic forms, e.g., enriched in the content of ²H, ³H, ¹¹C, ¹³C and/or ¹⁴C. In one particular embodiment, the compound is deuterated in at least one position. Such deuterated forms can be made by the procedure described in U.S. Pat. Nos. 5,846,514 and 6,334,997. As described in U.S. Pat. Nos. 5,846,514 and 6,334,997, deuteration can improve the metabolic stability and or efficacy, thus increasing the duration of action of drugs.

Unless otherwise stated, compounds described herein are intended to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of the present disclosure.

The compounds of the present disclosure optionally contain unnatural proportions of atomic isotopes at one or more atoms that constitute such compounds. For example, the compounds may be labeled with isotopes, such as for example, deuterium (²H), tritium (³H), iodine-125 (¹²⁵I) or carbon-14 (¹⁴C). Isotopic substitution with ²H, ¹¹C, ¹³C, ¹⁴C, ¹⁵C, ¹²N, ¹³N, ¹⁵N, ¹⁶N, ¹⁶O, ¹⁷O, ¹⁴F, ¹⁵F, ¹⁶F, ¹⁷F, ¹⁸F, ³³S, ³⁴S, ³⁵S, ³⁶S, ³⁵Cl, ³⁷Cl, ⁷⁹Br, ⁸¹Br, and ¹²⁵I are all contemplated. All isotopic variations of the compounds of the present invention, whether radioactive or not, are encompassed within the scope of the present invention.

In certain embodiments, the compounds disclosed herein have some or all of the ¹H atoms replaced with ²H atoms. The methods of synthesis for deuterium-containing compounds are known in the art and include, by way of non-limiting example only, the following synthetic methods.

Deuterium substituted compounds are synthesized using various methods such as described in: Dean, Dennis C.; Editor. Recent Advances in the Synthesis and Applications of Radiolabeled Compounds for Drug Discovery and Development. [In: Curr., Pharm. Des., 2000; 6(10)] 2000, 110 pp; George W.; Varma, Rajender S. The Synthesis of Radiolabeled Compounds via Organometallic Intermediates, Tetrahedron, 1989, 45(21), 6601-21; and Evans, E. Anthony. Synthesis of radiolabeled compounds, J. Radioanal. Chem., 1981, 64(1-2), 9-32.

Deuterated starting materials are readily available and are subjected to the synthetic methods described herein to provide for the synthesis of deuterium-containing compounds. Large numbers of deuterium-containing reagents and building blocks are available commercially from chemical vendors, such as Aldrich Chemical Co.

Compounds of the present invention also include crystalline and amorphous forms of those compounds, pharmaceutically acceptable salts, and active metabolites of these compounds having the same type of activity, including, for example, polymorphs, pseudopolymorphs, solvates, hydrates, unsolvated polymorphs (including anhydrates), conformational polymorphs, and amorphous forms of the compounds, as well as mixtures thereof.

Included in the present disclosure are salts, particularly pharmaceutically acceptable salts, of the compounds described herein. The compounds of the present disclosure that possess a sufficiently acidic, a sufficiently basic, or both functional groups, can react with any of a number of inorganic bases, and inorganic and organic acids, to form a salt. Alternatively, compounds that are inherently charged, such as those with a quaternary nitrogen, can form a salt with an appropriate counterion, e.g., a halide such as bromide, chloride, or fluoride, particularly bromide.

The compounds described herein may in some cases exist as diastereomers, enantiomers, or other stereoisomeric forms. The compounds presented herein include all diastereomeric, enantiomeric, and epimeric forms as well as the appropriate mixtures thereof. Separation of stereoisomers may be performed by chromatography or by forming diastereomers and separating by recrystallization, or chromatography, or any combination thereof (Jean Jacques, Andre Collet, Samuel H. Wilen, “Enantiomers, Racemates and Resolutions”, John Wiley And Sons, Inc., 1981, herein incorporated by reference for this disclosure). Stereoisomers may also be obtained by stereoselective synthesis.

The methods and compositions described herein include the use of amorphous forms as well as crystalline forms (also known as polymorphs). The compounds described herein may be in the form of pharmaceutically acceptable salts. As well, in some embodiments, active metabolites of these compounds having the same type of activity are included in the scope of the present disclosure. In addition, the compounds described herein can exist in unsolvated as well as solvated forms with pharmaceutically acceptable solvents such as water, ethanol, and the like. The solvated forms of the compounds presented herein are also considered to be disclosed herein.

In certain embodiments, compounds or salts of the compounds may be prodrugs, e.g., wherein a hydroxyl in the parent compound is presented as an ester or a carbonate, or carboxylic acid present in the parent compound is presented as an ester. The term “prodrug” is intended to encompass compounds which, under physiologic conditions, are converted into pharmaceutical agents of the present disclosure. One method for making a prodrug is to include one or more selected moieties which are hydrolyzed under physiologic conditions to reveal the desired molecule. In other embodiments, the prodrug is converted by an enzymatic activity of the host animal such as specific target cells in the host animal. For example, esters or carbonates (e.g., esters or carbonates of alcohols or carboxylic acids and esters of phosphonic acids) are preferred prodrugs of the present disclosure.

Prodrug forms of the herein described compounds are also described herein, wherein the prodrug is metabolized in vivo to produce a compound as set forth herein. With respect to the small molecules described herein, e.g., compounds of Formula I-IV or salts thereof, the terms administration of and administering a compound should be understood to mean providing a compound of the invention or a prodrug of the compound of the invention to the individual in need.

Prodrugs are often useful because, in some situations, they may be easier to administer than the parent drug. They may, for instance, be bioavailable by oral administration whereas the parent is not. Prodrugs may help enhance the cell permeability of a compound relative to the parent drug. The prodrug may also have improved solubility in pharmaceutical compositions over the parent drug. Prodrugs may be designed as reversible drug derivatives, for use as modifiers to enhance drug transport to site-specific tissues or to increase drug residence inside of a cell.

In certain embodiments, the prodrug may be converted, e.g., enzymatically or chemically, to the parent compound under the conditions within a cell. In certain embodiments, the parent compound comprises an acidic moiety, e.g., resulting from the hydrolysis of the prodrug, which may be charged under the conditions within the cell. In particular embodiments, the prodrug is converted to the parent compound once it has passed through the cell membrane into a cell. In certain embodiments, the parent compound has diminished cell membrane permeability properties relative to the prodrug, such as decreased lipophilicity and increased hydrophilicity.

In particular embodiments, the parent compound with the acidic moiety is retained within a cell for a longer duration than the same compound without the acidic moiety.

The parent compound, with an acidic moiety, may be retained within the cell, i.e., drug residence, for 10% or longer, such as 15% or longer, such as 20% or longer, such as 25% or longer, such as 30% or longer, such as 35% or longer, such as 40% or longer, such as 45% or longer, such as 50% or longer, such as 55% or longer, such as 60% or longer, such as 65% or longer, such as 70% or longer, such as 75% or longer, such as 80% or longer, such as 85% or longer, or even 90% or longer relative to the same compound without an acidic moiety.

In some embodiments, the design of a prodrug increases the lipophilicity of the pharmaceutical agent. In some embodiments, the design of a prodrug increases the effective water solubility. See, e.g., Fedorak et al., Am. J. Physiol., 269:G210-218 (1995); McLoed et al., Gastroenterol, 106:405-413 (1994); Hochhaus et al., Biomed. Chrom., 6:283-286 (1992); J. Larsen and H. Bundgaard, Int. J. Pharmaceutics, 37, 87 (1987); J. Larsen et al., Int. J. Pharmaceutics, 47, 103 (1988); Sinkula et al., J. Pharm. Sci., 64:181-210 (1975); T. Higuchi and V. Stella, Pro-drugs as Novel Delivery Systems, Vol. 14 of the A.C.S. Symposium Series; and Edward B. Roche, Bioreversible Carriers in Drug Design, American Pharmaceutical Association and Pergamon Press, 1987, all incorporated herein for such disclosure). According to another embodiment, the present disclosure provides methods of producing the above-defined compounds. The compounds may be synthesized using conventional techniques. Advantageously, these compounds are conveniently synthesized from readily available starting materials.

Synthetic chemistry transformations and methodologies useful in synthesizing the compounds described herein are known in the art and include, for example, those described in R. Larock, Comprehensive Organic Transformations (1989); T. W. Greene and P. G. M. Wuts, Protective Groups in Organic Synthesis, 2d. Ed. (1991); L. Fieser and M. Fieser, Fieser and Fieser's Reagents for Organic Synthesis (1994); and L. Paquette, ed., Encyclopedia of Reagents for Organic Synthesis (1995).

### Linkers

The TGFβR2 inhibitor compounds and salts described herein (such as the cyclic amino-pyrazinecarboxamide compounds) may be bound to a linker, e.g., a cleavable peptide linker or a non-cleavable linker. In certain embodiments, the linker is also bound to an antibody construct or targeting moiety and may be referred to as an antibody conjugate, a targeting moiety conjugate, or a conjugate. Linkers of the conjugates may not affect the binding of active portions of a conjugate, e.g., the antigen binding domains, Fc domains, target binding domains, antibodies, cyclic amino-pyrazinecarboxamide compounds or the like, to an antigen. A conjugate can comprise multiple linkers, each having one or more compounds (e.g., TGFβR2 inhibitor) attached. These linkers can be the same linkers or different linkers.

A linker can be short, flexible, rigid, cleavable, non-cleavable, hydrophilic, or hydrophobic. A linker can contain segments that have different characteristics, such as segments of flexibility or segments of rigidity. The linker can be chemically stable to extracellular environments, for example, chemically stable in the blood stream, or may include linkages that are not stable or selectively stable. The linker can include linkages that are designed to cleave and/or immolate or otherwise breakdown specifically or non-specifically inside cells. A cleavable linker can be sensitive to enzymes. A cleavable linker can be cleaved by enzymes such as proteases. A cleavable linker may comprise a valine-citrulline linker or a valine-alanine peptide. A valine-citrulline- or valine-alanine-containing linker can contain a pentafluorophenyl group. A valine-citrulline- or valine-alanine-containing linker can contain a maleimide or succinimide group. A valine-citrulline- or valine-alanine-containing linker can contain a para aminobenzoic acid (PABA) group. A valine-citrulline- or valine-alanine-containing linker can contain a PABA group and a pentafluorophenyl group. A valine-citrulline- or valine-alanine-containing linker can contain a PABA group and a maleimide or succinimide group.

A non-cleavable linker can be protease insensitive. A non-cleavable linker can be maleimidocaproyl linker. A maleimidocaproyl linker can comprise N-maleimidomethylcyclohexane-1-carboxylate. A maleimidocaproyl linker can contain a succinimide group. A maleimidocaproyl linker can contain pentafluorophenyl group. A linker can be a combination of a maleimidocaproyl group and one or more polyethylene glycol molecules. A linker can be a maleimide-PEG4 linker. A linker can be a combination of a maleimidocaproyl linker containing a succinimide group and one or more polyethylene glycol molecules. A linker can be a combination of a maleimidocaproyl linker and one or more polyethylene glycol molecules. A linker can contain maleimides linked to polyethylene glycol molecules in which the polyethylene glycol can allow for more linker flexibility or can be used lengthen the linker. A linker can be a (maleimidocaproyl)-(valine-citrulline)-(para-aminobenzyloxycarbonyl) linker. A linker can be a linker suitable for attachment to an engineered cysteine (THIOMAB), such as a (maleimidocaproyl)-(valine-citrulline)-(para-aminobenzyloxycarbonyl)-linker.

A linker can also comprise alkylene, alkenylene, alkynylene, polyether, polyester, polyamide group(s) and also, polyamino acids, polypeptides, cleavable peptides, or aminobenzylcarbamates. A linker can contain a lysine with an N-terminal amine acetylated, and a valine-citrulline cleavage site. A linker can be a link created by a microbial transglutaminase, wherein the link can be created between an amine-containing moiety and a moiety engineered to contain glutamine as a result of the enzyme catalyzing a bond formation between the acyl group of a glutamine side chain and the primary amine of a lysine chain. A linker can contain a reactive primary amine. A linker can be a Sortase A linker. A Sortase A linker can be created by a Sortase A enzyme fusing an LPXTG (SEQ ID NO:49) recognition motif to an N-terminal GGG motif to regenerate a native amide bond. The linker created can therefore link a moiety attached to the LPXTG (SEQ ID NO:49) recognition motif with a moiety attached to the N-terminal GGG motif.

In the conjugates, a compound or salt of any one of Formulas (I), (II), (II-a), (II-b), (II-c), (II-d), (III), (III-a), (III-b), (III-c), (III-d), (IV), (IV-a), (IV-b), (IV-c), and (IV-d) and Table 1 is linked to the antibody by way of a linker(s), also referred to herein as L³. L³, as used herein, may be selected from any of the linker moieties discussed herein. The linker linking the compound or salt to the antibody construct of a conjugate may be short, long, hydrophobic, hydrophilic, flexible or rigid, or may be composed of segments that each independently have one or more of the above-mentioned properties such that the linker may include segments having different properties. The linkers may be polyvalent such that they covalently link more than one compound or salt to a single site on the antibody construct, or monovalent such that covalently they link a single compound or salt to a single site on the antibody construct.

Linkers of the disclosure (L³) may have from about 10 to about 500 atoms in a linker, such as from about 10 to about 400 atoms, such as about 10 to about 300 atoms in a linker. In certain embodiments, linkers of the disclosure have from about 30 to about 400 atoms, such as from about 30 to about 300 atoms in the linker.

As will be appreciated by skilled artisans, the linkers may link a compound or salt of any one of Formulas (I), (II), (II-a), (II-b), (II-c), (II-d), (III), (III-a), (III-b), (III-c), (III-d), (IV), (IV-a), (IV-b), (IV-c), and (IV-d) and Table 1 to an antibody construct or targeting moiety by a covalent linkage between the linker and the antibody construct or targeting moiety and compound. As used herein, the expression “linker” is intended to include (i) unconjugated forms of the linker that include a functional group capable of covalently linking the linker to a cyclic amino-pyrazinecarboxamide compound and a functional group capable of covalently linking the linker to an antibody construct; (ii) partially conjugated forms of the linker that include a functional group capable of covalently linking the linker to an antibody construct and that is covalently linked to a compound(s) or salt(s) of any one of Formulas (I), (II), (II-a), (II-b), (II-c), (II-d), (III), (III-a), (III-b), (III-c), (III-d), (IV), (IV-a), (IV-b), (IV-c), and (IV-d) and Table 1, or vice versa; and (iii) fully conjugated forms of the linker that is covalently linked to both a compound or salt of any one of Formulas (I), (II), (II-a), (II-b), (II-c), (II-d), (III), (III-a), (III-b), (III-c), (III-d), (IV), (IV-a), (IV-b), (IV-c), and (IV-d) and Table 1 and an antibody construct or targeting moiety. Certain embodiments pertain to a conjugate formed by contacting an antibody construct or targeting moiety that binds a cell surface receptor or tumor associated antigen expressed on a tumor cell with a linker-compound described herein under conditions in which the linker-compound covalently links to the antibody construct or targeting moiety. Other embodiments pertain to a method of making a conjugate formed by contacting a linker-compound under conditions in which the linker-compound covalently links to the antibody construct or targeting moiety.

In certain embodiments, any one of the compounds or salts described in the section entitled “Compounds (TGFβR2 Inhibitors)” is covalently bound to a linker (L). The linker may be covalently bound to any position, valence permitting. The linker may comprise a reactive moiety, e.g., an electrophile that can react to form a covalent bond with a moiety of an antibody construct such as, for example, a lysine, serine, threonine, cysteine, tyrosine, aspartic acid, glutamine, a non-natural amino acid residue, or glutamic acid residue. In some embodiments, a compound or salt of a compound in the section entitled “Compounds (TGFβR2 Inhibitors)” herein is covalently bound through the linker to an antibody construct.

Exemplary polyvalent linkers that may be used to link a TGFβR2 Inhibitor (such as cyclic amino-pyrazinecarboxamide compounds of this disclosure) to an antibody construct or targeting moiety are described. For example, Fleximer® linker technology has the potential to enable high drug-to-antibody ratio (“DAR”) conjugates with good physicochemical properties. As shown below, the Fleximer® linker technology is based on incorporating drug molecules into a solubilizing poly-acetal backbone via a sequence of ester bonds. The methodology renders highly-loaded conjugates (DAR up to 20) whilst maintaining good physicochemical properties. This methodology could be utilized with cyclic amino-pyrazinecarboxamide compound as shown in the Scheme below.

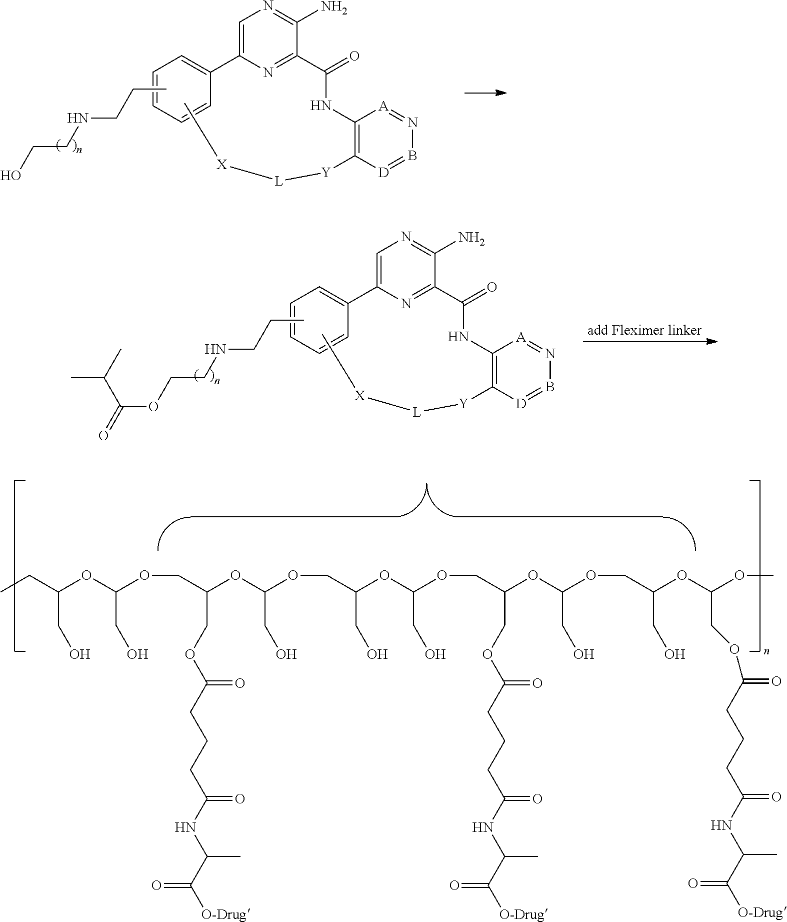

To utilize the Fleximer® linker technology depicted in the scheme above, an aliphatic alcohol can be present or introduced into the cyclic amino-pyrazinecarboxamide compound. The alcohol moiety is then conjugated to an alanine moiety, which is then synthetically incorporated into the Fleximer® linker. Liposomal processing of the conjugate in vitro releases the parent alcohol-containing drug.

By way of example and not limitation, some cleavable and noncleavable linkers that may be included in the conjugates are described below, in addition to those previously described.

Sulfamide linkers may be used to link many cyclic amino-pyrazinecarboxamide compounds to an antibody construct. Sulfamide linkers are as described herein and e.g., U.S. Patent Publication Number US 2019/0038765, the linkers of which are incorporated by reference herein.

Cleavable linkers can be cleavable in vitro and in vivo. Cleavable linkers can include chemically or enzymatically unstable or degradable linkages. Cleavable linkers can rely on processes inside the cell to liberate a cyclic amino-pyrazinecarboxamide compound, such as reduction in the cytoplasm, exposure to acidic conditions in the lysosome, or cleavage by specific proteases or other enzymes within the cell. Cleavable linkers can incorporate one or more chemical bonds that are either chemically or enzymatically cleavable while the remainder of the linker can be non-cleavable.

A linker can contain a chemically labile group such as hydrazone and/or disulfide groups. Linkers comprising chemically labile groups can exploit differential properties between the plasma and some cytoplasmic compartments. The intracellular conditions that can facilitate release of a cyclic amino-pyrazinecarboxamide compound for hydrazone containing linkers can be the acidic environment of endosomes and lysosomes, while the disulfide containing linkers can be reduced in the cytosol, which can contain high thiol concentrations, e.g., glutathione. The plasma stability of a linker containing a chemically labile group can be increased by introducing steric hindrance using substituents near the chemically labile group.

Acid-labile groups, such as hydrazone, can remain intact during systemic circulation in the blood's neutral pH environment (pH 7.3-7.5) and can undergo hydrolysis and can release the cyclic amino-pyrazinecarboxamide compound once the antibody conjugate or targeting moiety is internalized into mildly acidic endosomal (pH 5.0-6.5) and lysosomal (pH 4.5-5.0) compartments of the cell. This pH dependent release mechanism can be associated with nonspecific release of the drug. To increase the stability of the hydrazone group of the linker, the linker can be varied by chemical modification, e.g., substitution, allowing tuning to achieve more efficient release in the lysosome with a minimized loss in circulation.

Hydrazone-containing linkers can contain additional cleavage sites, such as additional acid-labile cleavage sites and/or enzymatically labile cleavage sites. Conjugates including exemplary hydrazone-containing linkers can include, for example, the following structures:

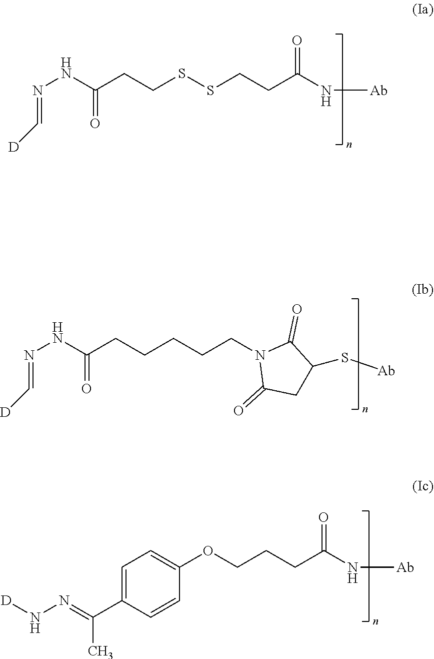

wherein D is a compound or salt of any one of Formulas (I), (II), (II-a), (II-b), (II-c), (II-d), (III), (III-a), (III-b), (III-c), (III-d), (IV), (IV-a), (IV-b), (IV-c), and (IV-d) and Ab is an antibody construct, respectively, and n represents the number of compound-bound linkers (LP) bound to the antibody construct. In certain linkers, such as linker (Ia), the linker can comprise two cleavable groups, a disulfide and a hydrazone moiety. For such linkers, effective release of the unmodified free cyclic amino-pyrazinecarboxamide compound can require acidic pH or disulfide reduction and acidic pH. Linkers such as (Ib) and (Ic) can be effective with a single hydrazone cleavage site.

Other acid-labile groups that can be included in linkers include cis-aconityl-containing linkers. cis-Aconityl chemistry can use a carboxylic acid juxtaposed to an amide bond to accelerate amide hydrolysis under acidic conditions.

Cleavable linkers can also include a disulfide group. Disulfides can be thermodynamically stable at physiological pH and can be designed to release the cyclic amino-pyrazinecarboxamide compound upon internalization inside cells, wherein the cytosol can provide a significantly more reducing environment compared to the extracellular environment. Scission of disulfide bonds can require the presence of a cytoplasmic thiol cofactor, such as (reduced) glutathione (GSH), such that disulfide-containing linkers can be reasonably stable in circulation, selectively releasing a cyclic amino-pyrazinecarboxamide compound in the cytosol. The intracellular enzyme protein disulfide isomerase, or similar enzymes capable of cleaving disulfide bonds, can also contribute to the preferential cleavage of disulfide bonds inside cells. GSH can be present in cells in the concentration range of 0.5-10 mM compared with a significantly lower concentration of GSH or cysteine, the most abundant low-molecular weight thiol, in circulation at approximately 5 μM. Tumor cells, where irregular blood flow can lead to a hypoxic state, can result in enhanced activity of reductive enzymes and therefore even higher glutathione concentrations. The in vivo stability of a disulfide-containing linker can be enhanced by chemical modification of the linker, e.g., use of steric hindrance adjacent to the disulfide bond.

Antibody conjugates containing cyclic amino-pyrazinecarboxamide compounds that include exemplary disulfide-containing linkers can include the following structures:

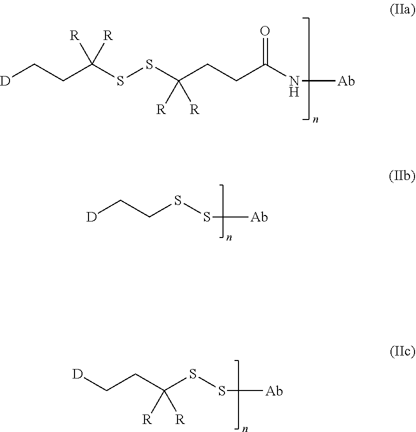

wherein D is a compound or salt of any one of Formulas (I), (II), (II-a), (II-b), (II-c), (II-d), (III), (III-a), (III-b), (III-c), (III-d), (IV), (IV-a), (IV-b), (IV-c), and (IV-d) and Ab is an antibody construct, respectively, n represents the number of compounds bound to linkers (L³) bound to the antibody construct and R is independently selected at each occurrence from hydrogen or alkyl, for example. Increasing steric hindrance adjacent to the disulfide bond can increase the stability of the linker. Structures such as (IIa) and (IIc) can show increased in vivo stability when one or more R groups is selected from a lower alkyl such as methyl.

Another type of linker that can be used is a linker that is specifically cleaved by an enzyme. For example, the linker can be cleaved by a lysosomal enzyme. Such linkers can be peptide-based or can include peptidic regions that can act as substrates for enzymes. Peptide based linkers can be more stable in plasma and extracellular milieu than chemically labile linkers.

Peptide bonds can have good serum stability, as lysosomal proteolytic enzymes can have very low activity in blood due to endogenous inhibitors and the unfavorably high pH value of blood compared to lysosomes. Release of a cyclic amino-pyrazinecarboxamide compound from an antibody construct can occur due to the action of lysosomal proteases, e.g., cathepsin and plasmin. These proteases can be present at elevated levels in certain tumor tissues. The linker can be cleavable by a lysosomal enzyme. The lysosomal enzyme can be, for example, cathepsin B, β-glucuronidase, or β-galactosidase.

The cleavable peptide can be selected from tetrapeptides such as Gly-Phe-Leu-Gly (SEQ ID NO: 235), Ala-Leu-Ala-Leu (SEQ ID NO: 236) or dipeptides such as Val-Cit, Val-Ala, and Phe-Lys. Dipeptides can have lower hydrophobicity compared to longer peptides.

A variety of dipeptide-based cleavable linkers can be used in the antibody constructs to form conjugates of a cyclic amino-pyrazinecarboxamide compound described herein.

Enzymatically cleavable linkers can include a self-immolative spacer to spatially separate the cyclic amino-pyrazinecarboxamide compound from the site of enzymatic cleavage. The direct attachment of a cyclic amino-pyrazinecarboxamide compound to a peptide linker can result in proteolytic release of an amino acid adduct of the cyclic amino-pyrazinecarboxamide compound, thereby impairing its activity. The use of a self-immolative spacer can allow for the elimination of the fully active, chemically unmodified cyclic amino-pyrazinecarboxamide compound upon amide bond hydrolysis.

One self-immolative spacer can be a bifunctional para-aminobenzyl alcohol group, which can link to the peptide through the amino group, forming an amide bond, while amine containing cyclic amino-pyrazinecarboxamide compounds can be attached through carbamate functionalities to the benzylic hydroxyl group of the linker (to give a p-amidobenzylcarbamate, PABC). The resulting pro-cyclic-amino-pyrazinecarboxamide compound can be activated upon protease-mediated cleavage, leading to a 1,6-elimination reaction releasing the unmodified cyclic amino-pyrazinecarboxamide compound, carbon dioxide, and remnants of the linker group. The following scheme depicts the fragmentation of p-amidobenzyl carbamate and release of the cyclic amino-pyrazinecarboxamide compound:

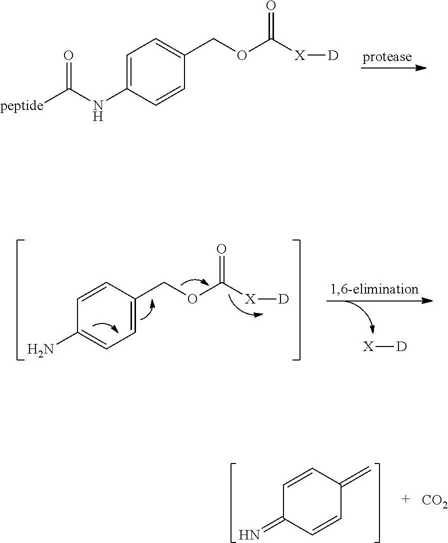

wherein X-D represents the unmodified cyclic amino-pyrazinecarboxamide compound.

Heterocyclic variants of this self-immolative group have also been described.

The enzymatically cleavable linker can be a β-glucuronic acid-based linker. Facile release of the cyclic amino-pyrazinecarboxamide compound can be realized through cleavage of the β-glucuronide glycosidic bond by the lysosomal enzyme β-glucuronidase. This enzyme can be abundantly present within lysosomes and can be overexpressed in some tumor types, while the enzyme activity outside cells can be low. β-Glucuronic acid-based linkers can be used to circumvent the tendency of an antibody construct conjugate of a cyclic amino-pyrazinecarboxamide compound to undergo aggregation due to the hydrophilic nature of β-glucuronides. In certain embodiments, β-glucuronic acid-based linkers can link an antibody construct to a hydrophobic cyclic amino-pyrazinecarboxamide compound. The following scheme depicts the release of a cyclic amino-pyrazinecarboxamide compound (D) from an antibody construct conjugate of a cyclic amino-pyrazinecarboxamide compound containing a β-glucuronic acid-based linker:

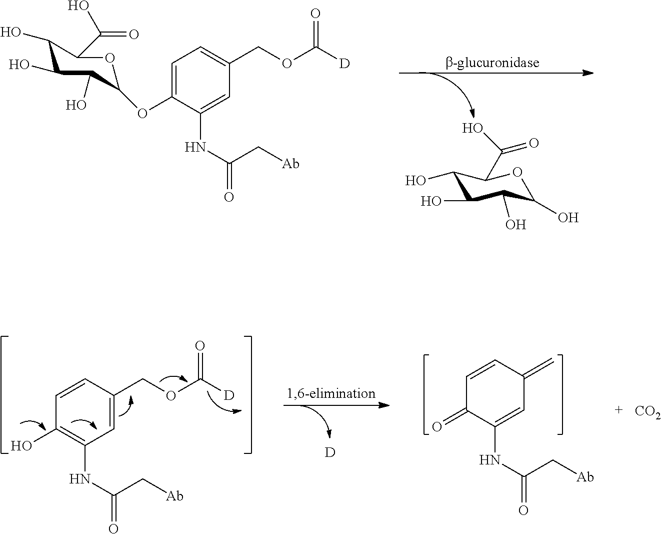

wherein Ab indicates the antibody construct.

A variety of cleavable β-glucuronic acid-based linkers useful for linking drugs such as auristatins, camptothecin and doxorubicin analogues, CBI minor-groove binders, and psymberin to antibodies have been described. These β-glucuronic acid-based linkers may be used in the conjugates. In certain embodiments, the enzymatically cleavable linker is a β-galactoside-based linker. β-Galactoside is present abundantly within lysosomes, while the enzyme activity outside cells is low.

Additionally, cyclic amino-pyrazinecarboxamide compounds containing a phenol group can be covalently bonded to a linker through the phenolic oxygen. One such linker relies on a methodology in which a diamino-ethane “Space Link” is used in conjunction with traditional “PABO”-based self-immolative groups to deliver phenols.

Cleavable linkers can include non-cleavable portions or segments, and/or cleavable segments or portions can be included in an otherwise non-cleavable linker to render it cleavable. By way of example only, polyethylene glycol (PEG) and related polymers can include cleavable groups in the polymer backbone. For example, a polyethylene glycol or polymer linker can include one or more cleavable groups such as a disulfide, a hydrazone or a dipeptide.

Other degradable linkages that can be included in linkers can include ester linkages formed by the reaction of PEG carboxylic acids or activated PEG carboxylic acids with alcohol groups on a cyclic amino-pyrazinecarboxamide compound, wherein such ester groups can hydrolyze under physiological conditions to release the cyclic amino-pyrazinecarboxamide compound. Hydrolytically degradable linkages can include, but are not limited to, carbonate linkages; imine linkages resulting from reaction of an amine and an aldehyde; phosphate ester linkages formed by reacting an alcohol with a phosphate group; acetal linkages that are the reaction product of an aldehyde and an alcohol; orthoester linkages that are the reaction product of a formate and an alcohol; and oligonucleotide linkages formed by a phosphoramidite group, including but not limited to, at the end of a polymer, and a 5′ hydroxyl group of an oligonucleotide.

A linker can contain an enzymatically cleavable peptide, for example, a linker comprising structural formula (IIIa), (IIIb), (IIIc), or (IIId):

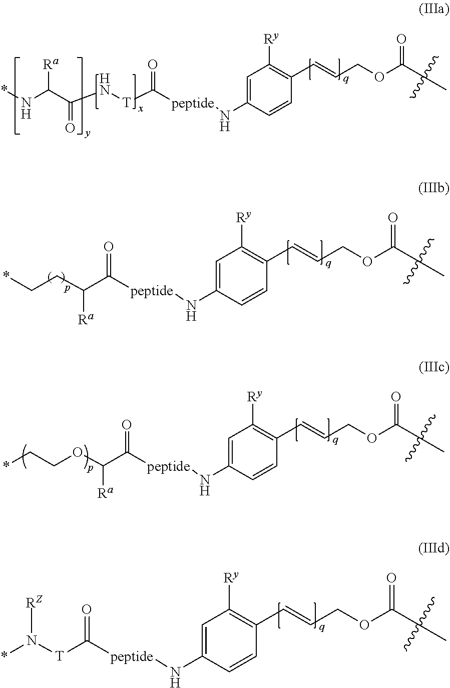

or a salt thereof, wherein: “peptide” represents a peptide (illustrated in N→C orientation, wherein peptide includes the amino and carboxy “termini”) that is cleavable by a lysosomal enzyme; T represents a polymer comprising one or more ethylene glycol units or an alkylene chain, or combinations thereof; R^{a} is selected from hydrogen, alkyl, sulfonate and methyl sulfonate; R^{y} is hydrogen or C₁₋₄ alkyl-(O)ᵣ—(C₁₋₄ alkylene)ₛ-G¹ or C₁₋₄ alkyl-(N)—[(C₁₋₄ alkylene)-G¹]₂; R^{z} is C₁₋₄ alkyl-(O)ᵣ—(C₁₋₄ alkylene)ₛ-G²; G¹ is SO₃H, CO₂H, PEG 4-32, or a sugar moiety; G² is SO₃H, CO₂H, or PEG 4-32 moiety; r is 0 or 1; s is 0 or 1; p is an integer ranging from 0 to 5; q is 0 or 1; x is 0 or 1; y is 0 or 1; [symbol] represents the point of attachment of the linker to a compound or salt of any one of Formulas (I), (II), (II-a), (II-b), (II-c), (II-d), (III), (III-a), (III-b), (III-c), (III-d), (IV), (IV-a), (IV-b), (IV-c), and (IV-d); and * represents the point of attachment to the remainder of the linker.

In certain embodiments, the peptide can be selected from natural amino acids, unnatural amino acids or combinations thereof. In certain embodiments, the peptide can be selected from a tripeptide or a dipeptide. In particular embodiments, the dipeptide can comprise L-amino acids and be selected from: Val-Cit; Cit-Val; Ala-Ala; Ala-Cit; Cit-Ala; Asn-Cit; Cit-Asn; Cit-Cit; Val-Glu; Glu-Val; Ser-Cit; Cit-Ser; Lys —Cit; Cit-Lys; Asp-Cit; Cit-Asp; Ala-Val; Val-Ala; Phe-Lys; Lys-Phe; Val-Lys; Lys-Val; Ala-Lys; Lys-Ala; Phe-Cit; Cit-Phe; Leu-Cit; Cit-Leu; Ile-Cit; Cit-Ile; Phe-Arg; Arg-Phe; Cit-Trp; and Trp-Cit, or salts thereof.

Exemplary embodiments of linkers according to structural formula (IIIa) are illustrated below (as illustrated, the linkers include a reactive group suitable for covalently linking the linker to an antibody construct):

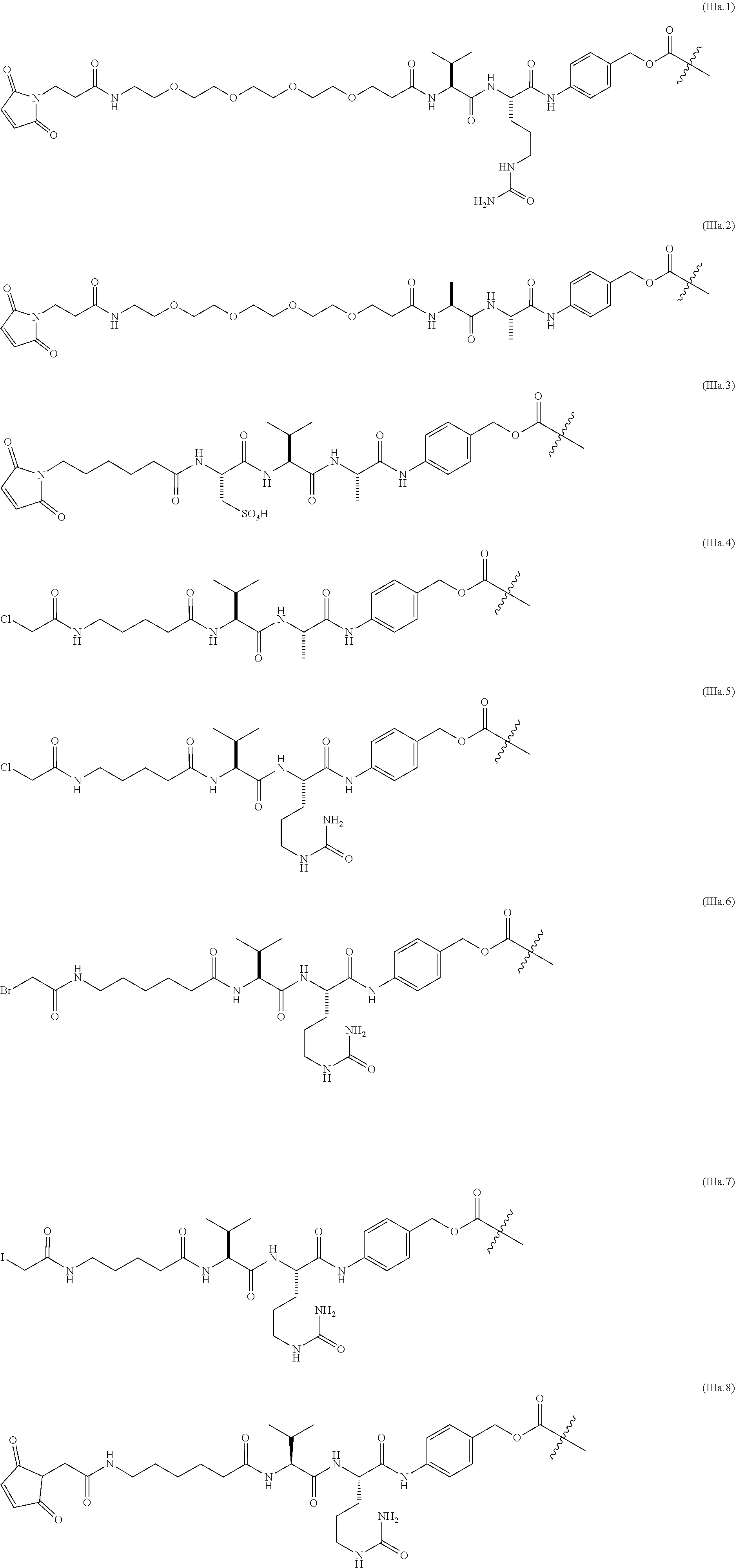

wherein [symbol] indicates an attachment site of a linker (L³) to a cyclic amino-pyrazinecarboxamide compound.

Exemplary embodiments of linkers according to structural formula (IIIb), (IIIc), or (IIId) that can be included in the conjugates can include the linkers illustrated below (as illustrated, the linkers include a reactive group suitable for covalently linking the linker to an antibody construct):

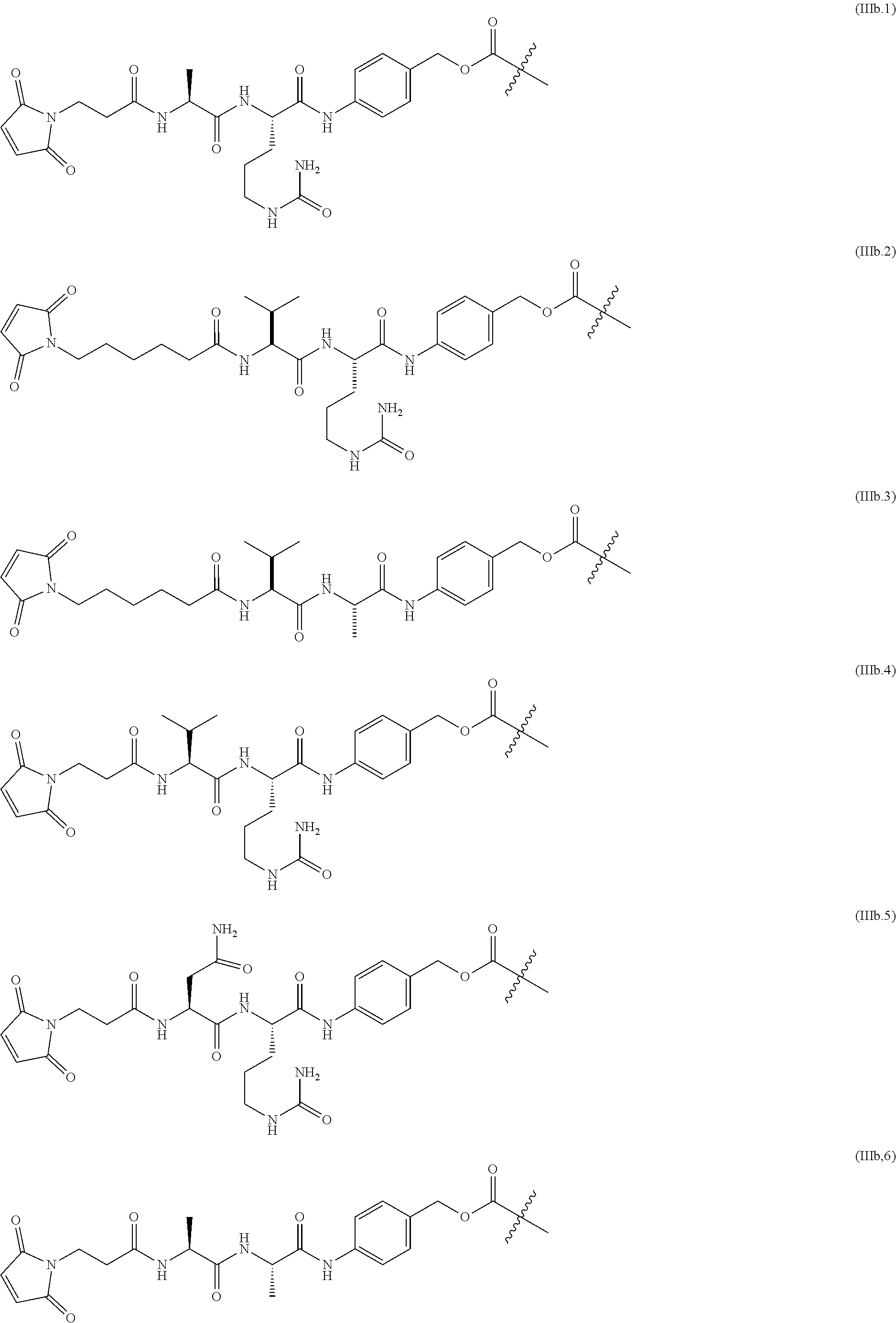 [SMILES: *C(=O)OCc1ccc(NC(=O)[C@H](CCCNC(N)=O)NC(=O)[C@H](C)NC(=O)CCN2C(=O)C=CC2=O)cc1 ; *C(=O)OCc1ccc(NC(=O)[C@H](CCCNC(N)=O)NC(=O)[C@@H](NC(=O)CCCCCN2C(=O)C=CC2=O)C(C)C)cc1 ; *C(=O)OCc1ccc(NC(=O)[C@H](C)NC(=O)[C@@H](NC(=O)CCCCCN2C(=O)C=CC2=O)C(C)C)cc1 ; *C(=O)OCc1ccc(NC(=O)[C@H](CCCNC(N)=O)NC(=O)[C@@H](NC(=O)CCN2C(=O)C=CC2=O)C(C)C)cc1 ; *C(=O)OCc1ccc(NC(=O)[C@H](CCCNC(N)=O)NC(=O)[C@H](CC(N)=O)NC(=O)CCN2C(=O)C=CC2=O)cc1 ; *C(=O)OCc1ccc(NC(=O)[C@H](C)NC(=O)[C@H](C)NC(=O)CCN2C(=O)C=CC2=O)cc1]

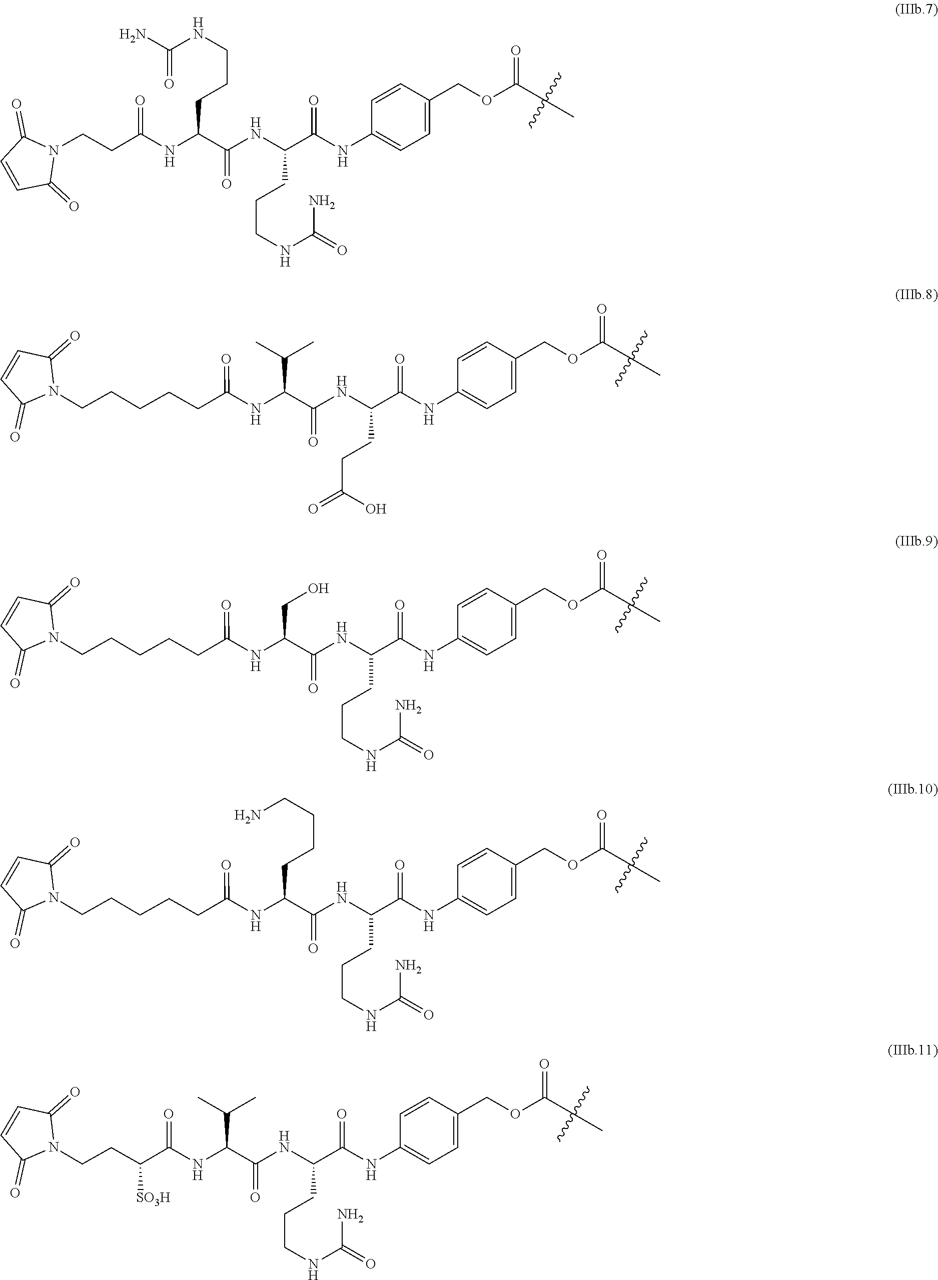 [SMILES: *C(=O)OCc1ccc(NC(=O)[C@H](CCCNC(N)=O)NC(=O)[C@H](CCCNC(N)=O)NC(=O)CCN2C(=O)C=CC2=O)cc1 ; *C(=O)OCc1ccc(NC(=O)[C@H](CCC(=O)O)NC(=O)[C@@H](NC(=O)CCCCCN2C(=O)C=CC2=O)C(C)C)cc1 ; *C(=O)OCc1ccc(NC(=O)[C@H](CCCNC(N)=O)NC(=O)[C@H](CO)NC(=O)CCCCCN2C(=O)C=CC2=O)cc1 ; *C(=O)OCc1ccc(NC(=O)[C@H](CCCNC(N)=O)NC(=O)[C@H](CCCCN)NC(=O)CCCCCN2C(=O)C=CC2=O)cc1 ; *C(=O)OCc1ccc(NC(=O)[C@H](CCCNC(N)=O)NC(=O)[C@@H](NC(=O)[C@@H](CCN2C(=O)C=CC2=O)S(=O)(=O)O)C(C)C)cc1]

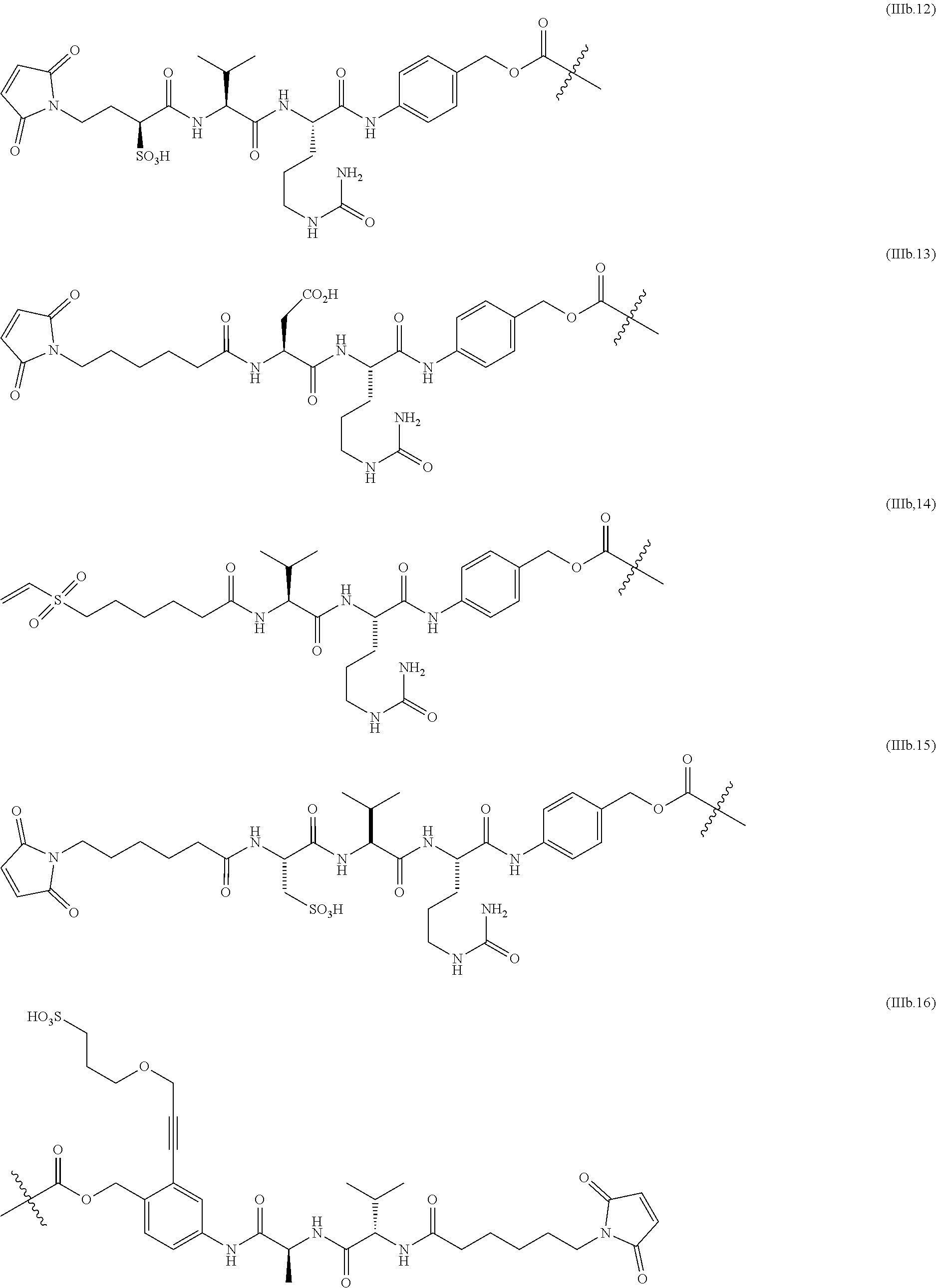 [SMILES: *C(=O)OCc1ccc(NC(=O)[C@H](CCCNC(N)=O)NC(=O)[C@@H](NC(=O)[C@H](CCN2C(=O)C=CC2=O)S(=O)(=O)O)C(C)C)cc1 ; *C(=O)OCc1ccc(NC(=O)[C@H](CCCNC(N)=O)NC(=O)[C@H](CC(=O)O)NC(=O)CCCCCN2C(=O)C=CC2=O)cc1 ; *C(=O)OCc1ccc(NC(=O)[C@H](CCCNC(N)=O)NC(=O)[C@@H](NC(=O)CCCCCS(=O)(=O)C=C)C(C)C)cc1 ; *C(=O)OCc1ccc(NC(=O)[C@H](CCCNC(N)=O)NC(=O)[C@@H](NC(=O)[C@H](CS(=O)(=O)O)NC(=O)CCCCCN2C(=O)C=CC2=O)C(C)C)cc1 ; *C(=O)OCc1ccc(NC(=O)[C@H](C)NC(=O)[C@@H](NC(=O)CCCCCN2C(=O)C=CC2=O)C(C)C)cc1C#CCOCCCS(=O)(=O)O]

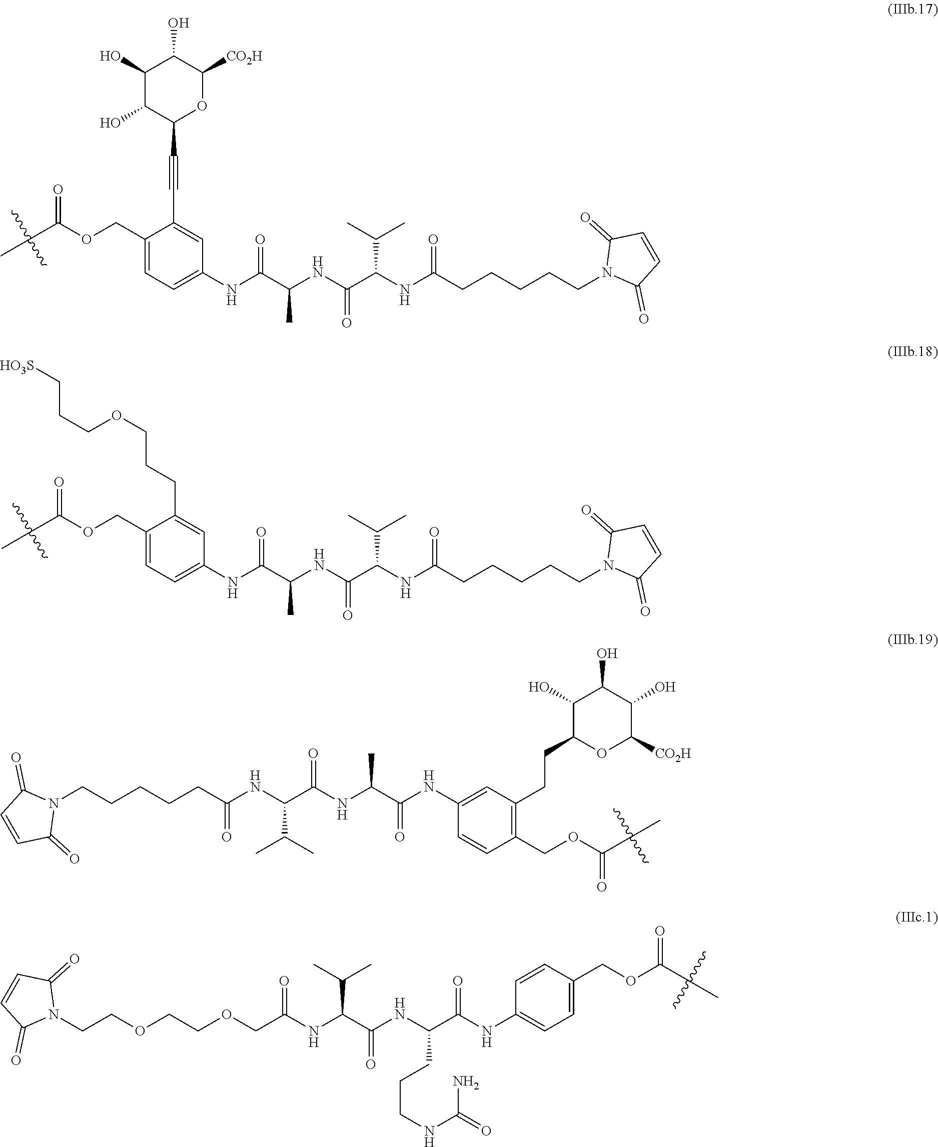 [SMILES: *C(=O)OCc1ccc(NC(=O)[C@H](C)NC(=O)[C@@H](NC(=O)CCCCCN2C(=O)C=CC2=O)C(C)C)cc1C#C[C@@H]1O[C@H](C(=O)O)[C@@H](O)[C@H](O)[C@H]1O ; *C(=O)OCc1ccc(NC(=O)[C@H](C)NC(=O)[C@@H](NC(=O)CCCCCN2C(=O)C=CC2=O)C(C)C)cc1CCCOCCCS(=O)(=O)O ; *C(=O)OCc1ccc(NC(=O)[C@H](C)NC(=O)[C@@H](NC(=O)CCCCCN2C(=O)C=CC2=O)C(C)C)cc1CC[C@@H]1O[C@H](C(=O)O)[C@@H](O)[C@H](O)[C@H]1O ; *C(=O)OCc1ccc(NC(=O)[C@H](CCCNC(N)=O)NC(=O)[C@@H](NC(=O)COCCOCCN2C(=O)C=CC2=O)C(C)C)cc1]

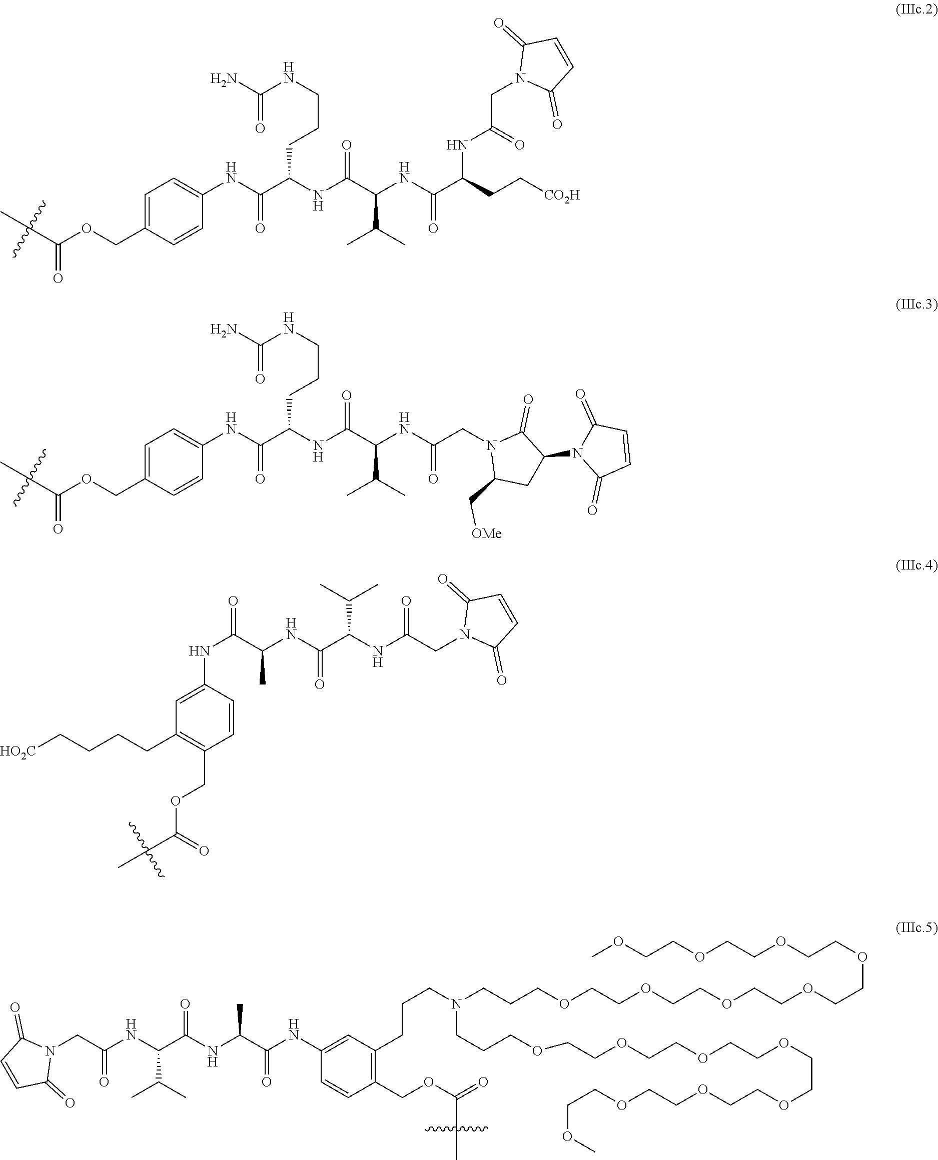 [SMILES: *C(=O)OCc1ccc(NC(=O)[C@H](CCCNC(N)=O)NC(=O)[C@@H](NC(=O)[C@H](CCC(=O)O)NC(=O)CN2C(=O)C=CC2=O)C(C)C)cc1 ; *C(=O)OCc1ccc(NC(=O)[C@H](CCCNC(N)=O)NC(=O)[C@@H](NC(=O)CN2C(=O)[C@@H](N3C(=O)C=CC3=O)C[C@H]2COC)C(C)C)cc1 ; *C(=O)OCc1ccc(NC(=O)[C@H](C)NC(=O)[C@@H](NC(=O)CN2C(=O)C=CC2=O)C(C)C)cc1CCCCC(=O)O ; *C(=O)OCc1ccc(NC(=O)[C@H](C)NC(=O)[C@@H](NC(=O)CN2C(=O)C=CC2=O)C(C)C)cc1CCCN(CCCOCCOCCOCCOCCOCCOCCOCCOC)CCCOCCOCCOCCOCCOCCOCCOCCOC]

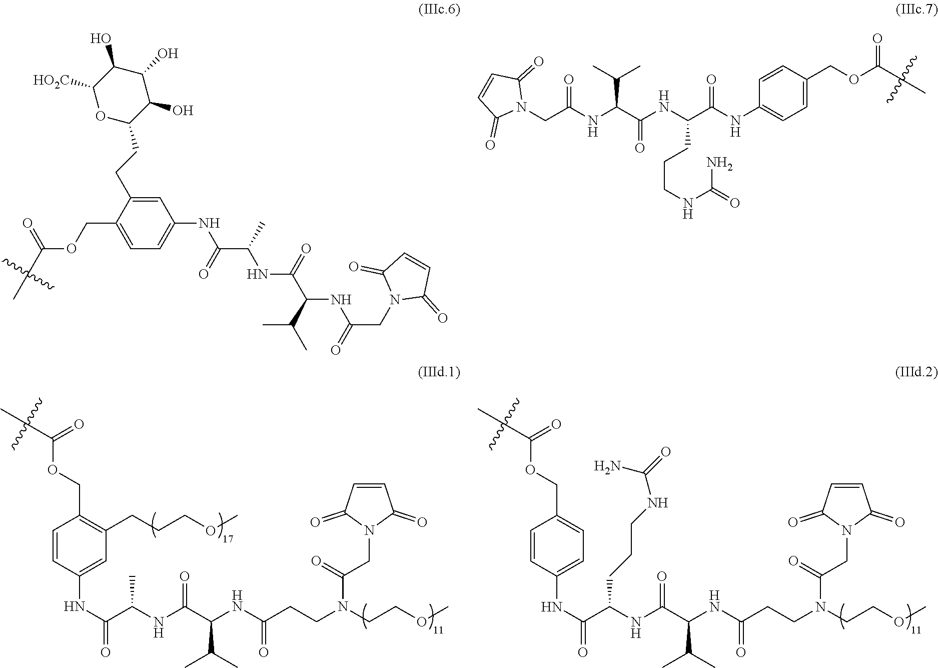

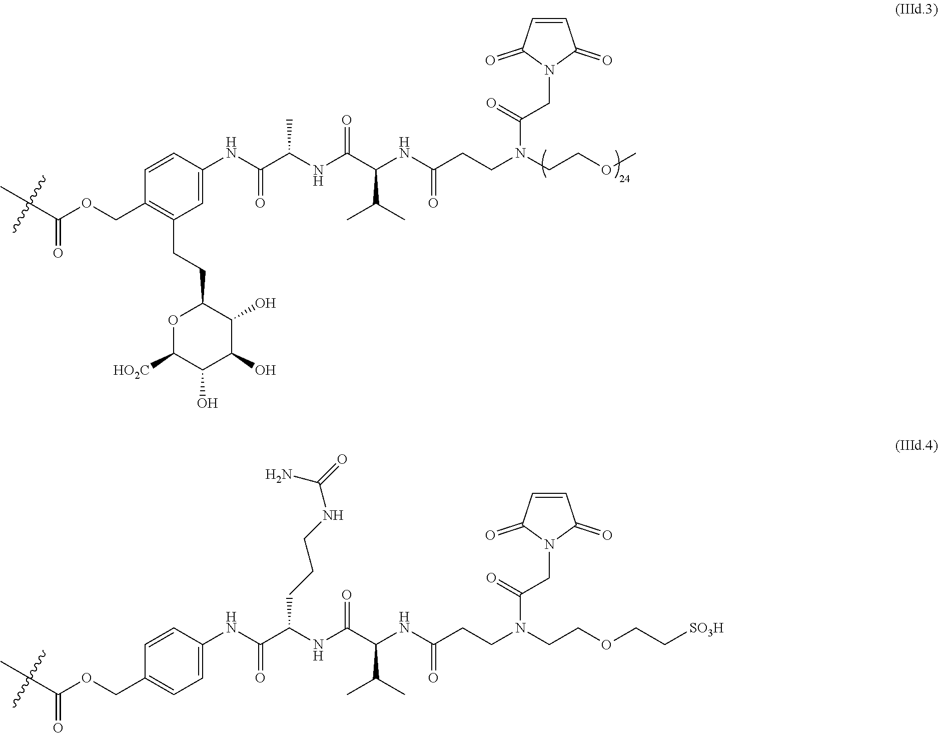

wherein [symbol] indicates an attachment site to a cyclic amino-pyrazinecarboxamide compound.

The linker can contain an enzymatically cleavable sugar moiety, for example, a linker comprising structural formula (IVa), (IVb), (IVc), (IVd), or (IVe):

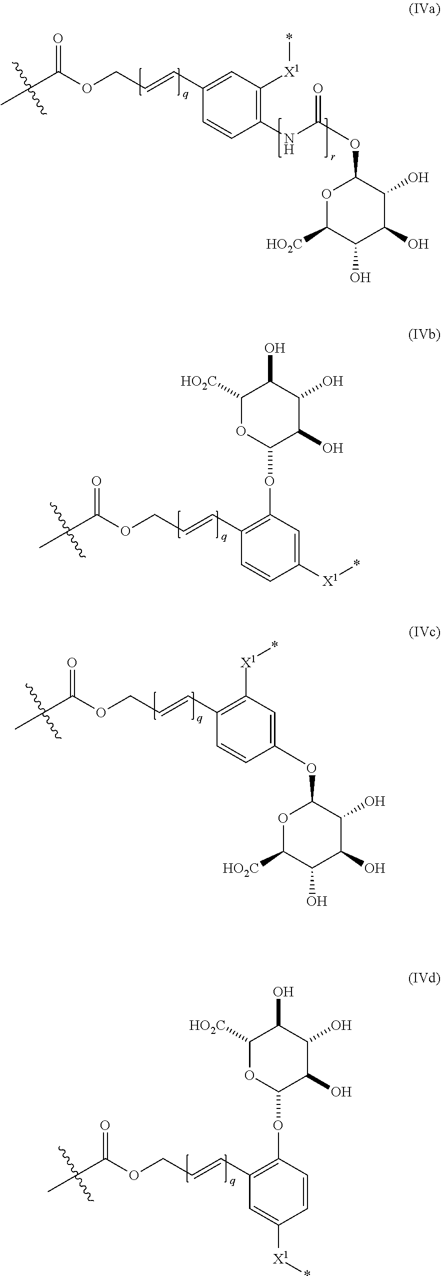

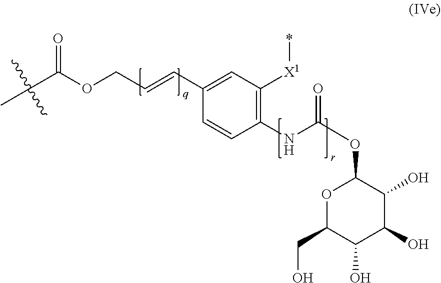

or a salt thereof, wherein: q is 0 or 1; r is 0 or 1; X¹ is CH₂, O or NH; [symbol] represents the point of attachment of the linker (L³) to the compound or salt of any one of Formulas (I), (II), (II-a), (II-b), (II-c), (II-d), (III), (III-a), (III-b), (III-c), (III-d), (IV), (IV-a), (IV-b), (IV-c), and (IV-d); and represents the point of attachment to the remainder of the linker.

Exemplary embodiments of linkers according to structural formula (IVa) that may be included in the antibody construct conjugates described herein can include the linkers illustrated below (as illustrated, the linkers include a group suitable for covalently linking the linker to an antibody construct):

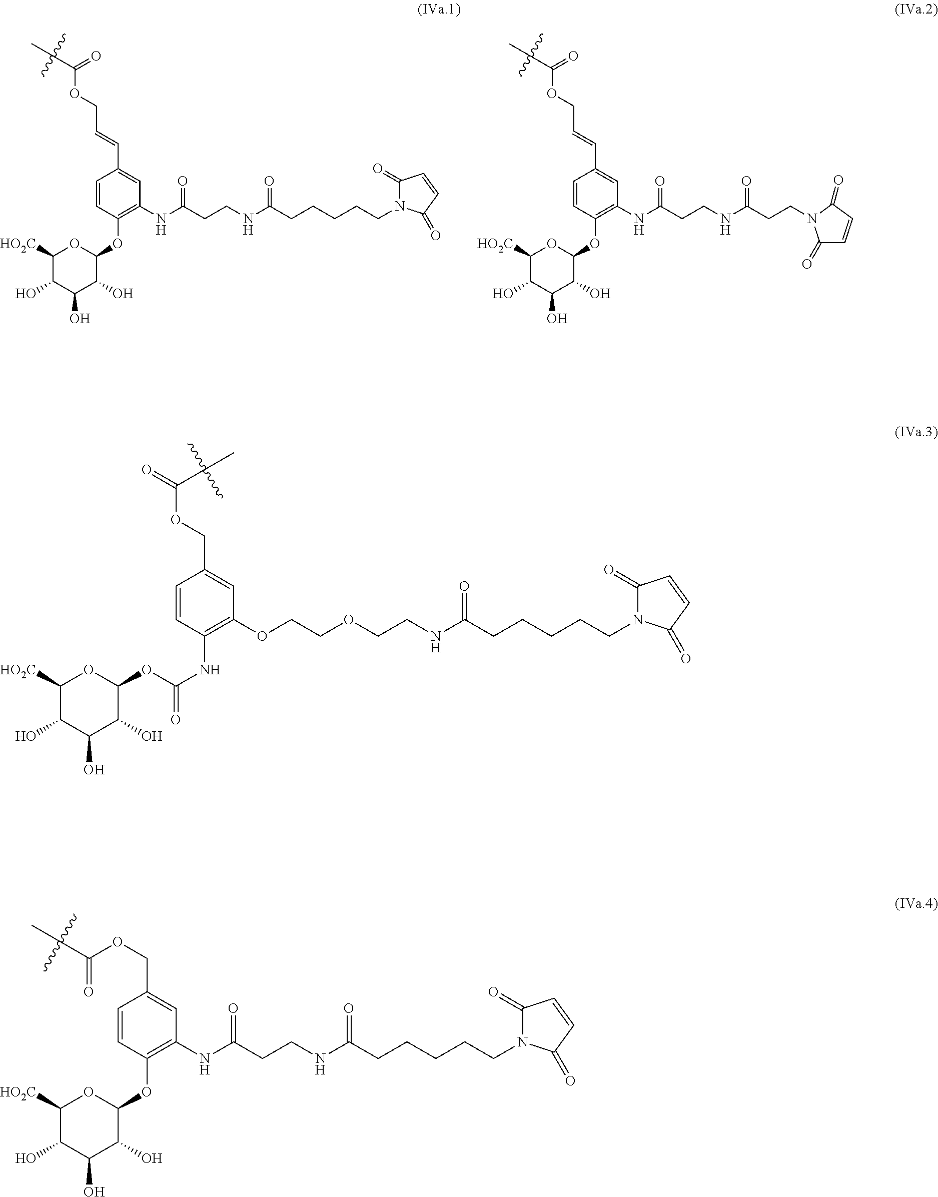

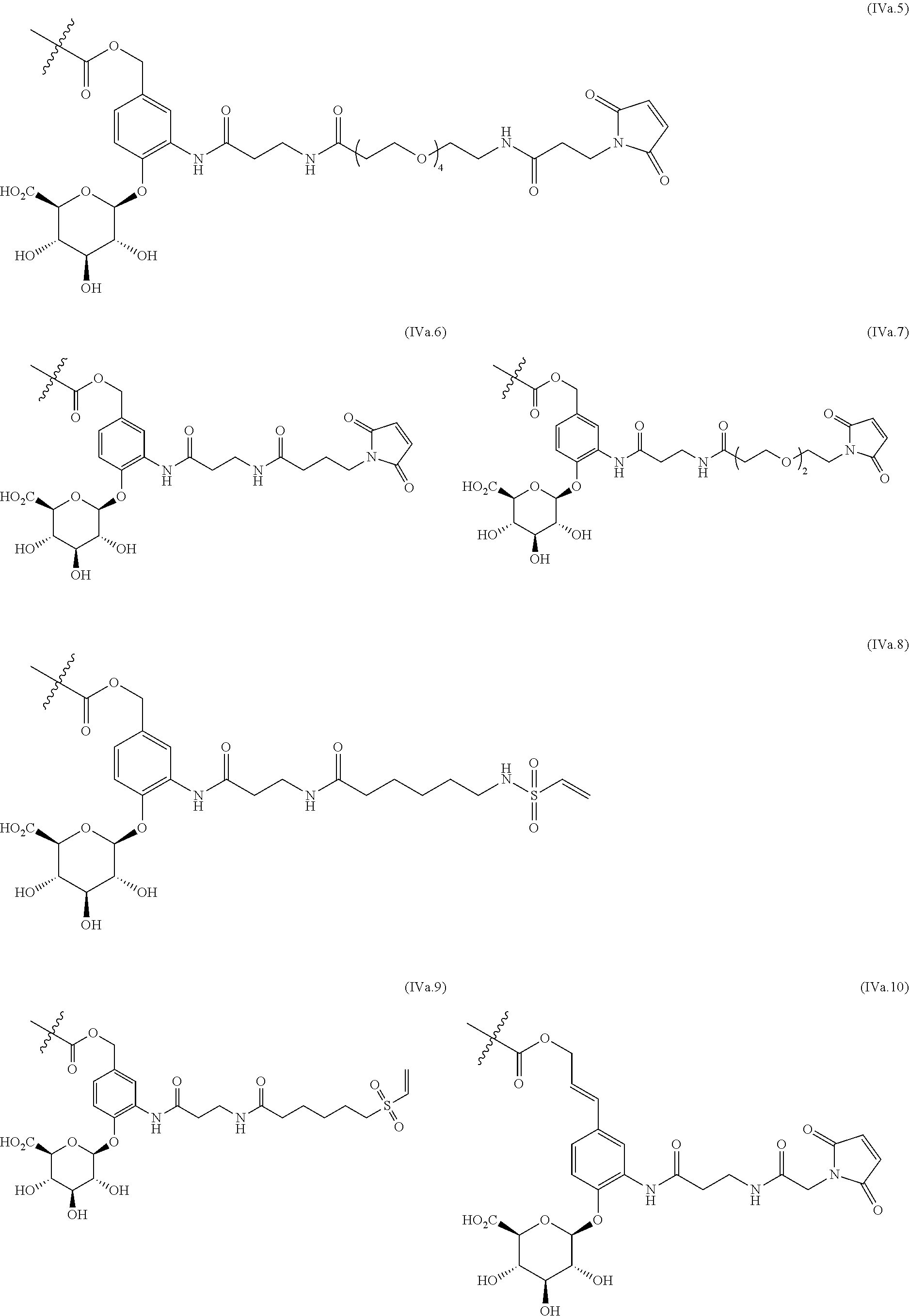

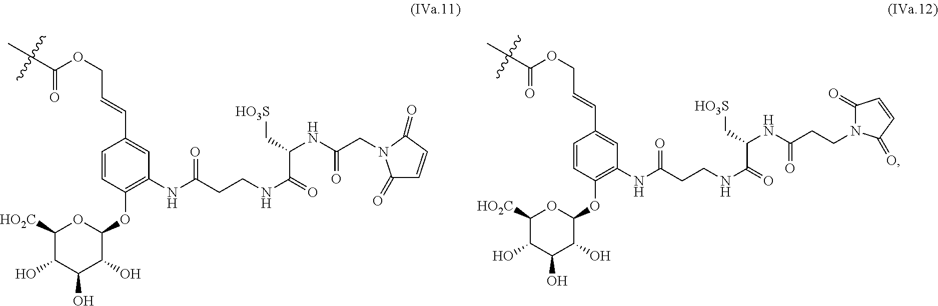

wherein [symbol] represents the point of attachment of the linker (L³) to the compound or salt of any one of Formulas (I), (II), (II-a), (II-b), (II-c), (II-d), (III), (III-a), (III-b), (III-c), (III-d), (IV), (IV-a), (IV-b), (IV-c), and (IV-d).

Exemplary embodiments of linkers according to structural formula (IVb) that may be included in the conjugates include the linkers illustrated below (as illustrated, the linkers include a group suitable for covalently linking the linker to an antibody construct):

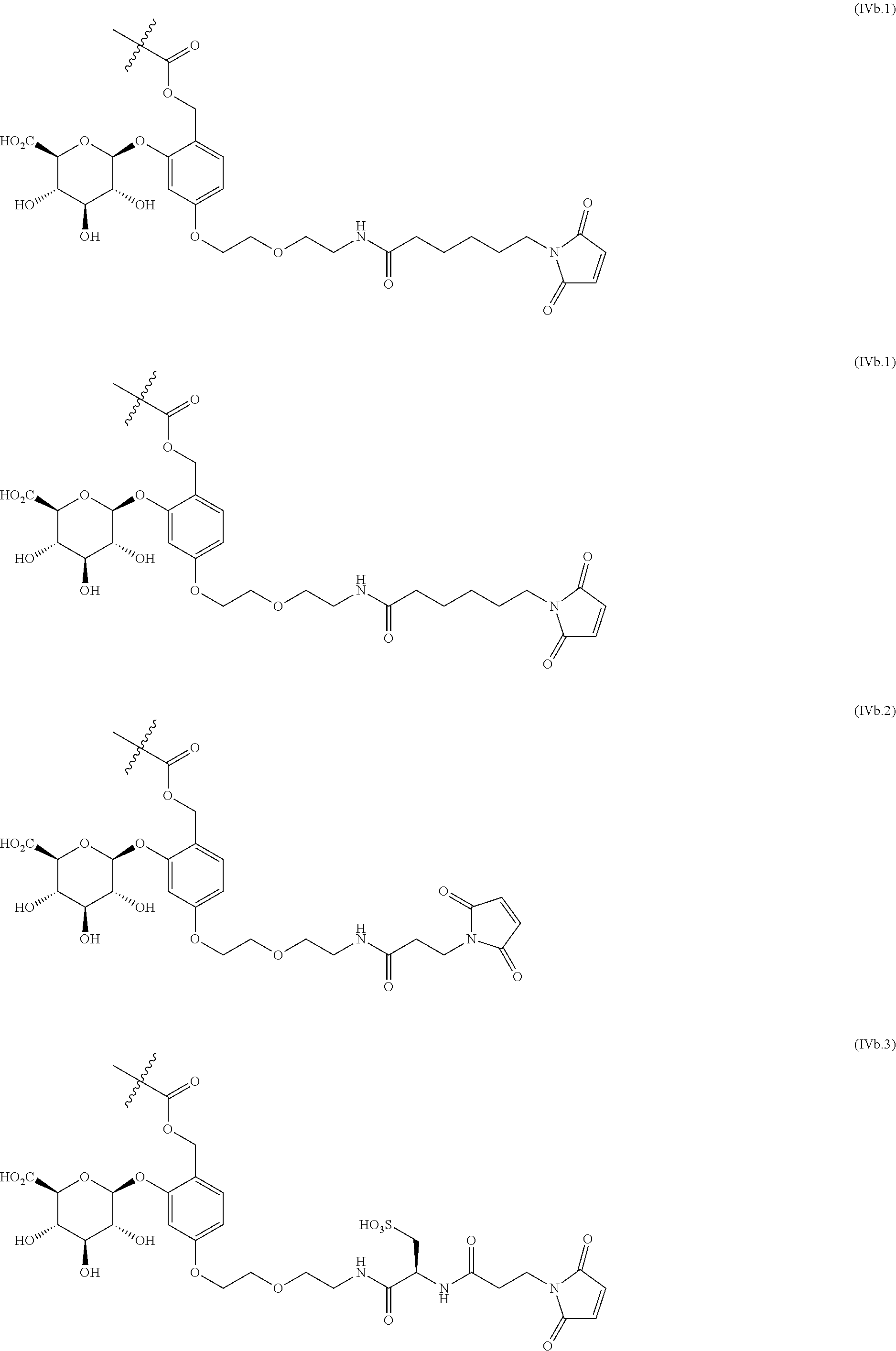

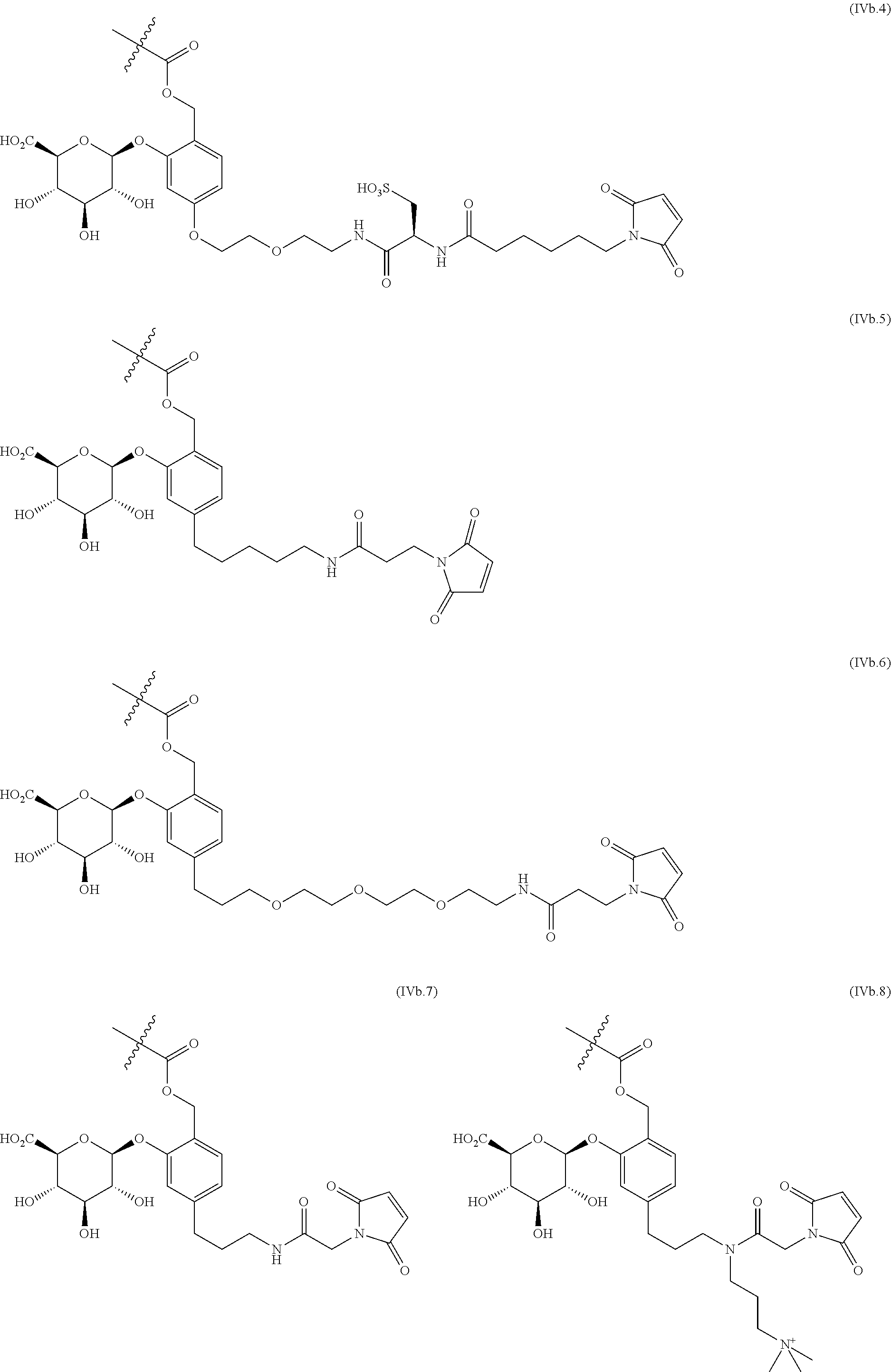

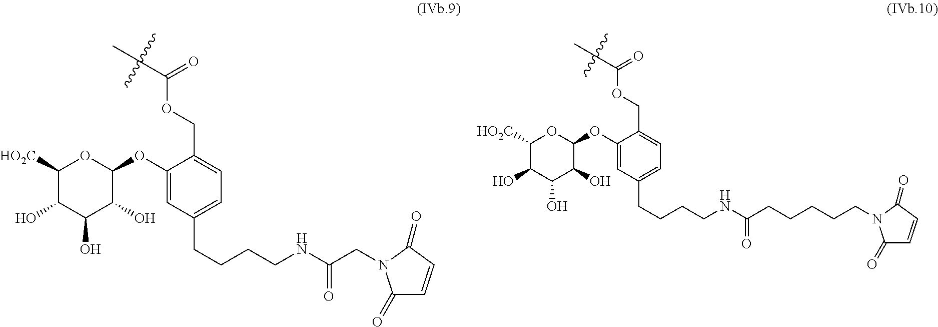

wherein [symbol] represents the point of attachment of the linker (L³) to a cyclic amino-pyrazinecarboxamide compound.

Exemplary embodiments of linkers according to structural formula (IVc) that may be included in the conjugates include the linkers illustrated below (as illustrated, the linkers include a group suitable for covalently linking the linker to an antibody construct):

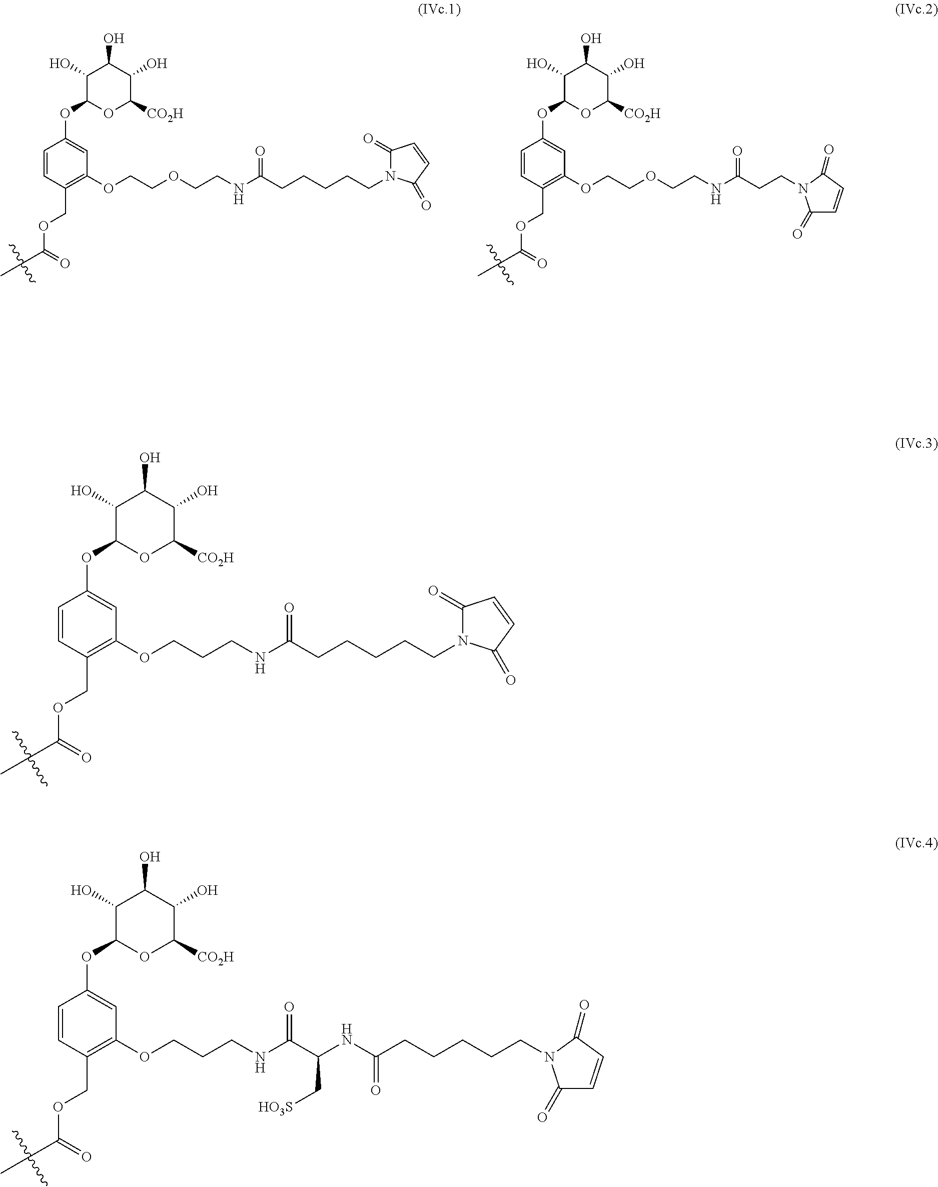

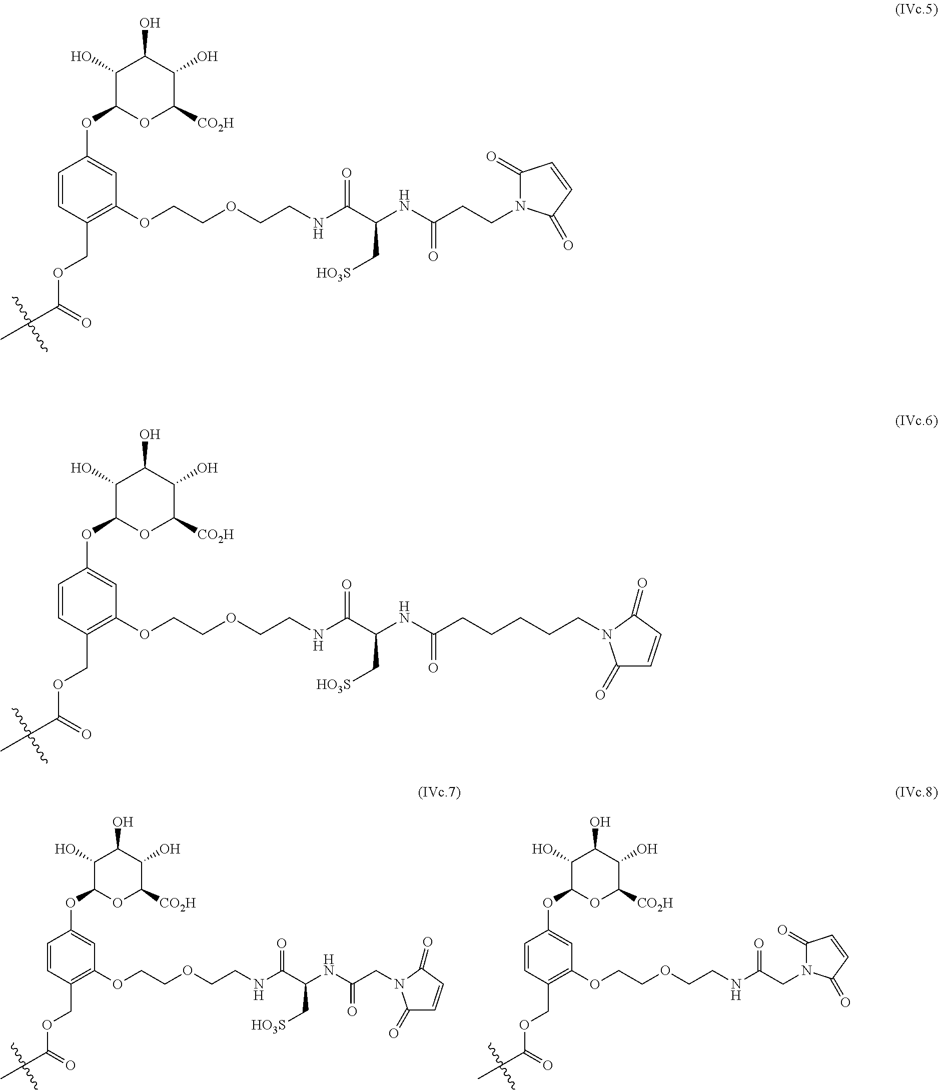

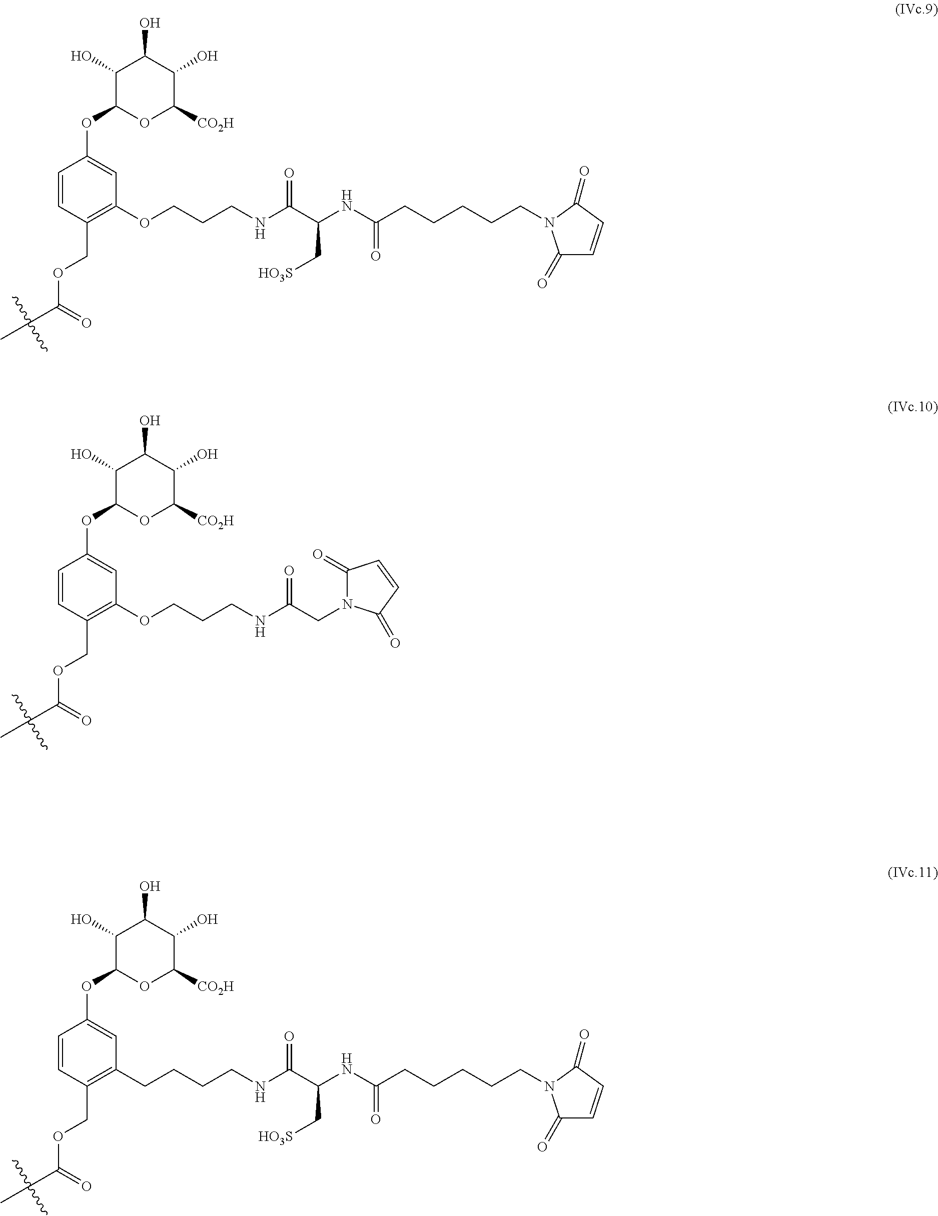

wherein [symbol] represents the point of attachment of the linker (L³) to a cyclic amino-pyrazinecarboxamide compound.

Exemplary embodiments of linkers according to structural formula (IVd) that may be included in the conjugates include the linkers illustrated below (as illustrated, the linkers include a group suitable for covalently linking the linker to an antibody):

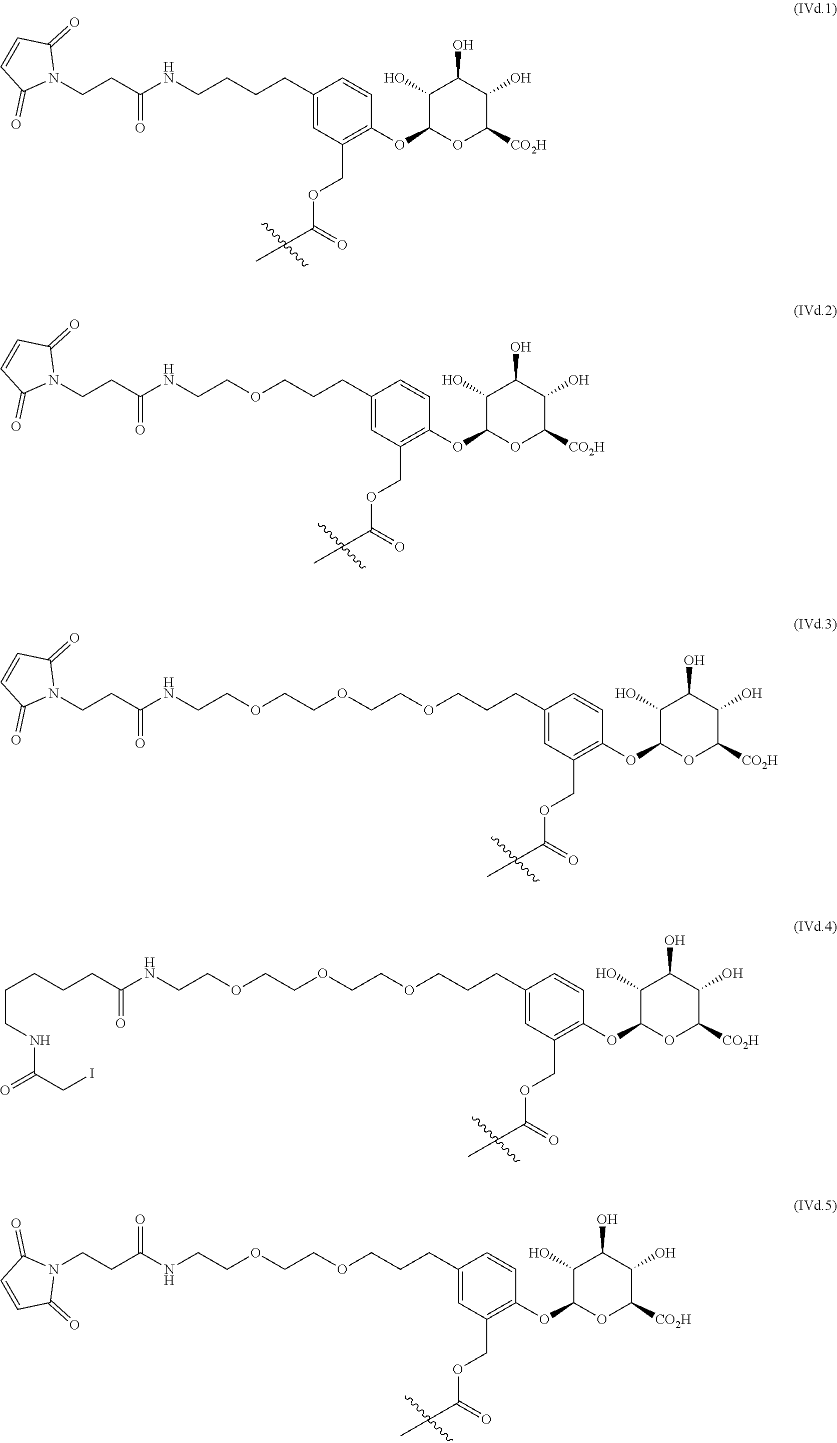

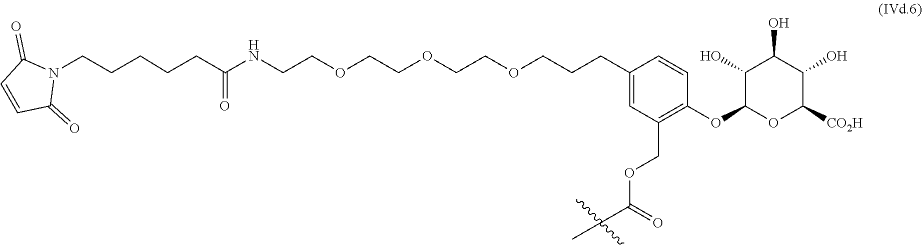

wherein [symbol] represents the point of attachment site of the linker (L³) to a cyclic amino-pyrazinecarboxamide compound.

Exemplary embodiments of linkers according to structural formula (IVe) that may be included in the conjugates include the linkers illustrated below (as illustrated, the linkers include a group suitable for covalently linking the linker to an antibody construct):

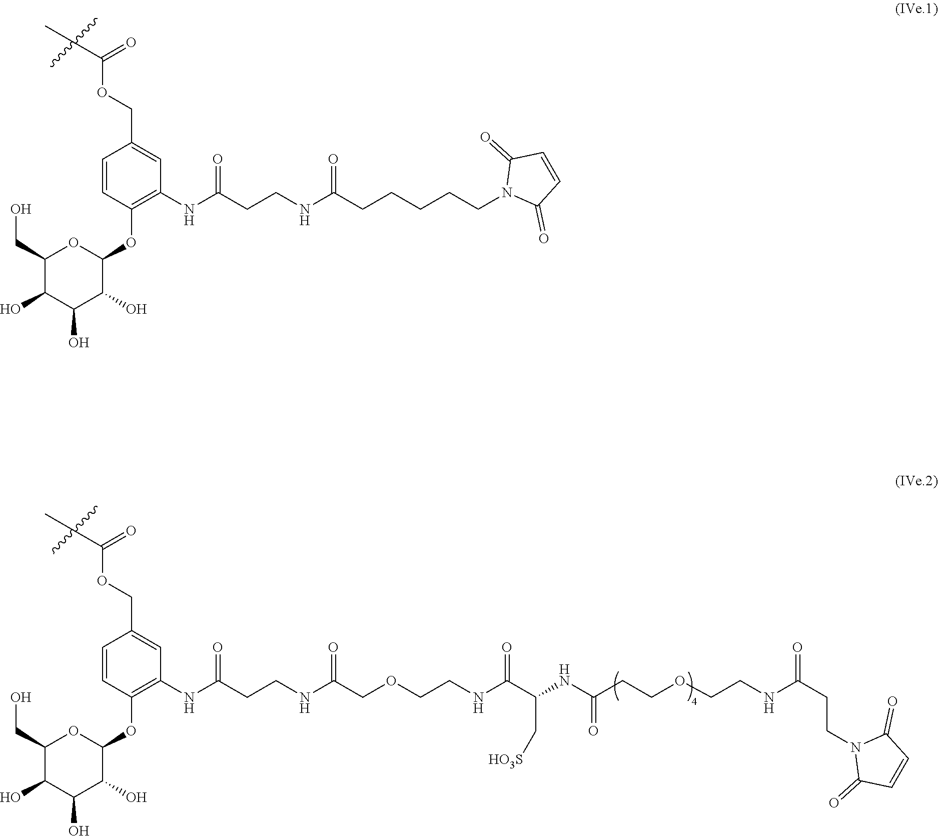

wherein [symbol] represents the point of attachment of the linker (L³) to a cyclic amino-pyrazinecarboxamide compound.

Although cleavable linkers can provide certain advantages, the linkers comprising the conjugate need not be cleavable. For non-cleavable linkers, the cyclic amino-pyrazinecarboxamide compound release may not depend on the differential properties between the plasma and some cytoplasmic compartments. The release of the cyclic amino-pyrazinecarboxamide compound can occur after internalization of the antibody conjugate via antigen-mediated endocytosis and delivery to lysosomal compartment, where the antibody construct can be degraded to the level of amino acids through intracellular proteolytic degradation. This process can release a cyclic amino-pyrazinecarboxamide compound derivative (a metabolite of the conjugate containing a non-cleavable linker-heterocyclic compound), which is formed by the cyclic amino-pyrazinecarboxamide compound, the linker, and the amino acid residue or residues to which the linker was covalently attached. The payload compound derivative from antibody construct cyclic amino-pyrazinecarboxamide compound conjugates with non-cleavable linkers can be more hydrophilic and less membrane permeable, which can lead to less bystander effects and less nonspecific toxicities compared to antibody conjugates with a cleavable linker. Antibody conjugates with non-cleavable linkers can have greater stability in circulation than antibody conjugates with cleavable linkers. Non-cleavable linkers can include alkylene chains, or can be polymeric, such as, for example, based upon polyalkylene glycol polymers, amide polymers, or can include segments of alkylene chains, polyalkylene glycols and/or amide polymers. The linker can contain a polyethylene glycol segment having from 1 to 6 ethylene glycol units.

The linker can be non-cleavable in vivo, for example, a linker according to the formulations below:

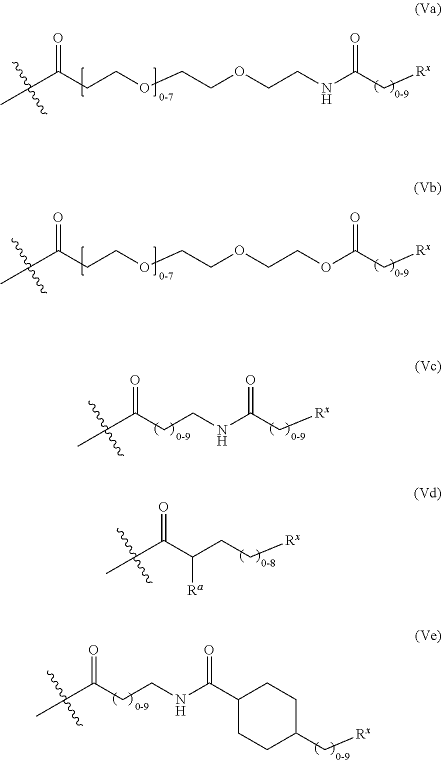

or salts thereof, wherein: R^{a} is selected from hydrogen, alkyl, sulfonate and methyl sulfonate; R^{x} is a reactive moiety including a functional group capable of covalently linking the linker to an antibody construct; and [symbol] represents the point of attachment of the linker (L³) to the compound or salt of any one of Formulas (I), (II), (II-a), (II-b), (II-c), (II-d), (III), (III-a), (III-b), (III-c), (III-d), (IV), (IV-a), (IV-b), (IV-c), and (IV-d).

Exemplary embodiments of linkers according to structural formula (Va)-(Ve) that may be included in the conjugates include the linkers illustrated below (as illustrated, the linkers include a group suitable for covalently linking the linker to an antibody construct, and [symbol] represents the point of attachment of the linker (L³) to the compound or salt of any one of Formulas (I), (II), (II-a), (II-b), (II-c), (II-d), (III), (III-a), (III-b), (III-c), (III-d), (IV), (IV-a), (IV-b), (IV-c), and (IV-d):

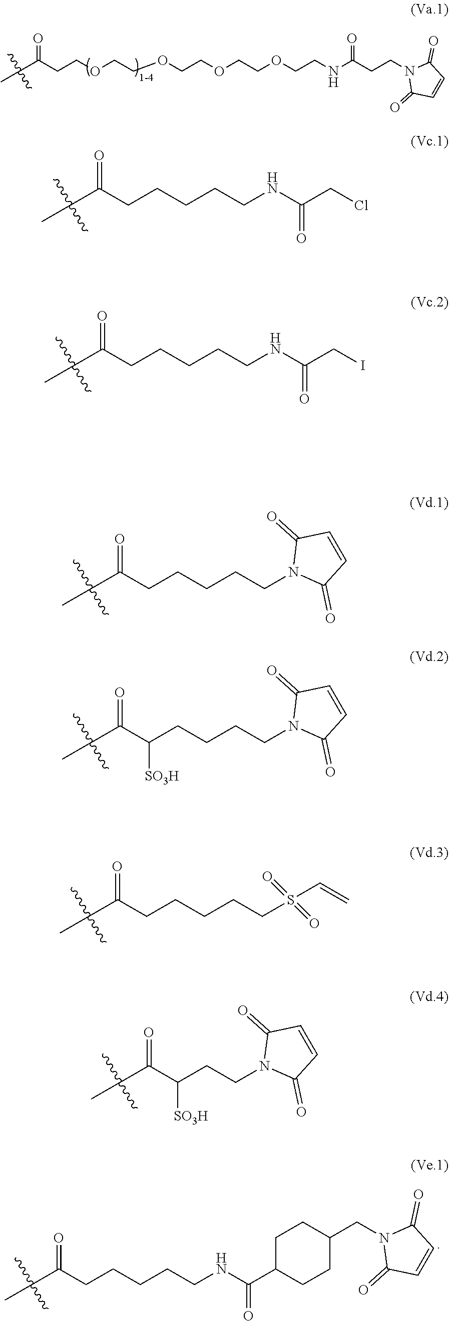

Attachment groups that are used to attach the linkers to an antibody construct can be electrophilic in nature and include, for example, maleimide groups, activated disulfides, active esters such as NHS esters and HOBt esters, haloformates, acid halides, alkyl, and benzyl halides such as haloacetamides. There are also emerging technologies related to “self-stabilizing” maleimides and “bridging disulfides” that can be used in accordance with the disclosure.

Maleimide groups are frequently used in the preparation of conjugates because of their specificity for reacting with thiol groups of, for example, cysteine groups of the antibody of a conjugate. The reaction between a thiol group of an antibody and a drug with a linker including a maleimide group proceeds according to the following scheme:

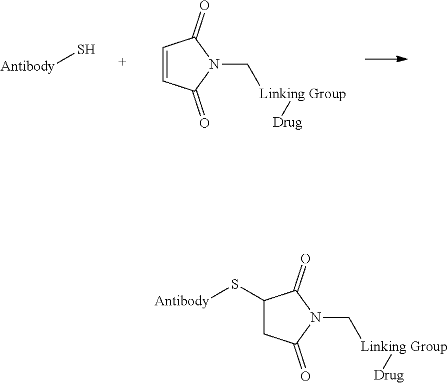

The reverse reaction leading to maleimide elimination from a thio-substituted succinimide may also take place. This reverse reaction is undesirable as the maleimide group may subsequently react with another available thiol group such as other proteins in the body having available cysteines. Accordingly, the reverse reaction can undermine the specificity of a conjugate. One method of preventing the reverse reaction is to incorporate a basic group into the linking group shown in the scheme above. Without wishing to be bound by theory, the presence of the basic group may increase the nucleophilicity of nearby water molecules to promote ring-opening hydrolysis of the succinimide group. The hydrolyzed form of the attachment group is resistant to deconjugation in the presence of plasma proteins. So-called “self-stabilizing” linkers provide conjugates with improved stability. A representative schematic is shown below:

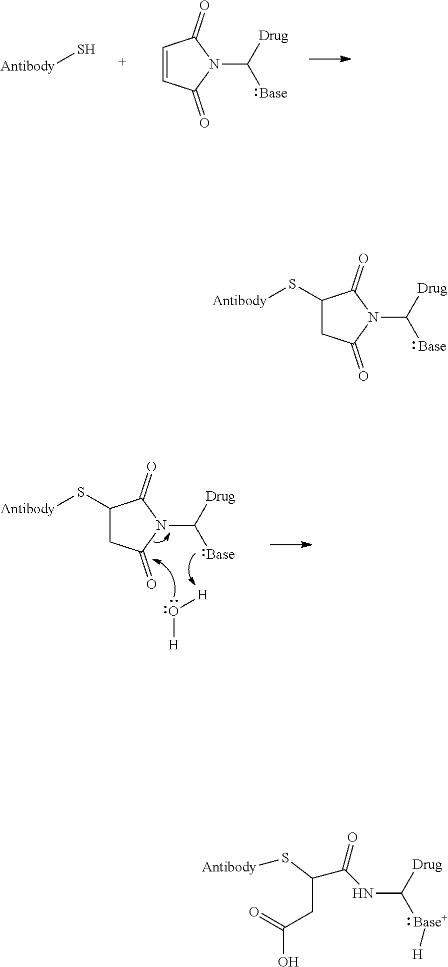

The hydrolysis reaction schematically represented above may occur at either carbonyl group of the succinimide group. Accordingly, two possible isomers may result, as shown below:

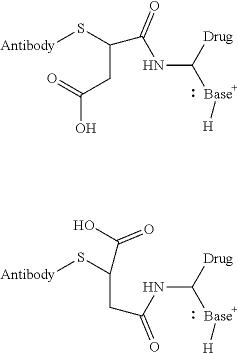

The identity of the base as well as the distance between the base and the maleimide group can be modified to tune the rate of hydrolysis of the thio-substituted succinimide group and optimize the delivery of a conjugate to a target by, for example, improving the specificity and stability of the conjugate.

Bases suitable for inclusion in a linker, e.g., any L³ with a maleimide group prior to conjugation to an antibody construct may facilitate hydrolysis of a nearby succinimide group formed after conjugation of the antibody construct to the linker. Bases may include, for example, amines (e.g., —N(R²⁶)(R²⁷), where R²⁶ and R²⁷ are independently selected from H and C₁₋₆ alkyl), nitrogen-containing heterocycles (e.g., a 3- to 12-membered heterocycle including one or more nitrogen atoms and optionally one or more double bonds), amidines, guanidines, and carbocycles or heterocycles substituted with one or more amine groups (e.g., a 3- to 12-membered aromatic or non-aromatic cycle optionally including a heteroatom such as a nitrogen atom and substituted with one or more amines of the type —N(R²⁶)(R²⁷), where R²⁶ and R²⁷ are independently selected from H or C₁₋₆ alkyl). A basic unit may be separated from a maleimide group by, for example, an alkylene chain of the form —(CH₂)ₘ—, where m is an integer from 0 to 10. An alkylene chain may be optionally substituted with other functional groups as described herein.

A linker (L³) with a maleimide group may include an electron withdrawing groups such as, but not limited to, —C(O)R, ═O, —CN, —NO₂, —CX₃, —X, —COOR, —CONR₂, —COR, —COX, —SO₂R, —SO₂OR, —SO₂NHR, —SO₂NR₂, —PO₃R², —P(O)(CH₃)NHR, —NO, —NR³⁺, —CR═CR₂, and —C≡CR, where each R is independently selected from H and C₁₋₆ alkyl and each X is independently selected from F, Br, Cl, and I. Self-stabilizing linkers may also include aryl, e.g., phenyl, or heteroaryl, e.g., pyridine, groups optionally substituted with electron withdrawing groups such as those described herein.

Examples of self-stabilizing linkers are provided in, e.g., U.S. Patent Publication Number 2013/0309256, the linkers of which are incorporated by reference herein. It will be understood that a self-stabilizing linker useful in conjunction with the compounds of the present invention may be equivalently described as unsubstituted maleimide-including linkers, thio-substituted succinimide-including linkers, or hydrolyzed, ring-opened thio-substituted succinimide-including linkers.

In certain embodiments, a linker of the disclosure (L³) comprises a stabilizing group selected from:

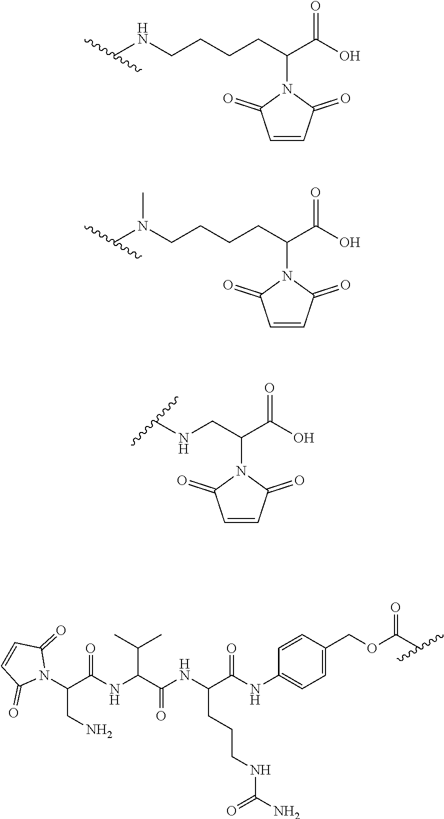 [SMILES: *NCCCCC(C(=O)O)N1C(=O)C=CC1=O ; *N(C)CCCCC(C(=O)O)N1C(=O)C=CC1=O ; *NCC(C(=O)O)N1C(=O)C=CC1=O ; *C(=O)OCc1ccc(NC(=O)C(CCCNC(N)=O)NC(=O)C(NC(=O)C(CN)N2C(=O)C=CC2=O)C(C)C)cc1]

In the scheme provided above, the bottom structure may be referred to as (maleimido)-DPR-Val-Cit-PAB, where DPR refers to diaminopropinoic acid, Val refers to valine, Cit refers to citrulline, and PAB refers to para-aminobenzylcarbonyl. [symbol] represent the point of attachment to compound or salt of any one of Formulas (I), (II), (II-a), (II-b), (II-c), (II-d), (III), (III-a), (III-b), (III-c), (III-d), (IV), (IV-a), (IV-b), (IV-c), and (IV-d).

A method for bridging a pair of sulfhydryl groups derived from reduction of a native hinge disulfide bond has been disclosed and is depicted in the schematic below. An advantage of this methodology is the ability to synthesize homogenous DAR4 conjugates by full reduction of IgGs (to give 4 pairs of sulfhydryls from interchain disulfides) followed by reaction with 4 equivalents of the alkylating agent. Conjugates containing “bridged disulfides” are also claimed to have increased stability.

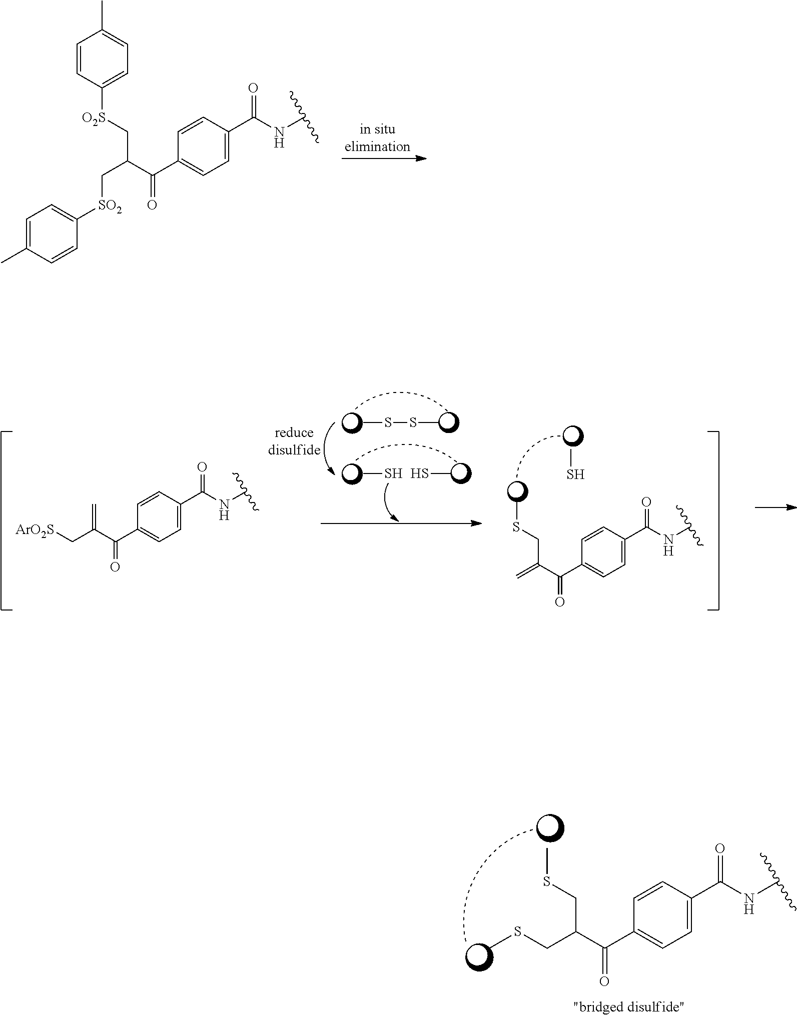

Similarly, as depicted below, a maleimide derivative that is capable of bridging a pair of sulfhydryl groups has been developed.

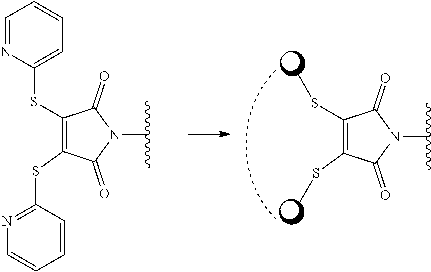 [SMILES: *N1C(=O)C(Sc2ccccn2)=C(Sc2ccccn2)C1=O ; *N1C(=O)C(SC)=C(SC)C1=O]

A linker of the disclosure, L³, can contain the following structural formulas (VIa), (VIb), or (VIc):

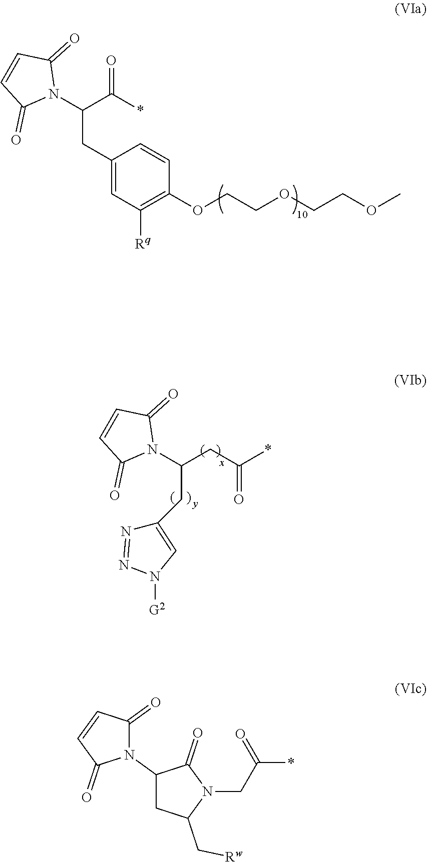

or salts thereof, wherein: R^{q} is H or —O—(CH₂CH₂O)₁₁—CH₃; x is 0 or 1; y is 0 or 1; G² is —CH₂CH₂CH₂SO₃H or —CH₂CH₂O—(CH₂CH₂O)₁₁—CH₃; R^{w} is —O—CH₂CH₂SO₃H or —NH(CO)—CH₂CH₂O—(CH₂CH₂O)₁₂—CH₃; and * represents the point of attachment to the remainder of the linker.

Exemplary embodiments of linkers according to structural formula (VIa) and (VIb) that can be included in the conjugates can include the linkers illustrated below (as illustrated, the linkers include a group suitable for covalently linking the linker to an antibody construct):

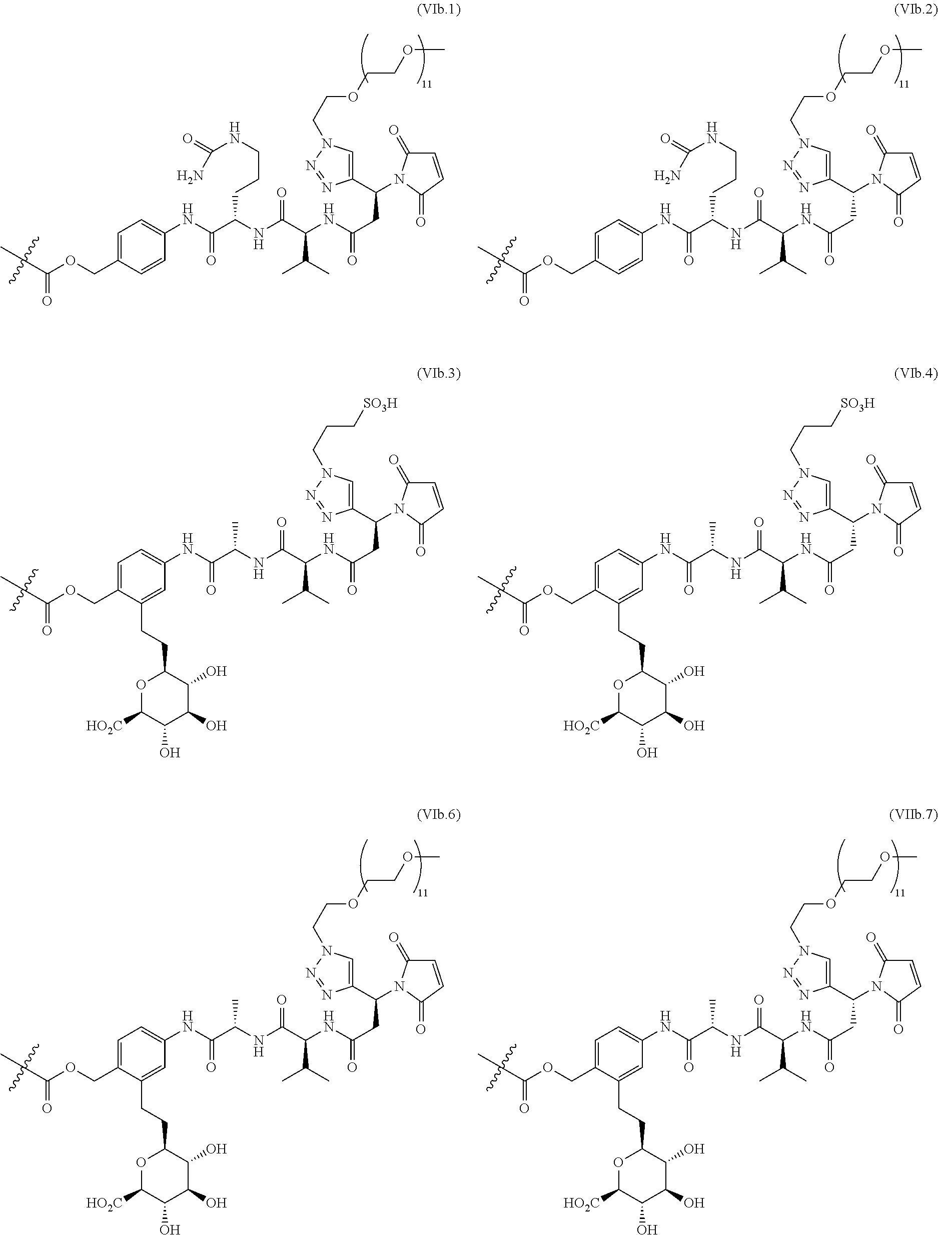

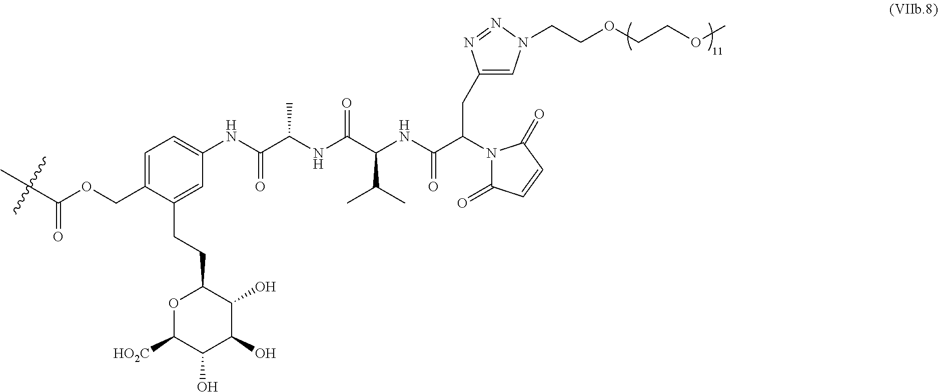

wherein [symbol] represents the point of attachment of the linker (L³) to the compound or salt of any one of Formulas (I), (II), (II-a), (II-b), (II-c), (II-d), (III), (III-a), (III-b), (III-c), (III-d), (IV), (IV-a), (IV-b), (IV-c), and (IV-d).

Exemplary embodiments of linkers according to structural formula (VIc) that can be included in the antibody construct conjugates can include the linkers illustrated below (as illustrated, the linkers include a group suitable for covalently linking the linker to an antibody construct):

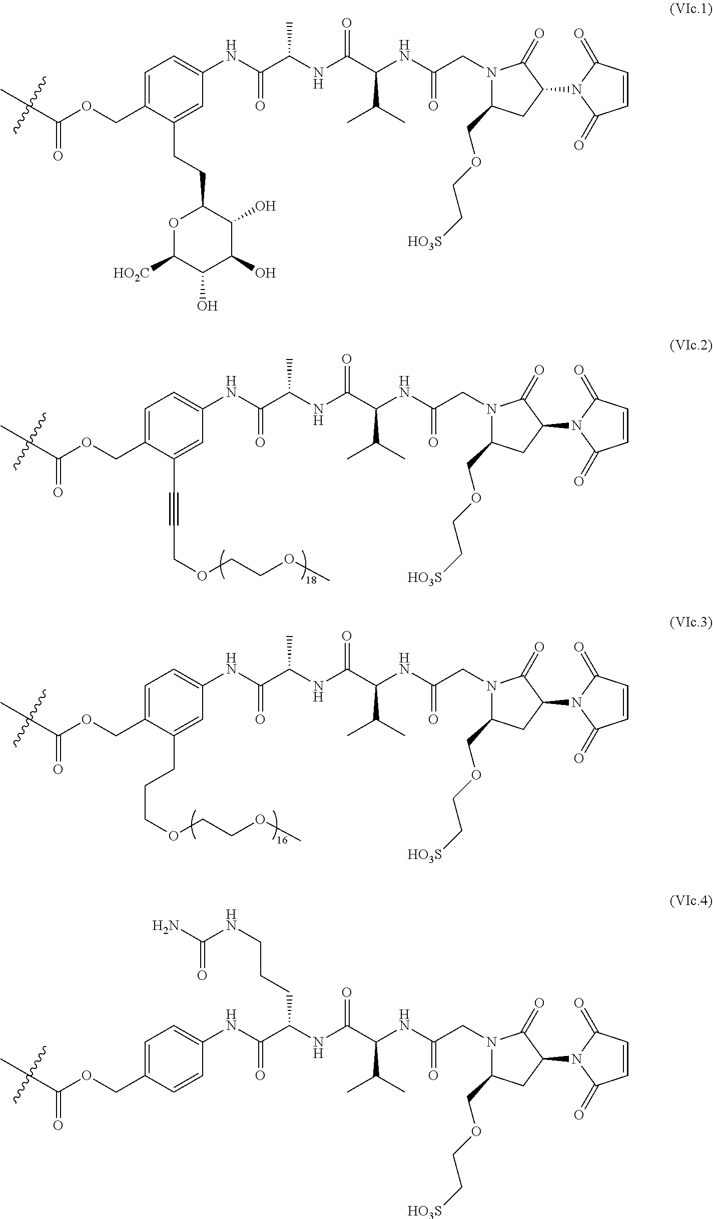

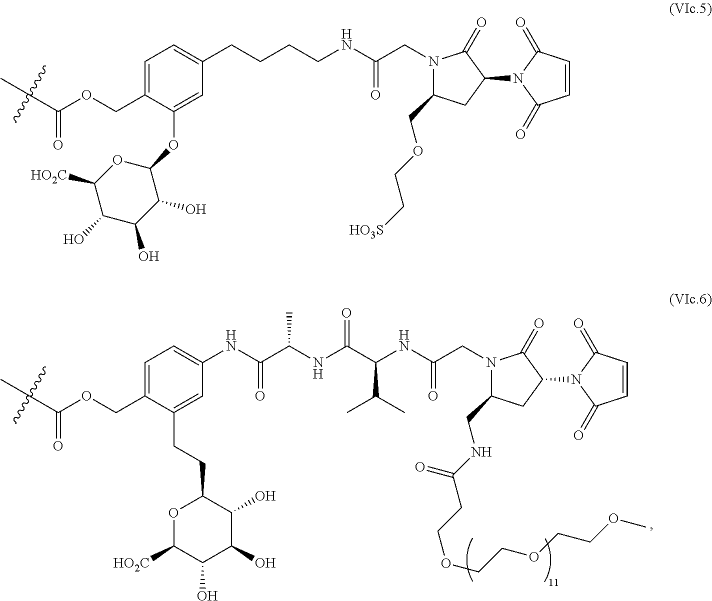

wherein [symbol] represents the point of attachment of the linker (L³) to the compound or salt of any one of Formulas (I), (II), (II-a), (II-b), (II-c), (II-d), (III), (III-a), (III-b), (III-c), (III-d), (IV), (IV-a), (IV-b), (IV-c), and (IV-d).

Some exemplary linkers (L³) are described in the following paragraphs. In some embodiments for a compound or salt of Formula (I), (II), (II-a), (II-b), (II-c), (II-d), (III), (III-a), (III-b), (III-c), (III-d), (IV), (IV-a), (IV-b), (IV-c), and (IV-d) and Table 1 wherein attachment of the linker is to a nitrogen of the compound and conjugation is to a cysteine residue of an antibody or targeting moiety, -L³ is represented by the formulas set forth in Table C below:

TABLE C

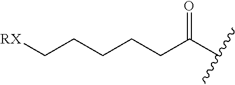

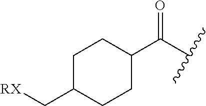

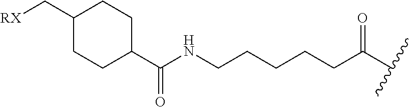

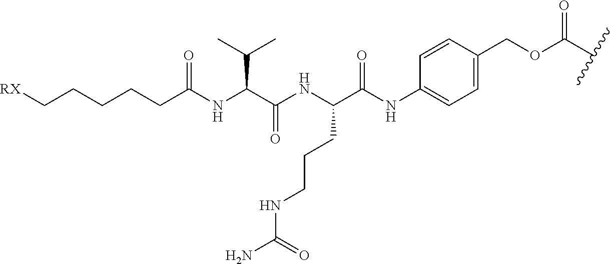

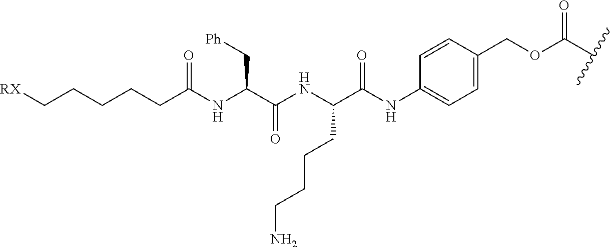

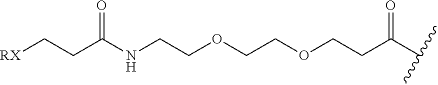

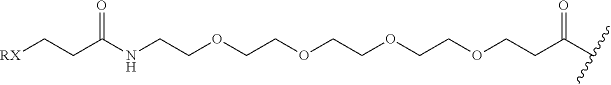

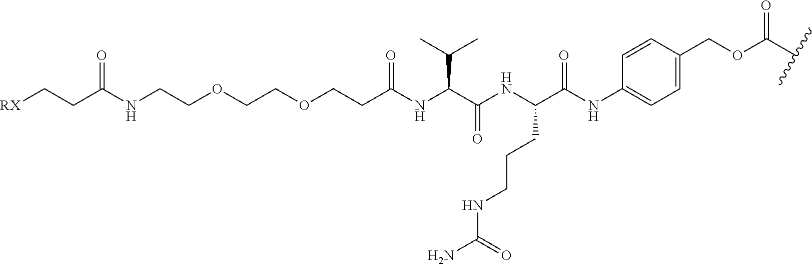

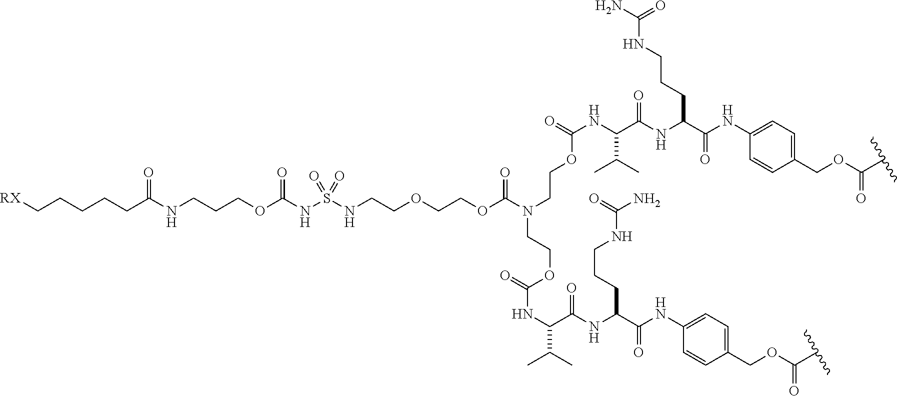

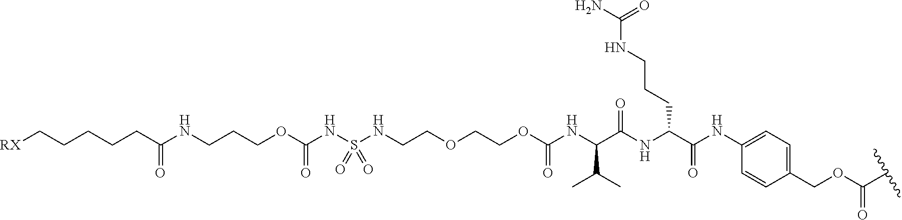

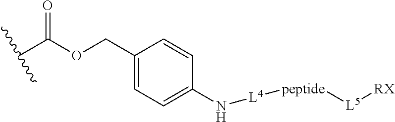

L⁴ represents the C-terminus of the peptide and L⁵ is selected from a bond, alkylene and
heteroalkylene, wherein L⁵ is optionally substituted with one or more groups independently
selected from R³, and R³⁰ is independently selected at each occurrence from halogen, —OH, —CN,
—O-alkyl, —SH, ═O, ═S, —NH₂, —NO₂; and C₁-C₁₀alkyl, C₂-C₁₀alkenyl, and C₂-C₁₀alkynyl,
each of which is independently optionally substituted at each occurrence with one or more
substituents selected from halogen, —OH, —CN, —O-alkyl, —SH, ═O, ═S, —NH₂, and —NO₂.

wherein

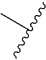

represents attachment to a nitrogen of a compound or salt of any one of Formulas (I), (II), (II-a), (II-b), (II-c), (II-d), (III), (III-a), (III-b), (III-c), (III-d), (IV), (IV-a), (IV-a), (IV-b), (IV-c), and (IV-d) and Table 1 and RX represents a reactive moiety. The reactive moiety may be selected, for example, from an electrophile, e.g., an α,β-unsaturated carbonyl, such as a maleimide, and a leaving group. For example, -L³ can be represented by the formulas set forth in Table D below:

TABLE D

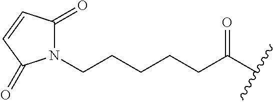 [SMILES: *C(=O)CCCCCN1C(=O)C=CC1=O]

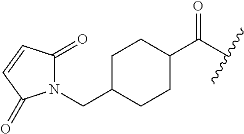 [SMILES: *C(=O)C1CCC(CN2C(=O)C=CC2=O)CC1]

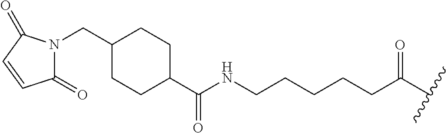 [SMILES: *C(=O)CCCCCNC(=O)C1CCC(CN2C(=O)C=CC2=O)CC1]

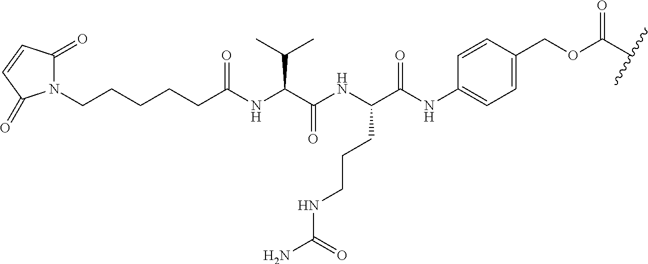 [SMILES: *C(=O)OCc1ccc(NC(=O)[C@H](CCCNC(N)=O)NC(=O)[C@@H](NC(=O)CCCCCN2C(=O)C=CC2=O)C(C)C)cc1]

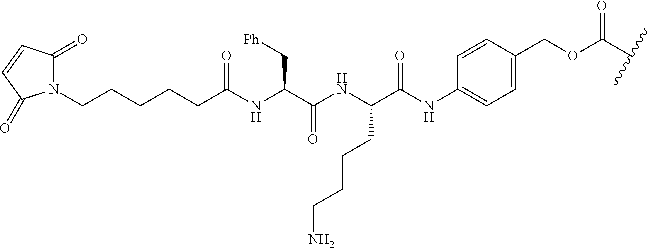 [SMILES: *C(=O)OCc1ccc(NC(=O)[C@H](CCCCN)NC(=O)[C@H](Cc2ccccc2)NC(=O)CCCCCN2C(=O)C=CC2=O)cc1]

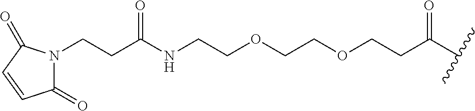 [SMILES: *C(=O)CCOCCOCCNC(=O)CCN1C(=O)C=CC1=O]

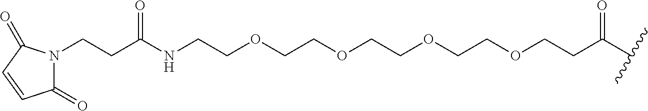 [SMILES: *C(=O)CCOCCOCCOCCOCCNC(=O)CCN1C(=O)C=CC1=O]

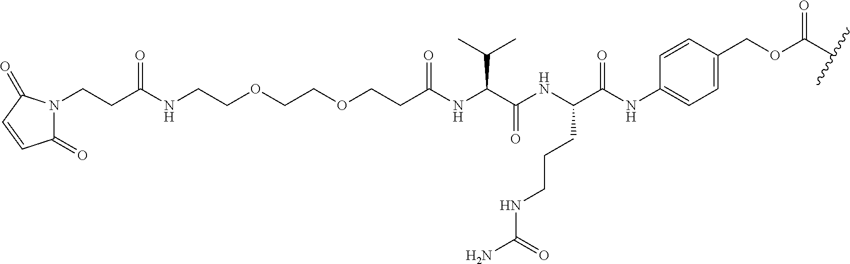 [SMILES: *C(=O)OCc1ccc(NC(=O)[C@H](CCCNC(N)=O)NC(=O)[C@@H](NC(=O)CCOCCOCCNC(=O)CCN2C(=O)C=CC2=O)C(C)C)cc1]

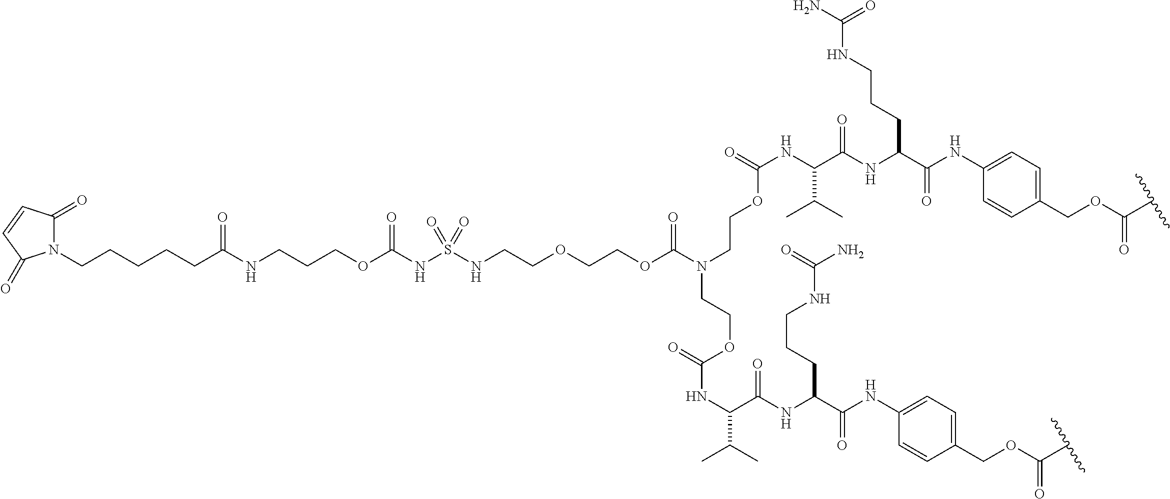 [SMILES: *C(=O)OCc1ccc(NC(=O)[C@H](CCCNC(N)=O)NC(=O)[C@@H](NC(=O)OCCN(CCOC(=O)N[C@H](C(=O)N[C@@H](CCCNC(N)=O)C(=O)Nc2ccc(COC(*)=O)cc2)C(C)C)C(=O)OCCOCCNS(=O)(=O)NC(=O)OCCCNC(=O)CCCCCN2C(=O)C=CC2=O)C(C)C)cc1]

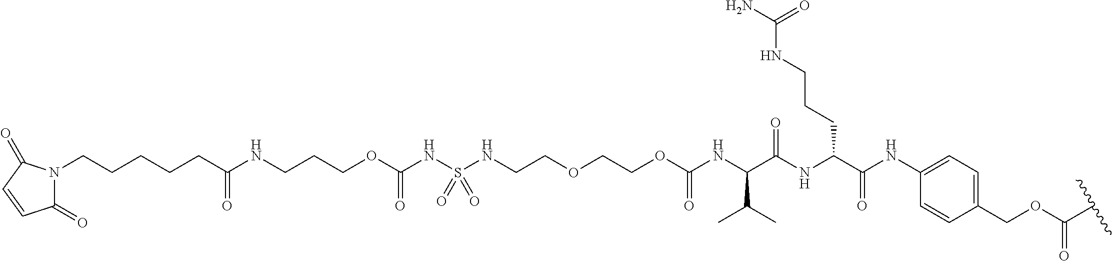 [SMILES: *C(=O)OCc1ccc(NC(=O)[C@@H](CCCNC(N)=O)NC(=O)[C@H](NC(=O)OCCOCCNS(=O)(=O)NC(=O)OCCCNC(=O)CCCCCN2C(=O)C=CC2=O)C(C)C)cc1]

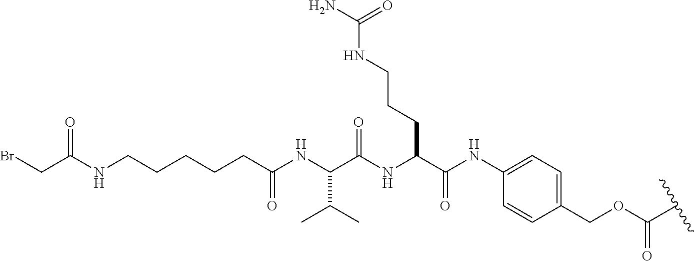 [SMILES: *C(=O)OCc1ccc(NC(=O)[C@H](CCCNC(N)=O)NC(=O)[C@@H](NC(=O)CCCCCNC(=O)CBr)C(C)C)cc1]

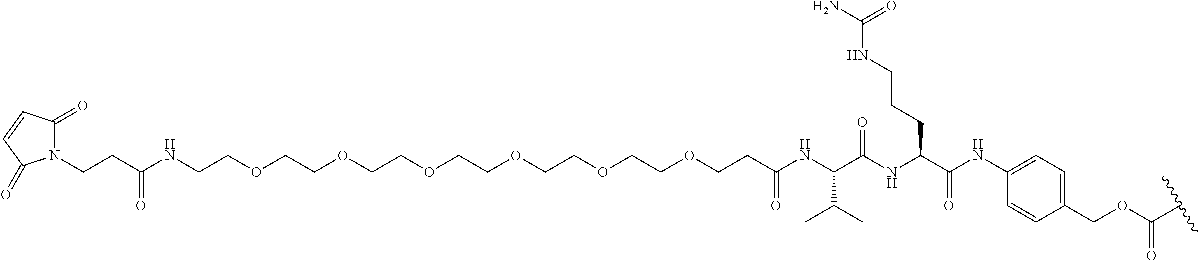 [SMILES: *C(=O)OCc1ccc(NC(=O)[C@H](CCCNC(N)=O)NC(=O)[C@@H](NC(=O)CCOCCOCCOCCOCCOCCOCCNC(=O)CCN2C(=O)C=CC2=O)C(C)C)cc1]

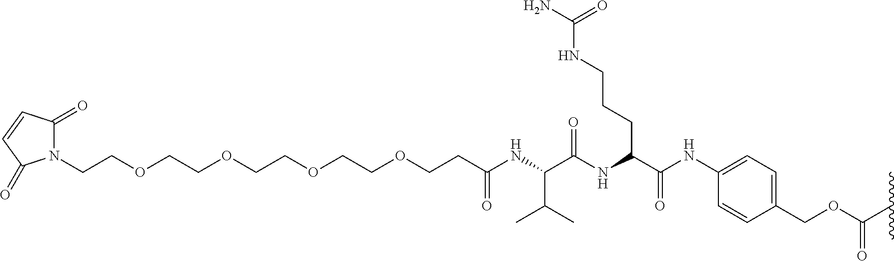

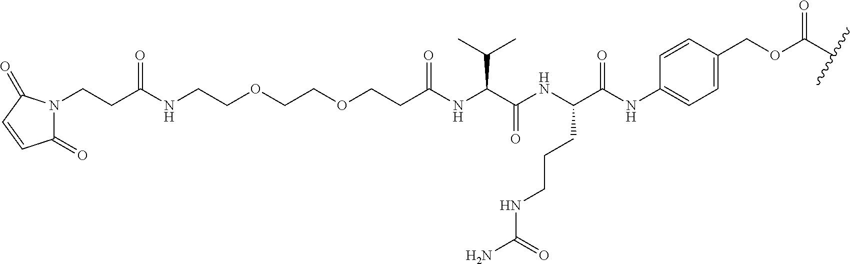 [SMILES: *C(=O)OCc1ccc(NC(=O)[C@H](CCCNC(N)=O)NC(=O)[C@@H](NC(=O)CCOCCOCCNC(=O)CCN2C(=O)C=CC2=O)C(C)C)cc1]

wherein

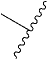

represents attachment to a nitrogen of a compound or salt of any one of Formulas (I), (II), (II-a), (II-b), (II-c), (II-d), (III), (III-a), (III-b), (III-c), (III-d), (IV), (IV-a), (IV-b), (IV-c), and (IV-d) and Table 1.

When conjugated to the cysteine residue of the antibody or targeting moiety, such linkers can be, for example, represented by the Formulas set forth in Table E below:

TABLE E

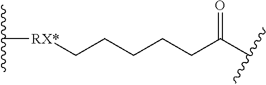

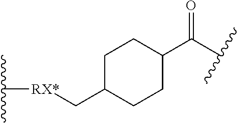

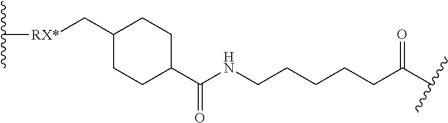

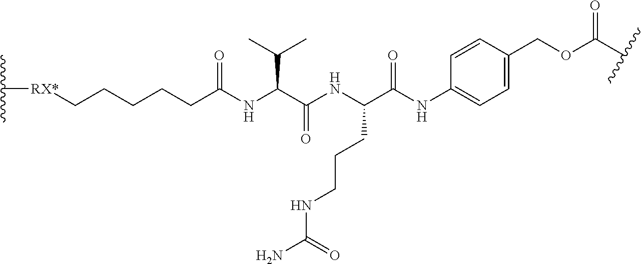

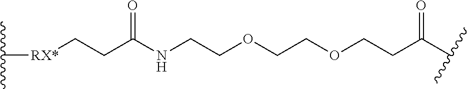

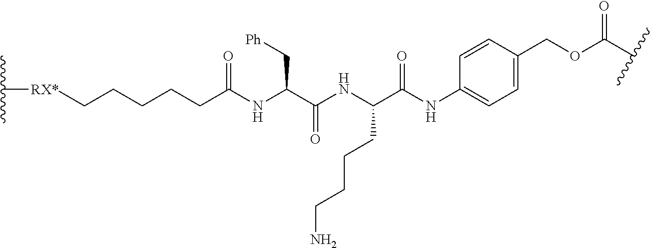

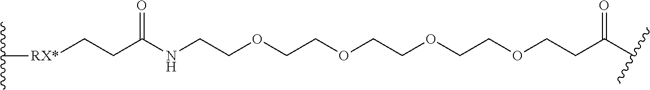

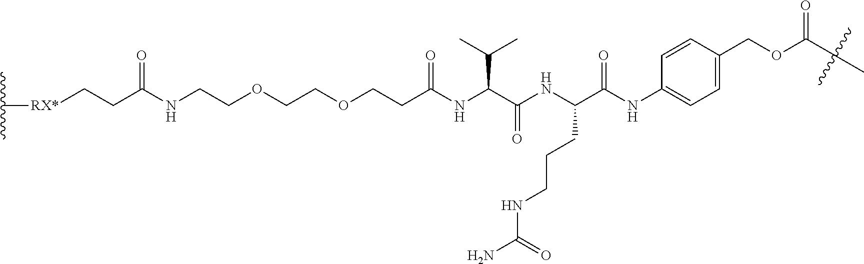

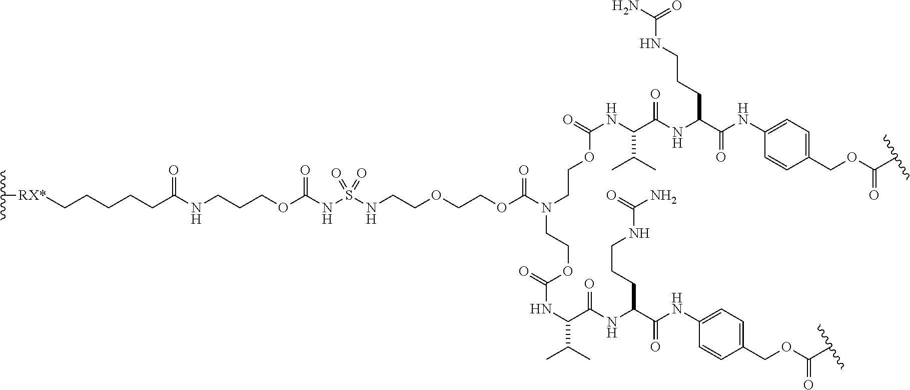

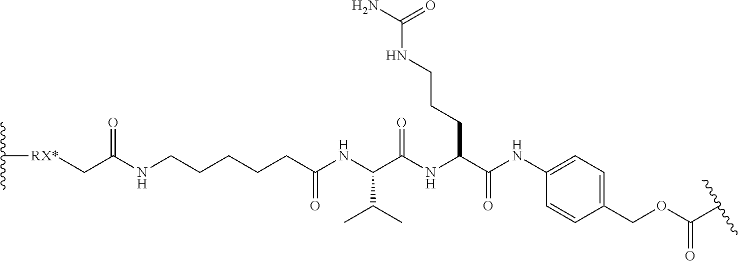

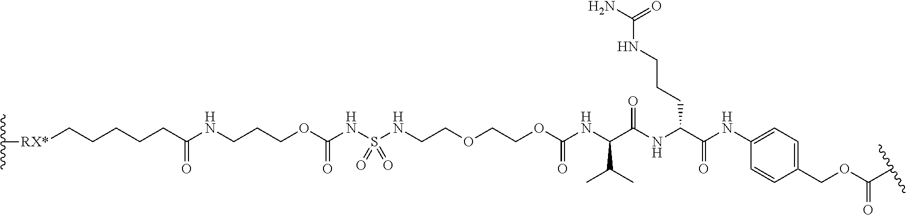

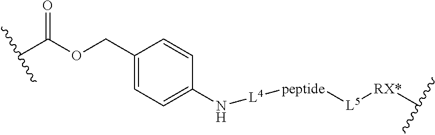

wherein RX* is a bond, a succinimide moiety, or a hydrolyzed succinimide moiety bound to a cysteine residue of the antibody construct, wherein

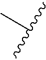

on RX* represents the point of attachment to such residue; L⁴ when present represents the C-terminus of the peptide and L⁵ is selected from a bond, alkylene and heteroalkylene, wherein L⁵ is optionally substituted with one or more groups independently selected from R³⁰; and R³⁰ when present is independently selected at each occurrence from halogen, —OH, —CN, —O-alkyl, —SH, ═O, ═S, —NH₂, —NO₂; and C₁-C₁₀alkyl, C₂-C₁₀alkenyl, and C₂-C₁₀alkynyl, each of which is independently optionally substituted at each occurrence with one or more substituents selected from halogen, —OH, —CN, —O-alkyl, —SH, ═O, ═S, —NH₂, and —NO₂. A particularly preferred pepide is val-ala or val-cit.

In some embodiments for a compound or salt of Formula (I), (II), (II-a), (II-b), (II-c), (II-d), (III), (III-a), (III-b), (III-c), (III-d), (IV), (IV-a), (IV-b), (IV-c), and (IV-d) and Table 1 wherein attachment of the linker is to a nitrogen of a compound or salt of any one of Formulas (I), (II), (II-a), (II-b), (II-c), (II-d), (III), (III-a), (III-b), (III-c), (III-d), (IV), (IV-a), (IV-b), (IV-c), and (IV-d) and Table 1 and conjugation is to a lysine residue of an antibody or other targeting moiety, -L³ is represented by the formulas set forth in Table F below:

TABLE F

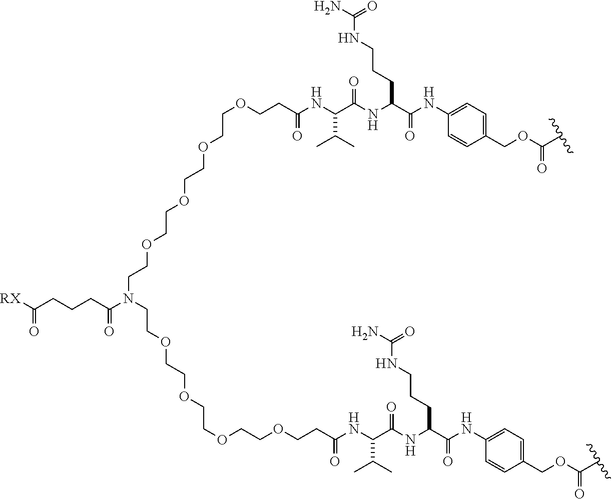

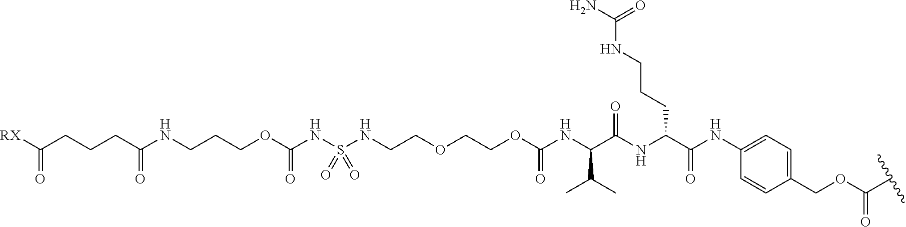

wherein

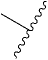

represents attachment to a nitrogen of a compound or salt of any one of Formulas (I), (II), (II-a), (II-b), (II-c), (II-d), (III), (III-a), (III-b), (III-c), (III-d), (IV), (IV-a), (IV-b), (IV-c), and (IV-d) and Table 1 and RX represents a reactive moiety. The reactive moiety may be selected from activated esters. For example, -L³ can be represented by the formulas set forth in Table G below:

TABLE G

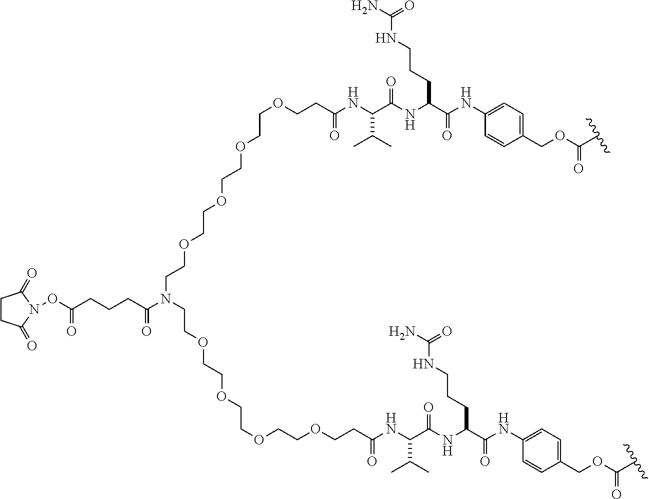 [SMILES: *C(=O)OCc1ccc(NC(=O)[C@H](CCCNC(N)=O)NC(=O)[C@@H](NC(=O)CCOCCOCCOCCOCCN(CCOCCOCCOCCOCCC(=O)N[C@H](C(=O)N[C@@H](CCCNC(N)=O)C(=O)Nc2ccc(COC(*)=O)cc2)C(C)C)C(=O)CCCC(=O)ON2C(=O)CCC2=O)C(C)C)cc1]

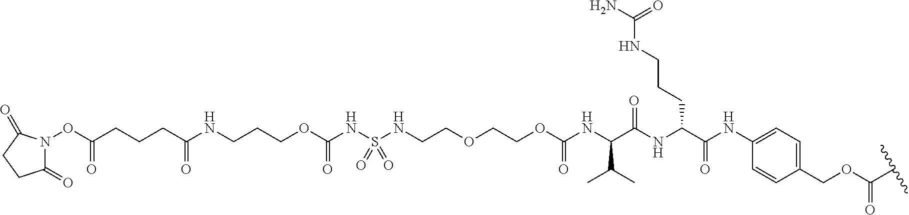 [SMILES: *C(=O)OCc1ccc(NC(=O)[C@@H](CCCNC(N)=O)NC(=O)[C@H](NC(=O)OCCOCCNS(=O)(=O)NC(=O)OCCCNC(=O)CCCC(=O)ON2C(=O)CCC2=O)C(C)C)cc1]

When conjugated to the lysine residue of an antibody or other targeting moiety, such linkers, can, for example, be represented by the Formulas set forth in Table H below wherein RX* is a bond to a nitrogen of the lysine residue of the antibody construct or targeting moiety, wherein

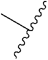

on RX* represents the point of attachment to such residue:

TABLE H

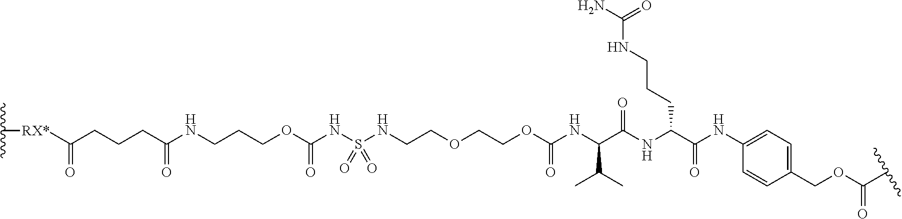

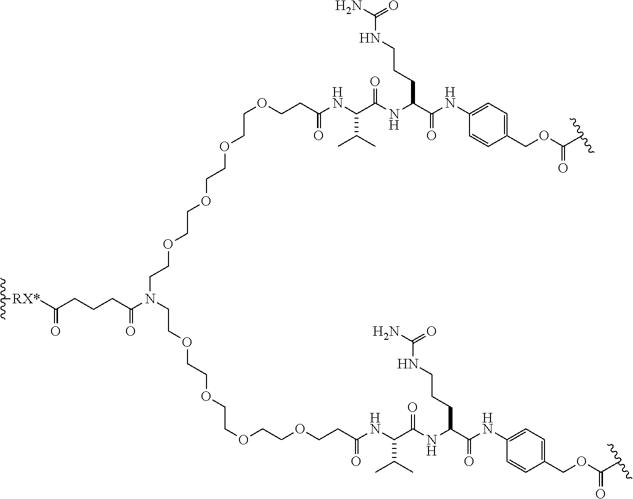

As noted,

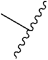

represents attachment to a nitrogen of a compound or salt of any one of Formulas (I), (II), (II-a), (II-b), (II-c), (II-d), (III), (III-a), (III-b), (III-c), (III-d), (IV), (IV-a), (IV-b), (IV-c), and (IV-d) and Table 1. In exemplary embodiments, the linkers described herein, including those in the preceding paragraphs, are attached to a compound of the present invention through R⁴ or R⁵. R⁴ or R⁵can be, for example, selected from any of the groups set forth in Table I and

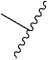

indicates attachment of R⁴ or R⁵ to the phenyl ring of the rest of the molecule.

TABLE I

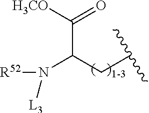

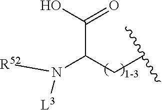

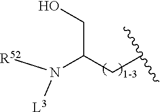

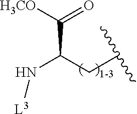

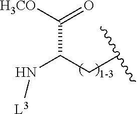

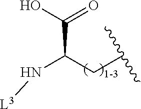

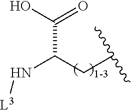

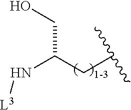

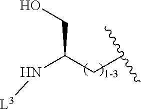

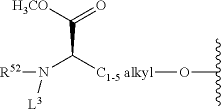

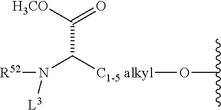

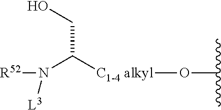

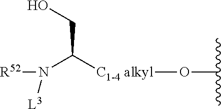

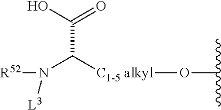

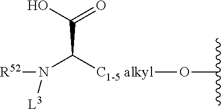

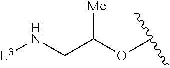

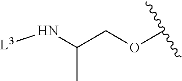

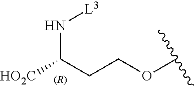

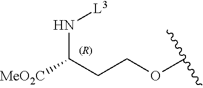

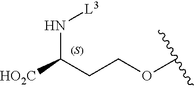

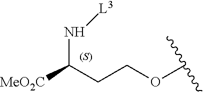

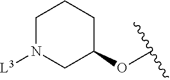

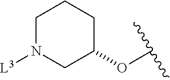

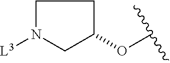

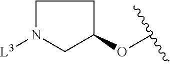

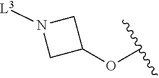

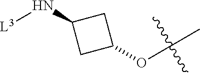

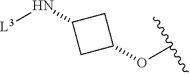

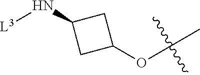

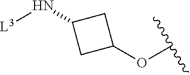

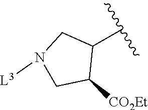

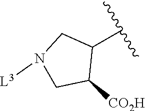

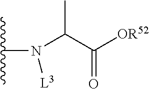

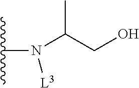

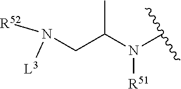

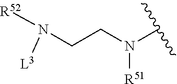

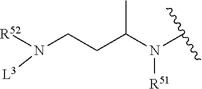

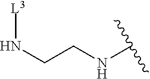

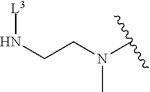

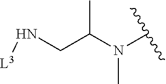

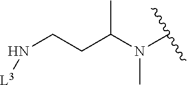

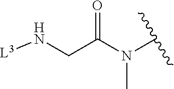

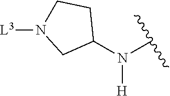

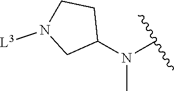

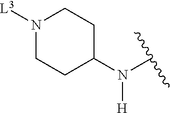

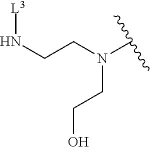

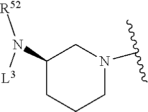

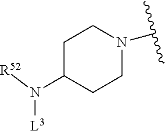

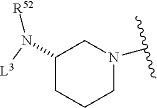

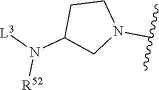

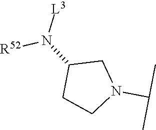

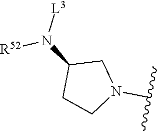

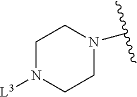

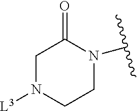

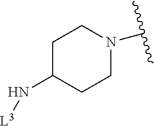

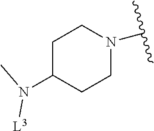

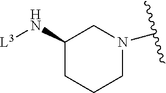

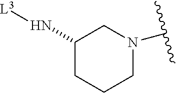

In some embodiments for a compound or salt of Formula (I), (II), (II-a), (II-b), (II-c), (II-d), (III), (III-a), (III-b), (III-c), (III-d), (IV), (IV-a), (IV-b), (IV-c), and (IV-d) wherein attachment of the linker is to a sulfur of a compound or salt of any one of Formulas (I), (II), (II-a), (II-b), (II-c), (II-d), (III), (III-a), (III-b), (III-c), (III-d), (IV), (IV-a), (IV-b), (IV-c), and (IV-d) and Table 1 and conjugation is to a lysine residue of an antibody or other targeting moiety, -L³ is represented by the formula set forth below in Table J:

TABLE J

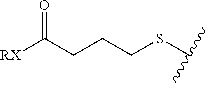

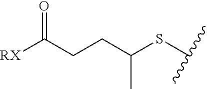

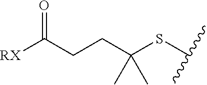

wherein

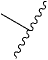

represents attachment to a sulfur of a compound or salt of any one of Formulas (I), (II), (II-a), (II-b), (II-c), (II-d), (III), (III-a), (III-b), (III-c), (III-d), (IV), (IV-a), (IV-b), (IV-c), and (IV-d) and Table 1 and RX represents a reactive moiety. The reactive moiety may be selected from an activated ester. For example, -L³ can be represented by the formulas:

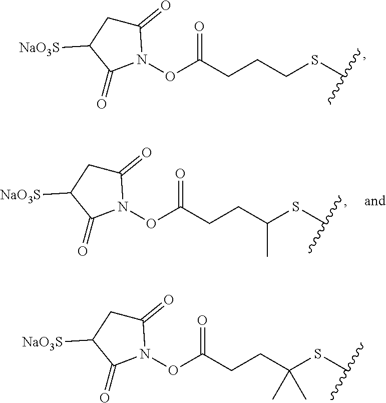

When conjugated to the lysine residue of an antibody or other targeting moiety, such linkers, can be represented by the following Formulas in Table K:

TABLE K

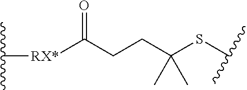

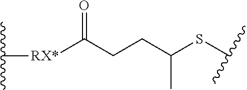

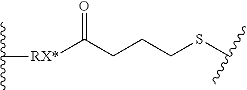

wherein RX* is a bound to a nitrogen of the lysine residue of the antibody construct or targeting moiety, wherein

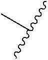

on RX* represents the point of attachment to such residue.

As noted,

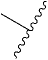

represents attachment to a sulfur atom of a compound or salt of any one of Formulas (I), (II), (II-a), (II-b), (II-c), (II-d), (III), (III-a), (III-b), (III-c), (III-d), (IV), (IV-a), (IV-b), (IV-c), and (IV-d) and Table 1. In exemplary embodiments, the linkers described herein, including those in the preceding paragraphs, are attached at a sulfur atom to a compound or salt. In other exemplary embodiments, exemplary linkers are attached at an oxygen atom of a compound or salt. In other exemplary embodiments, exemplary linkers are attached at a nitrogen atom of a compound or salt.

As is known by skilled artisans, the linker selected for a particular conjugate may be influenced by a variety of factors, including but not limited to, the site of attachment to the antibody construct (e.g., lys, cys or other amino acid residues), structural constraints of the drug pharmacophore and the lipophilicity of the drug. The specific linker selected for a conjugate should seek to balance these different factors for the specific antibody construct/drug combination.

The properties of the linker, or linker-compound, may also impact aggregation of the conjugate under conditions of use and/or storage. Typically, conjugates reported in the literature contain no more than 3-4 drug molecules per antibody molecule. Attempts to obtain higher drug-to-antibody ratios (“DAR”) often failed, particularly if both the drug and the linker were hydrophobic, due to aggregation of the conjugate. In many instances, DARs higher than about 3-4 could be beneficial as a means of increasing potency. In instances where the payload compound is more hydrophobic in nature, it may be desirable to select linkers that are relatively hydrophilic as a means of reducing conjugate aggregation, especially in instances where DARs greater than about 3-4 are desired. Thus, in certain embodiments, the linker incorporates chemical moieties that reduce aggregation of the conjugates during storage and/or use. A linker may incorporate polar or hydrophilic groups such as charged groups or groups that become charged under physiological pH to reduce the aggregation of the conjugates. For example, a linker may incorporate charged groups such as salts or groups that deprotonate, e.g., carboxylates, or protonate, e.g., amines, at physiological pH.

In particular embodiments, the aggregation of the conjugates during storage or use is less than about 40% as determined by size-exclusion chromatography (SEC). In particular embodiments, the aggregation of the conjugates during storage or use is less than 35%, such as less than about 30%, such as less than about 25%, such as less than about 20%, such as less than about 15%, such as less than about 10%, such as less than about 5%, such as less than about 4%, or even less, as determined by size-exclusion chromatography (SEC).

Exemplary Linker-Compounds of the present invention include those set forth in Tables 15, 16, and 17, and salts thereof (including pharmaceutically acceptable salts thereof.

### Pharmaceutical Formulations

The compositions and methods described herein may be considered useful as pharmaceutical compositions for administration to a subject in need thereof. Pharmaceutical compositions may comprise at least the compositions described herein and one or more pharmaceutically acceptable carriers, diluents, excipients, stabilizers, dispersing agents, suspending agents, and/or thickening agents. In certain embodiments, a composition comprises a conjugate having an antibody construct or a targeting moiety and a cyclic amino-pyrazinecarboxamide compound of this disclosure. In further embodiments, a composition comprises a conjugate having an antibody construct or a targeting moiety and a cyclic amino-pyrazinecarboxamide compound of this disclosure. In still further embodiments, a composition comprises a conjugate having an antibody construct, a target binding domain, and a cyclic amino-pyrazinecarboxamide compound of this disclosure. The composition may comprise any conjugate described herein. In some embodiments, the antibody construct is an anti-LRRC15 antibody. Exemplary conjugates of this disclosure may comprise an anti-LRRC15 antibody and a cyclic amino-pyrazinecarboxamide compound of this disclosure. In some embodiments, the antibody construct is an anti-ASGR1 antibody. Exemplary conjugates of this disclosure comprise an anti-ASGR1 antibody and a cyclic amino-pyrazinecarboxamide compound of this disclosure. In some embodiments, a targeting moiety is a GalNAc moiety or a structure of Formula (V) comprising two or three GalNAc moieties. In further embodiments, a conjugate comprises a targeting moiety and and a cyclic amino-pyrazinecarboxamide compound of this disclosure, wherein the targeting moiety is a GalNAc moiety or has a structure of Formula (V) comprising two or three GalNAc moieties. A pharmaceutical composition can comprise at least the compounds, conjugates, or salts described herein and one or more of buffers, antibiotics, steroids, carbohydrates, drugs (e.g., chemotherapy drugs), radiation, polypeptides, chelators, adjuvants and/or preservatives.

Pharmaceutical compositions may be formulated using one or more physiologically-acceptable carriers comprising excipients and auxiliaries. Formulation may be modified depending upon the route of administration chosen. Pharmaceutical compositions comprising a compound, conjugate, or salt may be manufactured, for example, by lyophilizing the compound, conjugate, or salt, or mixing, dissolving, emulsifying, encapsulating or entrapping the conjugate. The pharmaceutical compositions may also include the compounds, conjugates, or salts in a free-base form or pharmaceutically-acceptable salt form.

Methods for formulation of the conjugates may include formulating any of the compounds, salts or conjugates with one or more inert, pharmaceutically-acceptable excipients or carriers to form a solid, semi-solid, or liquid composition. Solid compositions may include, for example, powders, tablets, dispersible granules and capsules, and in some aspects, the solid compositions further contain nontoxic, auxiliary substances, for example wetting or emulsifying agents, pH buffering agents, and other pharmaceutically-acceptable additives. Alternatively, the compounds, salts or conjugates may be lyophilized or in powder form for re-constitution with a suitable vehicle, e.g., sterile pyrogen-free water, before use.

Pharmaceutical compositions of the conjugates may comprise at least one active ingredient (e.g., a compound, salt or conjugate and other agents). The active ingredients may be entrapped in microcapsules prepared, for example, by coacervation techniques or by interfacial polymerization (e.g., hydroxymethylcellulose or gelatin microcapsules and poly-(methylmethacylate) microcapsules, respectively), in colloidal drug-delivery systems (e.g., liposomes, albumin microspheres, microemulsions, nano-particles and nanocapsules) or in macroemulsions. Pharmaceutical compositions as often further may comprise more than one active compound (e.g., a compound, conjugate, or salt and other agents) as necessary for the particular indication being treated. The active compounds may have complementary activities that do not adversely affect each other. For example, the composition may comprise a chemotherapeutic agent, cytotoxic agent, cytokine, growth-inhibitory agent, anti-hormonal agent, anti-angiogenic agent, and/or cardioprotectant. Such molecules may be present in combination in amounts that are effective for the purpose intended.

The compositions and formulations may be sterilized. Sterilization may be accomplished by filtration through sterile filtration.

The compositions may be formulated for administration as an injection. Non-limiting examples of formulations for injection may include a sterile suspension, solution or emulsion in oily or aqueous vehicles. Suitable oily vehicles may include, but are not limited to, lipophilic solvents or vehicles such as fatty oils or synthetic fatty acid esters, or liposomes. Aqueous injection suspensions may contain substances which increase the viscosity of the suspension. The suspension may also contain suitable stabilizers. Injections may be formulated for bolus injection or continuous infusion. Alternatively, the compositions may be lyophilized or in powder form for reconstitution with a suitable vehicle, e.g., sterile pyrogen-free water, before use.

For parenteral administration, the compounds, conjugates, or salts may be formulated in a unit dosage injectable form (e.g., solution, suspension, emulsion) in association with a pharmaceutically acceptable parenteral vehicle. Such vehicles may be inherently non-toxic, and non-therapeutic. Vehicles may be water, saline, Ringer's solution, dextrose solution, and 5% human serum albumin. Non-aqueous vehicles such as fixed oils and ethyl oleate may also be used. Liposomes may be used as carriers. The vehicle may contain minor amounts of additives such as substances that enhance isotonicity and chemical stability (e.g., buffers and preservatives).

Sustained-release preparations may be also be prepared. Examples of sustained-release preparations may include semipermeable matrices of solid hydrophobic polymers that may contain the compound, conjugate, or salt, and these matrices may be in the form of shaped articles (e.g., films or microcapsules). Examples of sustained-release matrices may include polyesters, hydrogels (e.g., poly(2-hydroxyethyl-methacrylate), or poly(vinyl alcohol)), polylactides, copolymers of L-glutamic acid and γ ethyl-L-glutamate, non-degradable ethylene-vinyl acetate, degradable lactic acid-glycolic acid copolymers such as the LUPRON DEPOT™ (i.e., injectable microspheres composed of lactic acid-glycolic acid copolymer and leuprolide acetate), and poly-D-(−)-3-hydroxybutyric acid.

Pharmaceutical formulations may be prepared for storage by mixing a compound, conjugate, or salt with a pharmaceutically acceptable carrier, excipient, and/or a stabilizer. This formulation may be a lyophilized formulation or an aqueous solution. Acceptable carriers, excipients, and/or stabilizers may be nontoxic to recipients at the dosages and concentrations used. Acceptable carriers, excipients, and/or stabilizers may include buffers such as phosphate, citrate, and other organic acids; antioxidants including ascorbic acid and methionine; preservatives, polypeptides; proteins, such as serum albumin or gelatin; hydrophilic polymers; amino acids; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes; and/or non-ionic surfactants or polyethylene glycol.

Pharmaceutical formulations of the conjugates may have an average drug-antibody construct ratio (“DAR”) selected from about 1 to about 20 or from about 1 to about 10, wherein the drug is a compound or salt of any one of Formulas (I), (II), (II-a), (II-b), (II-c), (II-d), (III), (III-a), (III-b), (III-c), (III-d), (IV), (IV-a), (IV-b), (IV-c), and (IV-d). In certain embodiments, the average DAR of the formulation is from about 2 to about 8, such as from about 3 to about 8, such as from about 3 to about 7, such as about 3 to about 5 or such as about 2. In certain embodiments, a pharmaceutical formulation has an average DAR of about 3, about 3.5, about 4, about 4.5 or about 5.

### Therapeutic Applications

The compounds, conjugates, salts, compositions and methods of the present disclosure can be useful for a plurality of different subjects including, but are not limited to, a mammal, human, non-human mammal, a domesticated animal (e.g., laboratory animals, household pets, or livestock), non-domesticated animal (e.g., wildlife), dog, cat, rodent, mouse, hamster, cow, bird, chicken, fish, pig, horse, goat, sheep, rabbit, and any combination thereof.

The compounds, conjugates, salts, compositions and methods can be useful as a therapeutic, for example, a treatment that can be administered to a subject in need thereof. A therapeutic effect of the present disclosure can be obtained in a subject by reduction, suppression, remission, or eradication of a disease state, including, but not limited to, a symptom thereof. A therapeutic effect in a subject having a disease or condition, or pre-disposed to have or is beginning to have the disease or condition, can be obtained by a reduction, a suppression, a prevention, a remission, or an eradication of the condition or disease, or pre-condition or pre-disease state.

In practicing the methods described herein, therapeutically-effective amounts of the compounds, conjugates, salts, and compositions can be administered to a subject in need thereof, often for treating and/or preventing a condition or progression thereof. A pharmaceutical composition can affect the physiology of the subject, such as the immune system, an inflammatory response, or other physiologic affect. A therapeutically-effective amount can vary depending on the severity of the disease, the age and relative health of the subject, the potency of the compounds used, and other factors.

Treat and/or treating refer to any indicia of success in the treatment or amelioration of the disease or condition. Treating can include, for example, reducing, delaying or alleviating the severity of one or more symptoms of the disease or condition, or it can include reducing the frequency with which symptoms of a disease, defect, disorder, or adverse condition, and the like, are experienced by a patient. Treat can be used herein to refer to a method that results in some level of treatment or amelioration of the disease or condition, and can contemplate a range of results directed to that end, including but not restricted to prevention of the condition entirely.

Prevent, preventing and the like refer to the prevention of the disease or condition, e.g., tumor formation, in the patient. For example, if an individual at risk of developing a tumor or other form of cancer is treated with the methods of the present disclosure and does not later develop the tumor or other form of cancer, then the disease has been prevented, at least over a period of time, in that individual. Preventing can also refer to preventing re-occurrence of a disease or condition in a patient that has previously been treated for the disease or condition, e.g., by preventing relapse.

A therapeutically effective amount (also referred to as an effective amount) can be the amount of a composition (e.g., conjugate or compound) or an active component thereof sufficient to provide a beneficial effect or to otherwise reduce a detrimental non-beneficial event to the individual to whom the composition is administered. A therapeutically effective dose can be a dose that produces one or more desired or desirable (e.g., beneficial) effects for which it is administered, such administration occurring one or more times over a given period of time. An exact dose can depend on the purpose of the treatment, and can be ascertainable by one skilled in the art using known techniques and the teachings provided herein.

The conjugates that can be used in therapy can be formulated and dosages established in a fashion consistent with good medical practice taking into account the disease or condition to be treated, the condition of the individual patient, the site of delivery of the composition, the method of administration and other factors known to practitioners. The compositions can be prepared according to the description of preparation described herein.

Pharmaceutical compositions can be used in the methods described herein and can be administered to a subject in need thereof using a technique known to one of ordinary skill in the art which can be suitable as a therapy for the disease or condition affecting the subject. One of ordinary skill in the art would understand that the amount, duration and frequency of administration of a pharmaceutical composition to a subject in need thereof depends on several factors including, for example but not limited to, the health of the subject, the specific disease or condition of the patient, the grade or level of a specific disease or condition of the patient, the additional treatments the subject is receiving or has received, and the like.

The methods and compositions can be for administration to a subject in need thereof. Often, administration of the compositions can include routes of administration, non-limiting examples of administration routes include intravenous, intraarterial, subcutaneous, subdural, intramuscular, intracranial, intrasternal, intratumoral, or intraperitoneally. Additionally, a pharmaceutical composition can be administered to a subject by additional routes of administration, for example, by inhalation, oral, dermal, intranasal, or intrathecal administration.

Compositions and conjugates of the present disclosure can be administered to a subject in need thereof in a first administration, and in one or more additional administrations. The one or more additional administrations can be administered to the subject in need thereof minutes, hours, days, weeks or months following the first administration. Any one of the additional administrations can be administered to the subject in need thereof less than 21 days, or less than 14 days, less than 10 days, less than 7 days, less than 4 days or less than 1 day after the first administration. The one or more administrations can occur more than once per day, more than once per week or more than once per month. The administrations can be weekly, biweekly (every two weeks), every three weeks, monthly or bimonthly.

The compounds, conjugates, salts, compositions and methods provided herein may be useful for the treatment of a plurality of diseases, conditions, preventing a disease or a condition in a subject or other therapeutic applications for subjects in need thereof. Often the compounds, conjugates, salts, compositions, and methods provided herein may be useful for treatment of hyperplastic conditions, including but not limited to, neoplasms, cancers, tumors, or the like. The compounds, conjugates, salts, compositions, and methods provided herein may be useful in specifically targeting TGFβR1, TGFβR2, or combinations thereof and inhibiting the signaling or activities of TGFβ1, TGFβ2, TGFβ3, or combinations thereof. The compounds, salts, compositions and methods provided herein may be useful in inhibiting the signaling or activities of TGFβ1, TGFβ2, and/or TGFβ3, and/or directly inhibiting TGFβR1 and/or TGFβR2, or combinations thereof. In one embodiment, the compounds of the present disclosure activate or enhance an immune response. In another embodiment, the conjugates of the present disclosure activate or enhance an immune response.

A condition, such as a cancer, may be associated with expression of a molecule on the cancer cells. Often, the molecule expressed by the cancer cells may comprise an extracellular portion capable of recognition by the antibody construct of the conjugate. A molecule expressed by the cancer cells may be a tumor antigen. An antibody construct of the conjugate may recognize a tumor antigen.

In certain embodiments, the antigen binding domain specifically binds to an antigen that is at least 80% identical to an antigen on a T cell, a B cell, a stellate cell, an endothelial cell, a tumor cell, an APC, a fibroblast cell, a fibrocyte cell, a hepatocyte, or a cell associated with the pathogenesis of fibrosis. In certain embodiments, the antigen binding domain specifically binds to an antigen that is at least 80% identical to an antigen on a T cell, an APC, and/or a B cell. In certain embodiments, the antigen binding domain may specifically bind to an antigen that is at least 80% identical to an antigen selected from the group consisting of CTLA4, PD-1, OX40, LAG-3, GITR, GARP, CD25, CD27, PD-L1, TNFR2, ICOS, 41BB, CD70, CD73, CD38, or VTCN1. In certain embodiments, the antigen binding domain specifically binds to an antigen that is at least 80% identical to an antigen on a stellate cell, an endothelial cell, a fibroblast cell, a fibrocyte cell, or a cell associated with the pathogenesis of fibrosis or cancer. In certain embodiments, the antigen binding domain may specifically bind to an antigen that is at least 80% identical to an antigen selected from the group consisting of LRRC15, PDGFRβ, integrin αvβ1, integrin αvβ3, integrin αvβ6, integrin αvβ8, Endosialin, FAP, ADAM12, MMP14, PDPN, CDH11 and F2RL2, In certain embodiments, the antigen binding domain may specifically bind to an antigen that is at least 80% identical to an antigen selected from the group consisting of LRRC15, FAP, ADAM12, MMP14, PDPN, CDH11 and F2RL2, In certain embodiments, the antigen binding domain specifically binds to an antigen that is at least 80% identical to an antigen on a tumor cell, a tumor antigen. In certain embodiments, the antigen binding domain specifically binds to an antigen that is at least 80% identical to an antigen selected from the group consisting of MUC16, UPK1B, VTCN1, TMPRSS3, TMEM238, Clorf186, TMPRSS4, CLDN6, CLDN8, STRA6, MSLN or CD73.

In certain embodiments, the antigen binding domain specifically binds to an antigen on a T cell, a B cell, a stellate cell, an endothelial cell, a tumor cell, an APC, a fibroblast cell, a fibrocyte cell, or a cell associated with the pathogenesis of fibrosis. In certain embodiments, the antigen binding domain specifically binds to an antigen on a T cell, an APC, and/or a B cell. In certain embodiments, the antigen binding domain may specifically bind to an antigen selected from the group consisting of CTLA4, PD-1, OX40, LAG-3, GITR, GARP, CD25, CD27, PD-L1, TNFR2, ICOS, 41BB, CD70, CD73, CD38 or VTCN1. In certain embodiments, the antigen binding domain specifically binds to an antigen on a stellate cell, an endothelial cell, a fibroblast cell, a fibrocyte cell, or a cell associated with the pathogenesis of fibrosis or cancer. In certain embodiments, the antigen binding domain may specifically bind to an antigen selected from the group consisting of, PDGFRβ, integrin αvβ1, integrin αvβ3, integrin αvβ6, integrin αvβ8, Endosialin, FAP, ADAM12, LRRC15, MMP14, PDPN, CDH11 and F2RL2. In certain embodiments, the antigen binding domain may specifically bind to an antigen selected from the group consisting of FAP, ADAM12, LRRC15, MMP14, PDPN, CDH11 and F2RL2. In certain embodiments, the antigen binding domain specifically binds to an antigen on a tumor cell, a tumor antigen. In certain embodiments, the antigen binding domain specifically binds to an antigen selected from the group consisting of MUC16, UPK1B, VTCN1, TMPRSS3, TMEM238, Clorf186, TMPRSS4, CLDN6, CLDN8, STRA6, MSLN or CD73.

Additionally, such antigens may be derived from the following specific conditions and/or families of conditions, including but not limited to, cancers such as brain cancers, skin cancers, lymphomas, sarcomas, lung cancer, liver cancer, leukemias, uterine cancer, breast cancer, ovarian cancer, cervical cancer, bladder cancer, kidney cancer, hemangiosarcomas, bone cancers, blood cancers, testicular cancer, prostate cancer, stomach cancer, intestinal cancers, pancreatic cancer, and other types of cancers as well as pre-cancerous conditions such as hyperplasia or the like.

Non-limiting examples of cancers may include Acute lymphoblastic leukemia (ALL); Acute myeloid leukemia; Adrenocortical carcinoma; Astrocytoma, childhood cerebellar or cerebral; Basal-cell carcinoma; Bladder cancer; Bone tumor, osteosarcoma/malignant fibrous histiocytoma; Brain cancer; Brain tumors, such as, cerebellar astrocytoma, malignant glioma, ependymoma, medulloblastoma, visual pathway and hypothalamic glioma; Brainstem glioma; Breast cancer; Bronchial adenomas/carcinoids; Burkitt's lymphoma; Cerebellar astrocytoma; Cervical cancer; Cholangiocarcinoma; Chondrosarcoma; Chronic lymphocytic leukemia; Chronic myelogenous leukemia; Chronic myeloproliferative disorders; Colon cancer; Cutaneous T-cell lymphoma; Endometrial cancer; Ependymoma; Esophageal cancer; Eye cancers, such as, intraocular melanoma and retinoblastoma; Gallbladder cancer; Glioma; Hairy cell leukemia; Head and neck cancer; Heart cancer; Hepatocellular (liver) cancer; Hodgkin lymphoma; Hypopharyngeal cancer; Islet cell carcinoma (endocrine pancreas); Kaposi sarcoma; Kidney cancer (renal cell cancer); Laryngeal cancer; Leukemia, such as, acute lymphoblastic, acute myeloid, chronic lymphocytic, chronic myelogenous and, hairy cell; Lip and oral cavity cancer; Liposarcoma; Lung cancer, such as, non-small cell and small cell; Lymphoma, such as, AIDS-related, Burkitt; Lymphoma, cutaneous T-Cell, Hodgkin and Non-Hodgkin, Macroglobulinemia, Malignant fibrous histiocytoma of bone/osteosarcoma; Melanoma; Merkel cell cancer; Mesothelioma; Multiple myeloma/plasma cell neoplasm; Mycosis fungoides; Myelodysplastic syndromes; Myelodysplastic/myeloproliferative diseases; Myeloproliferative disorders, chronic; Nasal cavity and paranasal sinus cancer; Nasopharyngeal carcinoma; Neuroblastoma; Oligodendroglioma; Oropharyngeal cancer; Osteosarcoma/malignant fibrous histiocytoma of bone; Ovarian cancer; Pancreatic cancer; Parathyroid cancer; Pharyngeal cancer; Pheochromocytoma; Pituitary adenoma; Plasma cell neoplasia; Pleuropulmonary blastoma; Prostate cancer; Rectal cancer; Renal cell carcinoma (kidney cancer); Renal pelvis and ureter, transitional cell cancer; Rhabdomyosarcoma; Salivary gland cancer; Sarcoma, Ewing family of tumors; Sarcoma, Kaposi; Sarcoma, soft tissue; Sarcoma, uterine; Sezary syndrome; Skin cancer (non-melanoma); Skin carcinoma; Small intestine cancer; Soft tissue sarcoma; Squamous cell carcinoma; Squamous neck cancer with occult primary, metastatic; Stomach cancer; Testicular cancer; Throat cancer; Thymoma and thymic carcinoma; Thymoma; Thyroid cancer; Thyroid cancer, childhood; Uterine cancer; Vaginal cancer; Waldenström macroglobulinemia; Wilms tumor and any combination thereof.

Non-limiting examples of fibrosis or fibrotic diseases include adhesive capsulitis, arterial stiffness, arthrofibrosis, atrial fibrosis, cirrhosis, Crohn's disease, collagenous fibroma, chronic kidney disease including glomulosclerosis and interstial fibrosis, cystic fibrosis, Desmoid-type fibromatosis, Dupuytren's contracture, elastofibroma, endomyocardial fibrosis, fibroma of tendon sheath, glial scar, idiopathic pulmonary fibrosis (IPF), interstitial lung disease (ILD), keloid, mediastinal fibrosis, myelofibrosis, dilated cardiomyopathy, myocardial fibrosis, non-alcoholic fatty liver disease, nuchal fibroma, nephrogenic systemic fibrosis, old myocardial infarction, Peyronie's disease, pulmonary fibrosis, progressive massive fibrosis, non-alcoholic steatohepatitis (NASH), radiation-induced lung injury, retroperitoneal fibrosis, scar, scleroderma/systemic sclerosis.

The invention provides any therapeutic compound or composition disclosed herein for use in a method of treatment of the human or animal body by therapy. The invention further provides any therapeutic compound or composition disclosed herein for prevention or treatment of any condition disclosed herein, for example cancer, autoimmune disease, inflammation, sepsis, allergy, asthma, graft rejection, graft-versus-host disease, immunodeficiency or infectious disease (typically caused by an infectious pathogen). The invention also provides any therapeutic compound or composition disclosed herein for obtaining any clinical outcome disclosed herein for any condition disclosed herein, such as reducing tumour cells in vivo. The invention also provides use of any therapeutic compound or composition disclosed herein in the manufacture of a medicament for preventing or treating any condition disclosed herein.

### EXAMPLES

### General Synthetic Schemes and Examples

The following synthetic schemes are provided for purposes of illustration, not limitation. The following examples illustrate the various methods of making compounds described herein. It is understood that one skilled in the art may be able to make these compounds by similar methods or by combining other methods known to one skilled in the art. It is also understood that one skilled in the art would be able to make, in a similar manner as described below by using the appropriate starting materials and modifying the synthetic route as needed. In general, starting materials and reagents can be obtained from commercial vendors or synthesized according to sources known to those skilled in the art or prepared as described herein.

Unless otherwise noted, reagents and solvents were used as received from commercial suppliers. Anhydrous solvents and oven-dried glassware were used for synthetic transformations sensitive to moisture and/or oxygen. Yields were not optimized. Reaction times are approximate and were not optimized. Column chromatography and thin layer chromatography (TLC) were performed on silica gel unless otherwise noted. Spectra are given in ppm (δ) and coupling constants (J) are reported in Hertz (Hz). For proton spectra the solvent peak was used as the reference peak.

In some embodiments, compounds described herein are prepared as described in Scheme 1.

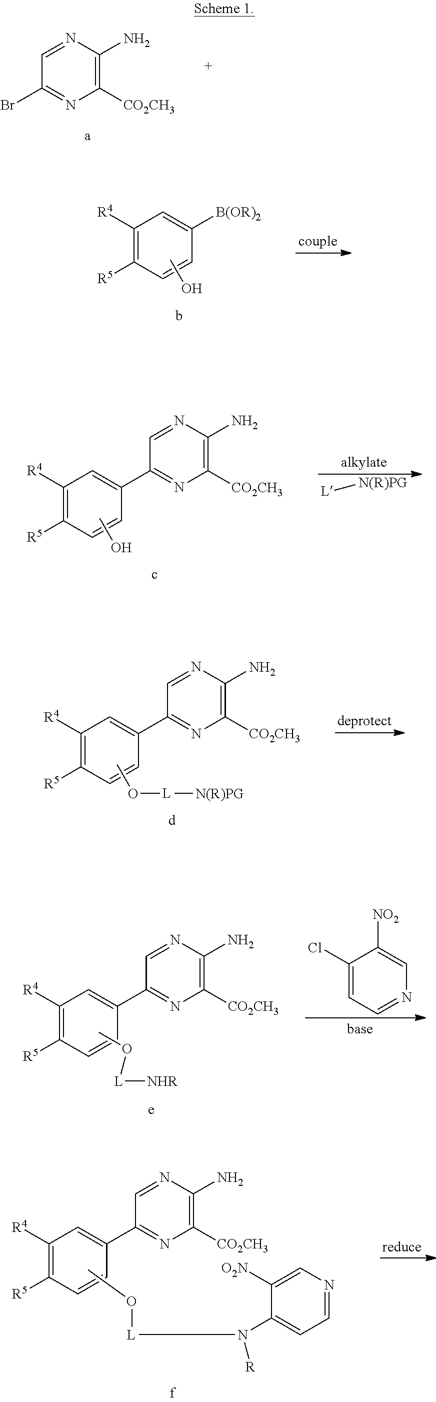

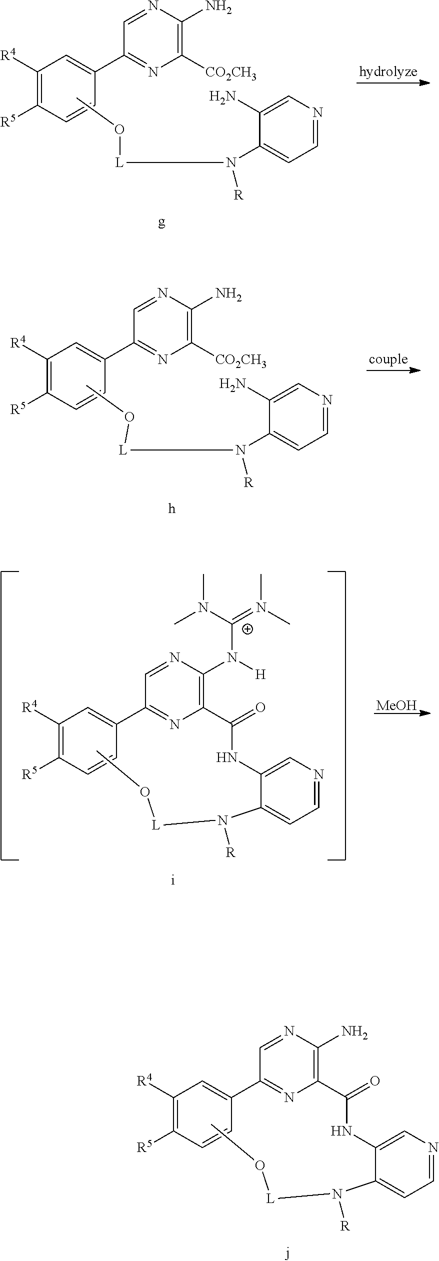

Methyl 3-amino-6-bromopyrazine-2-carboxylate (a) can be coupled to an appropriately substituted hydroxyphenylboronate or boronic acid in the presence of a palladium catalyst such as [1,1′-bis(diphenylphosphino)ferrocene]dichloropalladium(II) and a base such as sodium carbonate at elevated temperatures to afford biaryl intermediates (c). Alkylation of compounds (c) using a protected ω-amino alkylhalide or sulfonate ester in the presence of base such as sodium hydride or cesium carbonate or a protected ω-amino alkanol and triphenylphoshine /dialkylazodicarboxylate mixture can lead to ether compounds (d) which can be deprotected to terminal amine intermediates (e) using a strong acid such as HCl or TFA in cases where PG=Boc or catalytic hydrogenation in cases where PG=Cbz. Amines (e) can react with 4-chloro-3-nitropyridine to provide compounds (f) which can be reduced to amines (g) using standard conditions for the conversion of an aromatic nitro group to an aryl amine such as iron in ammonium chloride solution or a palladium catalyzed hydrogenation reaction. Hydrolysis of the carboxylic ester functional group can be effected by reacting intermediate (g) with a metal alkoxide base such as LiOH to provide carboxylic acids (h) which can undergo a macrolactamization reaction using an amide coupling reagent such as HATU. Intermediate macrolactams (i) can be converted to the desired targets (j) after brief exposure to methanol followed by purification.

### Example 1: Synthesis of 2⁵-amino-6-methyl-9-oxa-4,6-diaza-2(2,6)-pyrazina-5(3,4)-pyridina-1(1,3)-benzenacyclononaphan-3-one (Compound 11)

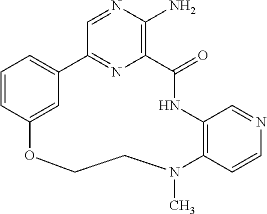 [SMILES: CN1CCOc2cccc(c2)-c2cnc(N)c(n2)C(=O)Nc2cnccc21]

### Step A: Preparation of Int 1.1a

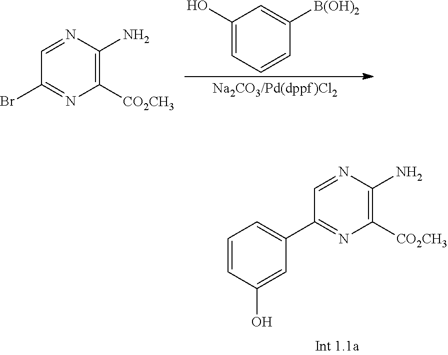 [SMILES: COC(=O)c1nc(Br)cnc1N ; OB(O)c1cccc(O)c1 ; COC(=O)c1nc(-c2cccc(O)c2)cnc1N]

Methyl 3-amino-6-bromopyrazine-2-carboxylate (2.0 g, 8.6 mmol), 3-hydroxyphenyl-boronic acid (1.3 g, 9.5 mmol) were dissolved in 20 mL of dioxane and 8.6 mL of 2M Na₂CO₃ solution. The mixture was degassed using nitrogen (5×) before the addition of Pd(dppf)Cl₂ (628 mg, 0.086 mmol). The reaction mixture was degassed again then heated at 90° C. for 2 h then cooled and filtered through a plug of Celite. The filtrate was diluted with EtOAc then washed with saturated NaHCO₃ solution (2×), water (1×) then brine. The organic extracts were dried over Na2SO4, evaporated and chromatographed (ISCO Gold; 0% to 60% EtOAc/dichloromethane) to afford 1.1 g of compound Int 1.1a as a yellow solid. ¹H NMR (DMSO-d⁶) δ 9.54 (s, 1H), 8.82 (s, 1H), 7.42-7.39 (m, 4H), 7.25 (t, J=8.0 Hz, 1H), 6.77 (m, 1H), 3.89 (s, 3H). M+H=246.2.

### Step B: Preparation of Int 1.1b

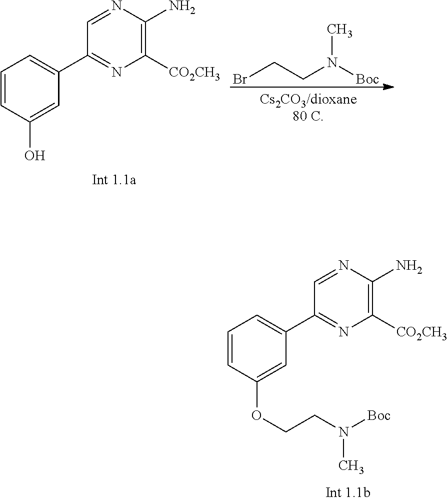 [SMILES: COC(=O)c1nc(-c2cccc(O)c2)cnc1N ; CN(CCBr)C(=O)OC(C)(C)C ; COC(=O)c1nc(-c2cccc(OCCN(C)C(=O)OC(C)(C)C)c2)cnc1N]

Compound Int 1.1a (1.1 g, 4.5 mmol) in 20 mL of dioxane treated with 4.39 g (13.5 mmol) of cesium carbonate and 1.1 g (4.5 mmol) of tert-butyl(2-bromoethyl)(methyl)carbamate and the mixture was heated to 80° C. for 8h. The reaction was cooled and quenched by the addition of water then extracted with EtOAc three times. The combined organic extracts were washed with brine then dried and evaporated to give 600 mg of a solid residue that was used directly without purification. M+H=403.2.

### Step C: Preparation of Int 1.1c

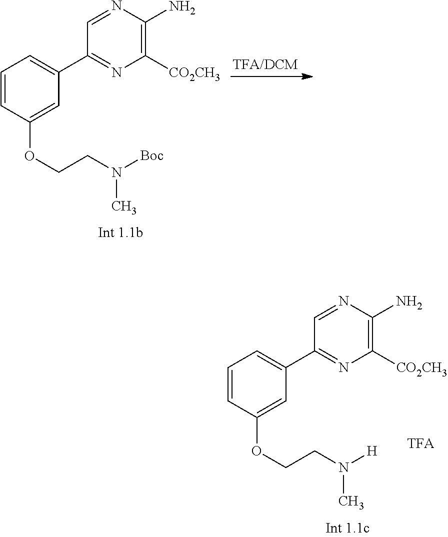 [SMILES: COC(=O)c1nc(-c2cccc(OCCN(C)C(=O)OC(C)(C)C)c2)cnc1N ; CNCCOc1cccc(-c2cnc(N)c(C(=O)OC)n2)c1]

Compound Int 1.1b (600 mg, 1.5 mmol) was dissolved in 15 mL of dichloromethane then treated with 5 mL of TFA and stirred at room temperature for 3h. The solvents were removed and the residue was covered in toluene and evaporated. This procedure was repeated three times to provide 440 mg of the desired compound Int 1.1c as the TFA salt. M+H=303.2.

### Step D: Preparation of Int 1.1d

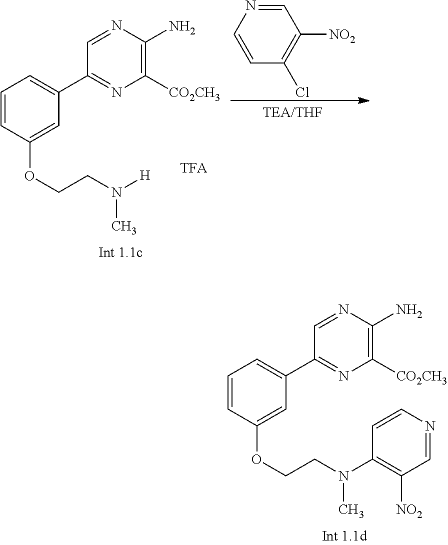 [SMILES: CNCCOc1cccc(-c2cnc(N)c(C(=O)OC)n2)c1 ; O=[N+]([O-])c1cnccc1Cl ; COC(=O)c1nc(-c2cccc(OCCN(C)c3ccncc3[N+](=O)[O-])c2)cnc1N]

Compound Int 1.1c (417 mg, 1.00 mmol) was dissolved in 10 mL of THF then treated with 0.70 mL (5.0 mmol) of triethylamine and 158 mg (1.0 mmol) of 4-chloro-3-nitropyridine and the mixture was heated at 40 C for 2 h. The mixture was cooled and diluted with 25 mL of EtOAc then washed with water (3×) and brine (1×). The organic extracts were dried over Na2SO4, evaporated and purified by reverse phase chromatography to afford 340 mg of compound Int 1.1d as a yellow solid. M+H=425.3.

### Step E: Preparation of Int 1.1e

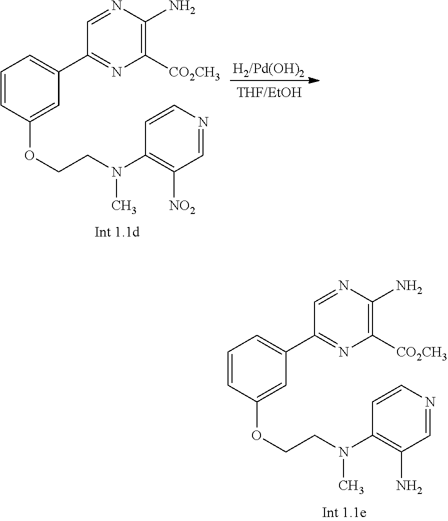 [SMILES: COC(=O)c1nc(-c2cccc(OCCN(C)c3ccncc3[N+](=O)[O-])c2)cnc1N ; COC(=O)c1nc(-c2cccc(OCCN(C)c3ccncc3N)c2)cnc1N]

Nitro compound Int 1.1d (340 mg, 0.80 mmol) was dissolved in 10 mL of THF and 10 mL of EtOH and the mixture was degassed. 100 mg of 20% Pd(OH)₂ was then added and the mixture was covered with a balloon containing hydrogen gas. The reaction was stirred at room temperature for 4h then filtered through Celite. The filtrate was evaporated to afford 220 mg of the desired product Int 1.1e as a tan colored solid. M+H=395.2.

### Step F: Preparation of Int 1.1f

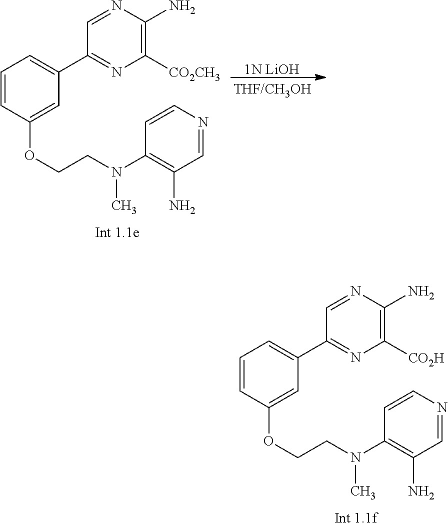 [SMILES: COC(=O)c1nc(-c2cccc(OCCN(C)c3ccncc3N)c2)cnc1N ; "1N LiOH" ; CN(CCOc1cccc(-c2cnc(N)c(C(=O)O)n2)c1)c1ccncc1N]

Compound Int 1.1e (200 mg, 0.5 mmol) was dissolved in 2 mL of THF and 1 mL of methanol at room temperature then treated with 2.2 equivalents (1.1 mL) of 1N LiOH. The mixture was stirred for 3h then evaporated to dryness. The residue was covered with 5 mL of toluene and evaporated. This procedure was repeated three times to leave 120 mg of the desired amino acid Int 1.1f which was used directly in the next step. M+H=381.1.

### Step G: Preparation of Compound 11

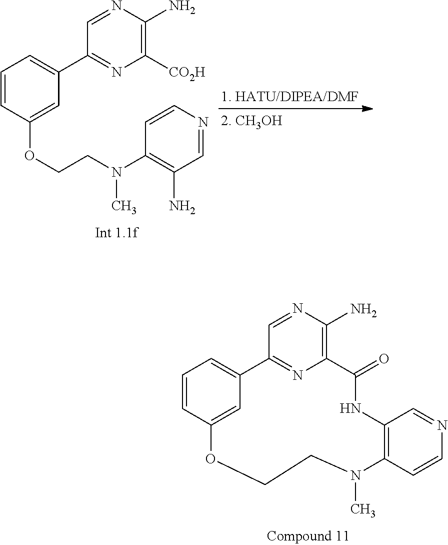 [SMILES: CN(CCOc1cccc(-c2cnc(N)c(C(=O)O)n2)c1)c1ccncc1N ; CN1CCOc2cccc(c2)-c2cnc(N)c(n2)C(=O)Nc2cnccc21]

A mixture containing 100 mg (0.26 mmol) of Int 1.1f in 13 mL of DMF was treated with 222 mg (0.58 mmol) of HATU and 0.18 mL (1.04 mmol) of NMM then stirred at room temperature for 4h. The reaction was then quenched with 1 mL of methanol and 1 mL of ammonium hydroxide solution and stirred for 1h. The solvents were removed and the residue was chromatographed by reverse phase chromatography to provide 32 mg of Compound 11 as a yellow solid. ¹H NMR (DMSO-d⁶) δ 12.0 (bs, 1H), 9.09 (s, 1H), 9.03 (s, 1H), 8.78 (s, 1H), 8.22 (s, 1H), 7.53 (d, J=7.8 Hz, 1H), 7.43 (s, 2H), 7.36 (t, J=7.8 Hz, 1H), 7.27 (s, 1H), 6.87 (dd, J=2.4, 7.8 Hz, 1H), 4.49 (m, 2H), 3.58 (m, 2H), 2.62 (s, 3H). M+H=363.1.

The compounds in Table 2 below were prepared in a manner analogous to that described above in Scheme 1 and for the synthesis of Compound 11 in Example 1.

TABLE 2

| Cmpd | Structure | ¹H NMR/LRMS |
|---|---|---|
| 1 | 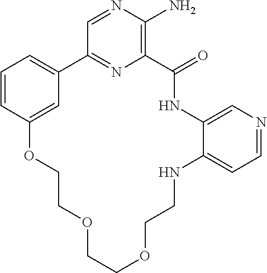 [SMILES: Nc1ncc2nc1C(=O)Nc1cnccc1NCCOCCOCCOc1cccc-2c1] | LRMS (ESI+) [M + H]⁺; 437.5 |
| 2 | 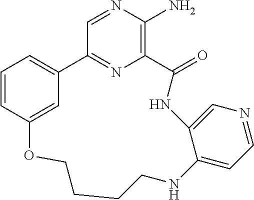 [SMILES: Nc1ncc2nc1C(=O)Nc1cnccc1NCCCCOc1cccc-2c1] | LRMS (ESI+) [M + H]⁺; 377.4 |
| 3 | 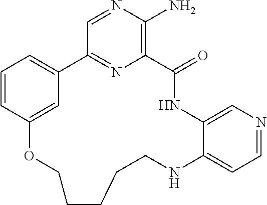 [SMILES: Nc1ncc2nc1C(=O)Nc1cnccc1NCCCCCOc1cccc-2c1] | LRMS (ESI+) [M + H]⁺; 391.4 |
| 4 | 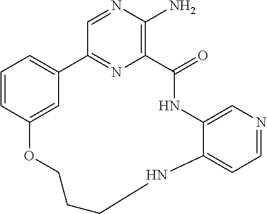 [SMILES: Nc1ncc2nc1C(=O)Nc1cnccc1NCCCOc1cccc-2c1] | ¹H NMR (300 MHz, DMSO-d₆) δ: 2.12 (s, 2H), 3.09 (s, 2H), 4.44 (s, 2H), 6.00 (s, 1H), 6.81-6.89 (m, 2H), 7.33-7.36 (m, 1H), 7.59 (s, 2H), 7.70 (d, J = 5.4 Hz, 1H), 8.12(d, J = 5.4 Hz, 1H), 8.22 (s, 1H), 9.05 (s, 2H), 10.98 (s, 1H). LRMS (ESI+) [M + H]⁺; 363.4 |
| 5 | 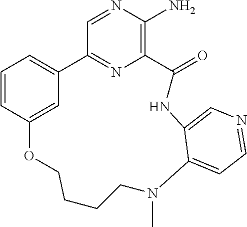 [SMILES: CN1CCCCOc2cccc(c2)-c2cnc(N)c(n2)C(=O)Nc2cnccc21] | ¹H NMR (300 MHz, DMSO-d₆) δ: 1.68 (s, 2H), 2.05 (s, 2H), 2.65 (s, 3H), 3.15 (s, 2H), 4.22 (s, 2H), 6.91 (m, 1H), 7.26-7.36 (m, 2H), 7.56-7.58 (m, 1H), 7.77-7.82 (m, 3H), 8.27 (s, 1H), 8.99 (s, 1H), 9.31 (s, 1H), 10.34 (s, 1H). LRMS (ESI+) [M + H]⁺; 391.4 |
| 6 | 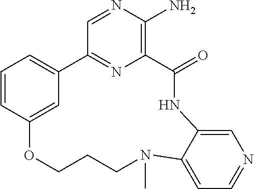 [SMILES: CN1CCCOc2cccc(c2)-c2cnc(N)c(n2)C(=O)Nc2cnccc21] | ¹H NMR (300 MHz, DMSO-d₆) δ: 2.05(s, 2H), 2.58 (s, 3H), 3.06 (t, J = 4.8 Hz, 2H), 4.35 (t, J = 8.8 Hz, 2H), 6.88-6.89 (m, 1H), 7.25 (d, J = 5.2 Hz, 1H), 7.37 (t, J = 7.6 Hz, 1H), 7.64-7.69 (m, 3H), 8.16 (s, 1H), 8.30 (d, J = 5.2 Hz, 1H,), 9.06 (s, 1H), 9.30 (s, 1H), 11.23 (s, 1H). LRMS (ESI+) [M + H]⁺; 377.4 |
| 7 | 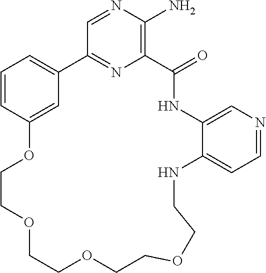 [SMILES: Nc1ncc2nc1C(=O)Nc1cnccc1NCCOCCOCCOCCOc1cccc-2c1] | ¹H NMR (400 MHz, Methanol-d₄) δ: 3.46- 3.47 (m, 2H), 3.59-3.70 (m, 6H), 3.70-3.76 (m, 4H), 3.85-3.87 (m, 2H), 4.27 (t, J = 4.4 Hz, 2H), 6.78 (d, J = 6.0 Hz, 1H), 6.98 (dd, J₁ = 8.4, J₂ = 2.8 Hz, 1H), 7.38 (t, J = 8.0 Hz, 1H), 7.50 (d, J = 7.6 Hz, 1H), 7.96 (s, 1H), 8.08 (d, J = 5.6 Hz, 1H), 8.19 (s, 1H), 8.78 (s, 1H). LRMS (ESI+) [M + H]⁺; 481.5 |
| 8 | 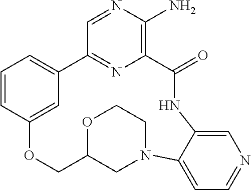 [SMILES: Nc1ncc2nc1C(=O)Nc1cnccc1N1CCOC(COc3cccc-2c3)C1] | ¹H NMR (300 MHz, DMSO-d₆) δ: 2.06-2.07 (m, 1H), 2.28-2.38 (m, 1H), 2.83-2.87 (m, 1H), 3.53-3.56 (m, 1H), 3.87-4.00 (m, 2H), 4.16-4.18 (m, 1H), 4.32-4.46 (m, 2H), 6.93- 6.96 (m, 1H), 7.25 (d, J = 12.3 Hz, 1H), 7.30-7.39 (m, 1H), 7.54 (d, J = 19.8 Hz, 2H), 7.75 (d, J = 15.6 Hz, 2H), 8.32 (s, 1H), 8.99 (s, 1H), 9.93 (s, 1H), 10.09 (s, 1H). LRMS (ESI+) [M + H]⁺; 405.4 |
| 9 | 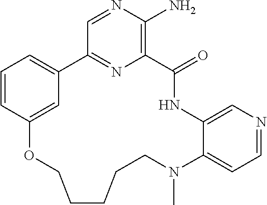 [SMILES: CN1CCCCCOc2cccc(c2)-c2cnc(N)c(n2)C(=O)Nc2cnccc21] | ¹H NMR (300 MHz, Methanol-d₄) δ: 1.37- 1.50 (m, 2H), 1.67-1.88 (m, 4H), 2.65 (s, 3H), 3.23 (t, J = 6.0 Hz, 2H), 4.19 (t, J = 7.2 Hz, 2H), 6.92-6.96 (m, 1H), 7.22 (d, J = 5.4 Hz, 1H), 7.33-7.38 (m, 1H), 7.47 (d, J = 7.8 Hz, 1H), 7.64 (s, 1H), 8.21 (d, J = 5.4 Hz, 1H), 8.79 (s, 1H), 9.39 (s, 1H). LRMS (ESI+) [M + H]⁺; 405.4 |
| 10 | 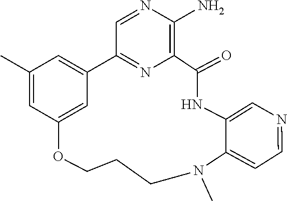 [SMILES: Cc1cc2cc(c1)-c1cnc(N)c(n1)C(=O)Nc1cnccc1N(C)CCCO2] | ¹H NMR (400 MHz, DMSO-d₆) δ: 2.02-2.07 (m, 2H), 2.34 (s, 3H), 2.55 (s, 3H), 3.03- 3.04 (m, 2H), 4.28-4.32 (m, 2H), 6.70 (s, 1H), 7.23 (d, J = 5.2 Hz, 1H), 7.51 (s, 1H), 7.61 (s, 2H), 7.94 (s, 1H), 8.28 (d, J = 5.2 Hz, 1H), 9.01 (s, 1H), 9.29 (s, 1H), 11.22 (s, 1H). LRMS (ESI+) [M + H]⁺; 391.4 |
| 12 | 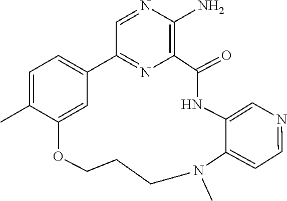 [SMILES: Cc1ccc2cc1OCCCN(C)c1ccncc1NC(=O)c1nc-2cnc1N] | ¹H NMR (400 MHz, Methanol-d₄) δ: 2.16- 2.18 (m, 2H), 2.24 (s, 3H), 2.70 (s, 3H), 3.15-3.18 (m, 2H), 4.40-4.45 (m, 2H), 7.21- 7.47 (m, 3H), 8.14-8.27 (m, 2H), 8.85 (s, 1H), 9.37 (s, 1H). LRMS (ESI+) [M + H]⁺; 391.4 |
| 13 | 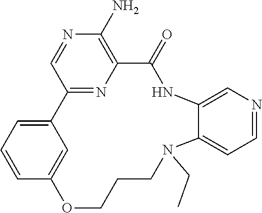 [SMILES: CCN1CCCOc2cccc(c2)-c2cnc(N)c(n2)C(=O)Nc2cnccc21] | ¹H NMR (300 MHz, DMSO-d₆) δ: 0.66 (t, J = 7.2 Hz, 3H), 2.01-2.06 (m, 2H), 3.03-3.10 (m, 4H), 4.29-4.34 (m, 2H), 6.85 (dd, J₁ = 8.4 Hz, J₂ = 2.4 Hz, 1H), 7.22 (d, J = 6.0 Hz, 1H), 7.33 (t, J = 7.8 Hz, 1H), 7.62-7.64 (m, 3H), 8.11 (s, 1H), 8.27 (d, J = 5.4 Hz, 1H), 9.00 (s, 1H), 9.30 (s, 1H), 11.19 (s, 1H). LRMS (ESI+) [M + H]⁺; 391.5 |
| 14 | 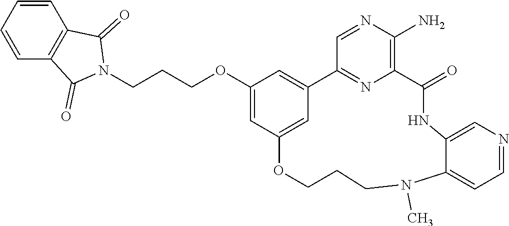 [SMILES: CN1CCCOc2cc(OCCCN3C(=O)c4ccccc4C3=O)cc(c2)-c2cnc(N)c(n2)C(=O)Nc2cnccc21] | LRMS (ESI+) [M + H]⁺; 580.6 |
| 16 | 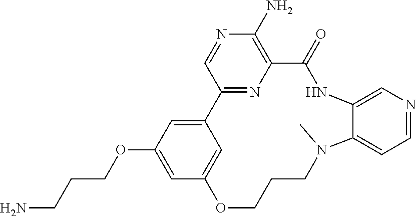 [SMILES: CN1CCCOc2cc(OCCCN)cc(c2)-c2cnc(N)c(n2)C(=O)Nc2cnccc21] | LRMS (ESI+) [M + H]⁺; 450.5 |
| 17 | 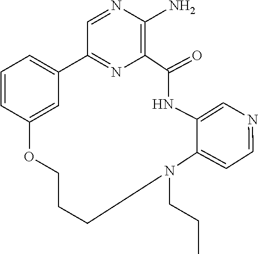 [SMILES: CCCN1CCCOc2cccc(c2)-c2cnc(N)c(n2)C(=O)Nc2cnccc21] | ¹H NMR (400 MHz, DMSO-d₆) δ: 0.50-0.58 (m, 3H), 1.11-1.19 (m, 2H), 2.03-2.05 (m, 2H), 2.92 (t, J = 7.5 Hz, 2H), 3.05 (s, 2H), 4.30 (t, J = 7.5 Hz, 2H), 6.84 (dd, J₁ = 8.1 Hz, J₂ = 2.1 Hz, 1H), 7.24-7.32 (m, 2H), 7.60-7.63 (m, 3H), 8.07 (s, 1H), 8.25 (d, J = 5.1 Hz, 1H), 9.00 (s, 1H), 9.34 (s, 1H), 11.14 (s, 1H). LRMS (ESI+) [M + H]⁺; 405.5 |
| 18 | 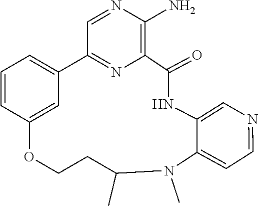 [SMILES: CC1CCOc2cccc(c2)-c2cnc(N)c(n2)C(=O)Nc2cnccc2N1C] | ¹H NMR (400 MHz, DMSO-d₆) δ: 1.06 (d, J = 5.6 Hz, 3H), 2.00-2.07 (m, 1H), 2.20 (s, 1H), 2.59 (s, 3H), 3.53-3.54 (m, 1H), 4.27- 4.37 (m, 2H), 6.88 (d, J = 7.6 Hz, 1H), 7.26 (d, J = 4.4 Hz, 1H), 7.37 (t, J = 7.6 Hz, 1H), 7.60-7.62 (m, 3H), 7.87 (s, 1H), 8.27 (d, J = 4.4 Hz, 1H), 9.01 (s, 1H), 9.58 (s, 1H), 10.64 (s, 1H). LRMS (ESI+) [M + H]⁺; 391.5 |
| 19 | 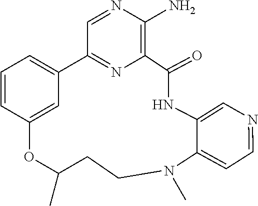 [SMILES: CC1CCN(C)c2ccncc2NC(=O)c2nc(cnc2N)-c2cccc(c2)O1] | ¹H NMR (400 MHz, DMSO-d₆) δ: 1.37 (d, J = 6.0 Hz, 3H), 1.69-1.71 (m, 1H), 2.08-2.26 (m, 1H), 2.51 (s, 3H), 3.10-3.36 (m, 2H), 4.66-4.67 (m, 1H), 6.86 (d, J = 8.0 Hz, 1H), 7.28-7.29 (m, 1H), 7.33-7.38 (m, 1H), 7.66 (d, J = 8.0 Hz, 3H), 8.07 (s, 1H), 8.30 (s, 1H), 9.05 (d, J = 5.2 Hz, 1H), 9.32 (s, 1H), 11.23 (d, J = 5.2 Hz, 1H). LRMS (ESI+) [M + H]⁺; 391.5 |
| 21 | 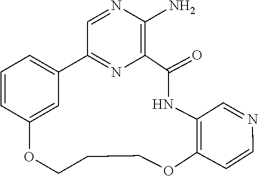 [SMILES: Nc1ncc2nc1C(=O)Nc1cnccc1OCCCOc1cccc-2c1] | ¹H NMR (400 MHz, DMSO-d₆) δ: 2.13 (s, 2H), 4.02-4.05 (m, 4H), 5.99 (d, J = 7.2 Hz, 1H), 6.45 (s, 1H), 6.78 (d, J = 7.2 Hz, 1H), 7.11 (s, 2H), 7.22-7.28 (m, 2H), 7.68 (d, J = 7.2 Hz, 1H), 8.11 (s, 1H), 8.45 (s, 1H), 9.14 (s, 1H). LRMS (ESI+) [M + H]⁺; 364.4 |
| 22 | 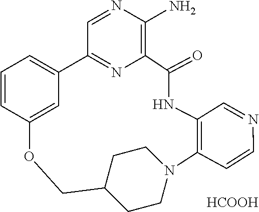 [SMILES: Nc1ncc2nc1C(=O)Nc1cnccc1N1CCC(CC1)COc1cccc-2c1 ; "HCOOH"] | ¹H NMR (400 MHz, Formic acid-d₂) δ: 0.40-0.50 (m, 2H), 0.64-0.68 (m, 1H), 1.19- 1.26 (m, 2H), 2.29 (t, J = 12.8 Hz, 2H), 2.55 (d, J = 7.2 Hz, 2H), 2.94 (d, J = 10.0 Hz, 2H), 6.04-6.06 (m, 1H), 6.70-6.84 (m, 3H), 7.42 (s, 1H), 7.68-7.69 (m, 1H), 7.87 (s, 1H), 8.64 (s, 1H). LRMS (ESI+) [M + H]⁺; 403.5 |
| 23 | 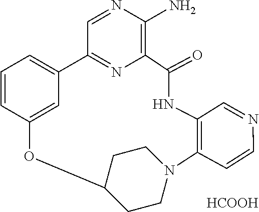 [SMILES: Nc1ncc2nc1C(=O)Nc1cnccc1N1CCC(CC1)Oc1cccc-2c1 ; "HCOOH"] | ¹H NMR (400 MHz, DMSO-d₆) δ: 2.04-2.08 (m, 2H), 2.30-2.33 (m, 2H), 2.89 (m, 2H), 2.95-3.00 (m, 2H), 4.55 (s, 1H), 7.05-7.07 (m, 1H), 7.26 (d, J = 5.2 Hz, 1H), 7.37 (t, J = 8.0 Hz, 1H), 7.56 (d, J = 7.6 Hz, 1H), 7.75 (s, 1H), 7.81-7.95 (m, 1H), 8.26-8.32 (m, 2H), 8.93 (s, 1H), 9.18 (s, 1H), 10.10 (s, 1H). LRMS (ESI+) [M + H]⁺; 389.4 |
| 24 | 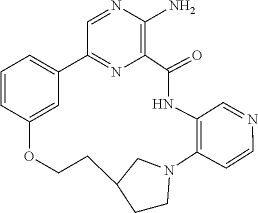 [SMILES: Nc1ncc2nc1C(=O)Nc1cnccc1N1CCC(CCOc3cccc-2c3)C1] | ¹H NMR (300 MHz, DMSO-d₆) δ: 1.69-1.74 (m, 2H), 1.90-2.38 (m, 4H), 3.33 (s, 1H), 3.99 (t, J = 9.0 Hz, 1H), 4.25-4.43 (m, 3H), 6.70 (d, J = 6.0 Hz, 1H), 6.87 (dd, J₁ = 8.1 Hz, J₂ = 2.1 Hz, 1H), 7.34 (t, J = 8.1 Hz, 1H), 7.47 (s, 2H), 7.65 (d, J = 7.8 Hz, 1H), 7.90 (s, 1H), 8.06 (d, J = 5.7 Hz, 1H), 8.53 (s, 1H), 9.00 (s, 1H), 10.09 (s, 1H). LRMS (ESI+) [M + H]⁺; 403.5 |
| 25 | 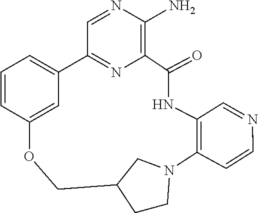 [SMILES: Nc1ncc2nc1C(=O)Nc1cnccc1N1CCC(COc3cccc-2c3)C1] | ¹H NMR (400 MHz, DMSO-d₆) δ: 1.95- 2.03 (m, 2H), 2.50-2.51 (m, 1H), 2.91(s, 1H), 3.03-3.06 (m, 1H), 3.54 (s, 1H), 3.92- 3.93 (m, 1H), 4.34-4.50 (m, 2H), 6.91 (dd, J₁ = 8.0 Hz, J₂ = 2.4 Hz, 1H), 7.14 (d, J = 4.8 Hz, 1H), 7.34 (t, J = 8.0 Hz, 1H), 7.62- 7.67 (m, 3H), 8.21 (d, J = 5.6 Hz, 2H), 9.03 (s, 1H), 9.43 (s, 1H), 10.80 (s, 1H). LRMS (ESI+) [M + H]⁺; 389.4 |
| 32 | 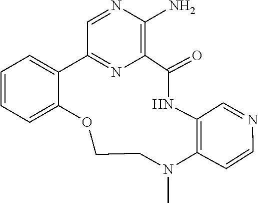 [SMILES: CN1CCOc2ccccc2-c2cnc(N)c(n2)C(=O)Nc2cnccc21] | ¹H NMR (300 MHz, DMSO-d₆) δ: 2.81 (s, 3H), 3.12 (s, 2H), 4.27-4.33 (m, 2H), 7.08- 7.14 (m, 1H), 7.24 (s, 1H), 7.39 (s, 2H), 7.53 (s, 2H), 7.98 (s, 1H), 8.29 (s, 1H), 8.84 (s, 1H), 9.74 (s, 1H), 11.41 (s, 1H). LRMS (ESI+) [M + H]⁺; 363.4 |
| 33 | 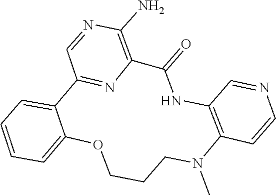 | ¹H NMR (400 MHz, DMSO-d₆) δ: 2.08-2.10 (m, 2H), 2.74 (s, 3H), 3.17 (t, J = 6.8 Hz, 2H), 4.20-4.22 (m, 2H), 7.06 (t, J = 7.2 Hz, 1H), 7.12 (d, J = 5.2 Hz, 1H), 7.23 (d, J = 8.0 Hz, 1H), 7.38-7.42 (m, 1H), 7.60-7.64 (m, 3H), 8.23 (d, J = 5.2 Hz, 1H), 8.60 (s, 1H), 9.14 (s, 1H), 10.32 (s, 1H). LRMS (ESI+) [M + H]⁺; 391.4 |
| 34 | 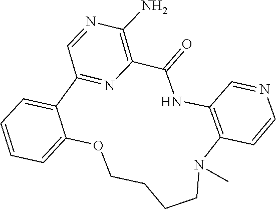 [SMILES: CN1CCCCOc2ccccc2-c2cnc(N)c(n2)C(=O)Nc2cnccc21] | ¹H NMR (400 MHz, Methanol-d₄) δ: 1.69- 1.71 (m, 2H), 1.97-2.05 (m, 2H), 2.82 (s, 3H), 3.04-3.08 (m, 2H), 4.15-4.17 (m, 2H), 7.01-7.05 (m, 2H), 7.11 (d, J = 8.0 Hz, 1H), 7.34-7.41 (m, 2H), 8.12 (d, J = 5.6 Hz, 1H), 8.23 (s, 1H), 8.95 (s, 1H). LRMS (ESI+) [M + H]⁺; 391.5 |
| 45 | 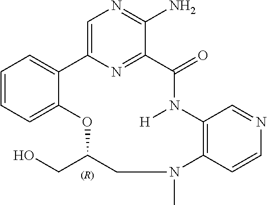 | ¹H NMR (400 MHz, DMSO-d₆) δ: 2.82-2.86 (m, 4H), 3.19-3.25 (m, 1H), 3.43-3.46 (m, 1H), 3.52-3.56 (m, 1H), 4.83-4.98 (m, 2H), 7.05 (t, J = 7.6 Hz, 1H), 7.30-7.47 (m, 5H), 7.80-7.83 (m, 1H), 8.28 (d, J = 5.2 Hz, 1H), 8.70 (s, 1H), 9.68 (s, 1H), 11.57 (s, 1H). LRMS (ESI+) [M + H]⁺; 391.5 |
| 46 | 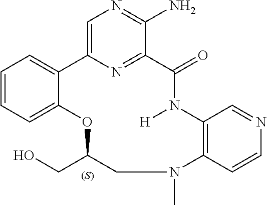 | ¹H NMR (300 MHz, DMSO-d₆) δ: 2.84-2.88 (m, 4H), 3.19-3.28 (m, 1H), 3.40-3.56 (m, 2H), 4.85 (t, J = 5.4 Hz, 1H), 4.99 (s, 1H), 7.05 (t, J = 7.2 Hz, 1H), 7.30-7.49 (m, 5H), 7.80-7.81 (m, 1H), 8.29 (d, J = 5.1 Hz, 1H), 8.72 (s, 1H), 9.69 (s, 1H), 11.59 (s, 1H). LRMS (ESI+) [M + H]⁺; 391.5 |
| 57 | 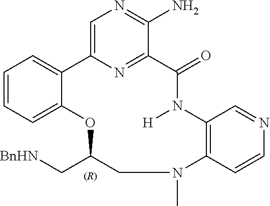 | ¹H NMR (400 MHz, Methanol-d₄) δ: 3.11- 3.14 (m, 4H), 3.27-3.28 (m, 1H), 3.31-3.41 (m, 1H), 3.79-3.86 (m, 1H), 4.10-4.17 (m, 2H), 5.53-5.57 (m, 1H), 7.16 (t, J = 7.6 Hz, 1H), 7.24 (d, J = 8.4 Hz, 1H), 7.29-7.41 (m, 6H), 7.81-7.83 (m, 2H), 8.45-8.47 (m, 1H), 8.69 (s, 1H), 9.87 (s, 1H). |
| 58 | 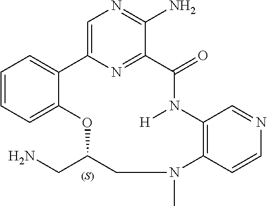 | ¹H NMR (400 MHz, Methanol-d₄) δ: 2.79- 2.83 (m, 2H), 2.84-2.90 (m, 4H), 3.39-3.50 (m, 1H), 5.03-5.07 (s, 1H), 7.07-7.09 (m, 1H), 7.29 (d, J = 8.4 Hz, 1H), 7.38-7.45 (m, 2H), 7.78 (dd, J₁ = 7.6 Hz, J₂ = 1.6 Hz, 1H), 8.24 (d, J = 5.2 Hz, 1H), 8.60 (s, 1H), 9.75 (s, 1H). |
| 59 | 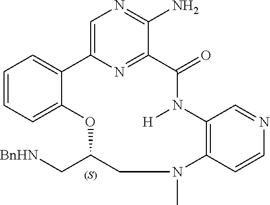 | ¹H NMR (300 MHz, Methanol-d₄) δ: 3.11- 3.15 (m, 3H), 3.26-3.27 (m, 1H), 3.31-3.33 (m, 2H), 3.78-3.86 (m, 1H), 4.13 (s, 2H), 5.52-5.57 (m, 1H), 7.13-7.18 (m, 1H), 7.23- 7.49 (m, 7H), 7.80-7.88 (m, 2H), 8.46 (d, J = 6.6 Hz, 1H), 8.70 (s, 1H), 9.87 (s, 1H). |
| 60 | 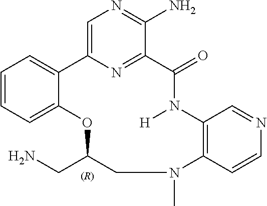 | ¹H NMR (400 MHz, Methanol-d₄) δ: 2.81- 2.84 (m, 2H), 2.87-2.95 (m, 4H), 3.39-3.49 (m, 1H), 5.04-5.06 (m, 1H), 7.09 (t, J = 7.6 Hz, 1H), 7.29 (d, J = 8.4 Hz, 1H), 7.36-7.44 (m, 2H), 7.76-7.78 (m, 1H), 8.23 (d, J = 4.8 Hz, 1H), 8.60 (s, 1H), 9.75 (s, 1H). |
| 61 | 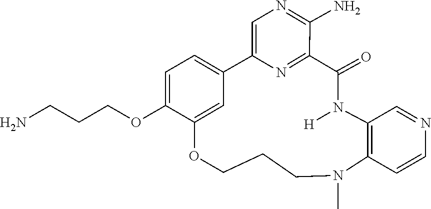 [SMILES: CN1CCCOc2cc(ccc2OCCCN)-c2cnc(N)c(n2)C(=O)Nc2cnccc21] | ¹H NMR (300 MHz, Methanol-d₄) δ: 2.16- 2.29 (m, 4H), 3.02 (s, 3H), 3.21-3.23 (m, 2H), 3.43-3.46 (m, 2H), 4.22-4.26 (m, 2H), 4.47-4.53 (m, 2H), 7.09 (d, J = 8.4 Hz, 1H), 7.56-7.66 (m, 2H), 8.13 (s, 1H), 8.39-8.41 (m, 1H), 8.91 (s, 1H), 9.38 (s, 1H). |
| 62 | 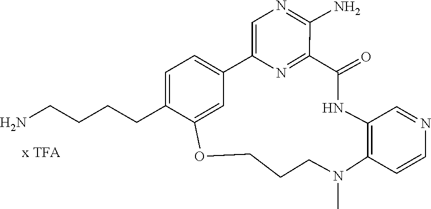 [SMILES: CN1CCCOc2cc(ccc2CCCCN)-c2cnc(N)c(n2)C(=O)Nc2cnccc21] | ¹H NMR (400 MHz, Methanol-d₄) δ: 1.73- 1.75 (m, 4H), 2.16-2.25 (m, 2H), 2.73-2.76 (m, 2H), 2.91-3.01 (m, 5H), 3.42-3.50 (m, 2H), 4.46-4.53 (m, 2H), 7.27-7.29 (m, 1H), 7.52-7.65 (m, 2H), 8.08-8.12 (m, 1H), 8.42 (d, J = 6.4 Hz, 1H), 8.94 (s, 1H), 9.39 (s, 1H). |
| 63 | 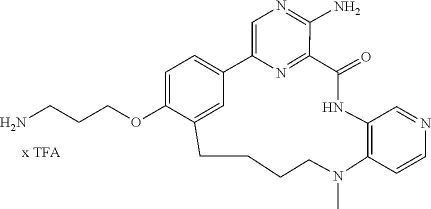 [SMILES: CN1CCCCc2cc(ccc2OCCCN)-c2cnc(N)c(n2)C(=O)Nc2cnccc21] | ¹H NMR (400 MHz, Methanol-d₄) δ: 1.60- 1.70 (m, 2H), 1.90-2.00 (m, 2H), 2.21-2.24 (m, 2H), 2.51-2.69 (m, 2H), 2.71 (s, 3H), 3.15-3.29 (m, 4H), 4.16-4.19 (m, 2H), 6.94 (d, J = 8.4 Hz, 1H), 7.41 (s, 1H), 7.64-7.69 (m, 1H), 8.09 (s, 1H), 8.22-8.39 (m, 1H), 8.68 (s, 1H), 9.30 (s, 1H). |
| 64 | 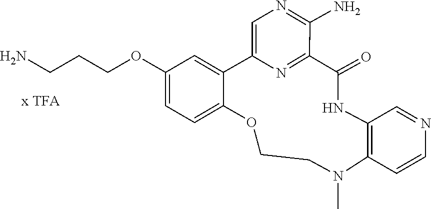 [SMILES: CN1CCOc2ccc(OCCCN)cc2-c2cnc(N)c(n2)C(=O)Nc2cnccc21] | ¹H NMR (400 MHz, Methanol-d₄) δ: 2.14- 2.21 (m, 2H), 3.10 (s, 3H), 3.18-3.22 (m, 2H), 3.61-3.64 (m, 2H), 4.15 (t, J = 5.6 Hz, 2H), 4.44-4.89 (m, 2H), 6.97-6.99 (m, 1H), 7.19 (d, J = 8.8 Hz, 1H), 7.32-7.33 (m, 1H), 7.38-7.40 (m, 1H), 8.23 (d, J = 6.4 Hz, 1H), 8.61 (s, 1H), 9.51 (s, 1H). |
| 65 | 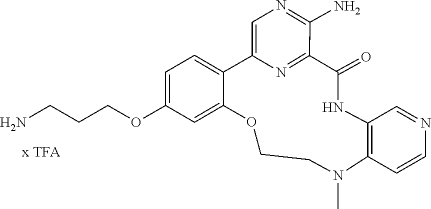 [SMILES: CN1CCOc2cc(OCCCN)ccc2-c2cnc(N)c(n2)C(=O)Nc2cnccc21] | ¹H NMR (400 MHz, Methanol-d₄) δ: 2.18- 2.25 (m, 2H), 3.02-3.08 (m, 3H), 3.20-3.24 (m, 2H), 3.49 (s, 2H), 4.15-4.25 (m, 2H), 4.34-4.35 (m, 2H), 6.63-6.66 (m, 1H), 6.74 (s, 1H), 7.52 (d, J = 6.8 Hz, 1H), 7.61 (d, J = 8.8 Hz, 1H), 8.29 (d, J = 6.8 Hz, 1H), 8.47 (s, 1H), 9.63 (s, 1H). |

In other embodiments, compounds described herein are prepared as described in Scheme 2.

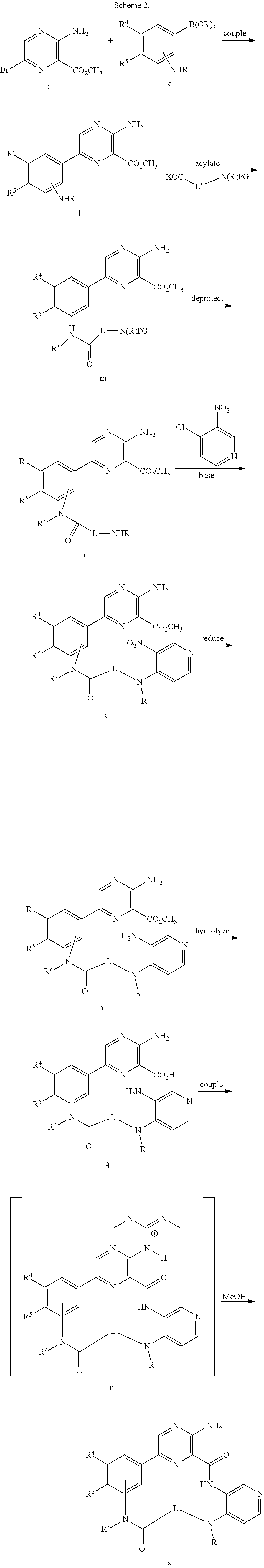

Methyl 3-amino-6-bromopyrazine-2-carboxylate (a) can be coupled to an appropriately substituted arylaminoboronate or boronic acid (k) in the presence of a palladium catalyst such as [1,1′-bis(diphenylphosphino)ferrocene]dichloropalladium(II) and a base such as sodium carbonate at elevated temperatures to afford biaryl intermediates (1). Acylation of compounds (1) using a protected ω-amino acid compound and an amide coupling agent such as BOP or HATU can provide amides (m) which can be deprotected to afford intermediates (n) using a strong acid such as HCl or TFA in cases where PG=Boc or catalytic hydrogenation in cases where PG=Cbz. Amines (n) can react with 4-chloro-3-nitropyridine to provide compounds (o) which can be reduced to amines (p) using standard conditions for the conversion of an aromatic nitro group to an aryl amine such as iron in ammonium chloride solution or a palladium catalyzed hydrogenation reaction. Hydrolysis of the carboxylic ester functional group can be effected by reacting intermediate (p) with a metal alkoxide base such as LiOH to provide carboxylic acids (q) which can undergo a macrolactamization using an amide coupling reagent such as HATU and a tertiary amine base such as DIPEA. Intermediate macrolactams (r) can be converted to the desired targets (s) after brief exposure to methanol followed by purification.

### Example 2: Preparation of 2⁵-amino-6-methyl-4,6,11-triaza-2(2,6)-pyrazina-5(3,4)-pyridina-1(1,3)-benzenacycloundecaphane-3,10-dione (Compound 28)

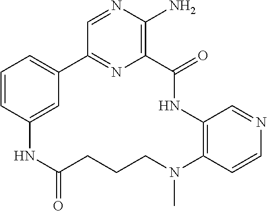 [SMILES: CN1CCCC(=O)Nc2cccc(c2)-c2cnc(N)c(n2)C(=O)Nc2cnccc21]

### Step A: Preparation of Int 2.1a

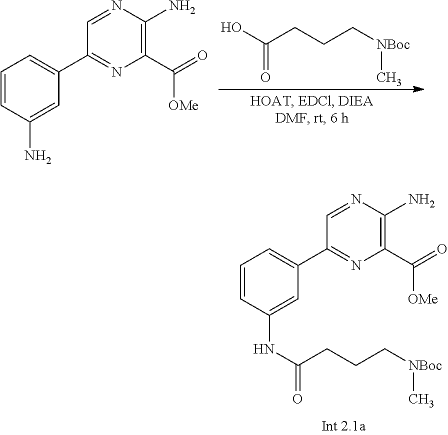

To a solution of 4-((tert-butoxycarbonyl)(methyl)amino)butanoic acid (695 mg, 3.2 mmol, 1.0 equiv) in DMF (10 mL) was added DIEA (1.7 mL, 9.6 mmol, 3.0 equiv). The solution was then treated with EDCI (990 mg, 6.4 mmol, 2.0 equiv), HOAT (870 mg, 6.4 mmol, 2.0 equiv), and methyl 3-amino-6-(3-aminophenyl)pyrazine-2-carboxylate (800 mg, 3.27 mmol, 1.5 equiv). The reaction was stirred at room temperature for 6 hrs and then diluted with water and extracted into EtOAc. The organic layer was dried over Na2SO4, filtered and concentrated. The resultant residue was purified via column chromatography (Silica) 0→100% EtOAc in dichloromethane to provide 600 mg (1.35 mmol, 42% yield) of compound Int 2.1a. M+H=444.5.

### Steps B-C: Preparation of Int 2.1c

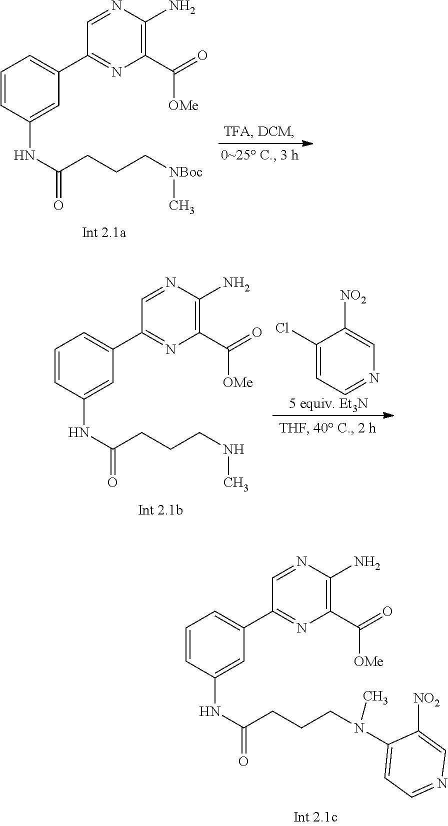

Compound Int 2.1a (700 mg, 1.6 mmol) was dissolved in 15 mL of dichloromethane then treated with 10 mL of TFA and stirred at room temperature for 3h. The solvents were removed and the residue was azeotroped from toluene three times to provide 530 mg of compound Int 2.1b as the TFA salt. M+H=344.2.

Compound Int 2.1b (530 mg, 1.5 mmol) was then dissolved in 15 mL of THF then treated with 0.91 mL (6.5 mmol) of triethylamine and 205 mg (1.3 mmol) of 4-chloro-3-nitropyridine and the mixture was heated at 40 C for 2 h. The mixture was cooled and diluted with 45 mL of EtOAc then washed with water (3×) and brine (1×). The organic extracts were dried over Na2SO4, evaporated and purified by reverse phase chromatography to afford 440 mg (1.0 mmol, 63% yield over two steps) of compound Int 2.1c as a yellow solid. M+H=436.4.

### Step D: Preparation of Int 2.1d

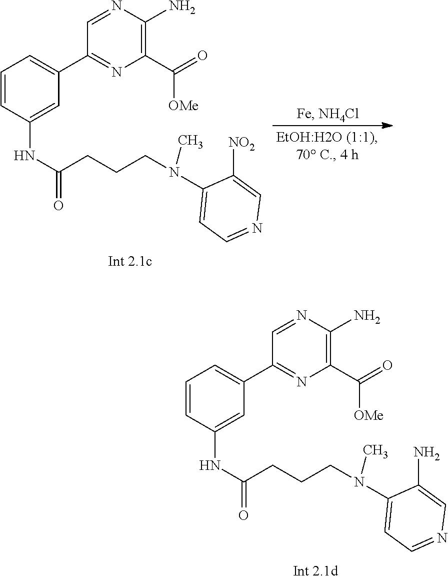 [SMILES: COC(=O)c1nc(-c2cccc(NC(=O)CCCN(C)c3ccncc3[N+](=O)[O-])c2)cnc1N ; COC(=O)c1nc(-c2cccc(NC(=O)CCCN(C)c3ccncc3N)c2)cnc1N]

To a solution of compound Int 2.1c (350 mg, 0.8 mmol) in a mixture of EtOH (8 mL) and NH₄Cl (sat. aq.) (8 mL), was added solid iron powder (450 mg, 8 mmol, 10 equiv). The resultant mixture was heated with vigorous stirring at 70° C., for 4 hrs. The reaction was then cooled to room temperature and filtered through a pad of celite and washed with MeOH. The solvent was removed via rotary evaporator and the residue was washed with water and extracted into dichloromethane. The organic layers were dried over Mg2SO4, filtered and concentrated. The resultant residue was purified via column chromatography (Silica) 0→15% MeOH in dichloromethane to provide 230 mg (0.53 mmol, 66% yield) of the compound Int 2.1d. M+H=436.2.

### Step E: Preparation of Int 2.1e

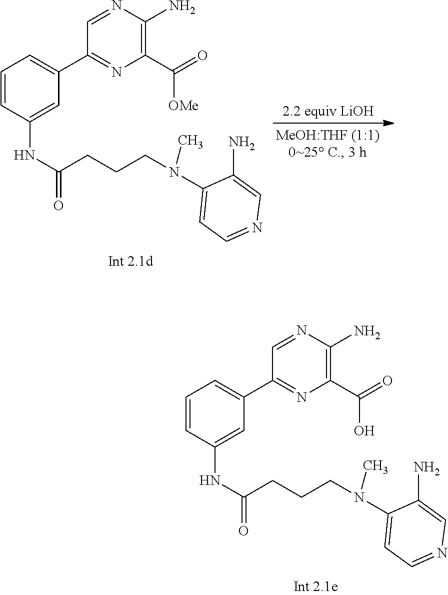 [SMILES: COC(=O)c1nc(-c2cccc(NC(=O)CCCN(C)c3ccncc3N)c2)cnc1N ; CN(CCCC(=O)Nc1cccc(-c2cnc(N)c(C(=O)O)n2)c1)c1ccncc1N]

Compound Int 2.1d (230 mg, 0.53 mmol) was dissolved in 2 mL of THF and 2 mL of methanol at room temperature then treated with 2.2 equivalents LiOH. The mixture was stirred for 3h then evaporated to dryness. The residue was azeotroped with toluene. This process was repeated three times, the residue was then purified via column chromatography (C18) 0→85% acetonitrile (0.1% TFA) in water (0.1% TFA), the pure fractions were pooled, frozen and dried via lyophilization to provide 110 mg (0.26 mmol, 50% yield) of compound Int 2.1e. M−H=420.2.

### Step F: Preparation of Compound 28

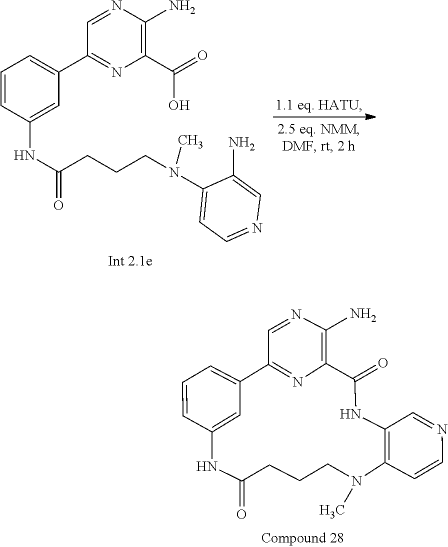 [SMILES: CN(CCCC(=O)Nc1cccc(-c2cnc(N)c(C(=O)O)n2)c1)c1ccncc1N ; CN1CCCC(=O)Nc2cccc(c2)-c2cnc(N)c(n2)C(=O)Nc2cnccc21]

A mixture containing 80 mg (0.19 mmol) of compound Int 2.1e in 10 mL of DMF was treated with 155 mg (0.41 mmol) of HATU and 0.13 mL (0.73 mmol) of NMM then stirred at room temperature for 4h. The reaction was then quenched with 1 mL of methanol and 1 mL of ammonium hydroxide solution and stirred for 1h. The solvents were removed and the residue was chromatographed by reverse phase chromatography to provide 24 mg of Compound 28 as a yellow solid. ¹H NMR (300 MHz, DMSO-d₆) δ: 2.06 (s, 5H), 3.21 (s, 4H), 7.20-7.25 (m, 3H), 7.67 (s, 2H), 7.82 (s, 1H), 7.92 (s, 1H), 8.25 (s, 1H), 8.46 (s, 1H), 8.61 (s, 1H), 9.37 (s, 1H), 10.41 (s, 1H), 13.90 (s, 1H). LRMS (ESI+) [M+H]⁺; 404.5.

The compounds in Table 3 below were prepared in a manner analogous to that described above in Scheme 2 and for the synthesis of Compound 28 in Example 2.

TABLE 3

| Cmpd | Structure | ¹H NMR/LRMS |
|---|---|---|
| 26 | 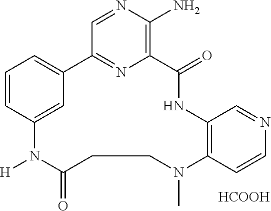 [SMILES: CN1CCC(=O)Nc2cccc(c2)-c2cnc(N)c(n2)C(=O)Nc2cnccc21 ; "HCOOH"] | ¹H NMR (300 MHz, DMSO-d₆ + D₂O) δ: 2.67- 2.78 (m, 5H), 3.59-3.69 (m, 2H), 6.70-6.94 (m, 1H), 7.05 (m, 1H), 7.25-7.31 (m, 2H), 7.90-7.98 (m, 1H), 8.17 (s, 1H), 8.24-8.26 (s, 1H), 8.47 (s, 1H), 9.15 (s, 1H). LRMS (ESI+) [M + H]⁺; 390.4 |
| 27 | 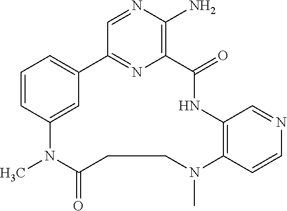 [SMILES: CN1CCC(=O)N(C)c2cccc(c2)-c2cnc(N)c(n2)C(=O)Nc2cnccc21] | ¹H NMR (400 MHz, DMSO-d₆) δ: 2.58 (s, 3H), 2.67-2.71 (m, 2H), 3.31 (s, 3H), 3.31-3.33 (m, 2H), 7.46-7.48 (m, 2H), 7.58 (t, J = 7.6 Hz, 1H), 7.69 (s, 2H), 7.89-7.95 (m, 2H), 8.32 (d, J = 5.2 Hz, 1H), 9.00 (s, 1H), 9.50 (s, 1H), 11.54 (s, 1H). LRMS (ESI+) [M + H]⁺; 404.5 |
| 29 | 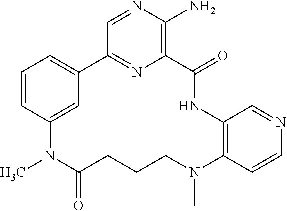 [SMILES: CN1CCCC(=O)N(C)c2cccc(c2)-c2cnc(N)c(n2)C(=O)Nc2cnccc21] | ¹H NMR (400 MHz, DMSO-d₆) δ: 1.87-1.90 (m, 2H), 2.45 (s, 3H), 2.63-2.66 (m, 2H), 3.09- 3.12 (m, 2H), 3.32 (s, 3H), 7.26 (d, J = 5.2 Hz, 1H), 7.38-7.40 (m, 1H), 7.55 (t, J = 7.6 Hz, 1H), 7.74 (s, 2H), 7.81 (d, J = 7.6 Hz, 1H), 8.03 (s, 1H), 8.25 (d, J = 5.6 Hz, 1H), 8.94 (s, 1H), 9.31 (s, 1H), 10.47 (s, 1H). LRMS (ESI+) [M + H]⁺; 418.5 |

In other embodiments, compounds described herein are prepared as described in Scheme 3.

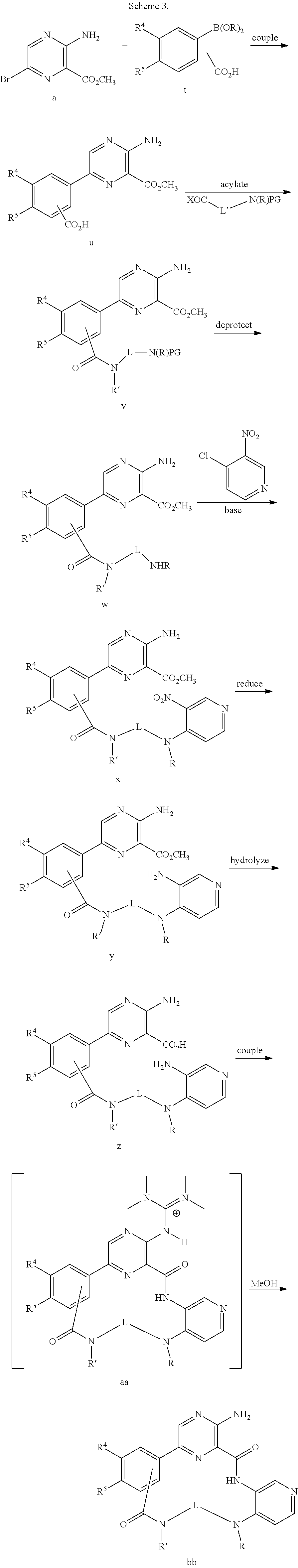

Methyl 3-amino-6-bromopyrazine-2-carboxylate (a) can be coupled to an appropriately substituted carboxy substituted boronate or boronic acid (t) in the presence of a palladium catalyst such as [1,1′-bis(diphenylphosphino)ferrocene]dichloropalladium(II) and a base such as sodium carbonate at elevated temperatures to afford biaryl intermediates (u). Acylation of compounds (u) using a mono-protected diamine and an amide coupling agent such as BOP or HATU can provide amides (v) which can be deprotected to afford intermediates (w) using a strong acid such as HCl or TFA in cases where PG=Boc or catalytic hydrogenation in cases where PG=Cbz. Amines (w) can react with 4-chloro-3-nitropyridine to provide compounds (x) which can be reduced to amines (y) using standard conditions for the conversion of an aromatic nitro group to an aryl amine such as iron in ammonium chloride solution or a palladium catalyzed hydrogenation reaction. Hydrolysis of the carboxylic ester functional group can be effected by reacting intermediate (y) with a metal alkoxide base such as LiOH to provide carboxylic acids (z) which can undergo a macrolactamization using an amide coupling reagent such as HATU and a tertiary amine base such as DIPEA. Intermediate macrolactams (aa) can be converted to the desired targets (bb) after brief exposure to methanol followed by purification.

### Example 3: Preparation of 2⁵-amino-6-methyl-4,6,9-triaza-2(2,6)-pyrazina-5(3,4)-pyridina-1(1,3)-benzenacyclodecaphane-3,10-dione (Compound 30)

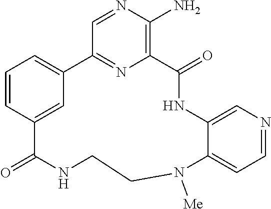 [SMILES: CN1CCNC(=O)c2cccc(c2)-c2cnc(N)c(n2)C(=O)Nc2cnccc21]

### Step A: Preparation of Int 3.1a

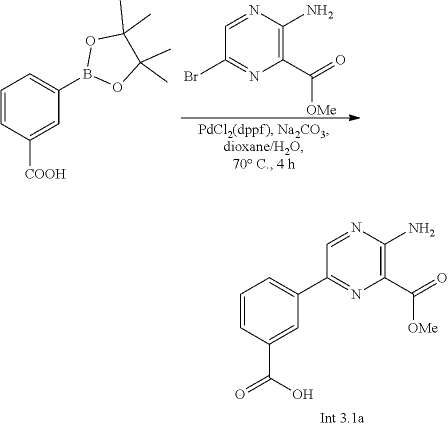

Methyl 3-amino-6-bromopyrazine-2-carboxylate (0.42 g, 1.8 mmol) and 3-(4,4,5,5-tetramethyl-1,3,2-dioxaborolan-2-yl)benzoic acid (0.5 g, 2.0 mmol, 1.1 equiv) were dissolved in 4.5 mL of dioxane and 1.8 mL of 2M Na₂CO₃ solution. The mixture was degassed using nitrogen (5×) before the addition of Pd(dppf)Cl₂ (130 mg, 0.018 mmol). The reaction mixture was degassed again then heated at 70° C. for 4h then cooled and filtered through a plug of Celite. The filtrate was diluted with EtOAc then washed with saturated NaHCO₃ solution (2×), water (1×) then brine. The organic extracts were dried over Na₂SO₄, evaporated and chromatographed (silica) 0%→60% EtOAc in dichloromethane) to afford 385 g (1.4 mmol, 70% yield) of compound Int 3.1a as a yellow solid. M+H=274.2.

### Step B: Preparation of Int 3.1b

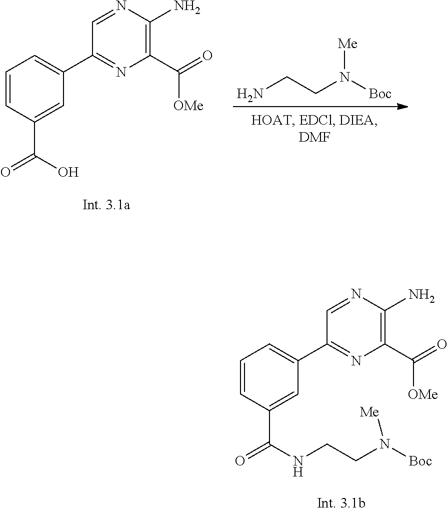 [SMILES: COC(=O)c1nc(-c2cccc(C(=O)O)c2)cnc1N ; CN(CCN)C(=O)OC(C)(C)C ; COC(=O)c1nc(-c2cccc(C(=O)NCCN(C)C(=O)OC(C)(C)C)c2)cnc1N]

To a solution of compound Int 3.1a (600 mg, 2.1 mmol, 1.0 equiv) in DMF (7 mL) was added DIEA (1.3 mL, 9.6 mmol, 3.0 equiv). The solution was then treated with EDCI (990 mg, 4.2 mmol, 2.0 equiv), HOAT (570 mg, 4.2 mmol, 2.0 equiv), and tert-butyl (2-aminoethyl)(methyl)carbamate (525 mg, 2.15 mmol, 1.5 equiv). The reaction was stirred at room temperature for 6 hrs and then diluted with water and extracted into EtOAc. The organic layer was dried over Na₂SO₄, filtered and concentrated. The resultant residue was purified via column chromatography (Silica) 0→100% EtOAc in dichloromethane to provide 540 mg (1.26 mmol, 60% yield) of compound Int 3.1b. M+H=430.5.

### Steps C-D: Preparation of Int 3.1d

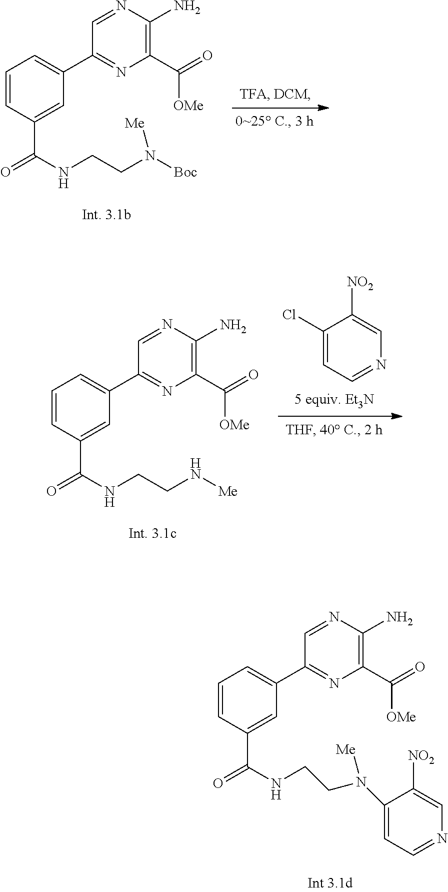 [SMILES: COC(=O)c1nc(-c2cccc(C(=O)NCCN(C)C(=O)OC(C)(C)C)c2)cnc1N ; CNCCNC(=O)c1cccc(-c2cnc(N)c(C(=O)OC)n2)c1 ; O=[N+]([O-])c1cnccc1Cl ; COC(=O)c1nc(-c2cccc(C(=O)NCCN(C)c3ccncc3[N+](=O)[O-])c2)cnc1N]

Compound Int 3.1b (700 mg, 1.62 mmol) was dissolved in 15 mL of dichloromethane then treated with 10 mL of TFA and stirred at room temperature for 3h. The solvents were removed and the residue was covered in toluene and evaporated. This procedure was repeated three times to provide 550 mg of compound Int 3.1c as the TFA salt. M+H=330.4.

Compound Int 3.1c (550 mg, 1.6 mmol) was dissolved in 20 mL of THF then treated with 1.40 mL (10.0 mmol) of triethylamine and 320 mg (2.0 mmol) of 4-chloro-3-nitropyridine and the mixture was heated at 40° C. for 2 h. The mixture was cooled and diluted with EtOAc and washed with water (3×) and brine (1×). The organic extracts were dried over Na₂SO₄, evaporated and purified by reverse phase chromatography to afford 400 mg of compound Int 3.1d as a yellow solid. M+H=438.4.

### Step E: Preparation of Int 3.1e

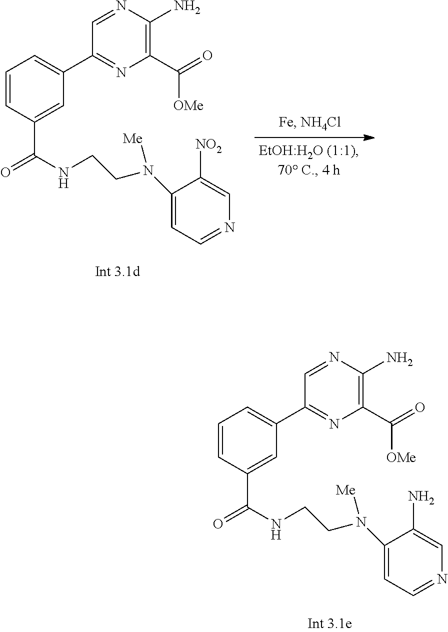 [SMILES: COC(=O)c1nc(-c2cccc(C(=O)NCCN(C)c3ccncc3[N+](=O)[O-])c2)cnc1N ; COC(=O)c1nc(-c2cccc(C(=O)NCCN(C)c3ccncc3N)c2)cnc1N]

To a solution of compound Int 3.1d (400 mg, 0.91 mmol) in a mixture of EtOH (10 mL) and NH₄Cl (sat. aq.) (10 mL), was added solid iron powder (450 mg, 8 mmol, 10 equiv). The resultant mixture was heated with vigorous stirring at 70° C., for 4 hrs. The reaction was then cooled to room temperature and filtered through a pad of celite and washed with MeOH. The solvent was removed via rotary evaporator and the residue was washed with water and extracted into dichloromethane. The organic layers were dried over Mg₂SO₄, filtered and concentrated. The resultant residue was purified via column chromatography (Silica) 0→15% MeOH in dichloromethane to provide 300 mg (0.53 mmol, 66% yield) of compound Int 3.1e. M+H=408.4.

### Step F: Preparation of Int 3.1f

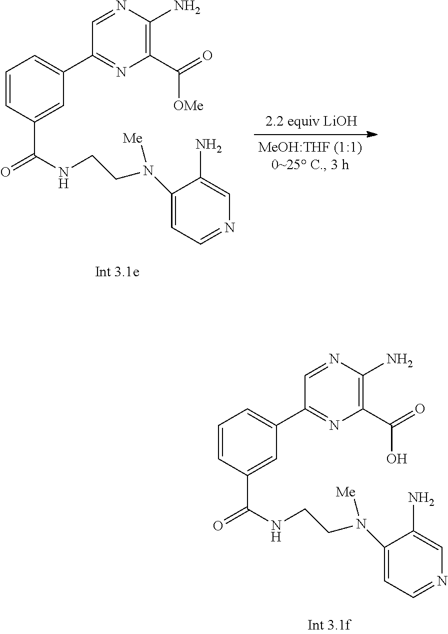 [SMILES: COC(=O)c1nc(-c2cccc(C(=O)NCCN(C)c3ccncc3N)c2)cnc1N ; CN(CCNC(=O)c1cccc(-c2cnc(N)c(C(=O)O)n2)c1)c1ccncc1N]

Compound Int 3.1e (300 mg, 0.5 mmol) was dissolved in 3 mL of THF and 1.5 mL of methanol at room temperature then treated with 2.2 equivalents LiOH. The mixture was stirred for 3h then evaporated to dryness. The residue was azeotroped with toluene. This process was repeated three times, the residue was then purified via column chromatography (C18) 0→85% acetonitrile (0.1% TFA) in water (0.1% TFA), the pure fractions were pooled, frozen and dried via lyophilization to provide 110 mg (0.27 mmol, 54% yield) of compound Int 3.1f. M−H=406.1.

### Step G: Preparation of Compound 30

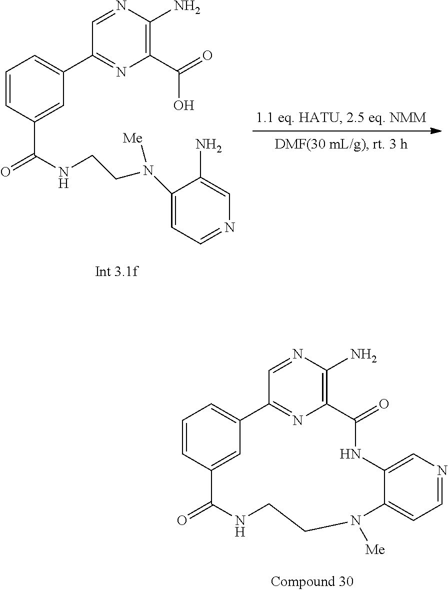 [SMILES: CN(CCNC(=O)c1cccc(-c2cnc(N)c(C(=O)O)n2)c1)c1ccncc1N ; CN1CCNC(=O)c2cccc(c2)-c2cnc(N)c(n2)C(=O)Nc2cnccc21]

A mixture containing 110 mg (0.27 mmol) of compound Int 3.1f in 3.3 mL of DMF was treated with 240 mg (0.62 mmol) of HATU and 0.195 mL (1.15 mmol) of NMM then stirred at room temperature for 4h. The reaction was then quenched with 1 mL of methanol and 1 mL of ammonium hydroxide solution and stirred for 1h. The solvents were removed, and the residue was chromatographed by reverse phase chromatography to provide 20 mg of Compound 30. ¹H NMR (300 MHz, DMSO-d₆) δ: 2.87 (s, 3H), 3.31-3.36 (m, 2H), 3.45-3.62 (m, 2H), 7.09-7.14 (m, 1H), 7.33-7.52 (m, 2H), 7.64-7.95 (m, 3H), 8.02 (s, 1H), 8.24-8.52 (m, 2H), 8.60 (s, 1H), 9.47 (s, 1H), 10.76 (m, 1H). LRMS (ESI+) [M+H]⁺: 390.4.

The compounds in Table 4 below were prepared in a manner analogous to that described above in Scheme 3 and for the synthesis of Compound 30 in Example 3.

TABLE 4

| Cmpd | Structure | ¹H NMR/LRMS |
|---|---|---|
| 31 | 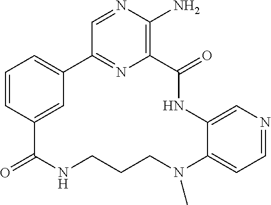 [SMILES: CN1CCCNC(=O)c2cccc(c2)-c2cnc(N)c(n2)C(=O)Nc2cnccc21] | ¹H NMR (300 MHz, DMSO-d₆) δ: 2.06 (s, 4H), 3.21(s, 3H), 3.54 (s, 2H), 7.18-7.26 (m, 3H), 7.69 (s, 2H), 7.83 (s, 1H), 7.93 (s, 1H), 8.26 (d, J = 6.9 Hz, 1H), 8.47 (s, 1H), 8.61 (s, 1H), 9.38 (s, 1H), 10.42 (s, 1H). LRMS (ESI+) [M + H]⁺; 404.5 |
| 53 | 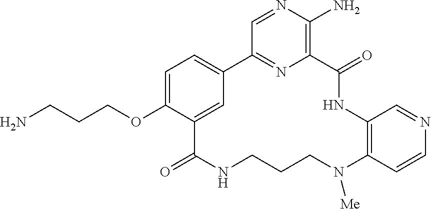 [SMILES: CN1CCCNC(=O)c2cc(ccc2OCCCN)-c2cnc(N)c(n2)C(=O)Nc2cnccc21] | LRMS (ESI+) [M + H]+; 477.5 |
| 54 | 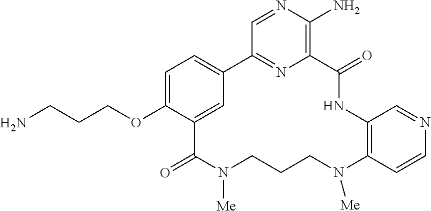 [SMILES: CN1CCCN(C)c2ccncc2NC(=O)c2nc(cnc2N)-c2ccc(OCCCN)c(c2)C1=O] | LRMS (ESI+) [M + H]+; 491.6 |
| 55 | 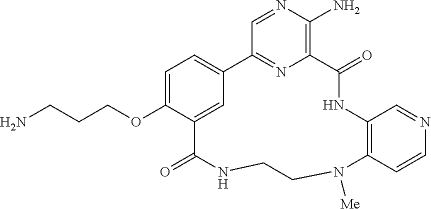 [SMILES: CN1CCNC(=O)c2cc(ccc2OCCCN)-c2cnc(N)c(n2)C(=O)Nc2cnccc21] | LRMS (ESI+) [M + H]+; 463.5 |
| 56 | 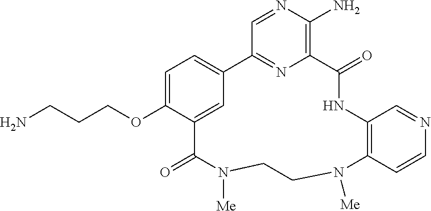 [SMILES: CN1CCN(C)c2ccncc2NC(=O)c2nc(cnc2N)-c2ccc(OCCCN)c(c2)C1=O] | LRMS (ESI+) [M + H]+; 477.5 |

In other embodiments, compounds described herein are prepared as described in Scheme 4.

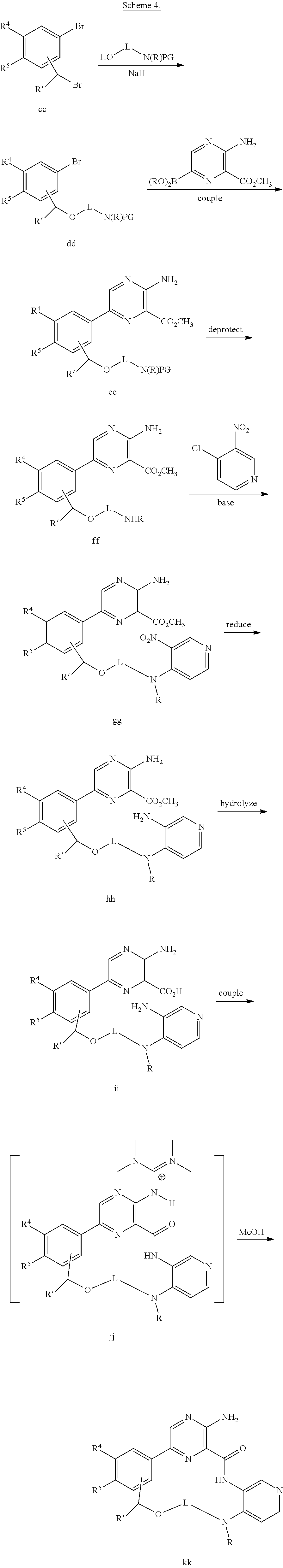

Benzylic halides (cc) can react with protected amino alcohols to provide ethers (dd) which can be coupled with methyl 3-amino-6-(4,4,5,5-tetramethyl-1,3,2-dioxaborolan-2-yl)-2-pyrazinecarboxylate in the presence of a palladium catalyst such as [1,1′-bis(diphenylphosphino)ferrocene]dichloropalladium(II) and a base such as sodium carbonate at elevated temperatures to afford biaryl intermediates (ee) which can be deprotected to afford intermediates (ff) using a strong acid such as HCl or TFA in cases where PG=Boc. Amines (ff) can react with 4-chloro-3-nitropyridine to provide compounds (gg) which can be reduced to amines (hh) using standard conditions for the conversion of an aromatic nitro group to an aryl amine such as iron in ammonium chloride solution. Hydrolysis of the carboxylic ester functional group can be effected by reacting intermediate (hh) with a metal alkoxide base such as LiOH to provide carboxylic acids (ii) which can undergo a macrolactamization using an amide coupling reagent such as HATU and a tertiary amine base such as DIPEA. Intermediate macrolactams (jj) can be converted to the desired targets (kk) after brief exposure to methanol followed by purification.

### Example 4: Preparation of 2⁵-amino-6-methyl-9-oxa-4,6-diaza-2(2,6)-pyrazina-5(3,4)-pyridina-1(1,3)-benzenacyclodecaphan-3-one (Compound 15)

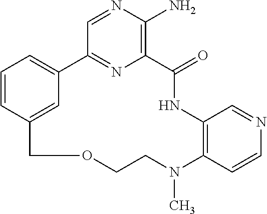 [SMILES: CN1CCOCc2cccc(c2)-c2cnc(N)c(n2)C(=O)Nc2cnccc21]

### Step A: Preparation of Int 4.1a

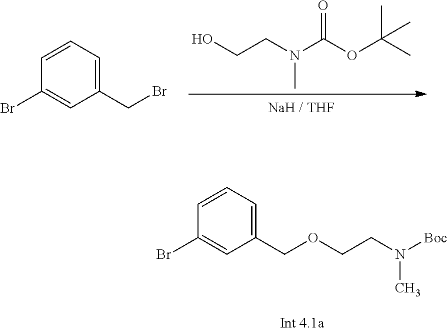 [SMILES: BrCc1cccc(Br)c1 ; CN(CCO)C(=O)OC(C)(C)C ; CN(CCOCc1cccc(Br)c1)C(=O)OC(C)(C)C]

To a solution of tert-butyl N-(2-hydroxyethyl)-N-methylcarbamate (2 g, 11.41 mmol) in THF (15 mL) at 0° C. was added NaH (450 mg, 18.75 mmol). The resulting solution was stirred for 2 h at 0° C. and then 3-bromobenzyl bromide (2.9 g, 11.69 mmol) was added dropwise with stirring at 0° C. The resulting solution was stirred for 4 h at room temperature and then quenched with saturated NH₄Cl solution. The resulting solution was extracted with ethyl acetate and the organic layers were combined, washed with saturated NaCl, dried over Na₂SO₄, filtered and concentrated. The residue was purified by chromatography (ethyl acetate/petroleum ether (1:10)) to afford 2.3 g of Int 4.1a as a white solid.

### Step B: Preparation of Int 4.1 b

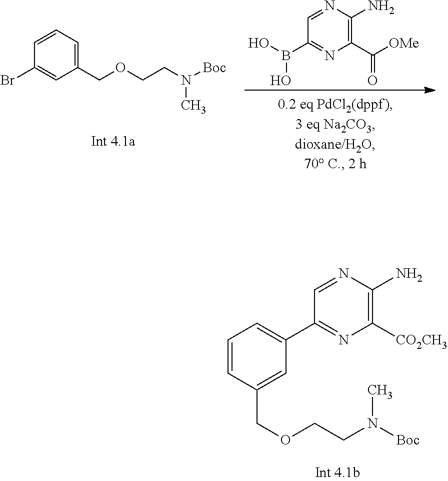 [SMILES: CN(CCOCc1cccc(Br)c1)C(=O)OC(C)(C)C ; COC(=O)c1nc(B(O)O)cnc1N ; COC(=O)c1nc(-c2cccc(COCCN(C)C(=O)OC(C)(C)C)c2)cnc1N]

A solution of 686 mg (2.0 mmol) of Int 2.1a and 560 mg (2.0 mmol) of methyl 3-amino-6-(4,4,5,5-tetramethyl-1,3,2-dioxaborolan-2-yl)-2-pyrazinecarboxylic acid were dissolved in 10 mL of dioxane and 3.0 mL of 2M Na₂CO₃ solution. The mixture was degassed using nitrogen (5×) before the addition of Pd(dppf)Cl₂ (295 mg, 0.4 mmol). The reaction mixture was degassed again then heated at 70° C. for 2 h then cooled and filtered through a plug of Celite. The filtrate was diluted with EtOAc then washed with saturated NaHCO₃ solution (2×), water (1×) then brine. The organic extracts were dried over Na₂SO₄, evaporated and chromatographed (ISCO Gold; 0% to 100% EtOAc /dichloromethane) to afford compound Int 4.1b as a yellow solid. M+H=417.2.

### Step C: Preparation of Int 4.1c

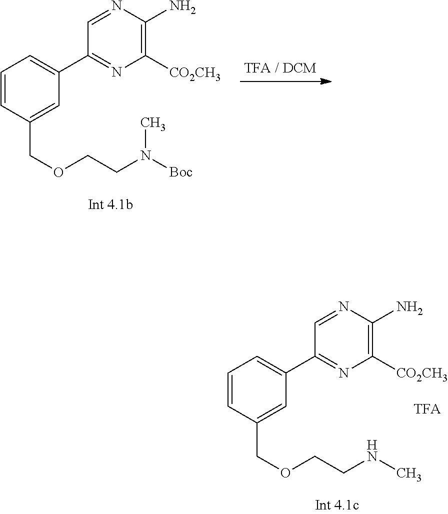 [SMILES: COC(=O)c1nc(-c2cccc(COCCN(C)C(=O)OC(C)(C)C)c2)cnc1N ; CNCCOCc1cccc(-c2cnc(N)c(C(=O)OC)n2)c1]

Compound Int 4.1b (310 mg, 0.75 mmol) was dissolved in 15 mL of dichloromethane then treated with 5 mL of TFA and stirred at room temperature for 3h. The solvents were removed and the residue was covered in toluene and evaporated. This procedure was repeated three times to provide 320 mg of the desired compound Int 4.1c as the TFA salt. M+H=317.2.

### Step D: Preparation of Int 4.1d

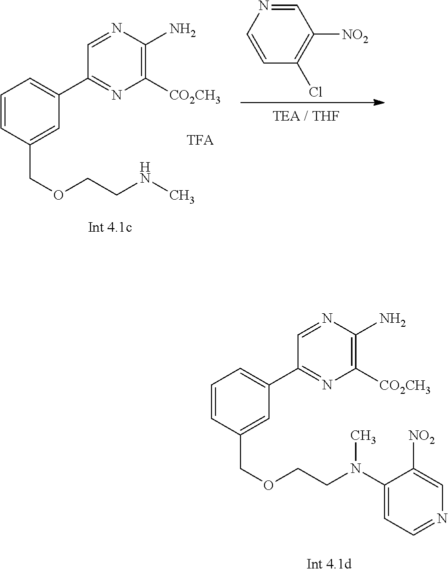 [SMILES: CNCCOCc1cccc(-c2cnc(N)c(C(=O)OC)n2)c1 ; O=[N+]([O-])c1cnccc1Cl ; COC(=O)c1nc(-c2cccc(COCCN(C)c3ccncc3[N+](=O)[O-])c2)cnc1N]

Compound Int 4.1c (320 mg, 0.99 mmol) was dissolved in 5 mL of THF then treated with 0.70 mL (5.0 mmol) of triethylamine and 158 mg (1.0 mmol) of 4-chloro-3-nitropyridine and the mixture was heated at 40° C. for 2 h. The mixture was cooled and diluted with 15 mL of EtOAc then washed with water (3×) and brine (1×). The organic extracts were dried over Na₂SO₄, evaporated and purified by reverse phase chromatography to afford 320 mg of compound Int 4.1d as a yellow solid. M+H=439.4.

### Step E: Preparation of Int 4.1e

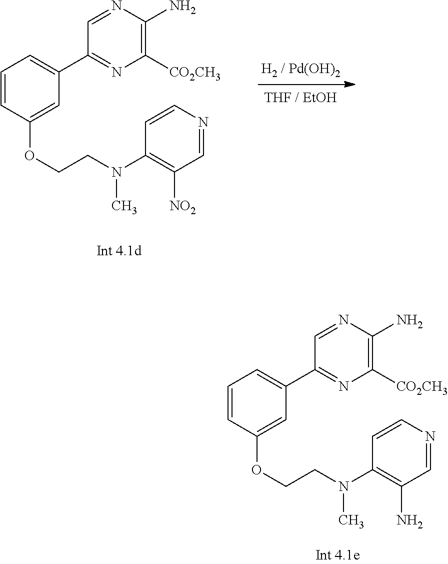 [SMILES: COC(=O)c1nc(-c2cccc(OCCN(C)c3ccncc3[N+](=O)[O-])c2)cnc1N ; COC(=O)c1nc(-c2cccc(OCCN(C)c3ccncc3N)c2)cnc1N]

Nitro compound Int 4.1d (320 mg, 0.73 mmol) was dissolved in 10 mL of THF and 10 mL of EtOH and the mixture was degassed. Fifty milligrams of 20% Pd(OH)₂ was then added and the mixture was covered with a balloon containing hydrogen gas. The reaction was stirred at room temperature for 2 h then filtered through Celite. The filtrate was evaporated to afford 160 mg of the desired product Int 4.1e as a tan colored solid. M+H=409.2.

### Step F: Preparation of Int 4.1f

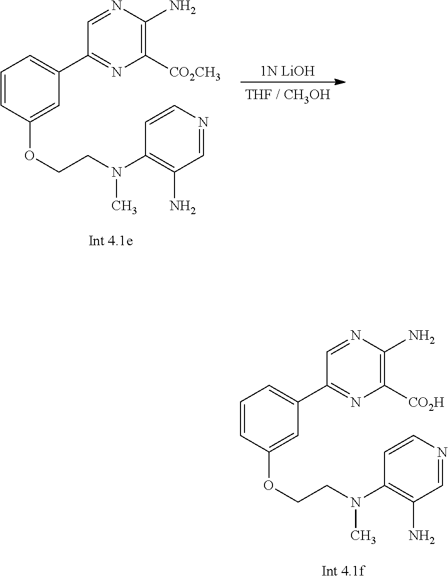 [SMILES: COC(=O)c1nc(-c2cccc(OCCN(C)c3ccncc3N)c2)cnc1N ; "1N LiOH" ; CN(CCOc1cccc(-c2cnc(N)c(C(=O)O)n2)c1)c1ccncc1N]

Compound Int 4.1e (160 mg, 0.39 mmol) was dissolved in 2 mL of THF and 1 mL of methanol at room temperature then treated with 2.5equivalents (1.0 mL) of 1N LiOH. The mixture was stirred for 2 h then evaporated to dryness. The residue was covered with 5 mL of toluene and evaporated. This procedure was repeated three times to leave 70 mg of the desired amino acid Int 4.1f which was used directly in the next step. M+H=395.1.

### Step G: Preparation of Compound 15

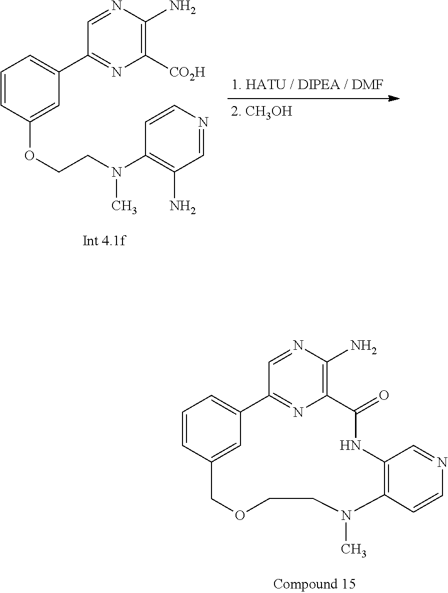 [SMILES: CN(CCOc1cccc(-c2cnc(N)c(C(=O)O)n2)c1)c1ccncc1N ; CN1CCOCc2cccc(c2)-c2cnc(N)c(n2)C(=O)Nc2cnccc21]

A mixture containing 40 mg (0.1 mmol) of Int 2.1f in 10 mL of DMF was treated with 85 mg (0.225 mmol) of HATU and 0.056 mL (0.4 mmol) of NMM then stirred at room temperature for 3h. The reaction was then quenched with 1 mL of methanol and 1 mL of ammonium hydroxide solution and stirred for 1h. The solvents were removed and the residue was chromatographed by reverse phase chromatography to provide Compound 15 as a yellow solid. ¹H NMR (DMSO-d⁶) δ 11.5 (bs, 1H), 9.42 (s, 1H), 9.06 (s, 1H), 8.88 (s, 1H), 8.65 (s, 1H), 7.96 (d, J=7.8 Hz, 1H), 7.63 (s, 2H), 7.43 (t, J=7.8 Hz, 1H), 7.26 (d, J=5.2 Hz, 1H), 7.18 (dd, J=2.4, 7.8 Hz, 1H), 4.73 (s, 2H), 3.88 (m, 2H), 2.78 (s, 3H). M+H=377.1.

The compounds in Table 5 below were prepared in a manner analogous to that described above in Scheme 4 and for the synthesis of Compound 15 in Example 4.

TABLE 5

| Cmpd | Structure | ¹H NMR/LRMS |
|---|---|---|
| 20 | 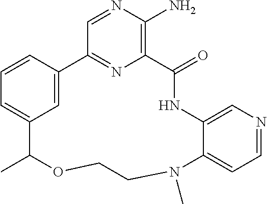 [SMILES: CC1OCCN(C)c2ccncc2NC(=O)c2nc(cnc2N)-c2cccc1c2] | ¹H NMR (400 MHz, DMSO-d₆) δ: 1.39 (d, J = 6.4 Hz, 3H), 2.84 (s, 3H), 3.18 (d, J = 13.2 Hz, 1H), 3.37-3.42 (m, 1H), 3.71-3.84 (m, 2H), 4.57-4.62 (m, 1H), 7.24 (t, J = 4.4 Hz, 2H), 7.43 (d, J = 7.6 Hz, 1H), 7.63 (s, 2H), 7.91 (d, J = 7.6 Hz, 1H), 8.28 (d, J = 5.2 Hz, 1H), 8.55 (s, 1H), 9.03 (s, 1H), 9.39 (s, 1H), 11.53 (s, 1H). |

In other embodiments, compounds described herein are prepared as described in Scheme 5.

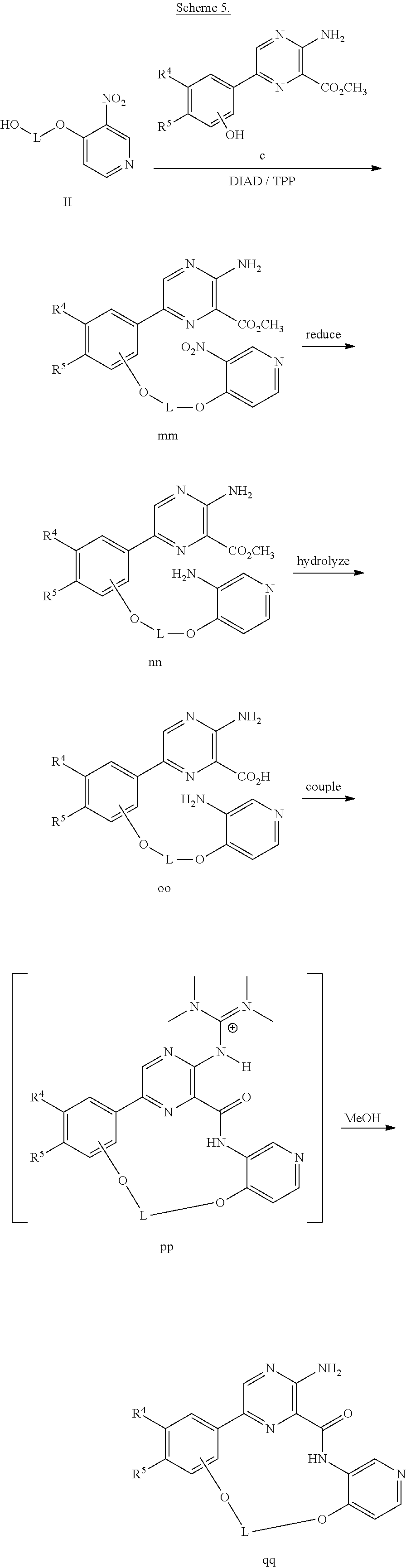

Alcohol intermediates (ll) can react with phenol intermediates (c) using well established Mitsunobu etherification conditions to provide compounds (mm) which can be converted to the amine derivative (nn) using standard conditions for the conversion of an aromatic nitro group to an aryl amine such as iron in ammonium chloride solution. Hydrolysis of the carboxylic ester functional group can be effected by reacting intermediate (nn) with a metal alkoxide base such as LiOH to provide carboxylic acids (pp) which can undergo a macrolactamization using an amide coupling reagent such as HATU and a tertiary amine base such as DIPEA. Intermediate macrolactams (pp) can be converted to the desired targets (qq) after brief exposure to methanol followed by purification.

### Example 5: Preparation of (4¹,4⁴-cis)-1⁵-amino-3,5-dioxa-7-aza-1(2,6)-pyrazina-6(4,3)-pyridina-2(1,3)-benzena-4(1,4)-cyclohexanacyclooctaphan-8-one (Compound 43)

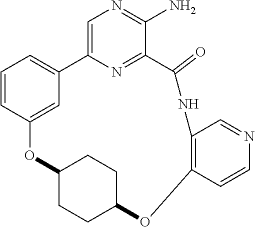

### Step A: Preparation of Int 5.1a

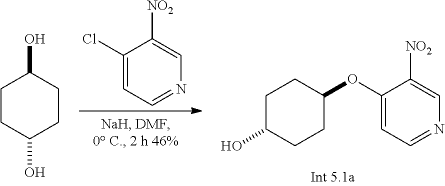

To a solution of trans-cyclohexane-1,4-diol (2.0 g, 17.2 mmol) in DMF (100 mL) at 0° C., was added solid sodium hydride (1.1 equiv., 18.9 mmol, 454 mg); after the effervescence subsided, solid 4-chloro-3-nitropyridine (1.1 equiv., 18.9 mmol, 3.0 g) was added to the reaction and the solution was allowed to warm to room temperature over 2 hrs. The product was then extracted into EtOAc and washed several times with water. The organic layer was concentrated and the residue was purified by reverse phase chromatography to provide 1.6 g (46% yield) of compound Int 5.1a. M+H=240.2.

### Step B: Preparation of Int 5.1b

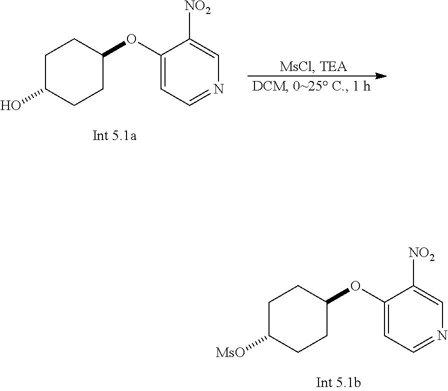 [SMILES: O=[N+]([O-])c1cnccc1O[C@H]1CC[C@H](O)CC1 ; CS(=O)(=O)O[C@H]1CC[C@H](Oc2ccncc2[N+](=O)[O-])CC1]

To a solution of compound Int 5.1a (1.6 g, 6.6 mmol) in DCM, at 0° C. was added Et₃N (1.05 mL, 1.1 equiv, 7.3 mmol) followed by methanesulfonyl chloride (830 mg, 1.1 equiv, 7.3 mmol). The solution was allowed to come to room temperature, the solvent was reduced under reduced pressure and the resultant residue compound Int 5.1b (1.47 g) was used in the subsequent step without additional purification. M+H=317.3.

### Step C: Preparation of Int 5.1c

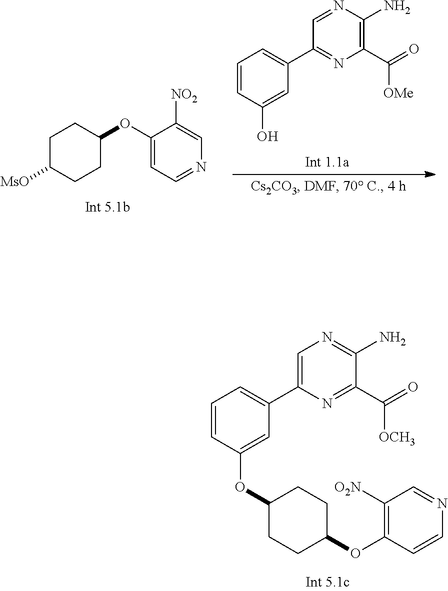

To a vessel containing methyl 3-amino-6-(3-hydroxyphenyl)pyrazine-2-carboxylate (Int 1.1a) (1.0 equiv., 0.42 mmol, 103 mg) and Cs₂CO₃ (3.0 equiv., 1.26 mmol, 410 mg), was added DMF (4 mL). To the stirring solution was then added compound Int 5.1b (1.5 equiv, 200 mg) and the reaction was stirred at 70° C. for 4 hrs. Upon completion of the reaction, the product was purified via reverse phase chromatography (direct loading) to provide 112 mg (49% yield, two steps) of compound Int 5.1c. M+H=466.5.

### Step D: Preparation of Int 5.1d

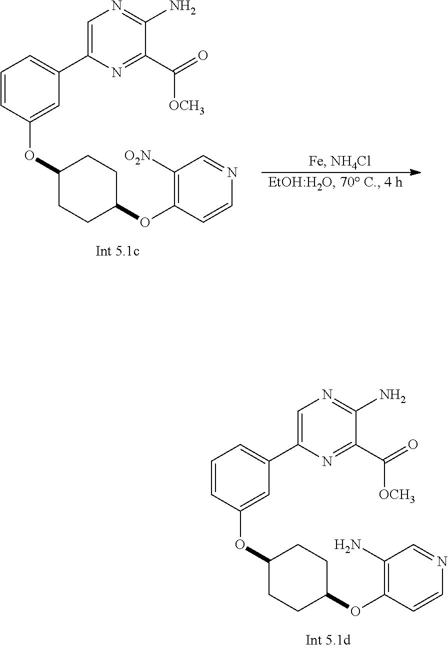

To a solution of compound Int 5.1c (112 mg, 0.24 mmol) in a mixture of EtOH (3 mL) and NH₄Cl (sat. aq.) (3 mL), was added solid iron powder (100 mg, 1.8 mmol, 10 equiv). The resultant mixture was heated with vigorous stirring at 70° C., for 4 hrs. The reaction was then cooled to room temperature and filtered through a pad of celite and washed with MeOH. The solvent was removed via rotary evaporator and the residue was washed with water and extracted into dichloromethane. The organic layers were dried over Mg₂SO₄, filtered and concentrated. The resultant residue was purified via column chromatography (Silica) 0→20% MeOH in dichloromethane to provide 70 mg (0.16 mmol, 67% yield) of compound Int 5.1d. M+H=436.5.

### Step E: Preparation of Compound 43

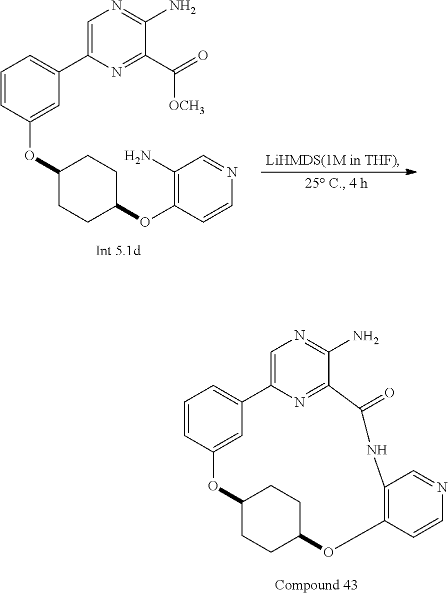

To a solution of compound Int 5.1d (20 mg, 0.046 mmol, 1.0 equiv) in dry THE (1 mL) with an inert atmosphere of nitrogen, was added LiHMDS (1M in THF, 0.23 mL, 0.23 mmol, 5.0 equiv). The resulting solution was stirred at 25° C. for 2 h, then quenched with 5 mL of water, extracted with 3×10 mL of ethyl acetate and the organic layers were combined and dried over anhydrous sodium sulfate, filtered, and concentrated under reduced pressure. The residue was purified by Flash-Prep-HPLC with the following conditions (IntelFlash-1): C18 Column; mobile phase, Water (0.5% NH₄HCO₃) and ACN (10.0% ACN up to 100.0% in 15 min); Detector, UV 254 nm. This resulted in 6.5 mg (35%) of Compound 43 as a yellow solid. M+H=403.2.

The compounds in Table 6 below were prepared in a manner analogous to that described above in Scheme 5 and for the synthesis of Compound 43 in Example 5.

TABLE 6

| Cmpd | Structure | ¹H NMR/LRMS |
|---|---|---|
| 35 | 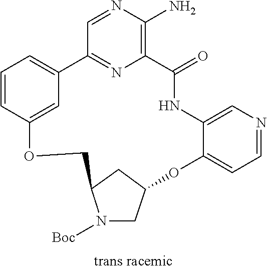 [SMILES: CC(C)(C)OC(=O)N1C[C@@H]2C[C@@H]1COc1cccc(c1)-c1cnc(N)c(n1)C(=O)Nc1cnccc1O2] | ¹H NMR (400 MHz, DMSO-d₆) δ: 1.06-1.22 (m, 10H), 2.50-2.51 (m, 1H), 2.80 (m, 1H), 3.32 (m, 1H), 3.96-4.27 (m, 2H), 4.27 (s, 1H), 5.45 (s, 1H), 6.75-6.95 (m, 1H), 7.19-7.32 (m, 2H), 7.56-7.59 (m, 2H), 7.75 (s, 2H), 8.30 (d, J = 4.4 Hz, 1H), 8.90-8.99 (m, 1H), 9.46-9.50 (m, 1H), 10.38-10.41 (m, 1H). LRMS (ESI+) [M − Boc + H]⁺: 405.6; [M + Na]⁺: 527.7 |
| 36 | 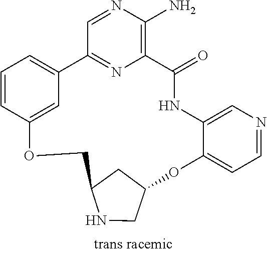 [SMILES: Nc1ncc2nc1C(=O)Nc1cnccc1O[C@@H]1CN[C@@H](COc3cccc-2c3)C1] | ¹H NMR (300 MHz, DMSO-d₆ + D₂O) δ: 2.30 (m, 1H), 2.74-2.78 (m, 1H), 3.49-3.64 (m, 4H), 3.90 (s, 1H), 5.83 (s, 1H), 6.89-6.91 (s, 1H), 7.07 (s, 1H), 7.59 (d, J = 4.2 Hz, 2H), 7.82 (d, J = 6.9 Hz, 1H), 8.63-8.65 (s, 2H), 9.55 (s, 1H). LRMS (ESI+) [M + H]⁺; 405.4 |
| 37 | 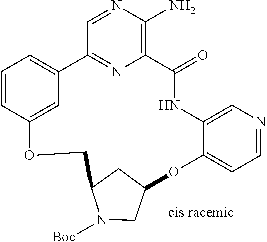 | ¹H NMR (400 MHz, DMSO-d₆) δ: 1.43 (s, 9H), 2.27-2.31 (m, 1H), 2.71-2.77 (m, 1H), 3.52 (s, 1H), 4.25-4.27 (m, 3H), 4.51 (s, 1H), 5.22 (s, 1H), 6.95 (d, J = 7.6 Hz, 1H), 7.13 (s, 1H), 7.36 (t, J = 8.0 Hz, 1H), 7.64-7.69 (m, 3H), 8.19-8.27 (m, 2H), 9.01 (s, 1H), 9.58 (s, 1H), 10.82 (s, 1H). LRMS (ESI+) [M − Boc + H]⁺: 405.6; [M + Na]⁺: 527.7 |
| 38 | 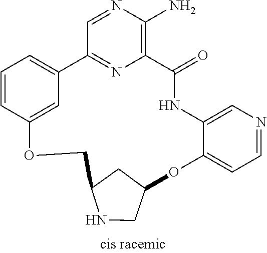 | ¹H NMR (400 MHz, Methanol-d₄) δ: 2.74- 2.81 (s, 1H), 2.89-2.97 (m, 1H), 3.92 (d, J = 4.8 Hz, 2H), 4.11-4.13 (m, 1H), 4.50-4.54 (m, 1H), 4.80-4.81 (m, 1H), 5.61-5.63 (m, 1H), 7.14-7.16 (m, 1H), 7.47 (t, J = 8.0 Hz, 1H), 7.58-7.63 (m, 2H), 8.23-8.25 (m, 1H), 8.50 (d, J = 6.4 Hz, 1H), 8.90 (s, 1H), 9.79 (s, 1H). LRMS (ESI+) [M + H]⁺; 405.4 |
| 39 | 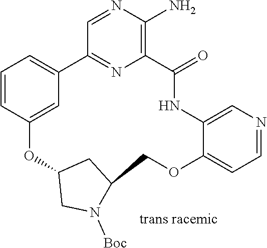 [SMILES: CC(C)(C)OC(=O)N1C[C@H]2C[C@H]1COc1ccncc1NC(=O)c1nc(cnc1N)-c1cccc(c1)O2] | ¹H NMR (400 MHz, DMSO-d₆) δ: 1.39-1.43 (m, 9H), 2.18-2.36 (m, 1H), 2.50-2.51 (m, 1H), 3.42-3.53 (m, 2H), 4.28-4.40 (m, 3H), 4.79 (s, 1H), 6.49-6.51 (m, 1H), 7.15-7.51 (m, 4H), 7.75 (s, 2H), 8.30-8.32 (m, 1H), 8.85 (s, 1H), 9.20-9.26 (m, 1H), 10.21 (d, J = 12.8 Hz, 1H). LRMS (ESI+) [M − Boc + H]⁺: 405.6; [M + Na]⁺: 527.7 |
| 40 | 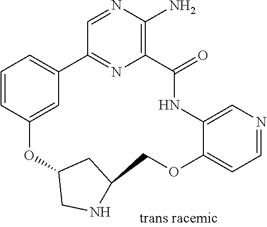 [SMILES: Nc1ncc2nc1C(=O)Nc1cnccc1OC[C@@H]1C[C@H](CN1)Oc1cccc-2c1] | ¹H NMR (300 MHz, DMSO-d₆ + D₂O) δ: 2.50- 2.55 (m, 2H), 3.06-3.10 (m, 1H), 3.30-3.35 (m, 1H), 4.33 (s, 2H), 4.66-4.74 (m, 2H), 6.55-6.58 (s, 1H), 7.30 (s, 1H), 7.44-7.47 (m, 1H), 7.66-7.68 (m, 2H), 8.63 (d, J = 6.6 Hz, 1H), 8.76 (s, 1H), 9.44 (s, 1H). LRMS (ESI+) [M + H]⁺; 405.4 |
| 41 | 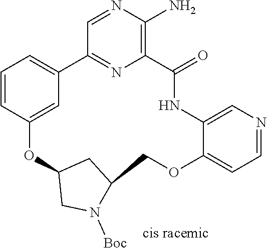 [SMILES: CC(C)(C)OC(=O)N1C[C@@H]2C[C@H]1COc1ccncc1NC(=O)c1nc(cnc1N)-c1cccc(c1)O2] | ¹H NMR (400 MHz, DMSO-d₆) δ: 1.48-1.56 (m, 9H), 1.93 (s, 1H), 2.50-2.58 (m, 1H), 3.54-3.77 (m, 2H), 4.24 (s, 1H), 4.55-4.61 (m, 3H), 7.15-7.16 (m, 1H), 7.17 (s, 1H), 7.47 (t, J = 8.0 Hz, 2H), 7.57 (s, 1H), 7.83 (d, J = 8.0 Hz, 1H), 8.04 (s, 1H), 8.21-8.39 (m, 1H), 8.99 (s, 1H), 9.25 (s, 1H), 11.50 (s, 1H). LRMS (ESI+) [M − Boc + H]⁺: 405.6; [M + Na]⁺: 527.7 |
| 42 | 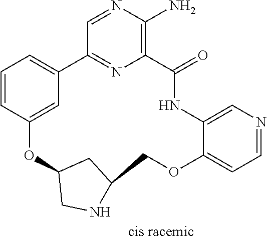 [SMILES: Nc1ncc2nc1C(=O)Nc1cnccc1OC[C@@H]1C[C@@H](CN1)Oc1cccc-2c1] | ¹H NMR (400 MHz, Methanol-d₄) δ: 2.28- 2.87 (m, 1H), 3.00-3.15 (m, 1H), 3.69-3.83 (m, 2H), 4.26 (s, 1H), 4.68-4.93 (m, 2H), 5.14-5.15 (m, 1H), 7.07-7.10 (m, 1H), 7.32 (s, 1H), 7.44 (t, J = 8.0 Hz, 1H), 7.61 (d, J = 8.0 Hz, 1H), 7.94 (s, 1H), 8.16-8.31 (m, 3H), 8.74 (s, 1H), 9.60 (s, 1H). LRMS (ESI+) [M + H]⁺; 405.4 |
| 44 | 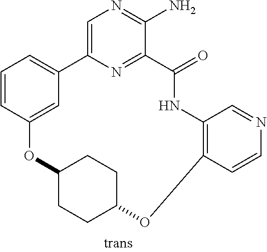 | ¹H NMR (400 MHz, Formic acid-d₂) δ: 0.01 (s, 2H), 0.31-0.34 (m, 4H), 0.43 (s, 2H), 2.51 (s, 2H), 3.41 (s, 1H), 3.38 (s, 1H), 5.58-5.66 (m, 2H), 5.78-5.83 (m, 2H), 6.44-6.49 (m, 2H), 6.79-6.88 (m, 2H), 7.93-7.98 (m, 1H). LRMS (ESI+) [M + H]⁺; 404.4 |
| 49 | 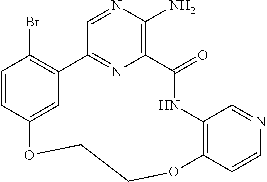 [SMILES: Nc1ncc2nc1C(=O)Nc1cnccc1OCCOc1ccc(Br)c-2c1] | LRMS (ESI+) [M + H]+; 429.2 |
| 50 | 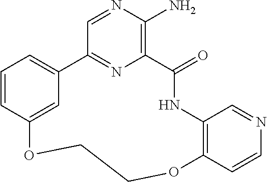 [SMILES: Nc1ncc2nc1C(=O)Nc1cnccc1OCCOc1cccc-2c1] | LRMS (ESI+) [M + H]+; 350.3 |

In other embodiments, compounds described herein are prepared as described in Scheme 6.

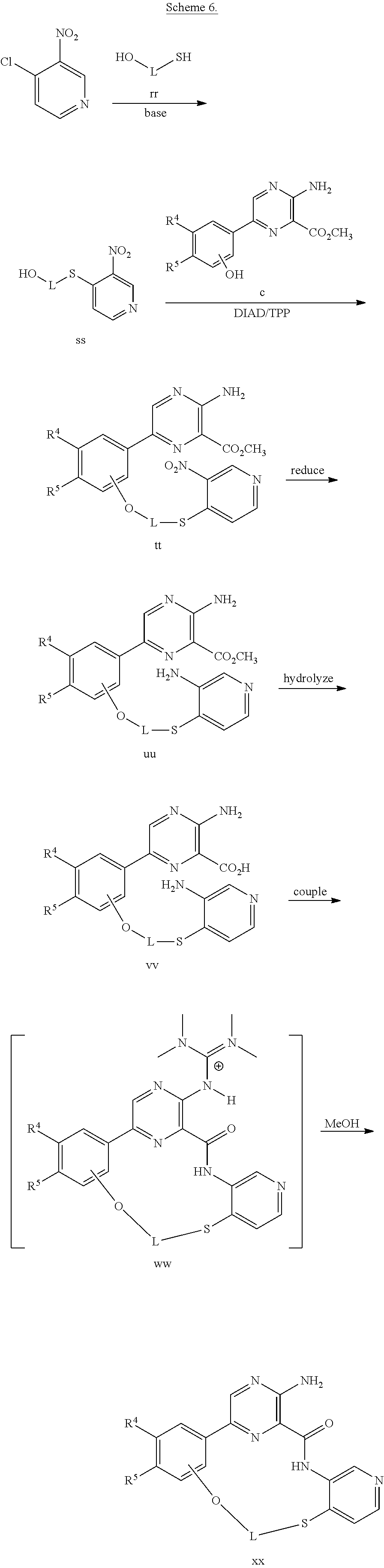

Alcohol intermediates (ss) can react with phenol intermediates (c) using well established Mitsunobu etherification conditions to provide compounds (tt) which can be converted to the amine derivative (uu) using standard conditions for the conversion of an aromatic nitro group to an aryl amine such as iron in ammonium chloride solution. Hydrolysis of the carboxylic ester functional group can be effected by reacting intermediate (uu) with a metal alkoxide base such as LiOH to provide carboxylic acids (vv) which can undergo a macrolactamization using an amide coupling reagent such as HATU and a tertiary amine base such as DIPEA. Intermediate macrolactams (ww) can be converted to the desired targets (xx) after brief exposure to methanol followed by purification.

### Example 6: Preparation of 2⁵-amino-1⁶-bromo-10-oxa-6-thia-4-aza-2(2,6)-pyrazina-5(3,4)-pyridina-1(1,3)-benzenacyclodecaphan-3-one (Compound 51)

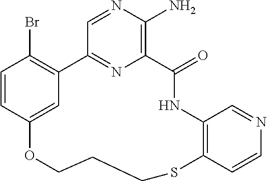 [SMILES: Nc1ncc2nc1C(=O)Nc1cnccc1SCCCOc1ccc(Br)c-2c1]

### Step A: Preparation of Int 6.1a

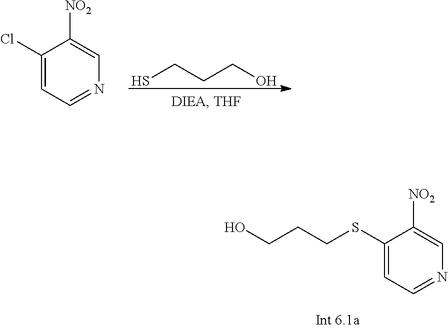 [SMILES: O=[N+]([O-])c1cnccc1Cl ; OCCCS ; O=[N+]([O-])c1cnccc1SCCCO]

To a solution of 3-mercaptopropan-1-ol (1.0 g, 1.0 equiv., 10.9 mmol) in THE (30 mL), was added 4-chloro-3-nitropyridine (1.1 equiv., 12.0 mmol, 1.88 g) followed by DIEA (1.3 equiv., 14.2 mmol, 2.4 mL). The resultant mixture was stirred for 15 hrs, diluted with EtOAc and washed with water. The organic layer was dried over Mg₂SO₄, filtered and concentrated. The resultant residue was purified via column chromatography (Silica) 0→60% EtOAc in dichloromethane to provide 963 mg (4.5 mmol, 41% yield) of compound Int 6.1a. M+H=215.2.

### Step B: Preparation of Int 6.1b

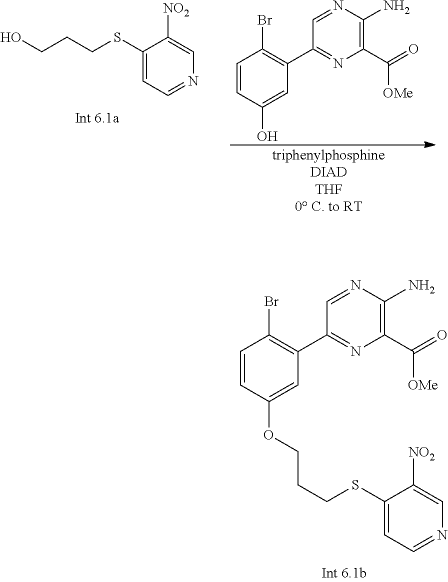 [SMILES: O=[N+]([O-])c1cnccc1SCCCO ; COC(=O)c1nc(-c2cc(O)ccc2Br)cnc1N ; COC(=O)c1nc(-c2cc(OCCCSc3ccncc3[N+](=O)[O-])ccc2Br)cnc1N]

To a mixture containing compound Int 6.1a (9.63 mg, 4.5 mmol), triphenylphosphine (1.18 g, 4.5 mmol) and methyl 3-amino-6-(2-bromo-5-hydroxyphenyl)pyrazine-2-carboxylate (969 mg, 3.0 mmol) in THE at 0° C., was added DIAD (0.88 mL, 4.5 mmol) dropwise. The reaction was stirred for 15 hrs, concentrated onto silica gel and purified via column chromatography (Silica) 0→50% EtOAc in hexanes to provide 489 mg (0.92 mmol, 31% yield) of compound Int 6.1b. M+H=520.2.

### Step C: Preparation of Int 6.1c

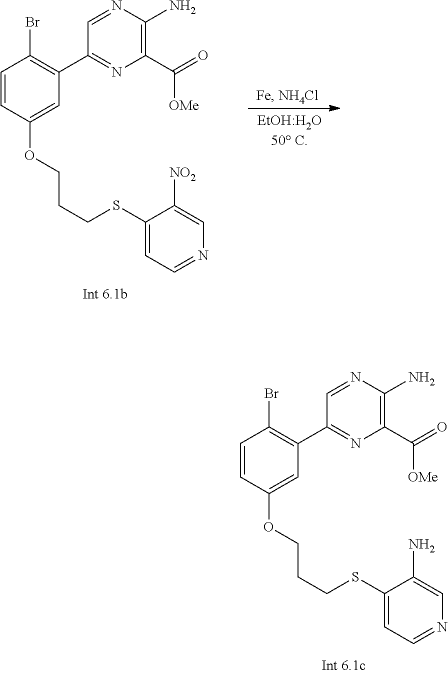 [SMILES: COC(=O)c1nc(-c2cc(OCCCSc3ccncc3[N+](=O)[O-])ccc2Br)cnc1N ; COC(=O)c1nc(-c2cc(OCCCSc3ccncc3N)ccc2Br)cnc1N]

To a solution of compound Int 6.1b (489 mg, 1.0 mmol) in a mixture of EtOH (10 mL) and NH₄Cl (sat. aq.) (10 mL), was added solid iron powder (560 mg, 10 mmol, 10 equiv). The resultant mixture was heated with vigorous stirring at 50° C., for 15 hrs. The reaction was then cooled to room temperature and filtered through a pad of celite and washed with MeOH. The solvent was removed via rotary evaporator and the residue was washed with water and extracted into dichloromethane. The organic layers were dried over Mg₂SO₄, filtered and concentrated. The resultant residue was purified via column chromatography (Silica) 0→10% MeOH in dichloromethane to provide 288 mg (0.59 mmol, 59% yield) of compound Int 6.1c. M+H=491.2.5.

### Step D: Preparation of Int 6.1d

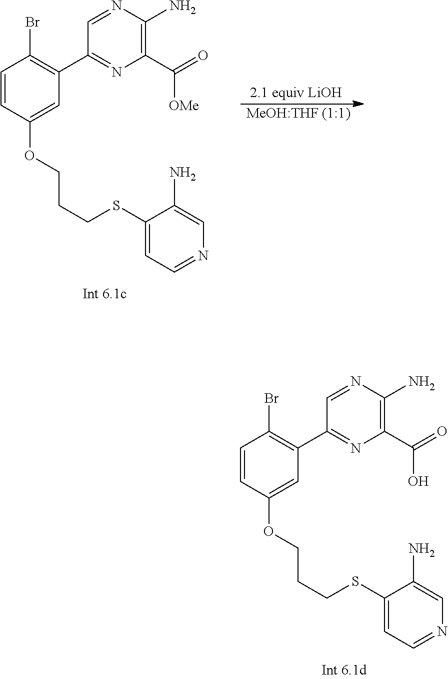 [SMILES: COC(=O)c1nc(-c2cc(OCCCSc3ccncc3N)ccc2Br)cnc1N ; Nc1cnccc1SCCCOc1ccc(Br)c(-c2cnc(N)c(C(=O)O)n2)c1]

Compound Int 6.1c (288 mg, 0.59 mmol) was dissolved in 4 mL of THF and 2 mL of methanol at room temperature then treated with 2.1 equivalents LiOH (1M aq). The mixture was stirred for 2 h then evaporated to dryness. The residue was azeotroped with toluene. This process was repeated three times, the residue compound Int 6.1d was dried under vacuum and used directly in next step. M−H=475.2.

### Step E: Preparation of Compound 51

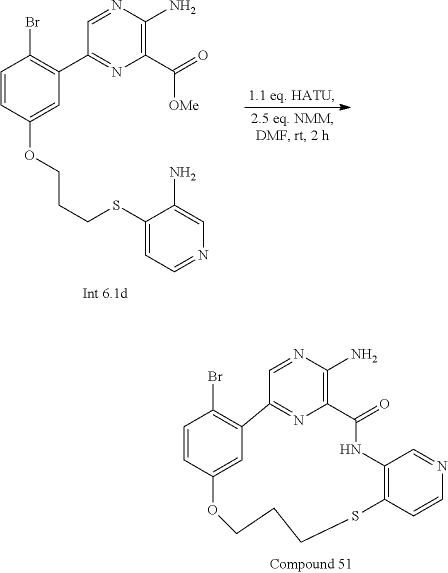 [SMILES: COC(=O)c1nc(-c2cc(OCCCSc3ccncc3N)ccc2Br)cnc1N ; Nc1ncc2nc1C(=O)Nc1cnccc1SCCCOc1ccc(Br)c-2c1]

A mixture containing 224 mg (0.47 mmol) of compound Int 6.1d in 25 mL of DMF was treated with 400 mg (1.06 mmol) of HATU and 0.262 mL (1.9 mmol) of NMM then stirred at room temperature for 15h. The reaction was then quenched with 2 mL of methanol and 0.5 mL of ammonium hydroxide solution and stirred for 1h. The solvents were removed, and the residue was chromatographed by reverse phase chromatography to provide Compound 51. LRMS (ESI+) [M+H]⁺: 559.4.

The compounds in Table 7 below were prepared in a manner analogous to that described above in Scheme 6 and for the synthesis of Compound 51 in Example 6.

TABLE 7

| Cmpd | Structure | ¹H NMR/LRMS |
|---|---|---|
| 52 | 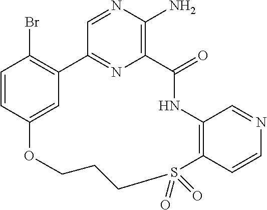 [SMILES: Nc1ncc2nc1C(=O)Nc1cnccc1S(=O)(=O)CCCOc1ccc(Br)c-2c1] | LRMS (ESI+) [M + H]⁺; 490.3 |

In other embodiments, compounds described herein are prepared as described in Scheme 7.

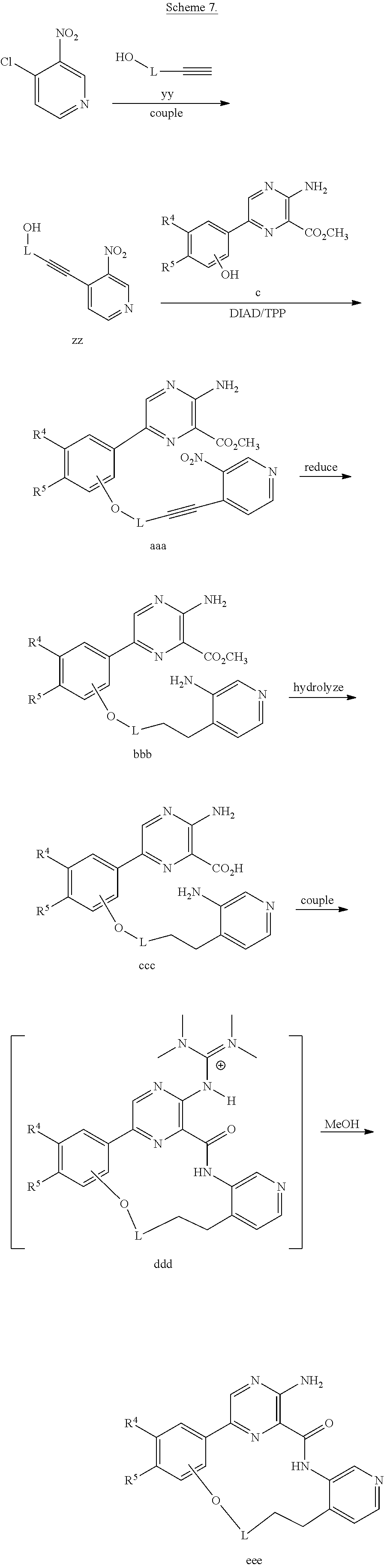

Alcohol intermediates (zz) can react with phenol intermediates (c) using well established Mitsunobu etherification conditions to provide compounds (aaa) which can be converted to the amine derivatives (bbb) using standard catalytic hydrogenation procedures. Hydrolysis of the carboxylic ester functional group can be effected by reacting intermediate (bbb) with a metal alkoxide base such as LiOH to provide carboxylic acids (ccc) which can undergo a macrolactamization using an amide coupling reagent such as HATU and a tertiary amine base such as DIPEA. Intermediate macrolactams (ddd) can be converted to the desired targets (eee) after brief exposure to methanol followed by purification.

### Example 7: Synthesis of Compound-Linkers

### Example 7.1 4-((S)-2-((S)-2-(6-(2,5-dioxo-2,5-dihydro-1H-pyrrol-1-yl)hexanamido)-3-methylbutanamido)-5-ureidopentanamido)benzyl (3-((2⁵-amino-6-methyl-3-oxo-10-oxa-4,6-diaza-2(2,6)-pyrazina-5(3,4)-pyridina-1(1,3)-benzenacyclodecaphane-15-yl)oxy)propyl)carbamate (Compound-Linker B-16)

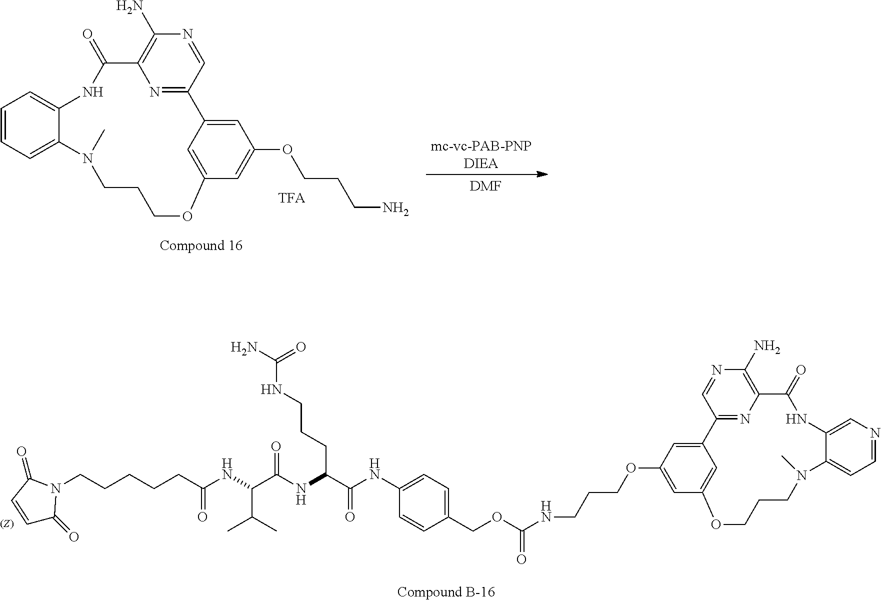

A solution containing 16.1 mg (0.028 mmol) of Compound 16 was dissolved in 4 mL of DMF and then treated with 23.1 mg (0.031 mmol) of mc-VC-PAB-PNP and 21 μL (0.114 mmol) of Hunig's base. The reaction was stirred at ambient temperature for 15 h and then purified without work-up using RP-HPLC. Product fractions were identified by LCMS and pooled then lyophilized to provide 15.1 mg of Compound-Linker B-16 as a white solid. LCMS (M+H)=1049.2

The compounds in Table 8 below were prepared in a manner analogous to that described above in Schemes A and/or B and for the synthesis of Compound B-16 in Example 7.1.

TABLE 8

| Cmpd | Structure | [M + H]⁺ |
|---|---|---|
| B-16a | 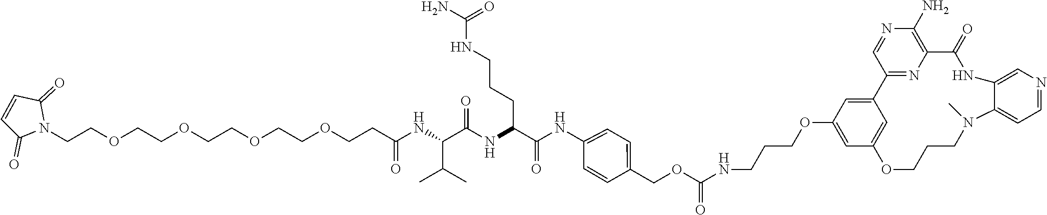 [SMILES: CC(C)[C@H](NC(=O)CCOCCOCCOCCOCCN1C(=O)C=CC1=O)C(=O)N[C@@H](CCCNC(N)=O)C(=O)Nc1ccc(COC(=O)NCCCOc2cc3cc(c2)-c2cnc(N)c(n2)C(=O)Nc2cnccc2N(C)CCCO3)cc1] | 1183.3 |
| B-61 | 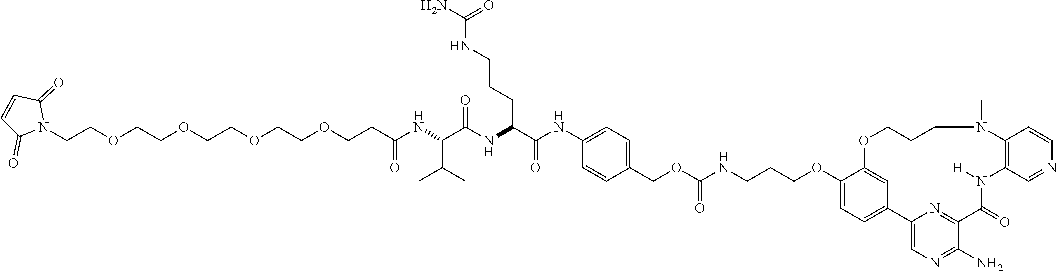 [SMILES: CC(C)[C@H](NC(=O)CCOCCOCCOCCOCCN1C(=O)C=CC1=O)C(=O)N[C@@H](CCCNC(N)=O)C(=O)Nc1ccc(COC(=O)NCCCOc2ccc3cc2OCCCN(C)c2ccncc2NC(=O)c2nc-3cnc2N)cc1] | 1183.3 |
| B-62 | 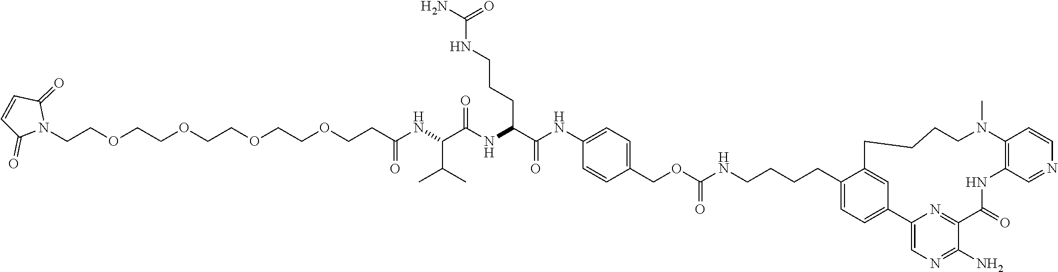 [SMILES: CC(C)[C@H](NC(=O)CCOCCOCCOCCOCCN1C(=O)C=CC1=O)C(=O)N[C@@H](CCCNC(N)=O)C(=O)Nc1ccc(COC(=O)NCCCCc2ccc3cc2CCCCN(C)c2ccncc2NC(=O)c2nc-3cnc2N)cc1] | 1179.4 |
| B-63 | 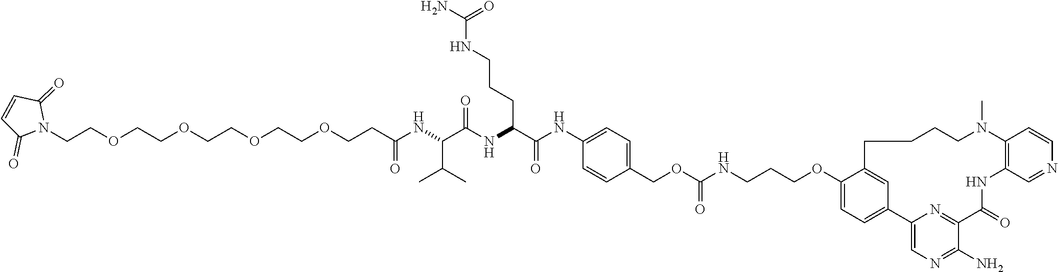 [SMILES: CC(C)[C@H](NC(=O)CCOCCOCCOCCOCCN1C(=O)C=CC1=O)C(=O)N[C@@H](CCCNC(N)=O)C(=O)Nc1ccc(COC(=O)NCCCOc2ccc3cc2CCCCN(C)c2ccncc2NC(=O)c2nc-3cnc2N)cc1] | 1181.4 |
| B-64 | 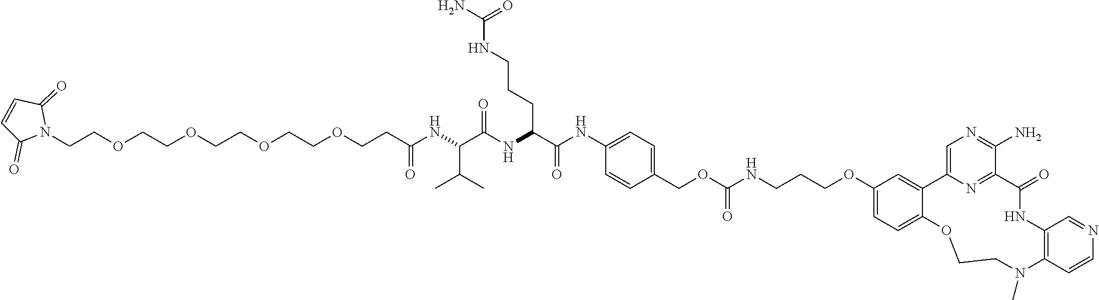 [SMILES: CC(C)[C@H](NC(=O)CCOCCOCCOCCOCCN1C(=O)C=CC1=O)C(=O)N[C@@H](CCCNC(N)=O)C(=O)Nc1ccc(COC(=O)NCCCOc2ccc3c(c2)-c2cnc(N)c(n2)C(=O)Nc2cnccc2N(C)CCO3)cc1] | 1169.3 |
| B-65 | 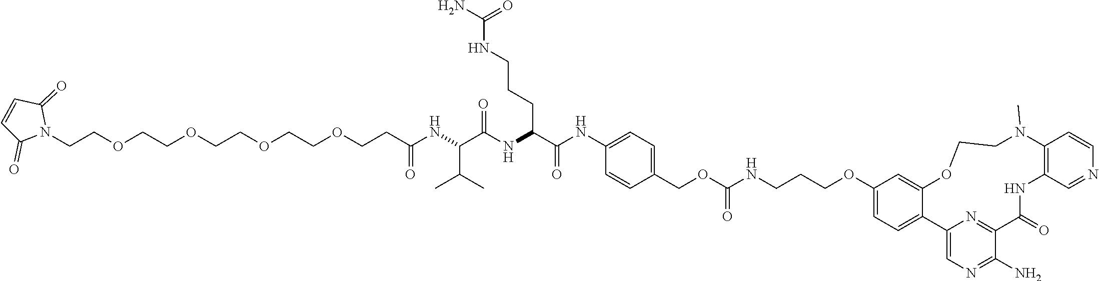 [SMILES: CC(C)[C@H](NC(=O)CCOCCOCCOCCOCCN1C(=O)C=CC1=O)C(=O)N[C@@H](CCCNC(N)=O)C(=O)Nc1ccc(COC(=O)NCCCOc2ccc3c(c2)OCCN(C)c2ccncc2NC(=O)c2nc-3cnc2N)cc1] | 1169.3 |

### Example II: TGFβ Reporter Assay

### Example IIA

### Materials and General Procedures

TGFβ/SMAD Singling Pathway SBE reporter cell line was obtained from BPS Bioscience. Cells were passed, expanded, and stored in liquid nitrogen as per the supplier's instructions with the exception that growth media is changed to DMEM-C with Geneticin (DMEM supplemented with 10% fetal bovine serum, 1×NEAA, 1 mM Pyruvate, 2 mM glutamine, 50 μg/mL penicillin, 50 U/mL streptomycin and 400 μg/mL of Geneticin). The assay media was MEM supplemented with 0.500 fetal bovine serum, 1×NEAA, 1 mM Pyruvate, 50 μg/mL penicillin and 50 U/mL streptomycin.

### General Procedure for In Vitro Small Molecule Screening—TGFβ Reporter Assay

Test samples (at desired concentrations diluted in assay media) were added to a 96-well assay plate, 20 μL per well. Reporter cells were harvested from the tissue culture flasks by incubation in small quantity of PBS at 37° C. for two minutes after the media in the flask is removed and cells rinsed with PBS. Cells were counted and diluted in the assay media at approximately 0.5×10⁶ cells/mL and then 80 μL/well of cells were added to the assay plate containing the 20 μL/well of test samples (or media only), and incubated for approximately 5-6 hours at 37° C. in a 5% CO₂ humidified incubator. After that time, 15 μL of TGFβ diluted to 12 ng/mL in the assay media was added to the plate. Controls included TGFβ titration (from 50 to 0 ng/mL) without inhibitors, and media only (without cells, inhibitor or TGFβ). Plates were incubated at 37° C. in a 5% CO₂ humidified incubator for 18 h. Luciferase substrate solution is subsequently added at 100 μL per well, incubated in the dark at room temperature for 15 min, and luminescence is measured using a luminometer. EC₅₀ values and curve fits were obtained using Prism (GraphPad Software).

Table 9 includes EC₅₀ values for representative compounds.

TABLE 9

| Compound | EC₅₀ |
|---|---|
| 1 | C |
| 2 | C |
| 3 | C |
| 4 | A |
| 5 | A |
| 6 | A |
| 7 | C |
| 8 | B |
| 9 | A |
| 10 | A |
| 11 | A |
| 12 | A |
| 13 | A |
| 14 | A |
| 15 | A |
| 16 | A |
| 17 | A |
| 19 | A |
| 20 | A |
| 49 | A |
| 18 | A |
| 25 | A |
| 50 | A |
| 51 | A |
| 52 | C |
| 23 | C |
| 26 | A |
| 22 | C |
| 53 | C |
| 54 | C |
| 55 | C |
| 56 | B |
| 24 | C |
| 27 | A |
| 28 | C |
| 21 | C |
| 30 | C |
| 31 | B |
| 34 | B |
| 41 | A |
| 35 | C |
| 37 | A |
| 42 | A |
| 36 | C |
| 38 | A |
| 39 | C |
| 40 | B |
| 43 | C |
| 57 | B |
| 58 | B |
| 59 | C |
| 60 | B |
| 61 | A |
| 62 | A |
| 63 | A |
| 64 | A |
| 65 | A |

1 nM < A ≤ 1000 nM;
1000 nM < B ≤ 5000 nM;
C > 5000 nM.

### Example IIB

The linker payloads in Table 8 were covalently attached to an anti-LRRC15 antibody. The LRRC15 antibody is the murine M25 antibody or a humanized variant thereof (see PCT Application Publication No. WO 2017/095805, incorporated herein by reference in its entirety). Conjugation to the linker-payload is via the interchain disulfides. The antibodies have either a wild-type Fc domain or a null Fc domain. The Fcnull mutations for human IgG1 are L234A, L236A, G237A, and K322A and the Fcnull mutations for murine IgG2a are L234A, L236A, G237A, K322A, and P329G; numbering by EU index.

The resultant antibody drug conjugates were tested via a cell reporter assay. HEK293 SMAD2p luciferase reporter cells transfected to stably express full length human LRRC15 were seeded in 96 well plates at 40,000 cells/well in an assay media of MEM +0.5% FBS, 1% NEAA, 1% NaPyr & 1% Pen/Strep. Conjugates and controls were added to wells in a dose titration ranging from 500 nM to 0.03 nM. After 24 hours of culture at 37° C. in a 5% CO2 environment human TGFβ1 was added (PeproTech Inc.) to a final concentration of 1.6 ng/ml followed by an additional 18 hour of culture. Luciferase Steady Glo reagent (Promega Corporation) was added as recommended by manufacturer. After incubating 10 minutes with shaking, SMAD2p activity was determined by measuring luminescence with an Envision Plate Reader (Perkin-Elmer Inc.) and an absolute EC₅₀ was determined using Prism Software v8.01 (GraphPad Inc.).

Table 10 includes EC₅₀ values for tha above noted representative conjugates. The potency of the antibody drug conjugates track proportionally with the activity observed for the small molecule activity within the small molecule cell-based reporter assay. The rank order of potency of the applicable antibody drug conjugate tracks with the observed activity within the small molecule cell-free enzymatic inhibition assay, and and even showed unexpectedly a degree of improved EC₅₀ values as compared to the compounds alone.

TABLE 10

| Conjugate | EC₅₀ |
|---|---|
| B-16 | A |
| B-16a | A |
| B-61 | A |
| B-62 | A |
| B-63 | A |
| B-64 | A |
| B-65 | A |

1 nM < A ≤ 1000 nM;
1000 nM < B ≤ 5000 nM;
C > 5000 nM.

### Example IIC

### General Procedure for TGFBR2 RBC Assay

Compounds of the disclosure were assayed by Reaction Biology Corp. using the TGFBR2 RBC enzyme assay.

Table 11 includes EC₅₀ values for selected compounds.

TABLE 11

| Compound | EC₅₀ |
|---|---|
| 1 | C |
| 2 | C |
| 3 | C |
| 4 | A |
| 5 | A |
| 6 | A |
| 7 | C |
| 8 | B |
| 9 | A |
| 10 | A |
| 11 | A |
| 12 | A |
| 13 | B |
| 14 | A |
| 15 | A |
| 16 | A |
| 17 | A |
| 19 | A |
| 20 | A |
| 49 | A |
| 18 | A |
| 25 | A |
| 50 | A |
| 51 | A |
| 23 | C |
| 26 | A |
| 22 | C |
| 53 | B |
| 55 | B |
| 56 | A |
| 27 | A |
| 21 | C |
| 30 | C |
| 31 | B |
| 34 | B |
| 35 | C |
| 37 | B |
| 42 | B |
| 36 | C |
| 38 | A |
| 39 | C |
| 40 | C |
| 43 | C |
| 57 | C |
| 58 | C |
| 59 | C |
| 60 | C |
| 61 | A |
| 62 | A |
| 63 | A |
| 64 | A |
| 65 | A |

1 nM < A ≤ 100 nM;
100 nM < B ≤ 1000 nM;
C > 1000 nM.

What is claimed is:

1. A compound of Formula (I): 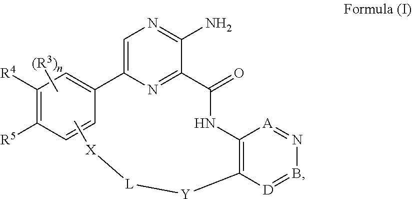
or a pharmaceutically acceptable salt thereof, wherein:
A, B, and D are each independently selected from N and C(R¹);
each R¹ is independently selected from hydrogen, halogen, cyano, —OH, —OR⁵⁰, —NR⁵¹R⁵¹, unsubstituted or substituted —C₁-C₆alkyl, unsubstituted or substituted cycloalkyl, and unsubstituted or substituted heterocycloalkyl;
each R³ is independently selected from R²⁰, R^{L}, and —O—R^{L};
n is 0, 1, or 2;
R⁴ is selected from hydrogen, R²⁰, R^{L}, and —O—R^{L};
R⁵ is selected from hydrogen, R²⁰, R^{L}, and —O—R^{L};
X is selected from —O—, —S—, —NR⁷—, —C(R⁸)₂—, —C(R⁸)₂—O—, —C(R⁸)₂—S—, —C(R⁸)₂—NR⁷—, —S(═O)₂—, —C(═O)—, —NR⁷—S(═O)₂—, and —NR⁷—C(═O)—;
R⁷ is selected from hydrogen, unsubstituted or substituted —C₁-C₆alkyl, and R^{L};
each R⁸ is independently selected from hydrogen, halogen, unsubstituted or substituted —C₁-C₆alkyl, and R^{L};
Y is selected from —O—, —S—, —NR⁹—, —C(R¹⁰)₂—, —S(═O)₂—, —C(═O)—, —S(═O)₂—NR⁹—, —C(═O)—NR⁹—, substituted or unsubstituted cycloalkylene, and substituted or unsubstituted heterocycloalkylene;
R⁹ is selected from hydrogen and unsubstituted or substituted —C₁-C₆alkyl;
each R¹⁰ is independently selected from hydrogen, halogen, and unsubstituted or substituted —C₁-C₆alkyl;
L is selected from a bond, substituted or unsubstituted C₁-C₁₀ alkylene, —[C(R¹¹)₂]_{q}—(W)—, substituted or unsubstituted C₂-C₁₀ alkenylene, substituted or unsubstituted C₂-C₁₀ alkynylene, and [(substituted or unsubstituted C₁-C₄ alkylene)-Z-]ₚ-(substituted or unsubstituted C₁-C₄ alkylene);
W is unsubstituted or substituted cycloalkylene or unsubstituted or substituted heterocycloalkylene;
each Z is independently selected from —O—, —S—, and —NR¹¹—;
each R¹¹ is independently selected from hydrogen and unsubstituted or substituted —C₁-C₆alkyl;
p is 1-5;
q is 0-10;
wherein if L is a bond, then Y is selected from substituted or unsubstituted cycloalkylene and substituted or unsubstituted heterocycloalkylene;
R^{L} is selected from -(unsubstituted or substituted C₁-C₆ alkylene)-OR¹², or -(unsubstituted or substituted C₁-C₆ alkylene)-N(R¹³)₂,
R¹² is selected from hydrogen, unsubstituted or substituted —C₁-C₆alkyl, unsubstituted or substituted —C₂-C₆ alkenyl, unsubstituted or substituted —C₂-C₆ alkynyl, unsubstituted or substituted cycloalkyl, and unsubstituted or substituted heterocycloalkyl;
each R¹³ is independently selected from hydrogen, —C(═O)R⁵⁰, —C(═O)OR⁵¹, —C(═O)NR⁵¹R⁵¹, unsubstituted or substituted —C₁-C₆alkyl, unsubstituted or substituted —C₂-C₆ alkenyl, unsubstituted or substituted —C₂-C₆ alkynyl, unsubstituted or substituted cycloalkyl, and unsubstituted or substituted heterocycloalkyl;
or two R¹³ on the same N atom are taken together with the N atom to which they are attached to form an unsubstituted or substituted N-containing heterocycle;
each R²⁰ is independently selected from halogen, —CN, —OH, —OR⁵⁰, —NR⁵¹R⁵¹, —C(═O)R⁵⁰, —OC(═O)R⁵⁰, —C(═O)OR⁵¹, —OC(═O)OR⁵¹, —C(═O)NR⁵¹R⁵¹, —OC(═O)NR⁵¹R⁵¹, —NR⁵¹C(═O)NR⁵¹R⁵¹, —NR⁵¹C(═O)R⁵⁰, —NR⁵¹C(═O)OR⁵¹, —SR⁵¹, —S(═O)R⁵⁰, —SO₂R⁵⁰, —SO₂NR⁵¹R⁵¹, —NHSO₂R⁵⁰, unsubstituted or substituted —C₁-C₆ alkyl, unsubstituted or substituted —C₂-C₆ alkenyl, unsubstituted or substituted —C₂-C₆ alkynyl, unsubstituted or substituted cycloalkyl, and unsubstituted or substituted heterocycloalkyl;
each R⁵⁰ is independently selected from unsubstituted or substituted —C₁-C₆ alkyl, unsubstituted or substituted cycloalkyl, unsubstituted or substituted heterocycloalkyl, unsubstituted or substituted aryl, unsubstituted or substituted heteroaryl, -(unsubstituted or substituted C₁-C₆alkylene)-cycloalkyl, -(unsubstituted or substituted C₁-C₆alkylene)-heterocycloalkyl, -(unsubstituted or substituted C₁-C₆alkylene)-aryl, and -(unsubstituted or substituted C₁-C₆alkylene)-heteroaryl; and
each R⁵¹ is independently selected from hydrogen, unsubstituted or substituted —C₁-C₆ alkyl, unsubstituted or substituted cycloalkyl, unsubstituted or substituted heterocycloalkyl, unsubstituted or substituted aryl, unsubstituted or substituted heteroaryl, -(unsubstituted or substituted C₁-C₆alkylene)-cycloalkyl, -(unsubstituted or substituted C₁-C₆alkylene)-heterocycloalkyl, -(unsubstituted or substituted C₁-C₆alkylene)-aryl, and -(unsubstituted or substituted C₁-C₆alkylene)-heteroaryl;
or two R⁵¹ on the same N atom are taken together with the N atom to which they are attached to form an unsubstituted or substituted N-containing heterocycle;
wherein when any of L, W, Y, R^{L}, R¹, R⁷, R⁸, R⁹, R¹⁰, R¹¹, R¹², R¹³, R²⁰, R⁵⁰, and R⁵¹ are substituted, substituents on the L, W, Y, R^{L}, R¹, R⁷, R⁸, R⁹, R¹⁰, R¹¹, R¹², R¹³, R²⁰, R⁵⁰, and R⁵¹ are independently selected at each occurrence from halogen, —CN, —NO₂, —OR⁵², —CO₂R⁵², —C(═O)R⁵³, —C(═O)NR⁵²R⁵², —NR⁵²R⁵², —NR⁵²C(═O)R⁵³, —NR⁵²C(═O)OR⁵², —SR⁵², —S(═O)R⁵³, —SO₂R⁵³, —SO₂NR⁵²R⁵², unsubstituted C₁-C₆ alkyl, unsubstituted C₁-C₆ haloalkyl, unsubstituted phenyl, unsubstituted 5- or 6-membered heteroaryl, unsubstituted monocyclic cycloalkyl, and unsubstituted monocyclic heterocycloalkyl; or two substituents on the same carbon atom are taken together to form a ═O or ═S;
each R⁵² is independently selected from hydrogen, unsubstituted C₁-C₆ alkyl, unsubstituted C₃-C₆ cycloalkyl, unsubstituted 3- to 6-membered heterocycloalkyl, unsubstituted phenyl, unsubstituted benzyl, unsubstituted 5-membered heteroaryl, and unsubstituted 6-membered heteroaryl;
or two R⁵² groups are taken together with the N atom to which they are attached to form an unsubstituted N-containing heterocycle; and
each R⁵³ is independently selected from unsubstituted C₁-C₆alkyl, unsubstituted C₃-C₆cycloalkyl, unsubstituted phenyl, unsubstituted benzyl, unsubstituted 5-membered heteroaryl, and unsubstituted 6-membered heteroaryl.

2. The compound of claim 1, or a pharmaceutically acceptable salt thereof, wherein:
each R¹ is hydrogen.

3. The compound of claim 1 or 2, or a pharmaceutically acceptable salt thereof, wherein:
n is 1 or 2.

4. The compound of claim 3, or a pharmaceutically acceptable salt thereof, wherein:
each R³ is independently selected from halogen, —CN, —OH, —OR⁵⁰, —NR⁵¹R⁵¹, —C(═O)R⁵⁰, —C(═O)NR⁵¹R⁵¹, —NR⁵¹C(═O)R⁵⁰, unsubstituted or substituted —C₁-C₆ alkyl, unsubstituted or substituted cycloalkyl, and unsubstituted or substituted heterocycloalkyl.

5. The compound of claim 3, or a pharmaceutically acceptable salt thereof, wherein:
at least one R³ is halogen.

6. The compound of claim 5, or a pharmaceutically acceptable salt thereof, wherein:
at least one R³ is bromine.

7. The compound of any one of claim 1 or 2, or a pharmaceutically acceptable salt thereof, wherein:
n is 0.

8. The compound of any one of claims 1-7, or a pharmaceutically acceptable salt thereof, wherein:
at least one of R³, R⁴, and R⁵ is halogen.

9. The compound of any one of claims 1-8, or a pharmaceutically acceptable salt thereof, A, B, and D are C(R¹).

10. The compound of claim 9 wherein A, B, and D are CH.

11. The compound of any one of claims 1-10, or a pharmaceutically acceptable salt thereof, wherein not more than one of A, B, and D is N.

12. The compound of claim 11, or a pharmaceutically acceptable salt thereof, wherein:
A is N and B and D are CH.

13. The compound of claim 11, or a pharmaceutically acceptable salt thereof, wherein:
B is N and A and D are CH.

14. The compound of claim 11, or a pharmaceutically acceptable salt thereof, wherein:
D is N and B and A are CH.

15. The compound of claim 1, wherein the compound of Formula (I) is represented by Formula (II): 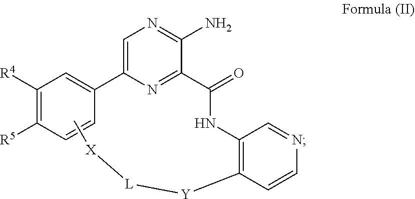
or a pharmaceutically acceptable salt thereof.

16. The compound of claim 15, wherein the compound of Formula (II), is represented by Formula (III): 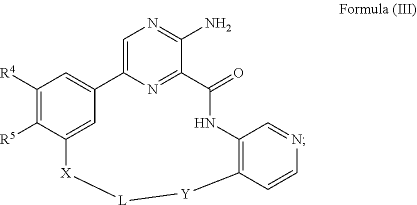
or a pharmaceutically acceptable salt thereof.

17. The compound of claim 15, wherein the compound of Formula (II), is represented by Formula (IV): 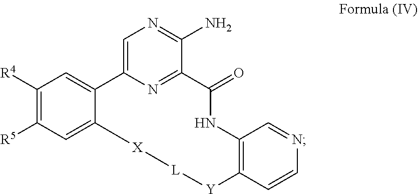
or a pharmaceutically acceptable salt thereof.

18. The compound of any one of claims 1-17, or a pharmaceutically acceptable salt thereof, wherein:
Y is selected from —O—, —S—, —NR⁹—, —C(R¹⁰)₂—, —S(═O)₂—, —C(═O)—, —S(═O)₂—NR⁹—, —C(═O)—NR⁹—, substituted or unsubstituted C₅ cycloalkylene, and substituted or unsubstituted 5 membered heterocycloalkylene ring;

19. The compound of any one of claims 1-17, or a pharmaceutically acceptable salt thereof, wherein:
Y is selected from —O—, —S—, —S(═O)₂—, —NR⁹—, —C(R¹⁰)₂—, and substituted or unsubstituted heterocycloalkylene;
R⁹ is selected from hydrogen and unsubstituted —C₁-C₆alkyl; and
each R¹⁰ is independently selected from hydrogen and unsubstituted —C₁-C₆alkyl.

20. The compound of any one of claims 1-17, or a pharmaceutically acceptable salt thereof, wherein:
Y is selected from —O—, —S—, —S(═O)₂—, —NR⁹—, —C(R¹⁰)₂—, and C₅ substituted or unsubstituted heterocycloalkylene;
R⁹ is selected from hydrogen and unsubstituted —C₁-C₆alkyl; and
each R¹⁰ is independently selected from hydrogen and unsubstituted —C₁-C₆alkyl;
provided that when Y is a substituted or unsubstituted 5 membered heterocycloalkylene ring, L is —CH₂—.

21. The compound of claim 20, or a pharmaceutically acceptable salt thereof, wherein:
Y is selected from —O—, —S—, —NR⁹—, and —CH₂—; and
R⁹ is selected from hydrogen and unsubstituted —C₁-C₆alkyl.

22. The compound of claim 21, or a pharmaceutically acceptable salt thereof, wherein:
Y is —NR⁹—; and
R⁹ is unsubstituted —C₁-C₆alkyl.

23. The compound of claim 22, or a pharmaceutically acceptable salt thereof, wherein:
Y is selected from —N(Et)- and —N(Me)-.

24. The compound of claim 22, or a pharmaceutically acceptable salt thereof, wherein:
Y is —N(Me)-.

25. The compound of any one of claims 1 to 17, or a pharmaceutically acceptable salt thereof, wherein:
Y is substituted or unsubstituted heterocycloalkylene.

26. The compound of claim 25, or a pharmaceutically acceptable salt thereof, wherein:
Y is unsubstituted heterocycloalkylene.

27. The compound of claim 25, or a pharmaceutically acceptable salt thereof, wherein:
Y is substituted or unsubstituted monocyclic heterocycloalkylene, wherein the heterocycloalkylene contains a nitrogen atom and optionally one other heteroatom selected from a nitrogen atom, oxygen atom, and sulfur atom.

28. The compound of any one of claims 1-17, or a pharmaceutically acceptable salt thereof, wherein:
Y is represented by 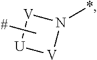
wherein:
# is the attachment point to L and * is the attachment point to the rest of the molecule;
each V is independently —(C(R²¹)₂)ᵣ—; wherein each r is independently 1-3;
each R²¹ is independently selected from hydrogen, halogen, —CN, —NO₂, —OR⁵², —CO₂R⁵², —C(═O)R⁵³, —C(═O)NR⁵²R⁵², —NR⁵²R⁵², —NR⁵²C(═O)R⁵³, —NR⁵²C(═O)OR⁵²—SR⁵², —S(═O)R⁵³, —SO₂R⁵³, —SO₂NR⁵²R⁵², —C₁-C₆ alkyl, —C₁-C₆ haloalkyl, phenyl, 5- or 6-membered heteroaryl, monocyclic cycloalkyl, and monocyclic heterocycloalkyl; or two R²¹ on the same carbon atom are taken together to form a ═O or ═S; and
U is selected from bond, —O—, —S—, and —NR²²—; wherein R²² is selected from hydrogen and unsubstituted —C₁-C₆alkyl.

29. The compound of claim 28, or a pharmaceutically acceptable salt thereof, wherein:
each R²¹ is independently selected from hydrogen, halogen, —OR⁵², —NR¹²R¹², —C₁-C₆ alkyl, and —C₁-C₆ haloalkyl; or two R²¹ on the same carbon atom are taken together to form a ═O.

30. The compound of any one of claims 1 to 17, or a pharmaceutically acceptable salt thereof, wherein:
Y is represented by 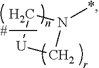
wherein:
each r is independently 1-3;
U is selected from bond, —O—, —S—, —NH— and —NMe-.

31. The compound of any one of claims 1 to 17, or a pharmaceutically acceptable salt thereof, wherein:
Y is selected from —NH—, —NMe-, —NEt-, —N(n-Pr)-, —CH₂—, —S—, —O—, —S(═O)₂—,

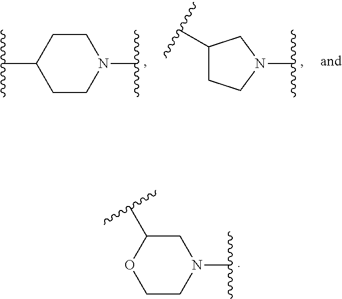 [SMILES: *C1CCN(*)CC1 ; *C1CCN(*)C1 ; *C1CN(*)CCO1]

32. The compound of any one of any one of claims 1 to 31 wherein when Y is a substituted or unsubstituted cyloalkylene or substituted or unsubstituted heterocycloalkylene, L is —CH₂—.

33. The compound of any one of claims 1-17, wherein the compound of Formula (I) is represented by Formula (IId): 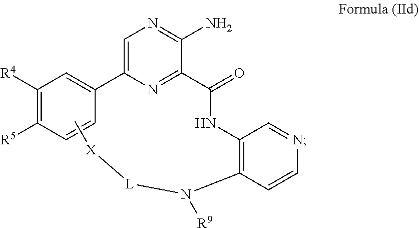
or a pharmaceutically acceptable salt thereof, wherein R⁹ is methyl or ethyl.

34. The compound of claim 33, wherein the compound of Formula (II), is represented by Formula (IIId): 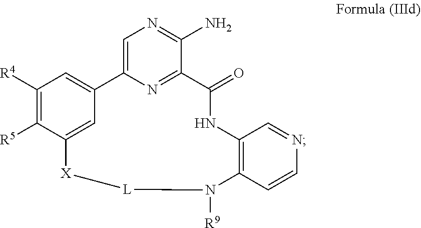
or a pharmaceutically acceptable salt thereof, wherein R⁹ is methyl or ethyl.

35. The compound of claim 33, wherein the compound of Formula (II), is represented by Formula (IVd): 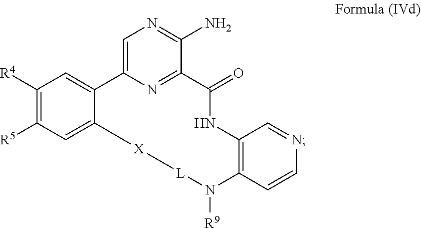
or a pharmaceutically acceptable salt thereof, wherein R⁹ is methyl or ethyl.

36. The compound of any one of claims 1-35, or a pharmaceutically acceptable salt thereof, wherein:
X is selected from —O—, —NR⁷—, —C(R⁸)₂—, —C(R⁸)₂—O—, —S(═O)₂—, and —NR⁷—C(═O)—;
R⁷ is selected from hydrogen, unsubstituted —C₁-C₆alkyl, and R^{L}; and
each R⁸ is independently selected from hydrogen, unsubstituted —C₁-C₆alkyl, and R^{L}.

37. The compound of claim 36, or a pharmaceutically acceptable salt thereof, wherein:
X is selected from —O—, —CH₂—, —CH₂—O—, —CH(R⁸)—O—, and —NR⁷—C(═O)—;
R⁷ is selected from hydrogen, unsubstituted —C₁-C₆alkyl, and R^{L}; and
Each R⁸ is independently selected from hydrogen, unsubstituted —C₁-C₆alkyl and R^{L}.

38. The compound of claim 36, or a pharmaceutically acceptable salt thereof, wherein:
X is selected from —O—, —CH₂—, —CH₂—O—, —CH(R⁸)—O—, and —NR⁷—C(═O)—;
R⁷ is selected from hydrogen and unsubstituted —C₁-C₆alkyl; and
each R⁸ is independently hydrogen or unsubstituted —C₁-C₆alkyl.

39. The compound of any one of claims 36-38, or a pharmaceutically acceptable salt thereof, wherein:
R⁷ and each R⁸ are independently selected from hydrogen and —CH₃.

40. The compound of any one of claims 1-35, or a pharmaceutically acceptable salt thereof, wherein:
X is selected from *—NR⁷—C(═O)—# and #—NR⁷—C(═O)—*;
wherein # is the attachment point to L and * is the attachment point to the rest of the molecule.

41. The compound of claim 40, or a pharmaceutically acceptable salt thereof, wherein:
X is *—NR⁷—C(═O)—#.

42. The compound of any one of claims 1-35, or a pharmaceutically acceptable salt thereof, wherein:
X is selected from —O—, —C(R⁸)₂—O—, and *—NR⁷—C(═O)—#;
wherein # is the attachment point to L and * is the attachment point to the rest of the molecule;
R⁷ and each R⁸ are independently selected from hydrogen and unsubstituted —C₁-C₆alkyl.

43. The compound of claim 42 wherein R⁷ and each R⁸ are independently selected from hydrogen and —CH₃.

44. The compound of claim 42, or a pharmaceutically acceptable salt thereof, wherein:
X is selected from —O— and —CH₂—O—.

45. The compound of any one of claims 1-35, or a pharmaceutically acceptable salt thereof, wherein:
X is selected from *—C(R⁸)₂—O—# and #—C(R⁸)₂—O—*;
wherein # is the attachment point to L and * is the attachment point to the rest of the molecule.

46. The compound of claim 45, or a pharmaceutically acceptable salt thereof, wherein:
X is #—C(R⁸)₂—O—*; wherein # is the attachment point to L and * is the attachment point to the rest of the molecule.

47. The compound of claim 46, or a pharmaceutically acceptable salt thereof, wherein:
each R⁸ is independently selected from hydrogen, —CH₃, or R^{L}.

48. The compound of any one of claims 1-47, or a pharmaceutically acceptable salt thereof, wherein:
L is selected from substituted or unsubstituted C₁-C₁₀ alkylene, —[C(R¹¹)₂]_{q}—(W)ₜ— and -[(substituted or unsubstituted C₁-C₄ alkylene)-Z]ₚ-(substituted or unsubstituted C₁-C₄ alkylene)-;
each Z is —O—;
p is 1-5; and
q is 1 to 10.

49. The compound of claim 48, or a pharmaceutically acceptable salt thereof, wherein:
L is selected from *—[C(R¹¹)₂]_{q}—(W)ₜ—# and #—[C(R¹¹)₂]_{q}—(W)ₜ—*, wherein # is the attachment point to L and * is the attachment point to the rest of the molecule.

50. The compound of claim 48, or a pharmaceutically acceptable salt thereof, wherein:
L is —[(CH₂CH₂)—O]ₚ—(CH₂CH₂)—; and
p is 1-3.

51. The compound of claim 48, or a pharmaceutically acceptable salt thereof, wherein:
L is an unsubstituted C₁-C₆ alkylene; or
L is a C₁-C₆ alkylene which is substituted by 1, 2, or 3 groups selected from halogen, —CN, —O—(C₁-C₆ alkyl), —C₁-C₆ alkyl, or —C₁-C₆ haloalkyl.

52. The compound of any one of claims 1 to 51 or a pharmaceutically acceptable salt thereof, wherein:
when X is in the meta position, L is a substituted or unsubstituted C₁-C₃ alkylene.

53. The compound of any one of claims 1 to 51, or a pharmaceutically acceptable salt thereof, wherein:
when X is in the ortho position, L is a substituted or an unsubstituted C₁-C₆ alkylene.

54. The compound of any one of claim 52 or 53, or a pharmaceutically acceptable salt thereof, wherein:
L is unsubstituted.

55. The compound of claim 48, or a pharmaceutically acceptable salt thereof, wherein:
L is selected from bond,

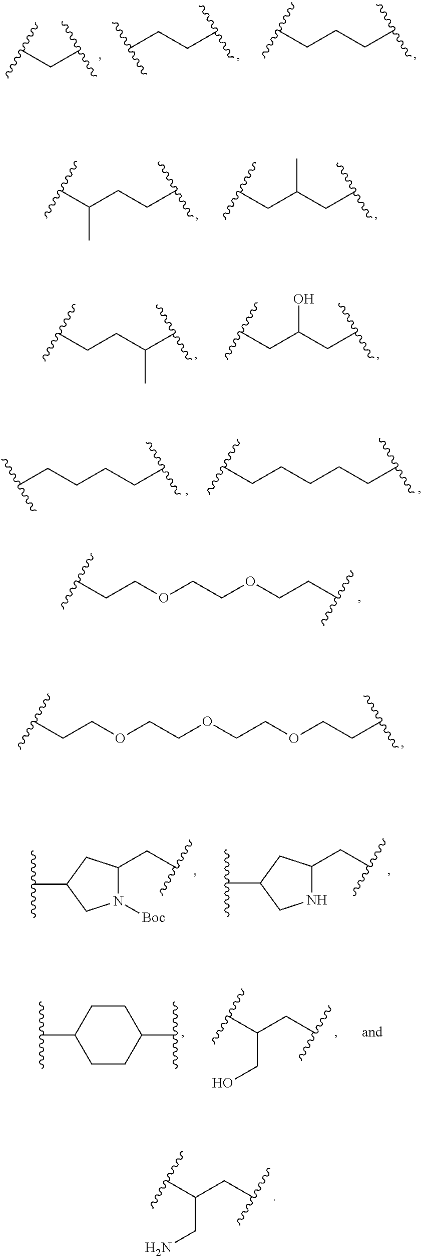

56. The compound of claim 55, or a pharmaceutically acceptable salt thereof, wherein:
L is selected from bond,

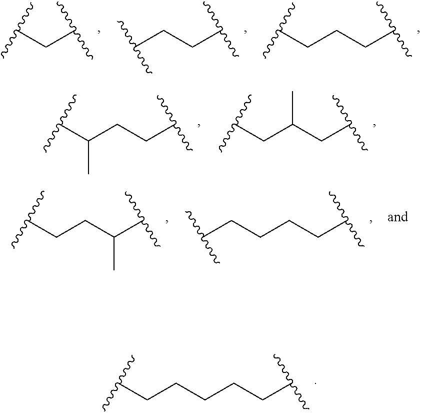

57. The compound of any one of claims 1-17, or a pharmaceutically acceptable salt thereof, wherein:
—X-L-Y— is selected from

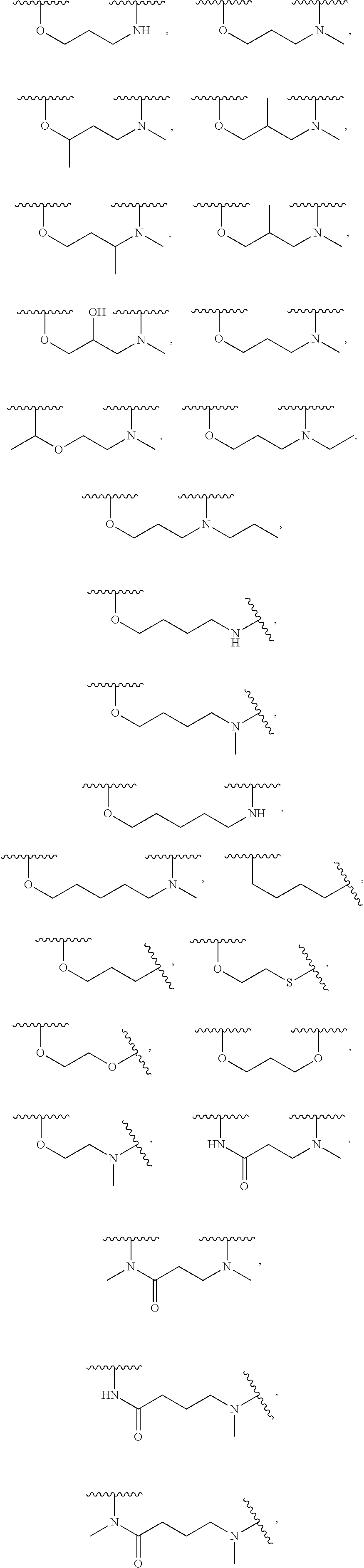

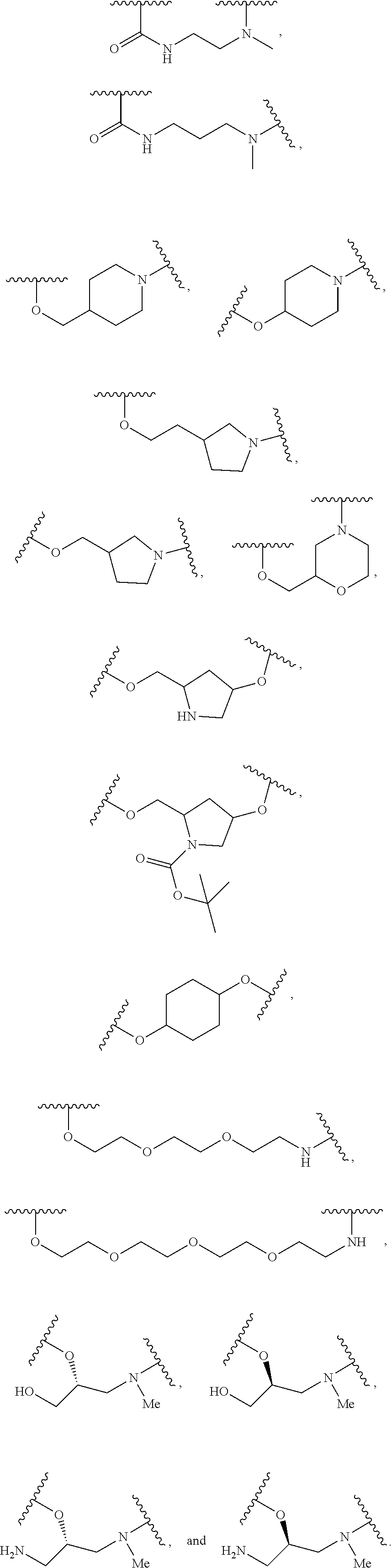

58. The compound of any one of claims 1-17 or a pharmaceutically acceptable salt thereof, wherein:
—X-L-Y— is selected from

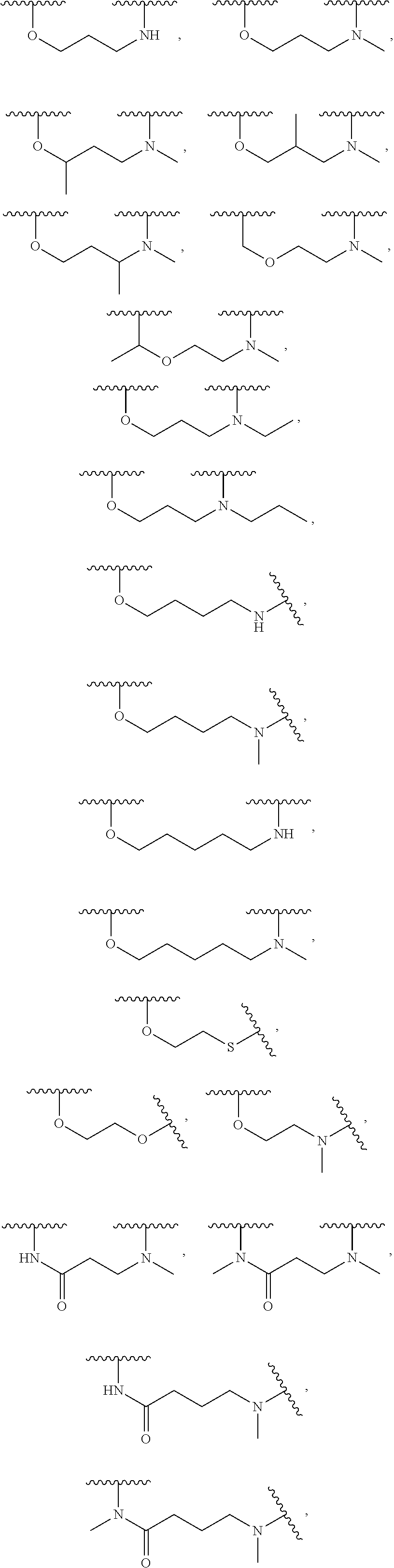

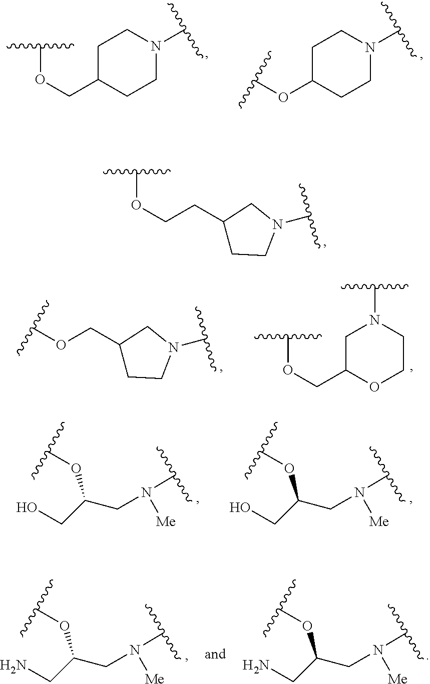

59. The compound of any one of claims 1-17, or a pharmaceutically acceptable salt thereof, wherein:
—X-L-Y— is selected from,

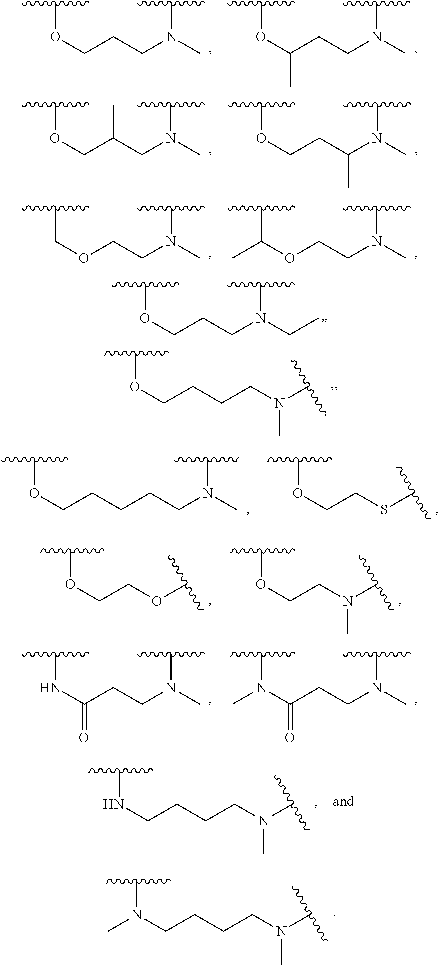

60. The compound of any one of claims 1-17, or a pharmaceutically acceptable salt thereof, wherein:
—X-L-Y— is selected from 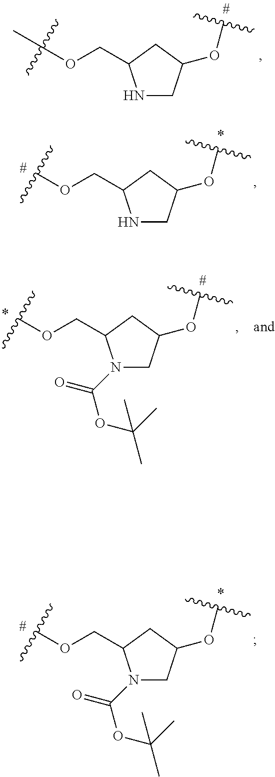 [SMILES: *OCC1CC(O*)CN1 ; *OCC1CC(O*)CN1 ; *OCC1CC(O*)CN1C(=O)OC(C)(C)C ; *OCC1CC(O*)CN1C(=O)OC(C)(C)C]
wherein # is the attachment point to L and * is the attachment point to the rest of the molecule.

61. The compound of any one of claims 1-17 wherein
X is selected from —O—, —C(R⁸)₂—, —C(R⁸)₂—O—, —NR⁷—C(═O)—;
R⁷ is selected from hydrogen and unsubstituted —C₁-C₆alkyl;
each R⁸ is independently selected from hydrogen or unsubstituted —C₁-C₆alkyl;
Y is selected from —O—, —S—, —NR⁹—, —C(R¹⁰)₂—, substituted or unsubstituted heterocycloalkylene;
R⁹ is selected from hydrogen and unsubstituted —C₁-C₆alkyl;
each R¹⁰ is hydrogen;
L is selected from a bond, substituted or unsubstituted C₁-C₆ alkylene, —[C(R¹¹)₂]_{q}—(W)—, and [(substituted or unsubstituted C₁-C₄ alkylene)-Z-]ₚ-(substituted or unsubstituted C₁-C₄ alkylene);
W is unsubstituted or substituted cyclohexylene, or substituted or unsubstituted pyrrolidinylene;
each Z is —O—;
each R¹¹ is hydrogen;
p is 1-5; and
q is 1;
wherein if L is a bond, then Y is substituted or unsubstituted heterocycloalkylene; and
wherein substituents are independently selected at each occurrence from halogen, —CN, —NO₂, —OR⁵², —CO₂R⁵², —C(═O)R⁵³, —C(═O)NR⁵²R⁵², —NR⁵²R⁵², —NR⁵²C(═O)R⁵³, —NR⁵²C(═O)OR⁵², —SR⁵², —S(═O)R⁵³, —SO₂R⁵³, —SO₂NR⁵²R⁵², unsubstituted C₁-C₆ alkyl, unsubstituted C₁-C₆ haloalkyl, unsubstituted phenyl, unsubstituted 5- or 6-membered heteroaryl, unsubstituted monocyclic cycloalkyl, and unsubstituted monocyclic heterocycloalkyl; or two substituents on the same carbon atom are taken together to form a ═O or ═S;
each R⁵² is independently selected from hydrogen, unsubstituted C₁-C₆ alkyl, unsubstituted C₃-C₆ cycloalkyl, unsubstituted 3- to 6-membered heterocycloalkyl, unsubstituted phenyl, unsubstituted benzyl, unsubstituted 5-membered heteroaryl, and unsubstituted 6-membered heteroaryl;
or two R⁵² groups are taken together with the N atom to which they are attached to form an unsubstituted N-containing heterocycle; and
each R⁵³ is independently selected from unsubstituted C₁-C₆alkyl, unsubstituted C₃-C₆cycloalkyl, unsubstituted phenyl, unsubstituted benzyl, unsubstituted 5-membered heteroaryl, and unsubstituted 6-membered heteroaryl.

62. The compound of claim 61 wherein:
Y is selected from —O—, —S—, —NR⁹—, —C(R¹⁰)₂—, substituted or unsubstituted morpholinylene, substituted or unsubstituted pyrrolidinylene, or substituted or unsubstituted piperidinylene.

63. The compound of any one of claims 1-17 wherein
X is selected from —O—, —C(R⁸)₂—O—, *—NR⁷—C(═O)—# wherein # is the attachment point to L and * is the attachment point to the rest of the molecule;
R⁷ is selected from hydrogen and unsubstituted —C₁-C₆alkyl;
each R⁸ is independently selected from hydrogen and unsubstituted —C₁-C₆alkyl;
Y is selected from —O—, —S—, —NR⁹—;
R⁹ is selected from methyl, ethyl and propyl;
L is selected from substituted or unsubstituted C₁-C₁₀ alkylene wherein the optional substituents are selected from —OH, —NH₂, or —NHCH₃.

64. The compound of any one of claims 1-17 wherein
X is selected from —O— and —C(R⁸)₂—O—;
each R⁸ is hydrogen;
Y is —NR⁹;
R⁹ is selected from methyl and ethyl
L is selected from unsubstituted C₁-C₆ alkylene.

65. The compound of any one of claims 1-17 wherein
X is selected from —O— and —C(R⁸)₂—O—;
each R⁸ is hydrogen or R^{L};
Y is —NR⁹;
R⁹ is selected from methyl and ethyl
L is selected from unsubstituted C₁-C₃ alkylene.

66. The compound of any one of claims 1-65, or a pharmaceutically acceptable salt thereof, wherein:
R⁴ is selected from hydrogen, halogen, —CN, —OH, —OR⁵⁰, —NR⁵¹R⁵¹, unsubstituted or substituted —C₁-C₆ alkyl, unsubstituted or substituted cycloalkyl, unsubstituted or substituted heterocycloalkyl, —O—R^{L}, and R^{L}.

67. The compound of claim 66, or a pharmaceutically acceptable salt thereof, wherein:
R⁴ is selected from hydrogen, unsubstituted —C₁-C₆ alkyl, —O—R^{L}, and R^{L}.

68. The compound of any one of claims 1-67, or a pharmaceutically acceptable salt thereof, wherein:
R⁵ is selected from hydrogen, halogen, —CN, —OH, —OR⁵⁰, —NR⁵¹R⁵¹, unsubstituted or substituted —C₁-C₆ alkyl, unsubstituted or substituted cycloalkyl, unsubstituted or substituted heterocycloalkyl, —O—R^{L}, and R^{L}.

69. The compound of claim 68, or a pharmaceutically acceptable salt thereof, wherein:
R⁵ is selected from hydrogen, unsubstituted —C₁-C₆ alkyl, —O—R^{L}, and R^{L}.

70. The compound of any one of claims 1-69, or a pharmaceutically acceptable salt thereof, wherein:
R^{L} is -(unsubstituted or substituted C₁-C₆ alkylene)-N(R¹³)₂.

71. The compound of claim 70, or a pharmaceutically acceptable salt thereof, wherein:
R^{L} is -(unsubstituted C₁-C₆ alkylene)-N(R¹³)₂; and
each R¹³ is independently selected from hydrogen, —C(═O)R⁵⁰, —C(═O)OR⁵¹, —C(═O)NR⁵¹R⁵¹, and unsubstituted or substituted —C₁-C₆alkyl;
or two R¹³ on the same N atom are taken together with the N atom to which they are attached to form an unsubstituted or substituted N-containing heterocycle.

72. The compound of claim 70, or a pharmaceutically acceptable salt thereof, wherein:
R^{L} is -(unsubstituted C₁-C₆ alkylene)-N(R¹³)₂; and
each R¹³ is independently selected from hydrogen and unsubstituted or substituted —C₁-C₆alkyl;
or two R¹³ on the same N atom are taken together with the N atom to which they are attached to form an unsubstituted or substituted N-containing heterocycle.

73. The compound of claim 70, or a pharmaceutically acceptable salt thereof, wherein:
R^{L} is -(unsubstituted C₁-C₆ alkylene)-NH₂.

74. The compound of claim 70, or a pharmaceutically acceptable salt thereof, wherein:
R^{L} is -(unsubstituted C₁-C₆ alkylene)-N(R¹³)₂; and
two R¹³ on the same N atom are taken together with the N atom to which they are attached to form a phthalimide.

75. The compound of any one of claims 1-74, or a pharmaceutically acceptable salt thereof, wherein:
one of R⁴ or R⁵ is selected from —O—R^{L} and R^{L}; and
each R¹³ is independently selected from hydrogen and unsubstituted or substituted —C₁-C₆alkyl; or
two R¹³ on the same N atom are taken together with the N atom to which they are attached to form a phthalimide.

76. The compound of claim 74 or 75, or a pharmaceutically acceptable salt thereof, wherein:
each R¹³ is independently selected from hydrogen and unsubstituted —C₁-C₆alkyl.

77. The compound of any one of claims 1 to 65, or a pharmaceutically acceptable salt thereof, wherein:
R⁴ is selected from hydrogen, unsubstituted —C₁-C₆alkyl, —O—R^{L}, and R^{L};
R⁵ is selected from hydrogen, unsubstituted —C₁-C₆alkyl, —O—R^{L}, and R^{L};
wherein when any of L, Y, W, and R^{L} are substituted, substituents on the L, Y, W, and R^{L} are independently selected at each occurrence from halogen, —CN, —NO₂, —OR⁵², —CO₂R⁵², —C(═O)R⁵³, —C(═O)N⁵²R⁵², —NR⁵²R⁵², —NR⁵²C(═O)R⁵³, unsubstituted C₁-C₆ alkyl, unsubstituted C₁-C₆ haloalkyl, unsubstituted phenyl, unsubstituted 5- or 6-membered heteroaryl, unsubstituted monocyclic cycloalkyl, and unsubstituted monocyclic heterocycloalkyl; or two substituents on the same carbon atom are taken together to form a ═O or ═S;
each R⁵² is independently selected from hydrogen, unsubstituted C₁-C₆ alkyl, unsubstituted C₃-C₆ cycloalkyl, unsubstituted 3- to 6-membered heterocycloalkyl, unsubstituted phenyl, unsubstituted benzyl, unsubstituted 5-membered heteroaryl, and unsubstituted 6-membered heteroaryl;
or two R⁵² groups are taken together with the N atom to which they are attached to form an unsubstituted N-containing heterocycle; and
each R⁵³ is independently selected from unsubstituted C₁-C₆alkyl, unsubstituted C₃-C₆cycloalkyl, unsubstituted phenyl, unsubstituted benzyl, unsubstituted 5-membered heteroaryl, and unsubstituted 6-membered heteroaryl.

78. The compound of claim 77, or a pharmaceutically acceptable salt thereof, wherein:
R⁴ is selected from hydrogen, —C₁-C₆alkyl, and —O—R^{L};
R⁵ is selected from hydrogen, —C₁-C₆alkyl, and —O—R^{L}; and
R^{L} is selected from -(unsubstituted C₁-C₆ alkylene)-NH₂ and -(unsubstituted C₁-C₆ alkylene)-OH.

79. The compound of any one of claims 1-69, or a pharmaceutically acceptable salt thereof, wherein:
R^{L} is

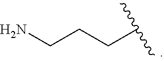 [SMILES: *CCCN]

80. The compound of any one of claims 1-77, or a pharmaceutically acceptable salt thereof, wherein:
one of R⁴ or R⁵ is selected from

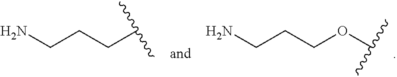 [SMILES: *CCCN ; *OCCCN]

81. The compound of claim 80, wherein the other of R⁴ and R⁵ is hydrogen.

82. The compound of claim 1, wherein the compound is selected from: 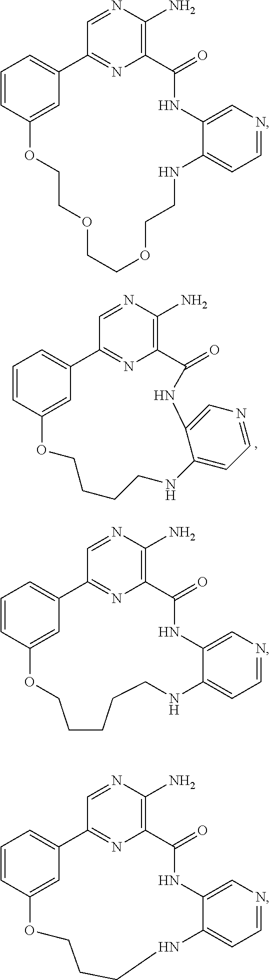 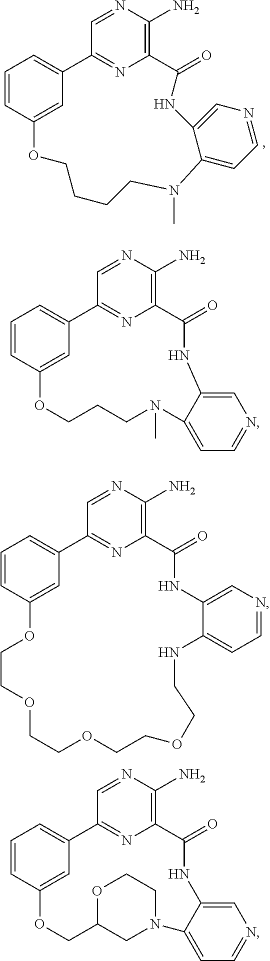 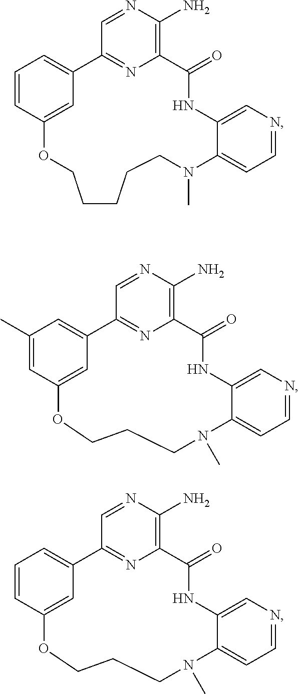 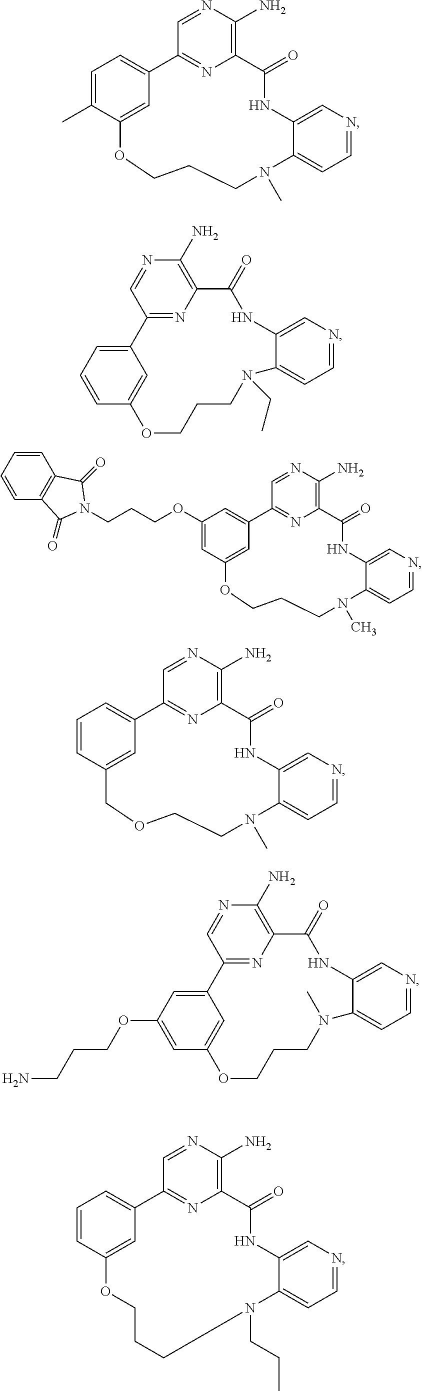 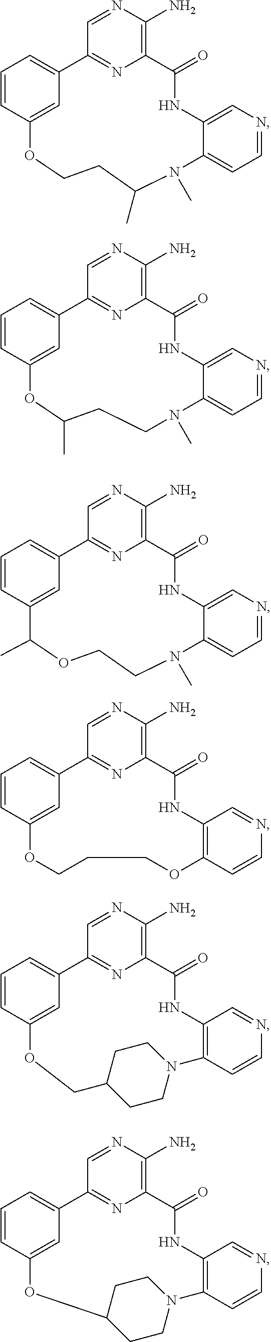 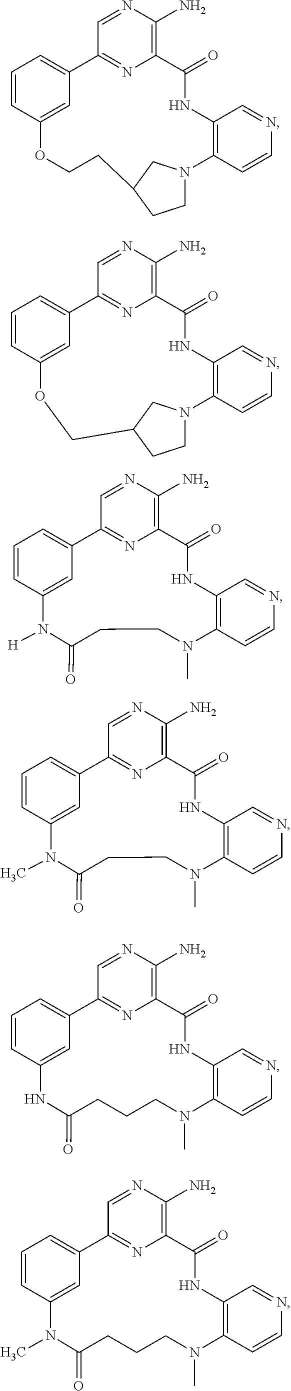 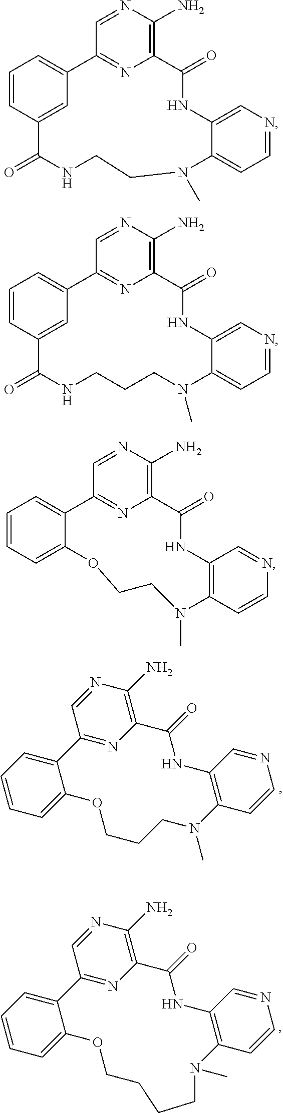 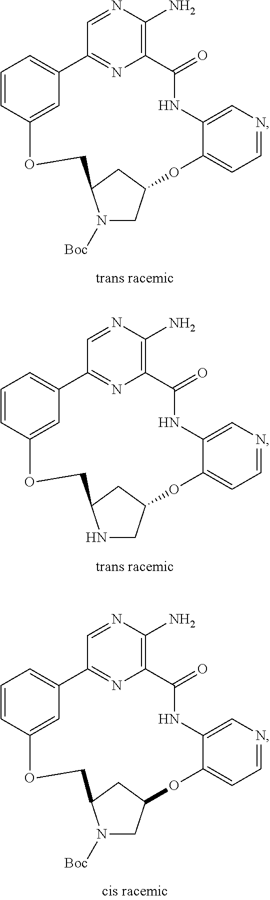 [SMILES: CC(C)(C)OC(=O)N1C[C@@H]2C[C@@H]1COc1cccc(c1)-c1cnc(N)c(n1)C(=O)Nc1cnccc1O2 ; Nc1ncc2nc1C(=O)Nc1cnccc1O[C@@H]1CN[C@@H](COc3cccc-2c3)C1 ; CC(C)(C)OC(=O)N1C[C@H]2C[C@@H]1COc1cccc(c1)-c1cnc(N)c(n1)C(=O)Nc1cnccc1O2] 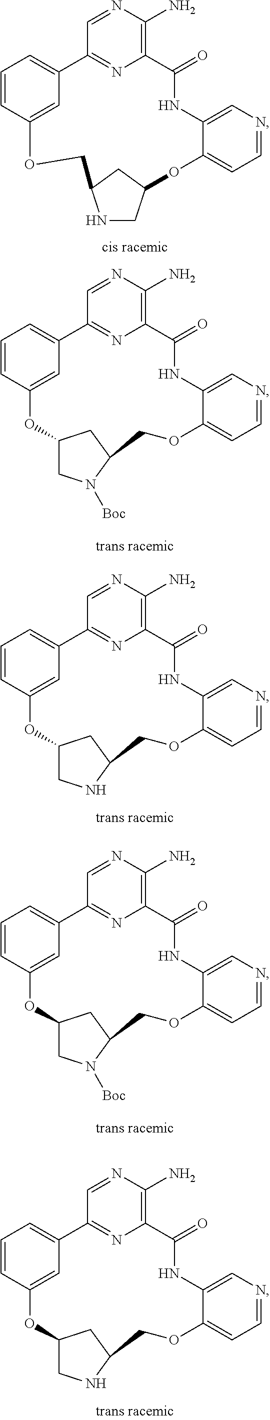 [SMILES: Nc1ncc2nc1C(=O)Nc1cnccc1O[C@H]1CN[C@@H](COc3cccc-2c3)C1 ; CC(C)(C)OC(=O)N1C[C@H]2C[C@H]1COc1ccncc1NC(=O)c1nc(cnc1N)-c1cccc(c1)O2 ; Nc1ncc2nc1C(=O)Nc1cnccc1OC[C@@H]1C[C@H](CN1)Oc1cccc-2c1 ; CC(C)(C)OC(=O)N1C[C@@H]2C[C@H]1COc1ccncc1NC(=O)c1nc(cnc1N)-c1cccc(c1)O2 ; Nc1ncc2nc1C(=O)Nc1cnccc1OC[C@@H]1C[C@@H](CN1)Oc1cccc-2c1] 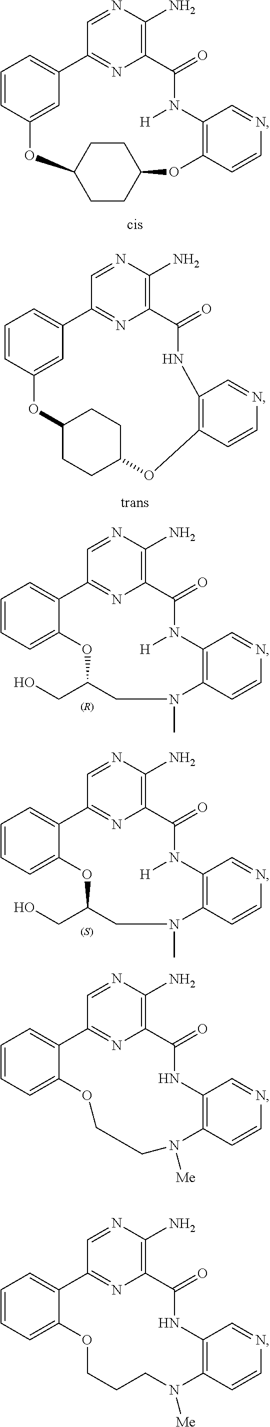 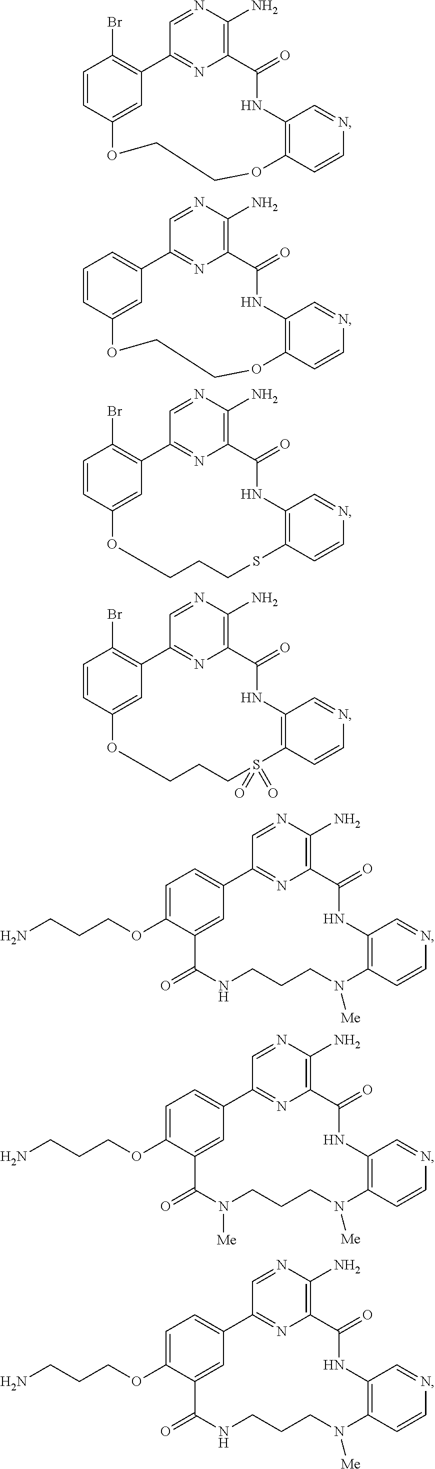 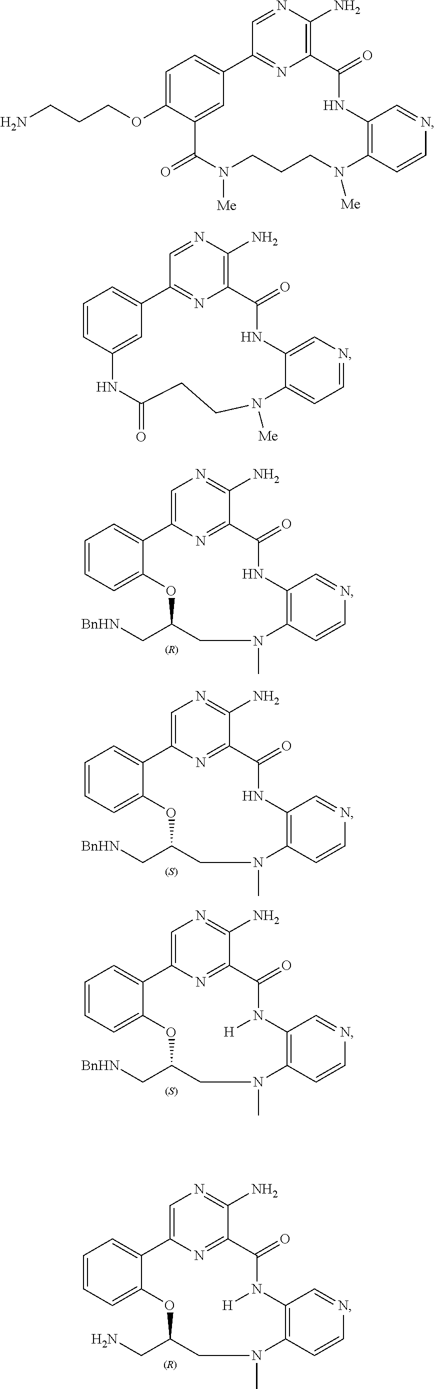 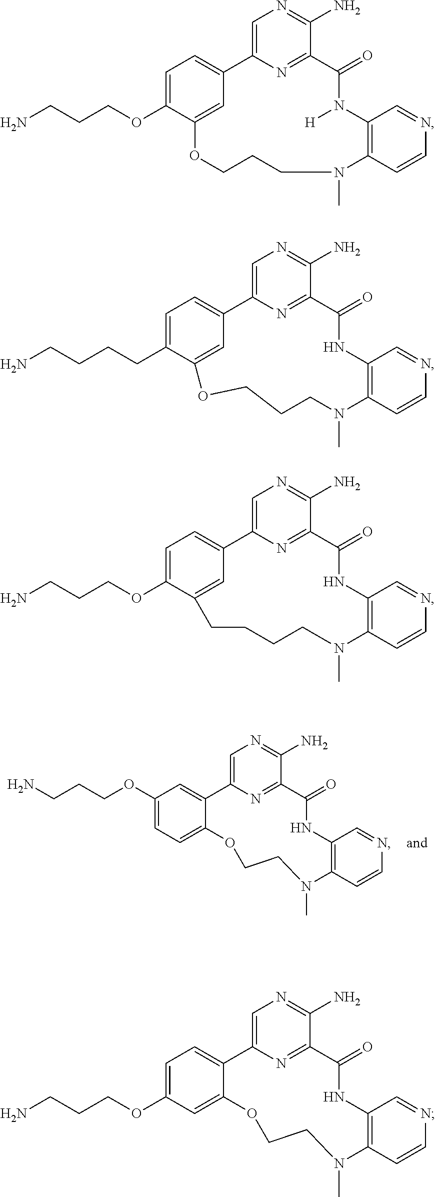
or a pharmaceutically acceptable salt of any one thereof.

83. A pharmaceutical composition comprising the compound or salt of any one of claims 1-82, and a pharmaceutically acceptable excipient.

84. A method for the treatment of cancer, autoimmune diseases, inflammation, sepsis, allergy, asthma, graft rejection, graft-versus-host disease, fibrosis, immunodeficiencies, or infectious disease comprising administering an effective amount of the compound or salt of any one of claims 1-82 or the pharmaceutical composition of claim 83 to a subject in need thereof.

85. A method for the treatment of cancer, comprising administering an effective amount of the compound or salt of any one of claims 1-82 or the pharmaceutical composition of claim 83 to a subject in need thereof.

86. A method for the treatment of fibrosis, comprising administering an effective amount of the compound or salt of any one of claims 1-82 or the pharmaceutical composition of claim 83 to a subject in need thereof.

87. A method for the treatment of fibrotic disease, comprising administering an effective amount of the compound or salt of any one of claims 1-82 or the pharmaceutical composition of claim 83 to a subject in need thereof.

88. A compound or salt of any one of claims 1-82 for use in a method of treating cancer.

89. A compound or salt of any one of claims 1-82 for use in a method of treating fibrosis.

90. A compound or salt of any one of claims 1-82 for use in a method of treating cancer, autoimmune diseases, inflammation, sepsis, allergy, asthma, graft rejection, graft-versus-host disease, fibrosis, immunodeficiencies, or infectious disease.

91. A conjugate represented by the following formula: 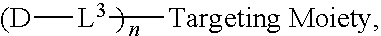
wherein L³ is a linker, D is a compound or salt of any one of claims 1-82, and n is from 1 to 20.

92. The conjugate of claim 91, wherein n ranges from 1 to about 10, from 2 to about 8, or from about 3 to about 5, or is about 4.

93. The conjugate of claim 91 or 92, the targeting moiety is an antibody.

94. The conjugate of any one of claims 91-93, wherein L³ comprises Val-Cit or Val-Ala.

95. The conjugate of any one of claims 91-94, wherein the targeting moiety or antibody specifically binds to a tumor antigen.

96. The conjugate of any one of claims 91-95, wherein the targeting moiety or antibody comprises a binding domain specific for LRRC15, an ASGR1, or an ASGR2.

97. The conjugate of any one of claims 91-95, wherein D is a compound from Table 1.

98. The conjugate of any one of claims 91-95, wherein D is any one of Compounds 7, 16, 26, 28, 30, 31, 36, 38, 40, 41, 53-56, and 61-65.


SEQUENCE LISTING (236 sequences)

SEQ ID NO: 1 - protein, 8 aa, Artificial Sequence
  source: /note=“Description of Artificial Sequence: Synthetic peptide”
GFTFSSYT

SEQ ID NO: 2 - protein, 8 aa, Artificial Sequence
  source: /note=“Description of Artificial Sequence: Synthetic peptide”
ISYDGNNK

SEQ ID NO: 3 - protein, 11 aa, Artificial Sequence
  source: /note=“Description of Artificial Sequence: Synthetic peptide”
ARTGWLGPFDY

SEQ ID NO: 4 - protein, 7 aa, Artificial Sequence
  source: /note=“Description of Artificial Sequence: Synthetic peptide”
QSVGSSY

SEQ ID NO: 5 - protein, 3 aa, Artificial Sequence
  source: /note=“Description of Artificial Sequence: Synthetic peptide”
SSY

SEQ ID NO: 6 - protein, 9 aa, Artificial Sequence
  source: /note=“Description of Artificial Sequence: Synthetic peptide”
QQYGSSPWT

SEQ ID NO: 7 - protein, 8 aa, Artificial Sequence
  source: /note=“Description of Artificial Sequence: Synthetic peptide”
GITFSNSG

SEQ ID NO: 8 - protein, 8 aa, Artificial Sequence
  source: /note=“Description of Artificial Sequence: Synthetic peptide”
IWYDGSKR

SEQ ID NO: 9 - protein, 6 aa, Artificial Sequence
  source: /note=“Description of Artificial Sequence: Synthetic peptide”
ATNDDY

SEQ ID NO: 10 - protein, 7 aa, Artificial Sequence
  source: /note=“Description of Artificial Sequence: Synthetic peptide”
QSVSSYL

SEQ ID NO: 11 - protein, 3 aa, Artificial Sequence
  source: /note=“Description of Artificial Sequence: Synthetic peptide”
DAS

SEQ ID NO: 12 - protein, 9 aa, Artificial Sequence
  source: /note=“Description of Artificial Sequence: Synthetic peptide”
QQSSNWPRT

SEQ ID NO: 13 - protein, 8 aa, Artificial Sequence
  source: /note=“Description of Artificial Sequence: Synthetic peptide”
GYTFTNYY

SEQ ID NO: 14 - protein, 8 aa, Artificial Sequence
  source: /note=“Description of Artificial Sequence: Synthetic peptide”
INPSNGGT

SEQ ID NO: 15 - protein, 13 aa, Artificial Sequence
  source: /note=“Description of Artificial Sequence: Synthetic peptide”
ARRDYRFDMGFDY

SEQ ID NO: 16 - protein, 10 aa, Artificial Sequence
  source: /note=“Description of Artificial Sequence: Synthetic peptide”
KGVSTSGYSY

SEQ ID NO: 17 - protein, 3 aa, Artificial Sequence
  source: /note=“Description of Artificial Sequence: Synthetic peptide”
LAS

SEQ ID NO: 18 - protein, 9 aa, Artificial Sequence
  source: /note=“Description of Artificial Sequence: Synthetic peptide”
QHSRDLPLT

SEQ ID NO: 19 - protein, 8 aa, Artificial Sequence
  source: /note=“Description of Artificial Sequence: Synthetic peptide”
GYTFTDSY

SEQ ID NO: 20 - protein, 8 aa, Artificial Sequence
  source: /note=“Description of Artificial Sequence: Synthetic peptide”
MYPDNGDS

SEQ ID NO: 21 - protein, 10 aa, Artificial Sequence
  source: /note=“Description of Artificial Sequence: Synthetic peptide”
VLAPRWYFSV

SEQ ID NO: 22 - protein, 6 aa, Artificial Sequence
  source: /note=“Description of Artificial Sequence: Synthetic peptide”
QDISNY

SEQ ID NO: 23 - protein, 3 aa, Artificial Sequence
  source: /note=“Description of Artificial Sequence: Synthetic peptide”
YTS

SEQ ID NO: 24 - protein, 9 aa, Artificial Sequence
  source: /note=“Description of Artificial Sequence: Synthetic peptide”
QQGHTLPPT

SEQ ID NO: 25 - protein, 8 aa, Artificial Sequence
  source: /note=“Description of Artificial Sequence: Synthetic peptide”
GFTFSSYD

SEQ ID NO: 26 - protein, 8 aa, Artificial Sequence
  source: /note=“Description of Artificial Sequence: Synthetic peptide”
IWYDGSNK

SEQ ID NO: 27 - protein, 12 aa, Artificial Sequence
  source: /note=“Description of Artificial Sequence: Synthetic peptide”
ARGSGNWGFFDY

SEQ ID NO: 28 - protein, 6 aa, Artificial Sequence
  source: /note=“Description of Artificial Sequence: Synthetic peptide”
QGISRW

SEQ ID NO: 29 - protein, 3 aa, Artificial Sequence
  source: /note=“Description of Artificial Sequence: Synthetic peptide”
AAS

SEQ ID NO: 30 - protein, 9 aa, Artificial Sequence
  source: /note=“Description of Artificial Sequence: Synthetic peptide”
QQYNTYPRT

SEQ ID NO: 31 - protein, 8 aa, Artificial Sequence
  source: /note=“Description of Artificial Sequence: Synthetic peptide”
GYTFTSYR

SEQ ID NO: 32 - protein, 8 aa, Artificial Sequence
  source: /note=“Description of Artificial Sequence: Synthetic peptide”
INPSTGYT

SEQ ID NO: 33 - protein, 9 aa, Artificial Sequence
  source: /note=“Description of Artificial Sequence: Synthetic peptide”
ARGGGVFDY

SEQ ID NO: 34 - protein, 6 aa, Artificial Sequence
  source: /note=“Description of Artificial Sequence: Synthetic peptide”
SSSISY

SEQ ID NO: 35 - protein, 3 aa, Artificial Sequence
  source: /note=“Description of Artificial Sequence: Synthetic peptide”
TTS

SEQ ID NO: 36 - protein, 9 aa, Artificial Sequence
  source: /note=“Description of Artificial Sequence: Synthetic peptide”
HQRSTYPLT

SEQ ID NO: 37 - protein, 5 aa, Artificial Sequence
  source: /note=“Description of Artificial Sequence: Synthetic peptide”
SYGMH

SEQ ID NO: 38 - protein, 17 aa, Artificial Sequence
  source: /note=“Description of Artificial Sequence: Synthetic peptide”
VIWYEGSNKYYADSVKG

SEQ ID NO: 39 - protein, 15 aa, Artificial Sequence
  source: /note=“Description of Artificial Sequence: Synthetic peptide”
GGSMVRGDYYYGMDV

SEQ ID NO: 40 - protein, 11 aa, Artificial Sequence
  source: /note=“Description of Artificial Sequence: Synthetic peptide”
RASQGISSALA

SEQ ID NO: 41 - protein, 7 aa, Artificial Sequence
  source: /note=“Description of Artificial Sequence: Synthetic peptide”
DASSLES

SEQ ID NO: 42 - protein, 9 aa, Artificial Sequence
  source: /note=“Description of Artificial Sequence: Synthetic peptide”
QQFNSYPYT

SEQ ID NO: 43 - protein, 5 aa, Artificial Sequence
  source: /note=“Description of Artificial Sequence: Synthetic peptide”
DYYWN

SEQ ID NO: 44 - protein, 16 aa, Artificial Sequence
  source: /note=“Description of Artificial Sequence: Synthetic peptide”
EINHRGSTNSNPSLKS

SEQ ID NO: 45 - protein, 12 aa, Artificial Sequence
  source: /note=“Description of Artificial Sequence: Synthetic peptide”
GYSDYEYNWFDP

SEQ ID NO: 46 - protein, 11 aa, Artificial Sequence
  source: /note=“Description of Artificial Sequence: Synthetic peptide”
RASQSISSYLA

SEQ ID NO: 47 - protein, 7 aa, Artificial Sequence
  source: /note=“Description of Artificial Sequence: Synthetic peptide”
DASNRAT

SEQ ID NO: 48 - protein, 9 aa, Artificial Sequence
  source: /note=“Description of Artificial Sequence: Synthetic peptide”
QQRSNWPLT

SEQ ID NO: 49 - protein, 5 aa, Artificial Sequence
  source: /note=“Description of Artificial Sequence: Synthetic peptide”
  MOD_RES (3)..(3): Any amino acid
LPXTG

SEQ ID NO: 50 - protein, 8 aa, Artificial Sequence
  source: /note=“Description of Artificial Sequence: Synthetic peptide”
GYSFSTYW

SEQ ID NO: 51 - protein, 8 aa, Artificial Sequence
  source: /note=“Description of Artificial Sequence: Synthetic peptide”
IYPGDSYT

SEQ ID NO: 52 - protein, 9 aa, Artificial Sequence
  source: /note=“Description of Artificial Sequence: Synthetic peptide”
ARGYGIFDY

SEQ ID NO: 53 - protein, 6 aa, Artificial Sequence
  source: /note=“Description of Artificial Sequence: Synthetic peptide”
NIGDQY

SEQ ID NO: 54 - protein, 3 aa, Artificial Sequence
  source: /note=“Description of Artificial Sequence: Synthetic peptide”
QDK

SEQ ID NO: 55 - protein, 11 aa, Artificial Sequence
  source: /note=“Description of Artificial Sequence: Synthetic peptide”
ATYTGFGSLAV

SEQ ID NO: 56 - protein, 8 aa, Artificial Sequence
  source: /note=“Description of Artificial Sequence: Synthetic peptide”
GYTFTDYN

SEQ ID NO: 57 - protein, 8 aa, Artificial Sequence
  source: /note=“Description of Artificial Sequence: Synthetic peptide”
INPNYEST

SEQ ID NO: 58 - protein, 9 aa, Artificial Sequence
  source: /note=“Description of Artificial Sequence: Synthetic peptide”
RDKGWYFDV

SEQ ID NO: 59 - protein, 5 aa, Artificial Sequence
  source: /note=“Description of Artificial Sequence: Synthetic peptide”
SSVKN

SEQ ID NO: 60 - protein, 3 aa, Artificial Sequence
  source: /note=“Description of Artificial Sequence: Synthetic peptide”
YTS

SEQ ID NO: 61 - protein, 9 aa, Artificial Sequence
  source: /note=“Description of Artificial Sequence: Synthetic peptide”
QQFTSSPYT

SEQ ID NO: 62 - protein, 10 aa, Artificial Sequence
  source: /note=“Description of Artificial Sequence: Synthetic peptide”
GFSLSTSGMG

SEQ ID NO: 63 - protein, 7 aa, Artificial Sequence
  source: /note=“Description of Artificial Sequence: Synthetic peptide”
IWWDDDK

SEQ ID NO: 64 - protein, 11 aa, Artificial Sequence
  source: /note=“Description of Artificial Sequence: Synthetic peptide”
ARLTGTRYFDY

SEQ ID NO: 65 - protein, 6 aa, Artificial Sequence
  source: /note=“Description of Artificial Sequence: Synthetic peptide”
QDINKF

SEQ ID NO: 66 - protein, 3 aa, Artificial Sequence
  source: /note=“Description of Artificial Sequence: Synthetic peptide”
YTS

SEQ ID NO: 67 - protein, 8 aa, Artificial Sequence
  source: /note=“Description of Artificial Sequence: Synthetic peptide”
LQYGNLWT

SEQ ID NO: 68 - protein, 8 aa, Artificial Sequence
  source: /note=“Description of Artificial Sequence: Synthetic peptide”
GYTFTDYS

SEQ ID NO: 69 - protein, 8 aa, Artificial Sequence
  source: /note=“Description of Artificial Sequence: Synthetic peptide”
INTETGEP

SEQ ID NO: 70 - protein, 12 aa, Artificial Sequence
  source: /note=“Description of Artificial Sequence: Synthetic peptide”
ATYYGSSYVPDY

SEQ ID NO: 71 - protein, 6 aa, Artificial Sequence
  source: /note=“Description of Artificial Sequence: Synthetic peptide”
QNVGTA

SEQ ID NO: 72 - protein, 3 aa, Artificial Sequence
  source: /note=“Description of Artificial Sequence: Synthetic peptide”
WTS

SEQ ID NO: 73 - protein, 8 aa, Artificial Sequence
  source: /note=“Description of Artificial Sequence: Synthetic peptide”
QYSDYPYT

SEQ ID NO: 74 - protein, 7 aa, Artificial Sequence
  source: /note=“Description of Artificial Sequence: Synthetic peptide”
GYTFTDY

SEQ ID NO: 75 - protein, 8 aa, Artificial Sequence
  source: /note=“Description of Artificial Sequence: Synthetic peptide”
WVDPEYGS

SEQ ID NO: 76 - protein, 12 aa, Artificial Sequence
  source: /note=“Description of Artificial Sequence: Synthetic peptide”
ARDDGSYSPFDY

SEQ ID NO: 77 - protein, 6 aa, Artificial Sequence
  source: /note=“Description of Artificial Sequence: Synthetic peptide”
QNINKY

SEQ ID NO: 78 - protein, 3 aa, Artificial Sequence
  source: /note=“Description of Artificial Sequence: Synthetic peptide”
YTS

SEQ ID NO: 79 - protein, 8 aa, Artificial Sequence
  source: /note=“Description of Artificial Sequence: Synthetic peptide”
LQYVNLLT

SEQ ID NO: 80 - protein, 6 aa, Artificial Sequence
  source: /note=“Description of Artificial Sequence: Synthetic peptide”
ENVVTY

SEQ ID NO: 81 - protein, 3 aa, Artificial Sequence
  source: /note=“Description of Artificial Sequence: Synthetic peptide”
GAS

SEQ ID NO: 82 - protein, 8 aa, Artificial Sequence
  source: /note=“Description of Artificial Sequence: Synthetic peptide”
QGYSYPYT

SEQ ID NO: 83 - protein, 118 aa, Artificial Sequence
  source: /note=“Description of Artificial Sequence: Synthetic polypeptide”
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSS

SEQ ID NO: 84 - protein, 108 aa, Artificial Sequence
  source: /note=“Description of Artificial Sequence: Synthetic polypeptide”
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIK

SEQ ID NO: 85 - protein, 113 aa, Artificial Sequence [sequence omitted]

SEQ ID NO: 86 - protein, 107 aa, Artificial Sequence [sequence omitted]

SEQ ID NO: 87 - protein, 120 aa, Artificial Sequence [sequence omitted]

SEQ ID NO: 88 - protein, 111 aa, Artificial Sequence [sequence omitted]

SEQ ID NO: 89 - protein, 118 aa, Artificial Sequence [sequence omitted]

SEQ ID NO: 90 - protein, 107 aa, Artificial Sequence [sequence omitted]

SEQ ID NO: 91 - protein, 121 aa, Artificial Sequence
  source: /note=“Description of Artificial Sequence: Synthetic polypeptide”
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSS

SEQ ID NO: 92 - protein, 108 aa, Artificial Sequence
  source: /note=“Description of Artificial Sequence: Synthetic polypeptide”
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIK

SEQ ID NO: 93 - protein, 123 aa, Artificial Sequence
  source: /note=“Description of Artificial Sequence: Synthetic polypeptide”
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSS

SEQ ID NO: 94 - protein, 106 aa, Artificial Sequence
  source: /note=“Description of Artificial Sequence: Synthetic polypeptide”
EIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIK

SEQ ID NO: 95 - protein, 117 aa, Artificial Sequence
  source: /note=“Description of Artificial Sequence: Synthetic polypeptide”
EVQLQQSGAELVKPGASVKISCKASGYTFTDYNMDWVKQSHGKSLEWIGDINPNYESTSYNQKFKGKATLTVDKSSSTAYMEVRSLTSEDTAVFYCARDKGWYFDVWGAGTTVTVSS

SEQ ID NO: 96 - protein, 106 aa, Artificial Sequence
  source: /note=“Description of Artificial Sequence: Synthetic polypeptide”
ENVLTQSPAIMSASLGEKVTMSCRASSSVKNMYWYQQKSDASPKLWIYYTSNLAPGVPARFSGSGSGNSYSLTISSMEGEDAATYYCQQFTSSPYTFGGGTKLELK

SEQ ID NO: 97 - protein, 119 aa, Artificial Sequence
  source: /note=“Description of Artificial Sequence: Synthetic polypeptide”
QVTLKESGPGILQPSQTLSLTCSFSGFSLSTSGMGVGWIRQPSGKGLEWLAHIWWDDDKFYNPSLKSQLTISKDTSRNQVFLKLTSVVTADTATYYCARLTGTRYFDYWGQGTTLTVSS

SEQ ID NO: 98 - protein, 106 aa, Artificial Sequence
  source: /note=“Description of Artificial Sequence: Synthetic polypeptide”
DVQMTQSPSSLSASLGGKVTITCKASQDINKFIAWYQHKPGKGPRLLIHYTSTLQPGIPSKFSGSGSGRDYSFSISNLEPEDIATYYCLQYGNLWTFGGGTKLEIT

SEQ ID NO: 99 - protein, 119 aa, Artificial Sequence
  source: /note=“Description of Artificial Sequence: Synthetic polypeptide”
QIQLVQSGPELKKPGETVKISCKASGYTFTDYSMHWVKQAPGKGLKWMGWINTETGEPTYADDFKGRFAFSSETSTSTAYLQINNLKNDDTTTYFCATYYGSSYVPDYWGQGTSLTVSS

SEQ ID NO: 100 - protein, 107 aa, Artificial Sequence
  source: /note=“Description of Artificial Sequence: Synthetic polypeptide”
DIVMTQSHKFMSTSVGDRVSITCKASQNVGTAVAWYQHKPGQSPKLLIYWTSSRHTGVPDRFTGSGSGTEFTLTISNVQSEDLADYFCHQYSDYPYTFGGGTKLEIK

SEQ ID NO: 101 - protein, 119 aa, Artificial Sequence
  source: /note=“Description of Artificial Sequence: Synthetic polypeptide”
EVQLQQSGPEVGRPGSSVKISCKASGYTFTDYIMHWVKQSPGQGLEWIGWVDPEYGSTDYAEKFKKKATLTADTSSNTAYIQLSSLTSEDTATYFCARDDGSYSPFDYWGQGVMVTVSS

SEQ ID NO: 102 - protein, 106 aa, Artificial Sequence
  source: /note=“Description of Artificial Sequence: Synthetic polypeptide”
DIQMTQSPPSLSASLGDKVTITCQASQNINKYIAWYQQKPGKAPRLLIRYTSTLESGTPSRFSGSGSGRDYSFSISNVESEDIASYYCLQYVNLLTFGAGTKLEIK

SEQ ID NO: 103 - protein, 107 aa, Artificial Sequence
  source: /note=“Description of Artificial Sequence: Synthetic polypeptide”
NIVMTQSPKSMSMSVGERVTLTCKASENVVTYVSWYQQKPEQSPKLLIYGASNRYTGVPDRFTGSGSATDFTLTISSVQAEDLADYHCGQGYSYPYTFGGGTKLEIK

SEQ ID NO: 104 - protein, 120 aa, Artificial Sequence
  source: /note=“Description of Artificial Sequence: Synthetic polypeptide”
EVQLVESGGGLVQPGGSLRLSCAASGFTFSDYAMSWVRQAPGKGLEWVAVISENGSDTYYADSVKGRFTISRDDSKNTLYLQMNSLRAEDTAVYYCARDRGGAVSYFDVWGQGTLVTVSS

SEQ ID NO: 105 - protein, 109 aa, Artificial Sequence
  source: /note=“Description of Artificial Sequence: Synthetic polypeptide”
DIQMTQSPSSLSASVGDRVTITCRASQDVSSYLAWYQQKPGKAPKLLIYAASSLESGVPSRFSGSGSGTDFTLTISSLQPEDFATYYCQQYNSLPYTFGQGTKVEIKRT

SEQ ID NO: 106 - protein, 117 aa, Artificial Sequence
  source: /note=“Description of Artificial Sequence: Synthetic polypeptide”
EVQLVQSGAEVKKPGASVKVSCKASGYTFTDSYMSWVRQAPGQGLEWIGDMYPDNGDSSYNQKFRERVTITRDTSTSTAYLELSSLRSEDTAVYYCVLAPRWYFSVWGQGTLVTVSS

SEQ ID NO: 107 - protein, 107 aa, Artificial Sequence
  source: /note=“Description of Artificial Sequence: Synthetic polypeptide”
DIQMTQSPSSLSASVGDRVTITCRASQDISNYLNWYQQKPGKAPKLLIYYTSRLRSGVPSRFSGSGSGTDFTLTISSLQPEDFATYYCQQGHTLPPTFGQGTKVEIK

SEQ ID NO: 108 - protein, 107 aa, Artificial Sequence
  source: /note=“Description of Artificial Sequence: Synthetic polypeptide”
EIVMTQSPATLSVSPGERATLSCKASQNVGTNVAWYQQKPGQAPRLLIYSASYRYSGIPARFSGSGSGTEFTLTISSLQSEDFAVYYCQQYNTDPLTFGGGTKVEIK

SEQ ID NO: 109 - protein, 119 aa, Artificial Sequence
  source: /note=“Description of Artificial Sequence: Synthetic polypeptide”
QVTLRESGPALVKPTQTLTLTCTFSGFSLSTSGMGVGWIRQPPGKALEWLAHIWWDDDKYYNPSLKSRLTISKDTSKNQVVLTMTNMDPVDTATYYCARTRRYFPFAYWGQGTLVTVSS

SEQ ID NO: 110 - protein, 119 aa, Artificial Sequence
  source: /note=“Description of Artificial Sequence: Synthetic polypeptide”
QVTLRESGPALVKPTQTLTLTCTFSGFSLSTSGMGVGWIRQPPGKALEWLAHIWWDDDKYYQPSLKSRLTISKDTSKNQVVLTMTNMDPVDTATYYCARTRRYFPFAYWGQGTLVTVSS

SEQ ID NO: 111 - protein, 120 aa, Artificial Sequence
  source: /note=“Description of Artificial Sequence: Synthetic polypeptide”
QVQLQQWGAGLLKPSETLSLTCAVYGGSFSDYYWNWIRQPPGKGLEWIGEINHRGSTNSNPSLKSRVTLSLDTSKNQFSLKLRSVTAADTAVYYCAFGYSDYEYNWFDPWGQGTLVTVSS

SEQ ID NO: 112 - protein, 107 aa, Artificial Sequence
  source: /note=“Description of Artificial Sequence: Synthetic polypeptide”
EIVLTQSPATLSLSPGERATLSCRASQSISSYLAWYQQKPGQAPRLLIYDASNRATGIPARFSGSGSGTDFTLTISSLEPEDFAVYYCQQRSNWPLTFGQGTNLEIK

SEQ ID NO: 113 - protein, 140 aa, Artificial Sequence
  source: /note=“Description of Artificial Sequence: Synthetic polypeptide”
MAVLALLFCLVTFPSCILSQVQLKESGPGLVAPSQSLSITCTVSGFSLTGYGINWVRQPPGKGLEWLGMIWSDGSTDYNSVLTSRLRISKDNSNSQVFLKMNSLQVDDTARYYCARDRNYYDYDGAMDYWGQGTSVTVSS

SEQ ID NO: 114 - protein, 121 aa, Artificial Sequence
  source: /note=“Description of Artificial Sequence: Synthetic polypeptide”
QVQLKESGPGLVAPSQSLSITCTVSGFSLTGYGINWVRQPPGKGLEWLGMIWSDGSTDYNSVLTSRLRISKDNSNSQVFLKMNSLQVDDTARYYCARDRNYYDYDGAMDYWGQGTSVTVSS

SEQ ID NO: 115 - protein, 127 aa, Artificial Sequence
  source: /note=“Description of Artificial Sequence: Synthetic polypeptide”
MKFPSQLLLFLLFRITGIICDIQVTQSSSYLSVSLGDRVTITCKASDHIKNWLAWYQQKPGIAPRLLVSGATSLEAGVPSRFSGSGSGKNFTLSITSLQTEDVATYYCQQYWSTPWTFGGGTTLEIR

SEQ ID NO: 116 - protein, 107 aa, Artificial Sequence
  source: /note=“Description of Artificial Sequence: Synthetic polypeptide”
DIQVTQSSSYLSVSLGDRVTITCKASDHIKNWLAWYQQKPGIAPRLLVSGATSLEAGVPSRFSGSGSGKNFTLSITSLQTEDVATYYCQQYWSTPWTFGGGTTLEIR

SEQ ID NO: 117 - protein, 126 aa, Artificial Sequence
  source: /note=“Description of Artificial Sequence: Synthetic polypeptide”
EVQLVQPGAELRNSGASVKVSCKASGYRFTSYYIDWVRQAPGQGLEWMGRIDPEDGGTKYAQKFQGRVTFTADTSTSTAYVELSSLRSEDTAVYYCARNEWETVVVGDLMYEYEYWGQGTQVTVSS

SEQ ID NO: 118 - protein, 107 aa, Artificial Sequence
  source: /note=“Description of Artificial Sequence: Synthetic polypeptide”
DIQMTQSPTSLSASLGDRVTITCQASQSISSYLAWYQQKPGQAPKLLIYGASRLQTGVPSRFSGSGSGTSFTLTISGLEAEDAGTYYCQQYDSLPVTFGQGTKVELK

SEQ ID NO: 119 - protein, 107 aa, Artificial Sequence
  source: /note=“Description of Artificial Sequence: Synthetic polypeptide”
DIQMTQSPSSLSASLGDRVTITCQASQSIVSYLAWYQQKPGQAPKLLIYGASRLQTGVPSRFSGSGSGTSFTLTISGLEAEDAGTYYCQQYASAPVTFGQGTGVELK

SEQ ID NO: 120 - protein, 107 aa, Artificial Sequence
  source: /note=“Description of Artificial Sequence: Synthetic polypeptide”
DIQMTQSPSSLSASLGDRVTITCQASQSISSYLAWYQQKPGQAPKLLIYGTSRLKTGVPSRFSGSGSGTSFTLTISGLEAEDAGTYYCQQYYSAPVTFGQGTKVELK

SEQ ID NO: 121 - protein, 107 aa, Artificial Sequence
  source: /note=“Description of Artificial Sequence: Synthetic polypeptide”
DIQMTQSPSSLSPSLGDRVTITCQASQTISSFLAWYHQKPGQPPKLLIYRASIPQTGVPSRFSGSGSGTSFTLTIGGLEAEDAGTYYCQQYVSAPPTFGQGTKVELK

SEQ ID NO: 122 - protein, 107 aa, Artificial Sequence
  source: /note=“Description of Artificial Sequence: Synthetic polypeptide”
DIQMTQSPSSLSASLGDRVTITCQASQSISSYLAWYQQKPGQAPNILIYGASRLKTGVPSRFSGSGSGTSFTLTISGLEAEDAGTYYCQQYASVPVTFGQGTKVELK

SEQ ID NO: 123 - protein, 121 aa, Artificial Sequence [sequence omitted]

SEQ ID NO: 124 - protein, 109 aa, Artificial Sequence [sequence omitted]

SEQ ID NO: 125 - protein, 114 aa, Artificial Sequence
  source: /note=“Description of Artificial Sequence: Synthetic polypeptide”
EVQLVESGGGLVQPGGSLRLSCAASGFTFSDYWMSWVRQAPGKGLEWVADIKNDGSYTNYAPSLTNRFTISRDNAKNSLYLQMNSLRAEDTAVYYCARELTGTWGQGTMVTVSS

SEQ ID NO: 126 - protein, 113 aa, Artificial Sequence
  source: /note=“Description of Artificial Sequence: Synthetic polypeptide”
DIVMTQSPDSLAVSLGERATINCKSSQSLLSSGNQKNYLAWYQQKPGQPPKLLIYYASTRQSGVPDRFSGSGSGTDFTLTISSLQAEDVAVYYCLQYDRYPFTFGQGTKLEIK

SEQ ID NO: 127 - protein, 116 aa, Artificial Sequence
  source: /note=“Description of Artificial Sequence: Synthetic polypeptide”
EVQLVQSGAEVKKPGESLRISCKGSGYSFSTYWISWVRQMPGKGLEWMGKIYPGDSYTNYSPSFQGQVTISADKSISTAYLQWSSLKASDTAMYYCARGYGIFDYWGQGTLVTVSS

SEQ ID NO: 128 - protein, 108 aa, Artificial Sequence
  source: /note=“Description of Artificial Sequence: Synthetic polypeptide”
SYELTQPPSVSVSPGQTASITCSGDNIGDQYAHWYQQKPGQSPVLVIYQDKNRPSGIPERFSGSNSGNTATLTISGTQAMDEADYYCATYTGFGSLAVFGGGTKLTVL

SEQ ID NO: 129 - protein, 107 aa, Artificial Sequence
  source: /note=“Description of Artificial Sequence: Synthetic polypeptide”
DIQMTQSPSSVSASVGDRVTITCRASQGISRLLAWYQQKPGKAPKLLIYVASSLQSGVPSRFSGSGSGTDFTLTISSLQPEDFATYYCQQANSFPWTFGQGTKVEIK

SEQ ID NO: 130 - protein, 125 aa, Artificial Sequence
  source: /note=“Description of Artificial Sequence: Synthetic polypeptide”
QVQLVQSGAEVKKPGASVKVSCKASGYTFTGYYMHWVRQAPGQGLEWMGWINPHSGGTNYAQKFQGRVTMTRDTSISTAYMELSRLRSDDTAVYYCARTYYYDSSGYYHDAFDIWGQGTMVTVSS

SEQ ID NO: 131 - protein, 117 aa, Artificial Sequence
  source: /note=“Description of Artificial Sequence: Synthetic polypeptide”
EVQLVESGGGLVQPGGSLRLSCAASGFTFSDYWMDWVRQAPGKGLVWVSNIDEDGSITEYSPFVKGRFTISRDNAKNTLYLQMNSLRAEDTAVYYCTRWGRFGFDSWGQGTLVTVSS

SEQ ID NO: 132 - protein, 111 aa, Artificial Sequence
  source: /note=“Description of Artificial Sequence: Synthetic polypeptide”
DIVMTQSPDSLAVSLGERATINCKSSQSLLSGSFNYLTWYQQKPGQPPKLLIFYASTRHTGVPDRFSGSGSGTDFTLTISSLQAEDVAVYYCHHHYNAPPTFGPGTKVDIK

SEQ ID NO: 133 - protein, 118 aa, Artificial Sequence
  source: /note=“Description of Artificial Sequence: Synthetic polypeptide”
QVQLVQSGAEVKKPGASVKVSCKASGYTFTNYGMNWVRQAPGQGLKWMGWINTYTGEPTYADAFKGRVTMTRDTSISTAYMELSRLRSDDTAVYYCARDYGDYGMDYWGQGTTVTVSS

SEQ ID NO: 134 - protein, 111 aa, Artificial Sequence
  source: /note=“Description of Artificial Sequence: Synthetic polypeptide”
DIVMTQSPDSLAVSLGERATINCRASKSVSTSGYSFMHWYQQKPGQPPKLLIYLASNLESGVPDRFSGSGSGTDFTLTISSLQAEDVAVYYCQHSREVPWTFGQGTKVEIK

SEQ ID NO: 135 - protein, 122 aa, Artificial Sequence
  source: /note=“Description of Artificial Sequence: Synthetic polypeptide”
QLQLQESGPGLVKPSETLSLTCTVSGGSITSSSYYWGWIRQPPGKGLEWIGSIYYRGSTNYNPSLKSRVTISVDSSKNQFYLKVSSVTAVDTAVYYCARQNGAARPSWFDPWGQGTLVTVSS

SEQ ID NO: 136 - protein, 108 aa, Artificial Sequence
  source: /note=“Description of Artificial Sequence: Synthetic polypeptide”
EIVLTQSPDTISLSPGERATLSCRASQSISSIYLAWYQQKPGQAPRLLIYGASSRVTGIPDRFSVSGSGTDFTLTISRLEPEDFAVYYCQHYGISPFTFGPGTKVDIR

SEQ ID NO: 137 - protein, 117 aa, Artificial Sequence
  source: /note=“Description of Artificial Sequence: Synthetic polypeptide”
EVQLLESGGGLVQPGGSLRLSCAASGFTFSSYAYSWVRQAPGKGLEWVSAISGSGGRTYYADSVKGRFTISRDNSKNTLYLQMNSLRAEDTAVYYCARLGYGRVDEWGRGTLVTVSS

SEQ ID NO: 138 - protein, 110 aa, Artificial Sequence
  source: /note=“Description of Artificial Sequence: Synthetic polypeptide”
QSVLTQPPSASGTPGQRVTISCSGSLSNIGRNPVNWYQQLPGTAPKLLIYLDNLRLSGVPDRFSGSKSGTSASLAISGLQSEDEADYYCATWDDSHPGWTFGGGTKLTVL

SEQ ID NO: 139 - protein, 107 aa, Artificial Sequence
  source: /note=“Description of Artificial Sequence: Synthetic polypeptide”
DIQMTQSPSSLSASVGDRVTITCRASQGISSWLAWYQQKPEKAPKSLIYAASSLQSGVPSRFSGSGSGTDFTLTISSLQPEDFATYYCQQYNSYPLTFGGGTKVEIK

SEQ ID NO: 140 - protein, 121 aa, Artificial Sequence
  source: /note=“Description of Artificial Sequence: Synthetic polypeptide”
QVQLVESGGGVVQPGRSLRLSCAASGFTFSNYGMHWVRQAPGKGLEWVAVILYDGSNKYYPDSVKGRFTISRDNSKNTLYLQMNSLRAEDTAVYYCARGGSSWYPDSFDIWGQGTMVTVSS

SEQ ID NO: 141 - protein, 122 aa, Artificial Sequence [sequence omitted]

SEQ ID NO: 142 - protein, 107 aa, Artificial Sequence [sequence omitted]

SEQ ID NO: 143 - protein, 117 aa, Artificial Sequence
  source: /note=“Description of Artificial Sequence: Synthetic polypeptide”
QVQLVESGGGVVQPGRSLRLSCAASGFTFSSYDMSWVRQAPGKGLEWVAKVSSGGGSTYYLDTVQGRFTISRDNSKNTLYLQMNSLRAEDTAVYYCARHLHGSFASWGQGTTVTVSS

SEQ ID NO: 144 - protein, 107 aa, Artificial Sequence
  source: /note=“Description of Artificial Sequence: Synthetic polypeptide”
EIVLTQSPATLSLSPGERATLSCQASQSISNFLHWYQQRPGQAPRLLIRYRSQSISGIPARFSGSGSGTDFTLTISSLEPEDFAVYYCQQSGSWPLTFGGGTKVEIK

SEQ ID NO: 145 - protein, 119 aa, Artificial Sequence
  source: /note=“Description of Artificial Sequence: Synthetic polypeptide”
QVQLVESGGGVVQPGRSRRLSCAASGFTFSRYTMHWVRQAPGKGLEWVAVISFDGSNKYYVDSVKGRFTISRDNSENTLYLQVNILRAEDTAVYYCAREARGSYAFDIWGQGTMVTVSS

SEQ ID NO: 146 - protein, 108 aa, Artificial Sequence
  source: /note=“Description of Artificial Sequence: Synthetic polypeptide”
EIVLTQSPATLSLSPGERATLSCRASQSVSSYLAWYQQKPGQAPRLLIYDASNRATGIPARFSGSGSGTDFTLTISSLEPEDFAVYYCQQRSNWPPFTFGPGTKVDIK

SEQ ID NO: 147 - protein, 116 aa, Artificial Sequence
  source: /note=“Description of Artificial Sequence: Synthetic polypeptide”
EVQLVESGGGLVQPGGSLRLSCAVSGFVFSRYWMSWVRQAPGKGLEWIGEINPDSSTINYTSSLKDRFTISRDNAKNSLYLQMNSLRAEDTAVYYCASLITTEDYWGQGTTVTVSS

SEQ ID NO: 148 - protein, 108 aa, Artificial Sequence
  source: /note=“Description of Artificial Sequence: Synthetic polypeptide”
EIVLTQSPSSLSLSPGERVTITCKASQDINSYLSWYQQKPGKAPKLLIYYANRLVDGVPARFSGSGSGQDYTLTISSLEPEDFAVYYCLQYDEFPYTFGGGTKLEIKR

SEQ ID NO: 149 - protein, 124 aa, Artificial Sequence
  source: /note=“Description of Artificial Sequence: Synthetic polypeptide”
QVQLQESGPGLVRPSQTLSLTCTASGYTFTDYVIHWVKQPPGRGLEWIGYINPYDDDTTYNQKFKGRVTMLVDTSSNTAYLRLSSVTAEDTAVYYCARRGNSYDGYFDYSMDYWGSGTPVTVSS

SEQ ID NO: 150 - protein, 108 aa, Artificial Sequence
  source: /note=“Description of Artificial Sequence: Synthetic polypeptide”
DIQMTQSPSSLSASVGDRVTITCRASQNVGTAVAWLQQTPGKAPKLLIYSASNRYTGVPSRFSGSGSGTDYTFTISSLQPEDIATYYCQQYTNYPMYTFGQGTKVQIK

SEQ ID NO: 151 - protein, 121 aa, Artificial Sequence
  source: /note=“Description of Artificial Sequence: Synthetic polypeptide”
QVQLQESGPGLVKPSQTLSLTCAISGDSVSSNSVTWNWIRQSPSRGLEWLGRTYYRSKWYNDYAVSVKGRITINPDTSKNQFYLQLKSVTPEDAAVYYCARDSSILYGDYWGQGTLVTVSS

SEQ ID NO: 152 - protein, 120 aa, Artificial Sequence
  source: /note=“Description of Artificial Sequence: Synthetic polypeptide”
QVQLQQSGPGLVKPSQTLSLTCAISGDSVSSNSVTWNWIRQSPSRGLEWLGRTYYRSKWYNDYAVSVKGRITINPDTSKNQFYLQLKSVTPEDAAVYYCARDSSILYGDYWGQGTLVTVS

SEQ ID NO: 153 - protein, 115 aa, Artificial Sequence
  source: /note=“Description of Artificial Sequence: Synthetic polypeptide”
QAVLTQPSSLSASPGASASLTCTLPSGINVGTYRIFWFQQKPGSPPQYLLSYKSDSDNHQGSGVPSRFSGSKDASANAGILLISGLQSEDEADYYCMIWHSSAWVFGGGTKLTVL

SEQ ID NO: 154 - protein, 122 aa, Artificial Sequence
  source: /note=“Description of Artificial Sequence: Synthetic polypeptide”
QVQLVQSGAEVKKPGASVKVSCKTSGYTFTDYYIHWVRQAPGQGLEWMGWINPNRGGTNYAQKFQGRVTMTRDTSIATAYMELSRLRSDDTAVYYCATASLKIAAVGTFDCWGQGTLVTVSS

SEQ ID NO: 155 - protein, 107 aa, Artificial Sequence
  source: /note=“Description of Artificial Sequence: Synthetic polypeptide”
SYELTQPPSVSVSPGQTARITCSGDALSKQYAFWFQQKPGQAPILVIYQDTKRPSGIPGRFSGSSSGTTVTLTISGAQADDEADYYCQSADSSGTYVFGTGTKVTVL

SEQ ID NO: 156 - protein, 116 aa, Artificial Sequence
  source: /note=“Description of Artificial Sequence: Synthetic polypeptide”
EVQLVETGGGVVQPGRSLRLSCAASGFSFSTHGMYWVRQPPGKGLEWVAVISYDGSDKKYADSVKGRFTISRDNSKNTVYLEMSSVRAEDTALYYCFCRRDAFDLWGQGTMVTVSS

SEQ ID NO: 157 - protein, 108 aa, Artificial Sequence
  source: /note=“Description of Artificial Sequence: Synthetic polypeptide”
SYVLTQPPSVSVSPGQTARITCSGDALPKKYAYWYQQKSGQAPVLVIYEDTKRPSGIPERFSGSSSGTMATLTISGAQVEDEADYYCYSTDSSGNYWVFGGGTEVTVL

SEQ ID NO: 158 - protein, 124 aa, Artificial Sequence
  source: /note=“Description of Artificial Sequence: Synthetic polypeptide”
EVQLVESGGGLVEPGGSLRLSCAASGFTFSDAWMNWVRQAPGKGLEWVGRIKTKSDGGTTDYAAPVRGRFSISRDDSKNTLFLEMNSLKTEDTAIYYCFITVIVVSSESPLDHWGQGTLVTVSS

SEQ ID NO: 159 - protein, 108 aa, Artificial Sequence
  source: /note=“Description of Artificial Sequence: Synthetic polypeptide”
SYELTQPPSVSVSPGQTARITCSGDELPKQYAYWYQQKPGQAPVLVIYKDRQRPSGIPERFSGSSSGTTVTLTISGVQAEDEADYYCQSAYSINTYVIFGGGTKLTVL

SEQ ID NO: 160 - protein, 125 aa, Artificial Sequence
  source: /note=“Description of Artificial Sequence: Synthetic polypeptide”
EVQLVESGGGLVKPGGSLRLSCAASGFTFSDYYMSWIRQAPGKGLEWISYISSGSSYTNYADSVKGRFTISRDNAKKSVYLEVNGLTVEDTAVYYCARVRYGDREMATIGGFDFWGQGTLVTVSS

SEQ ID NO: 161 - protein, 108 aa, Artificial Sequence
  source: /note=“Description of Artificial Sequence: Synthetic polypeptide”
SYELTQPPSVSVSPGQTARITCSGDALPKQYAYWYQQSPGQAPVLVIYKDSERPSGIPERFSGSSSGTTVTLTISGVQAEDEADYYCQSADSGGTSRIFGGGTKLTVL

SEQ ID NO: 162 - protein, 124 aa, Artificial Sequence
  source: /note=“Description of Artificial Sequence: Synthetic polypeptide”
QVQLQESGPGLVRSTETLSLTCLVSGDSINSHYWSWLRQSPGRGLEWIGYIYYTGPTNYNPSLKSRVSISLGTSKDQFSLKLSSVTAADTARYYCARNKVFWRGSDFYYYMDVWGKGTTVTVSS

SEQ ID NO: 163 - protein, 110 aa, Artificial Sequence
  source: /note=“Description of Artificial Sequence: Synthetic polypeptide”
EIVLTQSPGTLSLSLGERATLSCRASQSLANNYLAWYQQKPGQAPRLLMYDASTRATGIPDRFSGSGSGTDFTLTISRLEPEDFAVYYCQQFVTSHHMYIFGQGTKVEIK

SEQ ID NO: 164 - protein, 129 aa, Artificial Sequence
  source: /note=“Description of Artificial Sequence: Synthetic polypeptide”
HVQLQESGPGLVKPSETLSLTCTVSGGSISSNNYYWGWIRQTPGKGLEWIGSIYYSGSTNYNPSLKSRVTISVDTSKNQFSLKLSSVTAADTAVYYCARGARWQARPATRIDGVAFDIWGQGTMVTVSS

SEQ ID NO: 165 - protein, 129 aa, Artificial Sequence
  source: /note=“Description of Artificial Sequence: Synthetic polypeptide”
QVQLQESGPGLVKPSETLSLTCTVSGGSISSNNYYWGWIRQTPGKGLEWIGSIYYSGSTNYNPSLKSRVTISVDTSKNQFSLKLSSVTAADTAVYYCARGARWQARPATRIDGVAFDIWGQGTMVTVSS

SEQ ID NO: 166 - protein, 121 aa, Artificial Sequence
  source: /note=“Description of Artificial Sequence: Synthetic polypeptide”
EVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARDWSRSGYYLPDYWGQGTLVTVSS

SEQ ID NO: 167 - protein, 107 aa, Artificial Sequence
  source: /note=“Description of Artificial Sequence: Synthetic polypeptide”
ETTLTQSPGTLSLSPGERATLSCRASQTVTRNYLAWYQQKPGQAPRLLMYGASNRAAGVPDRFSGSGSGTDFTLTISRLEPEDFAVYYCQQFGSPYTFGQGTKVEIK

SEQ ID NO: 168 - protein, 112 aa, Artificial Sequence
  source: /note=“Description of Artificial Sequence: Synthetic polypeptide”
DVVMTQSPLSLPVTLGQPASISCRSSQSLLHSNGYNYLDWYLQRPGQSPHLLIFLGSNRASGVPDRFSGSGSGTDFTLKISRVEAEDVGIYYCMQALQTPPTFGQGTKVEIK

SEQ ID NO: 169-170 - skipped (2 entries)

SEQ ID NO: 171 - protein, 119 aa, Artificial Sequence; identical to SEQ ID NO: 145
  source: /note=“Description of Artificial Sequence: Synthetic polypeptide”

SEQ ID NO: 172 - protein, 108 aa, Artificial Sequence; identical to SEQ ID NO: 146
  source: /note=“Description of Artificial Sequence: Synthetic polypeptide”

SEQ ID NO: 173 - protein, 107 aa, Artificial Sequence [sequence omitted]

SEQ ID NO: 174 - protein, 118 aa, Artificial Sequence [sequence omitted]

SEQ ID NO: 175 - protein, 124 aa, Artificial Sequence
  source: /note=“Description of Artificial Sequence: Synthetic polypeptide”
QVQLQESGPGLVKPSQTLSLTCTVSGGSISSGVYYWTWIRQHPGNGLEWIGYIYYSGSTSYNPSLKSRVTISVDTSKKQFSLNLTSVTAADTAVYYCAREGPLRGDYYYGLDVWGQGTTVTVSS

SEQ ID NO: 176 - protein, 108 aa, Artificial Sequence
  source: /note=“Description of Artificial Sequence: Synthetic polypeptide”
EIVLTQSPGTLSLSPGERATLSCRAGQTISSRYLAWYQQKPGQAPRPLIYGASSRATGIPDRFSGSGSGTDFTLTISRLEPEDFAVYYCQQYGSSPRTFGQGTKVEIK

SEQ ID NO: 177 - protein, 128 aa, Artificial Sequence
  source: /note=“Description of Artificial Sequence: Synthetic polypeptide”
QVQLQESGPGLVKPSQTLSLTCTVSGGSISSGGYYWSWIRQHPGKGLEWIGYIYYSGSTYYNPSLKSRVTISVDTSKNQFSLKLSSVTAADTAMYYCARYRGPAAGRGDFYYFGMDVWGQGTTVTVSS

SEQ ID NO: 178 - protein, 113 aa, Artificial Sequence
  source: /note=“Description of Artificial Sequence: Synthetic polypeptide”
DIVMTQTPLSLSVTPGQPASIFCKSSQSLLNSDGKTYLCWYLQKPGQPPQLLIYEVSNRFSGVPDRFSGSGSGTDFTLKISRVEAEDVGVYYCMQGIQLPWAFFGQGTKVEIK

SEQ ID NO: 179 - protein, 125 aa, Artificial Sequence
  source: /note=“Description of Artificial Sequence: Synthetic polypeptide”
QVQLVESGGGVVQPGRSLRLSCAASGFTFSSYGMHWVRQAPGKGLEWVAVIWYGGSNKYYADSVKGRFTISRDNSKNTLYLQMNSLRAEDTAVYYCARDLAARRGDYYYYGMDVWGQGTTVTVSS

SEQ ID NO: 180 - protein, 107 aa, Artificial Sequence
  source: /note=“Description of Artificial Sequence: Synthetic polypeptide”
SSELTQDPVVSVALGQTVRITCQGDSLRSYYLSWYQQKPGQAPVLVIYGKNNRPSGIPDRFSGSNSGNTASLTITGAQAEDEADYYCNSRDSSGNHLFGGGTKLTVL

SEQ ID NO: 181 - protein, 125 aa, Artificial Sequence
  source: /note=“Description of Artificial Sequence: Synthetic polypeptide”
QVQLQESGPGLVKPSQTLSLTCTVSGGSISSGGYYWSWIRQHPGKGLEWIGYIYYSGRTYNNPSLKSRVTISVDTSKNQFSLKLSSVTAADTAVYYCARVATGRADYHFYAMDVWGQGTTVTVSS

SEQ ID NO: 182 - protein, 106 aa, Artificial Sequence
  source: /note=“Description of Artificial Sequence: Synthetic polypeptide”
SYELTQPSSVSVSPGQTARITCSGDVLAKKSARWFHQKPGQAPVLVIYKDSERPSGIPERFSGSSSGTTVTLTISGAQVEDEAAYYCYSAADNNLVFGGGTKLTVL

SEQ ID NO: 183 - protein, 119 aa, Artificial Sequence
  source: /note=“Description of Artificial Sequence: Synthetic polypeptide”
QVQLVESGGGVVQPGRSLRLSCAASGFTFSSYDMHWVRQAPGKGLEWVAVIWYDGSNKYYADSVKGRFTISRDNSKNTLYLQMNSLRAEDTAVYYCARGSGNWGFFDYWGQGTLVTVSS

SEQ ID NO: 184 - protein, 107 aa, Artificial Sequence
  source: /note=“Description of Artificial Sequence: Synthetic polypeptide”
DIQMTQSPSSLSASVGDRVTITCRASQGISRWLAWYQQKPEKAPKSLIYAASSLQSGVPSRFSGSGSGTDFTLTISSLQPEDFATYYCQQYNTYPRTFGQGTKVEIK

SEQ ID NO: 185 - protein, 116 aa, Artificial Sequence
  source: /note=“Description of Artificial Sequence: Synthetic polypeptide”
QVQLVQSGAEVKKPGSSVKVSCKASGYTFTSYRMHWVRQAPGQGLEWIGYINPSTGYTEYNQKFKDKATITADESTNTAYMELSSLRSEDTAVYYCARGGGVFDYWGQGTLVTVSS

SEQ ID NO: 186 - protein, 106 aa, Artificial Sequence
  source: /note=“Description of Artificial Sequence: Synthetic polypeptide”
DIQMTQSPSTLSASVGDRVTITCSASSSISYMHWYQQKPGKAPKLLIYTTSNLASGVPARFSGSGSGTEFTLTISSLQPDDFATYYCHQRSTYPLTFGQGTKVEVK

SEQ ID NO: 187 - protein, 124 aa, Artificial Sequence
  source: /note=“Description of Artificial Sequence: Synthetic polypeptide”
QVQLVESGGGVVQPGRSLRLSCAASGFTFSSYGMHWVRQAPGKGLEWVAVIWYEGSNKYYADSVKGRFTISRDNSKNTLYLQMNSLRAEDTAVYYCARGGSMVRGDYYYGMDVWGQGTTVTVSS

SEQ ID NO: 188 - protein, 107 aa, Artificial Sequence
  source: /note=“Description of Artificial Sequence: Synthetic polypeptide”
AIQLTQSPSSLSASVGDRVTITCRASQGISSALAWYQQKPGKAPKLLIYDASSLESGVPSRFSGSGSGTDFTLTISSLQPEDFATYYCQQFNSYPYTFGQGTKLEIK

SEQ ID NO: 189 - protein, 120 aa, Artificial Sequence
  source: /note=“Description of Artificial Sequence: Synthetic polypeptide”
EVQLVQSGAEVKKPGASVKVSCKASGYKFSSYWIEWVKQAPGQGLEWIGEILPGSDTTNYNEKFKDRATFTSDTSINTAYMELSRLRSDDTAVYYCARDRGNYRAWFGYWGQGTLVTVSS

SEQ ID NO: 190 - protein, 107 aa, Artificial Sequence
  source: /note=“Description of Artificial Sequence: Synthetic polypeptide”
DIQMTQSPSSLSASVGDRVTITCRASQDISNYLNWYQQKPGGAVKFLIYYTSRLHSGVPSRFSGSGSGTDYTLTISSLQPEDFATYFCQQGEALPWTFGGGTKVEIK

SEQ ID NO: 191 - protein, 119 aa, Artificial Sequence
  source: /note=“Description of Artificial Sequence: Synthetic polypeptide”
EVQLVQSGAEVKKPGSSVKVSCKASGFTFTDYYIHWVKQAPGQGLEWIGLVYPYIGGTNYNQKFKGKATLTVDTSTTTAYMEMSSLRSEDTAVYYCARGDNKYDAMDYWGQGTTVTVSS

SEQ ID NO: 192 - protein, 110 aa, Artificial Sequence
  source: /note=“Description of Artificial Sequence: Synthetic polypeptide”
DIVLTQSPDSLAVSLGERATINCRASQSVSTSSYSYMHWYQQKPGQPPKLLIKYASSLESGVPDRFSGSGSGTDFTLTISSLQAEDVAVYYCEQSWEIRTFGGGTKVEIK

SEQ ID NO: 193 - protein, 121 aa, Artificial Sequence
  source: /note=“Description of Artificial Sequence: Synthetic polypeptide”
EVQLVQSGAEVKKPGSSVKVSCKASGYTFTNYWMHWVKQAPGQGLEWIGMIHPNSGSTKHNEKFRGKATLTVDESTTTAYMELSSLRSEDTAVYYCARSDFGNYRWYFDVWGQGTTVTVSS

SEQ ID NO: 194 - protein, 107 aa, Artificial Sequence
  source: /note=“Description of Artificial Sequence: Synthetic polypeptide”
EIVLTQSPATLSLSPGERATLSCRASQSSSNNLHWYQQKPGQAPRVLIKYVSQSISGIPARFSGSGSGTDFTLTISSLEPEDFAVYFCQQSNSWPFTFGQGTKLEIK

SEQ ID NO: 195 - protein, 119 aa, Artificial Sequence
  source: /note=“Description of Artificial Sequence: Synthetic polypeptide”
EVQLVQSGAEVKKPGSSVKVSCKASGFTFTDYYIHWVKQAPGQGLEWIGLVYPYIGGSSYNQQFKGKATLTVDTSTSTAYMELSSLRSEDTAVYYCARGDNNYDAMDYWGQGTTVTVSS

SEQ ID NO: 196 - protein, 110 aa, Artificial Sequence
  source: /note=“Description of Artificial Sequence: Synthetic polypeptide”
DIVLTQSPDSLAVSLGERATISCRASQSVSTSTYNYMHWYQQKPGQPPKLLVKYASNLESGVPDRFSGSGSGTDFTLTISSLQAEDVAVYYCHHTWEIRTFGGGTKVEIK

SEQ ID NO: 197 - protein, 120 aa, Artificial Sequence
  source: /note=“Description of Artificial Sequence: Synthetic polypeptide”
EVQLVESGGGLVQPGGSLRLSCAVSGFSLTSYGVHWVRQATGKGLEWLGVIWAGGSTNYNSALMSRLTISKENAKSSVYLQMNSLRAGDTAMYYCATHMITEDYYGMDYWGQGTTVTVSS

SEQ ID NO: 198 - protein, 112 aa, Artificial Sequence
  source: /note=“Description of Artificial Sequence: Synthetic polypeptide”
DIVMTQSPDSLAVSLGERATINCKSSQSLLNSRTRKNYLAWYQQKPGQSPKLLIYWASTRESGVPDRFSGSGSGTDFTLTISSLQAEDVAVYYCKQSYNLPTFGGGTKVEIK

SEQ ID NO: 199 - protein, 5 aa, Artificial Sequence
  source: /note=“Description of Artificial Sequence: Synthetic peptide”
DYYIH

SEQ ID NO: 200 - protein, 17 aa, Artificial Sequence
  source: /note=“Description of Artificial Sequence: Synthetic peptide”
LVYPYIGGTNYNQKFKG

SEQ ID NO: 201 - protein, 10 aa, Artificial Sequence
  source: /note=“Description of Artificial Sequence: Synthetic peptide”
GDNKYDAMDY

SEQ ID NO: 202 - protein, 15 aa, Artificial Sequence
  source: /note=“Description of Artificial Sequence: Synthetic peptide”
RASQSVSTSSYSYMH

SEQ ID NO: 203 - protein, 7 aa, Artificial Sequence
  source: /note=“Description of Artificial Sequence: Synthetic peptide”
YASSLES

SEQ ID NO: 204 - protein, 8 aa, Artificial Sequence
  source: /note=“Description of Artificial Sequence: Synthetic peptide”
EQSWEIRT

SEQ ID NO: 205 - protein, 5 aa, Artificial Sequence
  source: /note=“Description of Artificial Sequence: Synthetic peptide”
NYWMH

SEQ ID NO: 206 - protein, 17 aa, Artificial Sequence
  source: /note=“Description of Artificial Sequence: Synthetic peptide”
MIHPNSGSTKHNEKFRG

SEQ ID NO: 207 - protein, 12 aa, Artificial Sequence
  source: /note=“Description of Artificial Sequence: Synthetic peptide”
SDFGNYRWYFDV

SEQ ID NO: 208 - protein, 11 aa, Artificial Sequence
  source: /note=“Description of Artificial Sequence: Synthetic peptide”
RASQSSSNNLH

SEQ ID NO: 209 - protein, 7 aa, Artificial Sequence
  source: /note=“Description of Artificial Sequence: Synthetic peptide”
YVSQSIS

SEQ ID NO: 210 - protein, 9 aa, Artificial Sequence
  source: /note=“Description of Artificial Sequence: Synthetic peptide”
QQSNSWPFT

SEQ ID NO: 211 - protein, 5 aa, Artificial Sequence
  source: /note=“Description of Artificial Sequence: Synthetic peptide”
DYYIH

SEQ ID NO: 212 - protein, 17 aa, Artificial Sequence
  source: /note=“Description of Artificial Sequence: Synthetic peptide”
LVYPYIGGSSYNQQFKG

SEQ ID NO: 213 - protein, 10 aa, Artificial Sequence
  source: /note=“Description of Artificial Sequence: Synthetic peptide”
GDNNYDAMDY

SEQ ID NO: 214 - protein, 15 aa, Artificial Sequence
  source: /note=“Description of Artificial Sequence: Synthetic peptide”
RASQSVSTSTYNYMH

SEQ ID NO: 215 - protein, 7 aa, Artificial Sequence
  source: /note=“Description of Artificial Sequence: Synthetic peptide”
YASNLES

SEQ ID NO: 216 - protein, 8 aa, Artificial Sequence
  source: /note=“Description of Artificial Sequence: Synthetic peptide”
HHTWEIRT

SEQ ID NO: 217 - protein, 5 aa, Artificial Sequence
  source: /note=“Description of Artificial Sequence: Synthetic peptide”
SYGVH

SEQ ID NO: 218 - protein, 16 aa, Artificial Sequence
  source: /note=“Description of Artificial Sequence: Synthetic peptide”
VIWAGGSTNYNSALMS

SEQ ID NO: 219 - protein, 12 aa, Artificial Sequence
  source: /note=“Description of Artificial Sequence: Synthetic peptide”
HMITEDYYGMDY

SEQ ID NO: 220 - protein, 17 aa, Artificial Sequence
  source: /note=“Description of Artificial Sequence: Synthetic peptide”
KSSQSLLNSRTRKNYLA

SEQ ID NO: 221 - protein, 7 aa, Artificial Sequence
  source: /note=“Description of Artificial Sequence: Synthetic peptide”
WASTRES

SEQ ID NO: 222 - protein, 8 aa, Artificial Sequence
  source: /note=“Description of Artificial Sequence: Synthetic peptide”
KQSYNLPT

SEQ ID NO: 223 - protein, 5 aa, Artificial Sequence
  source: /note=“Description of Artificial Sequence: Synthetic peptide”
NYWLG

SEQ ID NO: 224 - protein, 17 aa, Artificial Sequence
  source: /note=“Description of Artificial Sequence: Synthetic peptide”
DIYPGGGNTYYNEKLKG

SEQ ID NO: 225 - protein, 11 aa, Artificial Sequence
  source: /note=“Description of Artificial Sequence: Synthetic peptide”
WGDKKGNYFAY

SEQ ID NO: 226 - protein, 12 aa, Artificial Sequence
  source: /note=“Description of Artificial Sequence: Synthetic peptide”
TASSSVYSSYLH

SEQ ID NO: 227 - protein, 7 aa, Artificial Sequence
  source: /note=“Description of Artificial Sequence: Synthetic peptide”
STSNLAS

SEQ ID NO: 228 - protein, 8 aa, Artificial Sequence
  source: /note=“Description of Artificial Sequence: Synthetic peptide”
HQYHRSPT

SEQ ID NO: 229 - protein, 5 aa, Artificial Sequence
  source: /note=“Description of Artificial Sequence: Synthetic peptide”
NFGMN

SEQ ID NO: 230 - protein, 17 aa, Artificial Sequence
  source: /note=“Description of Artificial Sequence: Synthetic peptide”
WINLYTGEPTFADDFKG

SEQ ID NO: 231 - protein, 13 aa, Artificial Sequence
  source: /note=“Description of Artificial Sequence: Synthetic peptide”
KGETYYRYDGFAY

SEQ ID NO: 232 - protein, 16 aa, Artificial Sequence
  source: /note=“Description of Artificial Sequence: Synthetic peptide”
RSSKSLLHSNGNTHLY

SEQ ID NO: 233 - protein, 7 aa, Artificial Sequence
  source: /note=“Description of Artificial Sequence: Synthetic peptide”
RMSNLAS

SEQ ID NO: 234 - protein, 9 aa, Artificial Sequence
  source: /note=“Description of Artificial Sequence: Synthetic peptide”
MQLLEYPYT

SEQ ID NO: 235 - protein, 4 aa, Artificial Sequence
  source: /note=“Description of Artificial Sequence: Synthetic peptide”
GFLG

SEQ ID NO: 236 - protein, 4 aa, Artificial Sequence
  source: /note=“Description of Artificial Sequence: Synthetic peptide”
ALAL